(19) United States
(12) Patent Application Publication
(10) Pub. No.: US 2023/0234935 A1
(43) Pub. Date: Jul. 27, 2023

(54) KINASE INHIBITOR COMPOUNDS AND COMPOSITIONS AND METHODS OF USE

(71) Applicant: ICAHN SCHOOL OF MEDICINE AT MOUNT SINAI, New York, NY (US)

(72) Inventors: Robert J. DEVITA, New York, NY (US); Chalada SUEBSUWONG, New York, NY (US); Kunal KUMAR, New York, NY (US); Roberto J. SANCHEZ, New York, NY (US); Andrew F. STEWART, New York, NY (US); Peng WANG, New York, NY (US); Michael B. LAZARUS, New York, NY (US)

(21) Appl. No.: 18/003,088

(22) PCT Filed: Jun. 25, 2021
(86) PCT No.: PCT/US2021/039132

§ 371 Date: Dec. 22, 2022

Related U.S. Application Data
(60) Provisional application No. 63/044,664, filed on Jun. 26, 2020.

(51) Int. Cl. C07D 401/04; C07D 471/04; C07D 403/04; C07D 213/74; C07D 403/12; C07D 403/14; C07D 401/14; C07D 487/04; C07D 417/04; C07F 5/02; C07D 413/12; C07D 413/10; A61K 45/06

(52) U.S. Cl. CPC C07D 401/04; C07D 471/04; C07D 403/04; C07D 213/74; C07D 403/12; C07D 403/14; C07D 401/14; C07D 487/04; C07D 417/04; C07F 5/025; C07D 413/12; C07D 413/10; A61K 45/06

(57) ABSTRACT

Disclosed herein are kinase inhibitor compounds having the structure (I) or a stereoisomer, pharmaceutically acceptable salt, oxide, or solvate thereof, where R¹, R², X, L, Q, and Y are as defined herein. Also disclosed are compositions containing the kinase inhibitor compounds, methods of inhibiting activity of a kinase in a cell, methods of increasing cell proliferation in a population of pancreatic beta cells, methods of treating a subject for a condition associated with insufficient insulin secretion, and methods of treating a subject for a neurological disorder.

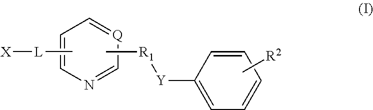

This application claims the priority benefit of U.S. Provisional Patent Application Ser. No. 63/044,664, filed Jun. 26, 2020, which is hereby incorporated by reference in its entirety.

This invention was made with government support under grant number R01 DK105015 awarded by the National Institute of Diabetes and Digestive and Kidney Diseases (NIDDK), and grant number R01 DK116904-01 awarded by the National Institutes of Health. The government has certain rights in the invention.

### FIELD OF THE INVENTION

The present invention relates to kinase inhibitor compounds and compositions and methods of use thereof.

### BACKGROUND OF THE INVENTION

The Dual-Specificity Tyrosine-Regulated kinases (“DYRKs”) belong to the CMCG family of eukaryotic protein kinases, which include the CDK-like kinases (CLKs), Glycogen Synthase Kinase 3 (GSK3), Cyclin Dependent Kinases (CDKs), and Mitogen-Activated Protein Kinases (MAPKs). DYRK family proteins self-activate by autophosphorylation of the conserved tyrosine residue in the activation loop, then subsequently phosphorylate substrates only on serine and threonine residues (Lochhead et al., “Activation-Loop Autophosphorylation is Mediated by a Novel Transitional Intermediate Form of DYRKs,” Cell 121(6):925-936 (2005); Walte et al., “Mechanism of Dual Specificity Kinase Activity of DYRK1A,” FEBS J. 280(18):4495-4511 (2013); and Becker et al., “Activation, Regulation, and Inhibition of DYRK1A,” FEBS J. 278(2):246-256 (2011)). The DYRK family consists of five subtypes, including 1A, 1B, 2, 3, and 4. Among them, DYRK1A is the most extensively studied subtype. It is ubiquitously expressed and has been shown to play an important role in brain development and function (Becker et al., “DYRK1A: A Potential Drug Target for Multiple Down Syndrome Neuropathologies,” CNS Neurol. Disord.: Drug Targets 13(1):26-33 (2014)), neurodegenerative diseases (Wegiel et al., “The Role of DYRK1A in Neurodegenerative Diseases,” FEBS J. 278(2):236-245 (2011) and Smith et al., “Recent Advances in the Design, Synthesis, and Biological Evaluation of Selective DYRK1A Inhibitors: A New Avenue for a Disease Modifying Treatment of Alzheimer's?,” ACS Chem. Neurosci. 3(11):857-872 (2012)), tumorigenesis, apoptosis (Ionescu et al., “DYRK1A Kinase Inhibitors With Emphasis on Cancer,” Mini-Rev. Med. Chem. 12(13):1315-1329 (2012) and Fernandez-Martinez et al., “DYRK1A: The Double-Edged Kinase as a Protagonist in Cell Growth and Tumorigenesis,” Mol. Cell. Oncol. 2 (1):e970048 (2015)), and human pancreatic j-cell proliferation (Wang et al., “A High-Throughput Chemical Screen Reveals That Harmine-Mediated Inhibition of DYRK1A Increases Human Pancreatic Beta Cell Replication,” Nat. Med. 21(4):383-388 (2015); Shen et al., “Inhibition of DYRK1A and GSK3B Induces Human j-cell Proliferation,” Nat. Commun. 6:8372 (2015); Rachdi et al., “Dyrk1A Induces Pancreatic R Cell Mass Expansion and Improves Glucose Tolerance,” Cell Cycle 13(14):2221-2229 (2014); and Dirice et al., “Inhibition of DYRK1A Stimulates Human Beta-Cell Proliferation,” Diabetes 65:(6):1660-1671 (2016)).

Regulated expression of DYRK1A during fetal, postnatal life, as well as in adults, is essential for normal neuronal development and brain function. DYRK1A is located in the Down Syndrome Critical region (“DSCR”) on human chromosome 21, a genomic region that has an important role in pathogenesis of Down Syndrome (“DS”), one of the most common and frequent human genetic disorders (Becker et al., “Activation, Regulation, and Inhibition of DYRK1A,” FEBS J. 278(2):246-256 (2011) and Becker et al., “Structural and Functional Characteristics of Dyrk, a Novel Subfamily of Protein Kinases With Dual Specificity,” Prog. Nucleic Acid Res. Mol. Biol. 62:1-17 (1999)). Overexpression of DYRK1A in mouse and drosophila models mimics the neurodevelopmental abnormalities associated with DS (Becker et al., “DYRK1A: A Potential Drug Target for Multiple Down Syndrome Neuropathologies,” CNS Neurol. Disord.: Drug Targets 13(1):26-33 (2014); Wegiel et al., “The Role of DYRK1A in Neurodegenerative Diseases,” FEBS J. 278(2):236-245 (2011); Park et al., “Function and Regulation of Dyrk1A: Towards Understanding Down Syndrome,” Cell. Mol. Life Sci. 66(20):3235-3240 (2009); and Ogawa et al., “Development of a Novel Selective Inhibitor of the Down Syndrome-Related Kinase Dyrk1A,” Nat. Commun. 1: Article Number 86 (2010)). Recent evidences has also implicated DYRK1A in the tau dysfunction and tau pathology of Alzheimer's disease (“AD”), dementia with Lewy bodies, and Parkinson's disease (Wegiel et al., “The Role of DYRK1A in Neurodegenerative Diseases,” FEBS J. 278(2):236-245 (2011); Smith et al., “Recent Advances in the Design, Synthesis, and Biological Evaluation of Selective DYRK1A Inhibitors: A New Avenue for a Disease Modifying Treatment of Alzheimer's?,” ACS Chem. Neurosci. 3(11):857-872 (2012); and Stotani et al., “DYRK1A Inhibition as Potential Treatment for Alzheimer's Disease,” Future Med. Chem. 8(6):681-696 (2016)). It has been reported that DYRK1A is overexpressed in various tumors such as, ovarian cancer, colon cancer, lung cancer, and pancreatic cancer, signifying its role in tumorigenesis and uncontrolled cell proliferation (Ionescu et al., “DYRK1A Kinase Inhibitors With Emphasis on Cancer,” Mini-Rev. Med. Chem. 12(13):1315-1329 (2012) and Fernandez-Martinez et al., “DYRK1A: The Double-Edged Kinase as a Protagonist in Cell Growth and Tumorigenesis,” Mol. Cell. Oncol. 2 (1):e970048 (2015)). Inhibition of DYRK1A leads to destabilized EGFR and reduced EGFR-dependent tumor growth in glioblastoma (Pozo et al., “Inhibition of DYRK1A Destabilizes EGFR and Reduces EGFR-Dependent Glioblastoma Growth,” J. Clin. Invest. 123(6):2475-2487 (2013)). Also, DYRK1A inhibition induces activation of caspase-9 which leads to massive apoptosis in specific cancer cell types (Seifert et al., “DYRK1A Phosphorylates Caspase 9 at an Inhibitory Site and is Potently Inhibited in Human Cells by Harmine,” FEBS J. 275(24):6268-6280 (2008)). Recently, DYRK1A has been shown to be involved in molecular pathways relevant to human j-cell proliferation, making it a potential therapeutic target for j-cell regeneration in Type 1 and Type 2 diabetes (Wang et al., “A High-throughput Chemical Screen Reveals That Harmine-Mediated Inhibition of DYRK1A Increases Human Pancreatic Beta Cell Replication,” Nat. Med. 21(4):383-388 (2015); Shen et al., “Inhibition of DYRK1A and GSK3B Induces Human j-cell Proliferation,” Nat. Commun. 6:8372 (2015); Rachdi et al., “Dyrk1A Induces Pancreatic R Cell Mass Expansion and Improves Glucose Tolerance,” Cell Cycle 13(14):2221-2229 (2014); and Dirice et al., “Inhibition of DYRK1A Stimulates Human Beta-cell Proliferation,” Diabetes 65:(6):1660-1671 (2016)). DYRK1A inhibition has been proposed to drive p-cell proliferation by inducing translocation of the nuclear factor of activated T cells (“NFAT”) family of transcription factors to the nucleus, allowing access to the promoters of genes, which subsequently activate human j-cell proliferation (Wang et al., “A High-throughput Chemical Screen Reveals That Harmine-Mediated Inhibition of DYRK1A Increases Human Pancreatic Beta Cell Replication,” Nat. Med. 21(4):383-388 (2015) and Rachdi et al., “Dyrk1A Induces Pancreatic R Cell Mass Expansion and Improves Glucose Tolerance,” Cell Cycle 13(14):2221-2229 (2014)).

Because of its involvement in neurodegenerative disease, cancer, and diabetes, DYRK1A has attracted increasing interest as a potential therapeutic target. A significant amount of work has been carried out to not only understand its underlying role in diseases, but also in identifying novel DYRK1A inhibitors (Becker et al., “Activation, Regulation, and Inhibition of DYRK1A,” FEBS J. 278(2):246-256 (2011); Becker et al., “DYRK1A: A Potential Drug Target for Multiple Down Syndrome Neuropathologies,” CNS Neurol. Disord.: Drug Targets 13(1):26-33 (2014); Wegiel et al., “The Role of DYRK1A in Neurodegenerative Diseases,” FEBS J. 278(2):236-245 (2011); Smith et al., “Recent Advances in the Design, Synthesis, and Biological Evaluation of Selective DYRK1A Inhibitors: A New Avenue for a Disease Modifying Treatment of Alzheimer's?,” ACS Chem. Neurosci. 3(11):857-872 (2012); Ionescu et al., “DYRK1A Kinase Inhibitors with Emphasis on Cancer,” Mini-Rev. Med. Chem. 12(13):1315-1329 (2012); Fernandez-Martinez et al., “DYRK1A: The Double-Edged Kinase as a Protagonist in Cell Growth and Tumorigenesis,” Mol. Cell. Oncol. 2 (1):e970048 (2015); Wang et al., “A High-throughput Chemical Screen Reveals That Harmine-Mediated Inhibition of DYRK1A Increases Human Pancreatic Beta Cell Replication,” Nat. Med. 21(4):383-388 (2015); Shen et al., “Inhibition of DYRK1A and GSK3B Induces Human j-cell Proliferation,” Nat. Commun. 6:8372 (2015); and Dirice et al., “Inhibition of DYRK1A Stimulates Human Beta-cell Proliferation,” Diabetes 65:(6):1660-1671 (2016)).

Several DYRK1A inhibitors from natural sources as well as small molecule drug discovery programs have been identified and characterized. Among all the DYRK1A inhibitors, harmine and its analogues (β-carbolines) are the most commonly studied and remain the most potent and orally bioavailable class of inhibitors covered to date (Becker et al., “Activation, Regulation, and Inhibition of DYRK1A,” FEBS J. 278(2):246-256 (2011) and Smith et al., “Recent Advances in the Design, Synthesis, and Biological Evaluation of Selective DYRK1A Inhibitors: A New Avenue for a Disease Modifying Treatment of Alzheimer's?,” ACS Chem. Neurosci. 3(11):857-872 (2012)).

Apart from harmine, EGCg and other flavan-3-ols (Guedj et al., “Green Tea Polyphenols Rescue of Brain Defects Induced by Overexpression of DYRK1A,” PLoS One 4(2):e4606 (2009) and Bain et al., “The Specificities of Protein Kinase Inhibitors: An Update,” Biochem. J. 371(1):199-204 (2003)), leucettines (Tahtouh et al., “Selectivity, Cocrystal Structures, and Neuroprotective Properties of Leucettines, a Family of Protein Kinase Inhibitors Derived from the Marine Sponge Alkaloid Leucettamine B,” J. Med. Chem. 55(21):9312-9330 (2012) and Naert et al., “Leucettine L41, a DYRK1A-preferential DYRKs/CLKs Inhibitor, Prevents Memory Impairments and Neurotoxicity Induced by Oligomeric A025-35 Peptide Administration in Mice,” Eur. Neuropsychopharmacol. 25(11):2170-2182 (2015)), quinalizarine (Cozza et al., “Quinalizarin as a Potent, Selective and Cell-permeable Inhibitor of Protein Kinase CK2,” Biochem. J. 421(3):387-395 (2009)), peltogynoids Acanilol A and B (Ahmadu et al, “Two New Peltogynoids from Acacia nilotica Delile with Kinase Inhibitory Activity,” Planta Med. 76(5):458-460 (2010)), benzocoumarins (dNBC) (Sarno et al., “Structural Features Underlying the Selectivity of the Kinase Inhibitors NBC and dNBC: Role of a Nitro Group that Discriminates Between CK2 and DYRK1A,” Cell. Mol. Life Sci. 69(3):449-460 (2012)), and indolocarbazoles (Starosporine, rebeccamycin and their analogues) (Sanchez et al., “Generation of Potent and Selective Kinase Inhibitors by Combinatorial Biosynthesis of Glycosylated Indolocarbazoles,” Chem. Commun. 27:4118-4120 (2009), are other natural products that have been shown to inhibit DYRK1A and other kinases.

Among the other scaffolds identified from small molecule drug discovery attempts, INDY (Ogawa et al., “Development of a Novel Selective Inhibitor of the Down Syndrome-Related Kinase Dyrk1A,” Nat. Commun. 1: Article Number 86 (2010)), DANDY (Gourdain et al., “Development of DANDYs, New 3,5-Diaryl-7-Azaindoles Demonstrating Potent DYRK1A Kinase Inhibitory Activity,” J. Med Chem. 56(23):9569-9585 (2013)), FINDY (Kii et al., “Selective Inhibition of the Kinase DYRK1A by Targeting its Folding Process,” Nat. Commun. 7:11391 (2016)), pyrazolidine-diones (Koo et al., “QSAR Analysis of Pyrazolidine-3,5-Diones Derivatives as Dyrk1A Inhibitors,” Bioorg. Med Chem. Lett. 19(8):2324-2328 (2009); Kim et al., “Putative Therapeutic Agents for the Learning and Memory Deficits of People with Down Syndrome,” Bioorg. Med Chem. Lett. 16(14):3772-3776 (2006)), amino-quinazolines (Rosenthal et al., “Potent and Selective Small Molecule Inhibitors of Specific Isoforms of Cdc2-Like Kinases (Clk) and Dual Specificity Tyrosine-Phosphorylation-Regulated Kinases (Dyrk),” Bioorg. Med Chem. Lett. 21(10):3152-3158 (2011)), meriolins (Giraud et al., “Synthesis, Protein Kinase Inhibitory Potencies, and In Vitro Antiproliferative Activities of Meridianin Derivatives,” J. Med Chem. 54(13):4474-4489 (2011); Echalier et al., “Meriolins (3-(Pyrimidin-4-yl)-7-Azaindoles): Synthesis, Kinase Inhibitory Activity, Cellular Effects, and Structure of a CDK2/Cyclin A/Meriolin Complex,” J. Med Chem. 51(4):737-751 (2008); and Akue-Gedu et al., “Synthesis and Biological Activities of Aminopyrimidyl-Indoles Structurally Related to Meridianins,” Bioorg. Med Chem. 17(13):4420-4424 (2009)), pyridine and pyrazines (Kassis et al., “Synthesis and Biological Evaluation of New 3-(6-hydroxyindol-2-yl)-5-(Phenyl) Pyridine or Pyrazine V-Shaped Molecules as Kinase Inhibitors and Cytotoxic Agents,” Eur. J. Med Chem. 46(11):5416-5434 (2011)), chromenoidoles (Neagoie et al., “Synthesis of Chromeno[3,4-b]indoles as Lamellarin D Analogues: A Novel DYRK1A Inhibitor Class,” Eur. J. Med Chem. 49:379-396 (2012)), 11H-indolo[3,2-c]quinoline-6-carboxylic acids, 37 thiazolo[5,4-f]quinazolines (EHT 5372) (Foucourt et al., “Design and Synthesis of Thiazolo[5,4-f]quinazolines as DYRK1A Inhibitors, Part I.,” Molecules 19(10):15546-15571 (2014) and Coutadeur et al., “A Novel DYRK1A (Dual Specificity Tyrosine Phosphorylation-Regulated Kinase 1A) Inhibitor for the Treatment of Alzheimer's Disease: Effect on Tau and Amyloid Pathologies In Vitro,” J. Neurochem. 133(3):440-451 (2015)), and 5-iodotubercidin (Dirice et al., “Inhibition of DYRK1A Stimulates Human Beta-cell Proliferation,” Diabetes 65:(6):1660-1671 (2016) and Annes et al., “Adenosine Kinase Inhibition Selectively Promotes Rodent and Porcine Islet 3-cell Replication,” Proc. Natl. Acad Sci. 109(10):3915-3920 (2012)) showed potent DYRK1A activity with varying degrees of kinase selectivity.

Most of these compounds are non-selective inhibitors of DYRK1A and exhibit pharmacological side effects, such as CNS activity or apoptosis, thereby limiting their therapeutic utility and potential for pharmaceutical development. This non-selectivity may be attributed to the fact that all these DYRK1A inhibitors are Type I kinase inhibitors, which bind to a highly conserved ATP binding pocket.

The present invention is directed to overcoming deficiencies in the art.

### SUMMARY OF THE INVENTION

One aspect of the present invention relates to a compound of formula (I) having the following structure:

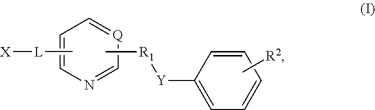

or a stereoisomer, pharmaceutically acceptable salt, oxide, or solvate thereof, wherein

X is selected from the group consisting of

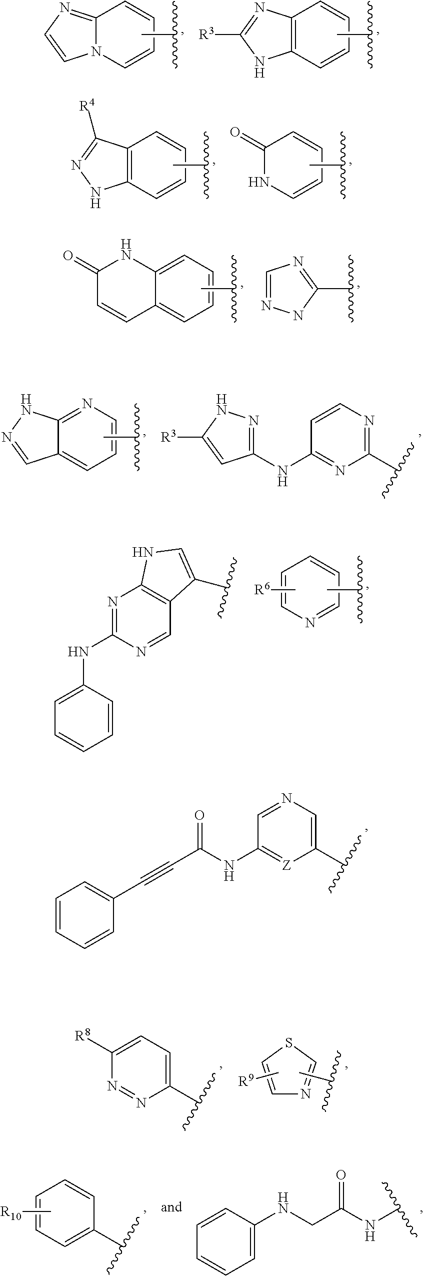

L is selected from the group consisting of a bond,

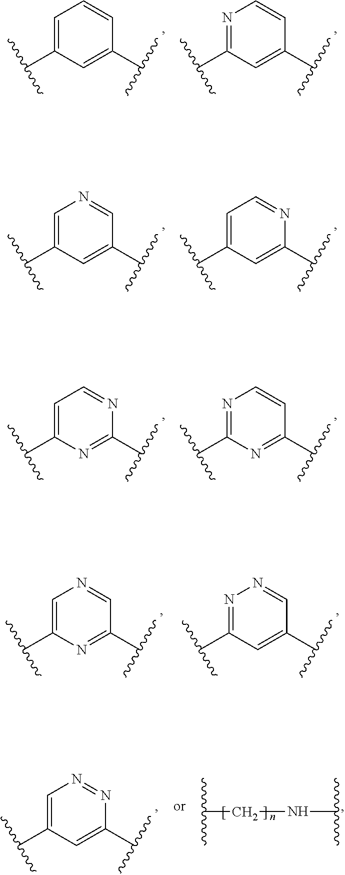

wherein n is an integer between 0-6;

Q is selected from the group consisting of CH and N;

R¹ is optionally present, and when present is selected from the group consisting of NH and branched or unbranched C₁-C₆ alkyl;

Y is selected from the group consisting of branched or unbranched C₁-C₆ alkyl and NH;

R² is absent or present, and when present is selected from the group consisting of one or more of halogen, alkyl, alkoxy, CF₃, OPh, OCF₃, CN, CONH₂, and COOCH₃;

R³ is selected from the group consisting of C₁-C₆ alkoxy and NH₂;

R⁴ is selected from the group consisting of H, NH₂, NHPh, COOC(CH₃)₃, COOH, CONH₂, CONHCH₃, NHCONH₂, and CF₃;

R⁵ is branched or unbranched C₁-C₆ alkyl;

R⁶ is selected from the group consisting of

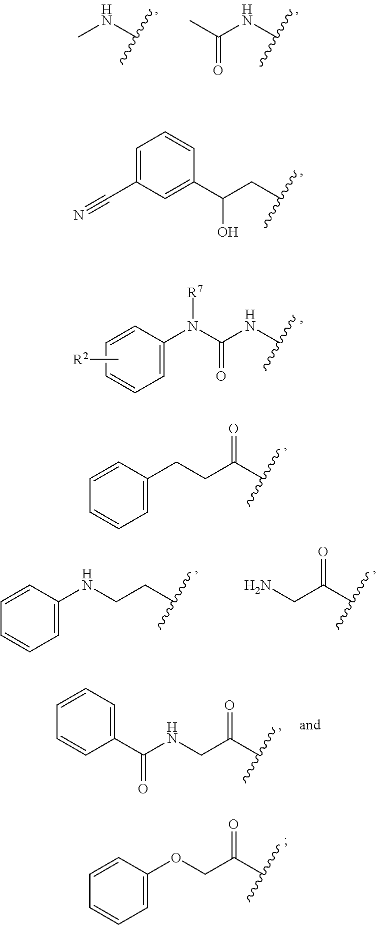

R⁷ is selected from the group consisting of H and Boc;

R⁸ is

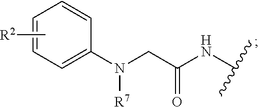

R⁹ is selected from the group consisting of NH₂ and

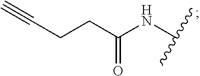 [SMILES: *NC(=O)CCC#C]

R¹⁰ is one or more of halogen,

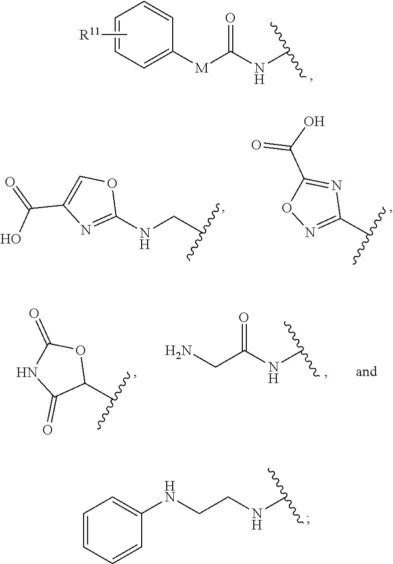

and

R¹¹ is one or more of halogen and

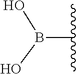 [SMILES: *B(O)O]

Z is CH or N; and

M is optionally present and when present is —NHCH₂— or —CH₂CH₂—.

Another aspect of the present invention relates to a method of inhibiting activity of a kinase in a cell. This method involves contacting the cell with a compound of formula (I) as described herein under conditions effective to inhibit activity of the kinase in the cell.

A further aspect of the present invention relates to a method of increasing cell proliferation in a population of pancreatic beta cells. This method involves contacting a population of pancreatic beta cells with a compound of formula (I) as described herein under conditions effective to increase cell proliferation in the population of pancreatic beta cells.

Another aspect of the present invention relates to a composition comprising a compound of formula (I) as described herein and a carrier.

An additional aspect of the present invention relates to a method of treating a subject for a condition associated with insufficient insulin secretion. This method involves administering to a subject in need of treatment for a condition associated with an insufficient level of insulin secretion a compound or composition as described herein.

A further aspect of the present invention relates to a method of treating a subject for a neurological disorder. This method involves administering to a subject in need of treatment for a neurological disorder a compound of formula (I) as described herein under conditions effective to treat the subject for the condition.

Although efforts have been made toward the discovery of potent and selective DYRK1A inhibitors, most of them are still in early stages of lead identification.

Described herein infra is the identification and evaluation of a highly potent and novel class of kinase inhibitor compounds.

### BRIEF DESCRIPTION OF THE DRAWINGS

FIG. 1 is a schematic illustration showing the synthesis of halo heterocyclic intermediate compounds.

FIG. 2 is a schematic illustration showing the general synthesis of modified hinge binder DYRK1A inhibitors.

### DETAILED DESCRIPTION OF THE INVENTION

One aspect of the present invention relates to a compound of formula (I) having the following structure:

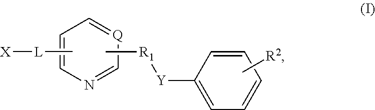

or a stereoisomer, pharmaceutically acceptable salt, oxide, or solvate thereof, wherein

X is selected from the group consisting of

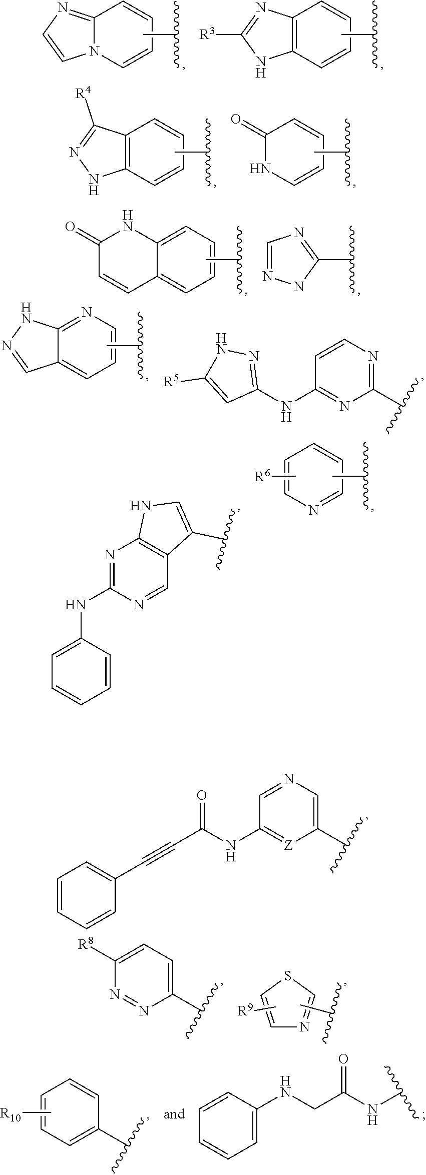

L is selected from the group consisting of a bond,

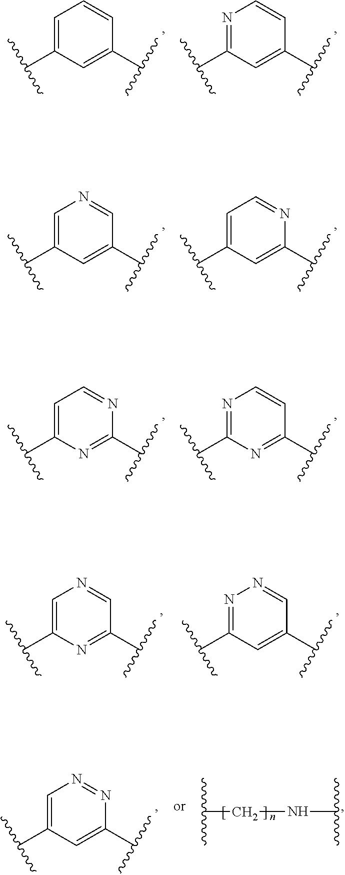

wherein n is an integer between 0-6;

Q is selected from the group consisting of CH and N;

R¹ is optionally present, and when present is selected from the group consisting of NH and branched or unbranched C₁-C₆ alkyl;

Y is selected from the group consisting of branched or unbranched C₁-C₆ alkyl and NH;

R² is absent or present, and when present is selected from the group consisting of one or more of halogen, alkyl, alkoxy, CF₃, OPh, OCF₃, CN, CONH₂, and COOCH₃;

R³ is selected from the group consisting of C₁-C₆ alkoxy and NH₂;

R⁴ is selected from the group consisting of H, NH₂, NHPh, COOC(CH₃)₃, COOH, CONH₂, CONHCH₃, NHCONH₂, and CF₃;

R⁵ is branched or unbranched C₁-C₆ alkyl;

R⁶ is selected from the group consisting of

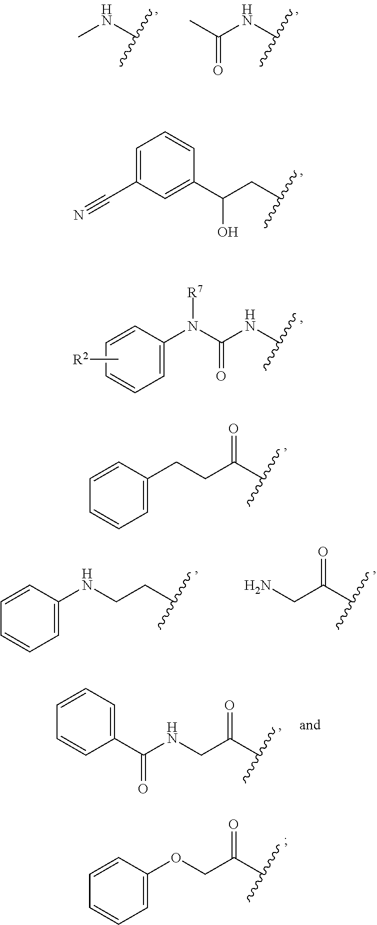

R⁷ is selected from the group consisting of H and Boc;

R⁸ is

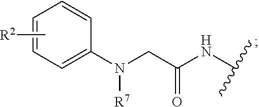

R⁹ is selected from the group consisting of NH₂ and

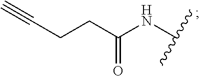 [SMILES: *NC(=O)CCC#C]

R¹⁰ is one or more of halogen,

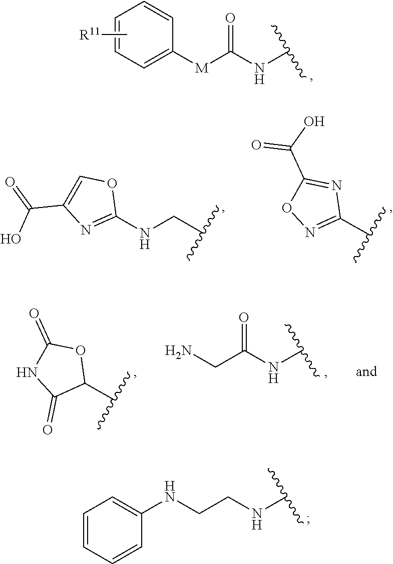

and

R¹¹ is one or more of halogen and

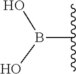 [SMILES: *B(O)O]

Z is CH or N; and

M is optionally present and when present is —NHCH₂— or —CH₂CH₂—.

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs.

As used herein, the term “halogen” means fluoro, chloro, bromo, or iodo.

The term “alkyl” means an aliphatic hydrocarbon group which may be straight or branched having about 1 to about 6 carbon atoms in the chain (or the number of carbons designated by “Cₙ-Cₙ”, where n is the numerical range of carbon atoms). Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, and 3-pentyl.

The term “alkoxy” means groups of from 1 to 6 carbon atoms of a straight, branched, or cyclic configuration and combinations thereof attached to the parent structure through an oxygen. Examples include methoxy, ethoxy, propoxy, isopropoxy, butoxy, cyclopropyloxy, cyclohexyloxy, and the like. Alkoxy also includes methylenedioxy and ethylenedioxy in which each oxygen atom is bonded to the atom, chain, or ring from which the methylenedioxy or ethylenedioxy group is pendant so as to form a ring. Thus, for example, phenyl substituted by alkoxy may be, for example,

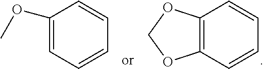

The term “substituted” means that one or more hydrogen(s) on a designated atom is replaced with a selection from the indicated group, provided that the designated atom's normal valency is not exceeded. “Unsubstituted” atoms bear all of the hydrogen atoms dictated by their valency. When a substituent is oxo (i.e., ═O), then 2 hydrogens on the atom are replaced. Combinations of substituents and/or variables are permissible only if such combinations result in stable compounds. By “stable compound” it is meant a compound that is sufficiently robust to survive isolation to a useful degree of purity from a reaction mixture and formulation into an efficacious therapeutic agent.

By “compound(s) of the invention” and equivalent expressions, it is meant compounds herein described, which expression includes the prodrugs, the pharmaceutically acceptable salts, the oxides, and the solvates, e.g. hydrates, where the context so permits.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. The present invention is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. All tautomeric forms are also intended to be included.

As would be understood by a person of ordinary skill in the art, the recitation of “a compound” is intended to include salts, solvates, oxides, and inclusion complexes of that compound as well as any stereoisomeric form, or a mixture of any such forms of that compound in any ratio. Thus, in accordance with some embodiments of the invention, a compound as described herein, including in the contexts of pharmaceutical compositions, methods of treatment, and compounds per se, is provided as the salt form.

The term “solvate” refers to a compound in the solid state, where molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

Inclusion complexes are described in Remington, The Science and Practice of Pharmacy, 19th Ed. 1:176-177 (1995), which is hereby incorporated by reference in its entirety. The most commonly employed inclusion complexes are those with cyclodextrins, and all cyclodextrin complexes, natural and synthetic, are specifically encompassed by the present invention.

The term “pharmaceutically acceptable salt” refers to salts prepared from pharmaceutically acceptable non-toxic acids or bases including inorganic acids and bases and organic acids and bases.

The term “pharmaceutically acceptable” means it is, within the scope of sound medical judgment, suitable for use in contact with the cells of humans and lower animals without undue toxicity, irritation, allergic response and the like, and are commensurate with a reasonable benefit/risk ratio.

In one embodiment of the compound of formula (I), X is

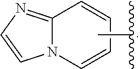

Compounds of this embodiment include, without limitation

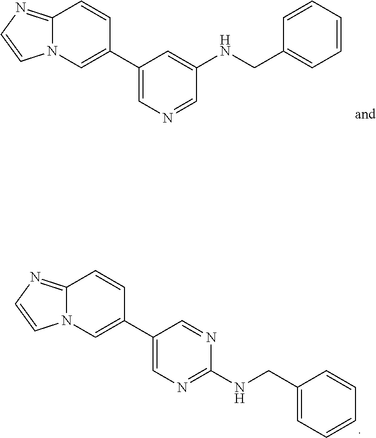 [SMILES: c1ccc(CNc2cncc(-c3ccc4nccn4c3)c2)cc1 ; c1ccc(CNc2ncc(-c3ccc4nccn4c3)cn2)cc1]

In a further embodiment of the compound of formula (I), X is

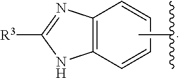

Compounds of this embodiment include, without limitation and

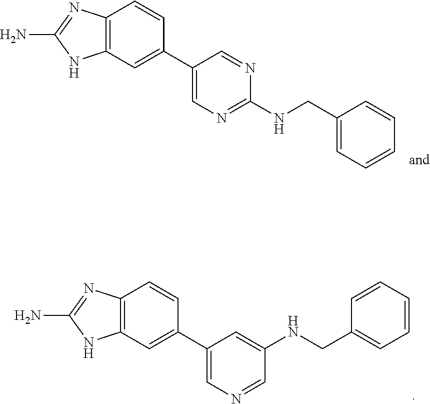 [SMILES: Nc1nc2ccc(-c3cnc(NCc4ccccc4)nc3)cc2[nH]1 ; Nc1nc2ccc(-c3cncc(NCc4ccccc4)c3)cc2[nH]1]

In some embodiments of the compound of formula (I), X is

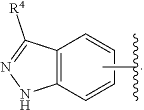

Exemplary compounds of this embodiment include, but are not limited to,

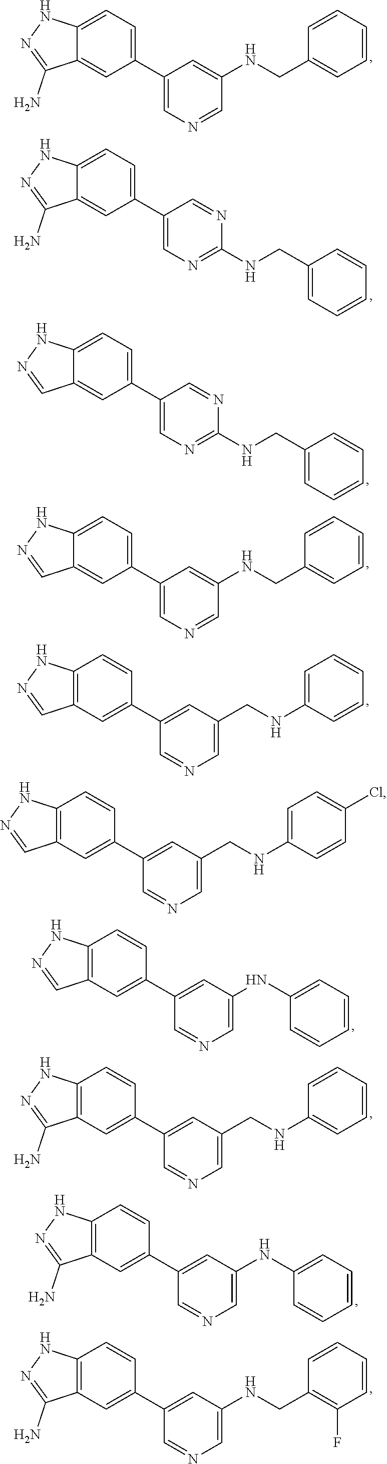

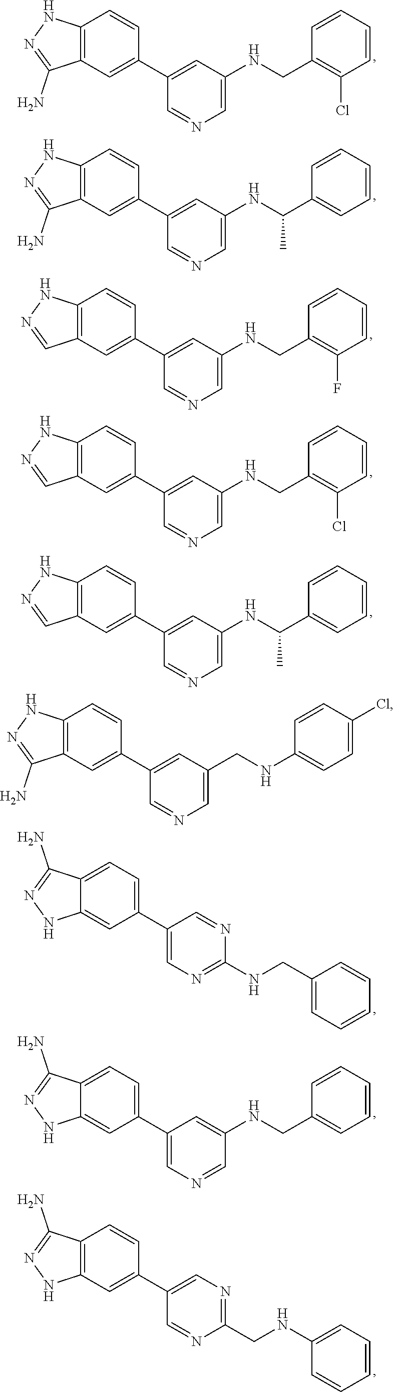

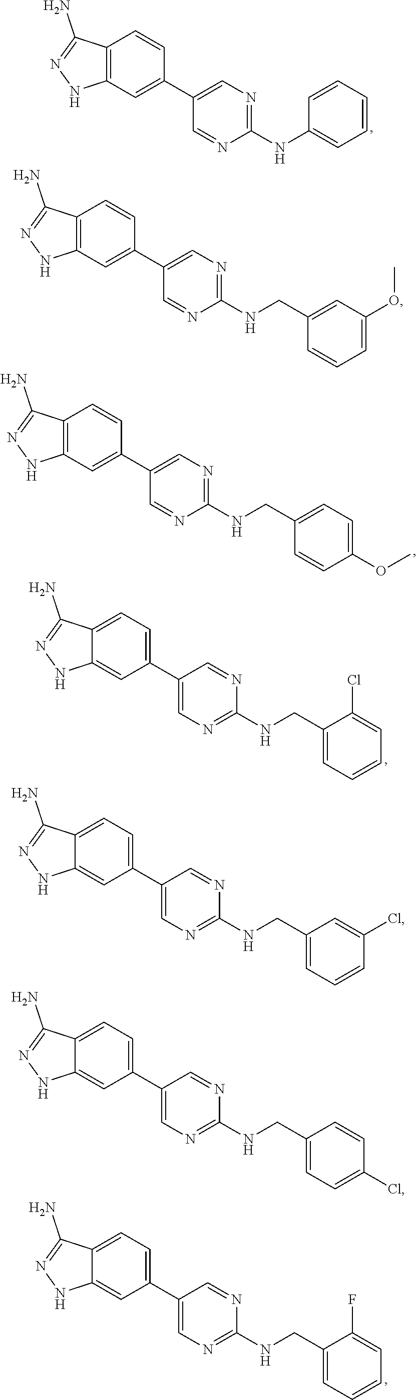

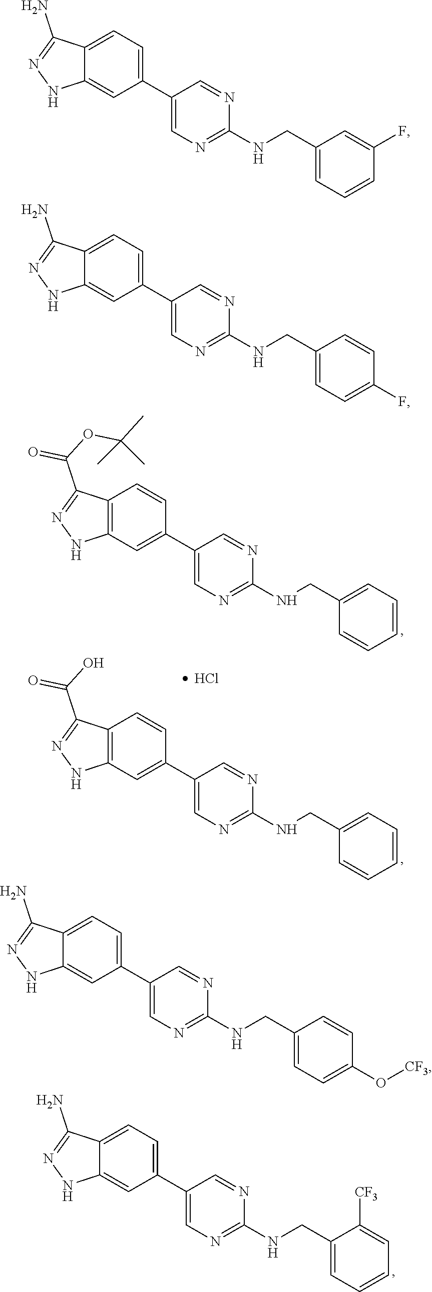

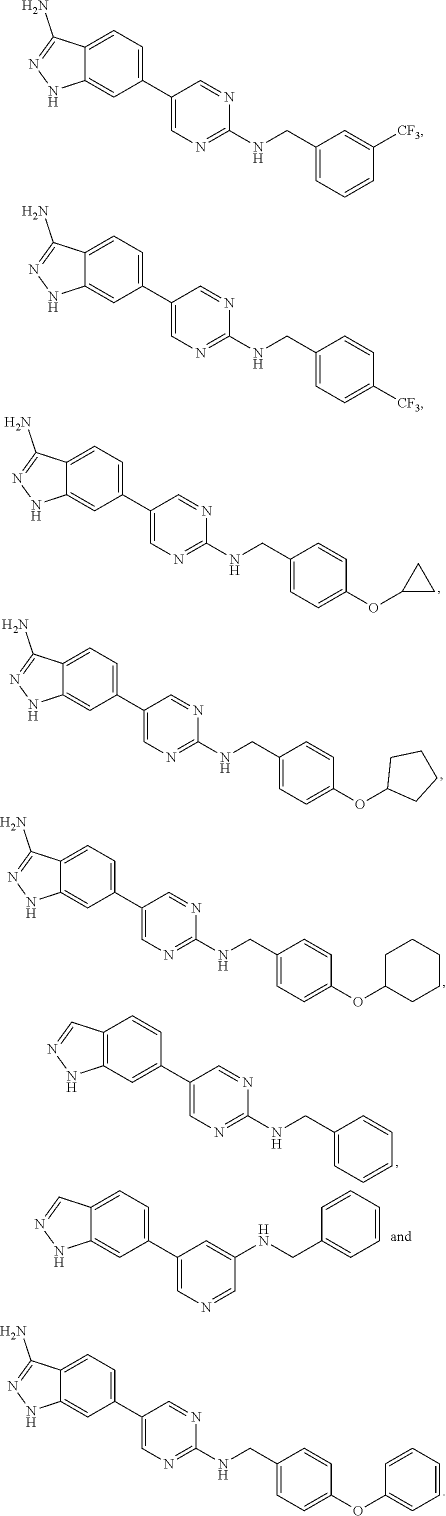

In another embodiment of the compound of formula (I), X is

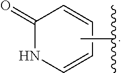

Compounds of this embodiment include, without limitation

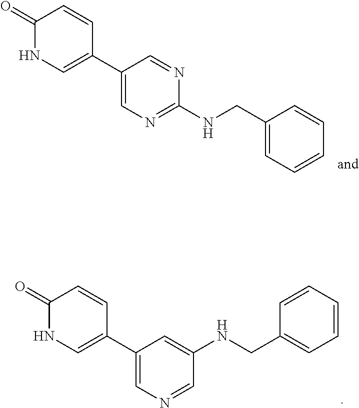 [SMILES: O=c1ccc(-c2cnc(NCc3ccccc3)nc2)c[nH]1 ; O=c1ccc(-c2cncc(NCc3ccccc3)c2)c[nH]1]

In yet another embodiment of the compound of formula (I), X is

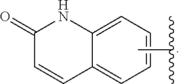

In accordance with this embodiment, the compound may be selected from

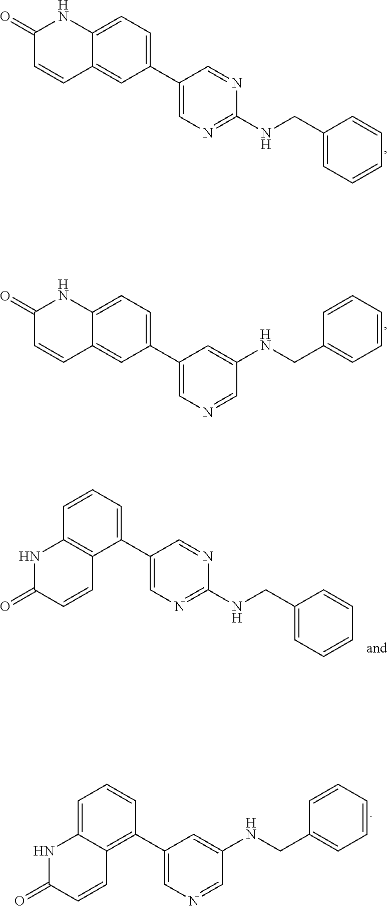 [SMILES: O=c1ccc2cc(-c3cnc(NCc4ccccc4)nc3)ccc2[nH]1 ; O=c1ccc2cc(-c3cncc(NCc4ccccc4)c3)ccc2[nH]1 ; O=c1ccc2c(-c3cnc(NCc4ccccc4)nc3)cccc2[nH]1 ; O=c1ccc2c(-c3cncc(NCc4ccccc4)c3)cccc2[nH]1]

In a further embodiment of the compound of formula (I), X is

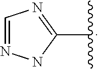 [SMILES: *c1ncn[nH]1]

An exemplary compound of this embodiment is

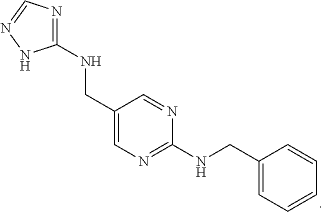 [SMILES: c1ccc(CNc2ncc(CNc3ncn[nH]3)cn2)cc1]

In another embodiment of the compound of formula (I), X is

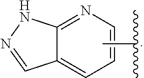

Exemplary compounds of this embodiment include, but are not limited to,

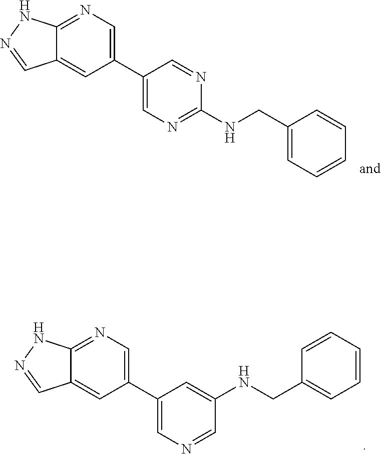 [SMILES: c1ccc(CNc2ncc(-c3cnc4[nH]ncc4c3)cn2)cc1 ; c1ccc(CNc2cncc(-c3cnc4[nH]ncc4c3)c2)cc1]

In a further embodiment of the compound of formula (I), X is

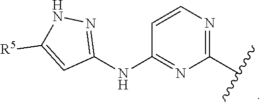

Compounds of this embodiment include, without limitation

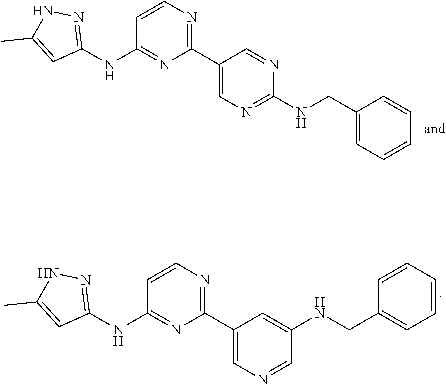 [SMILES: Cc1cc(Nc2ccnc(-c3cnc(NCc4ccccc4)nc3)n2)n[nH]1 ; Cc1cc(Nc2ccnc(-c3cncc(NCc4ccccc4)c3)n2)n[nH]1]

In some embodiments of the compound of formula (I), X is

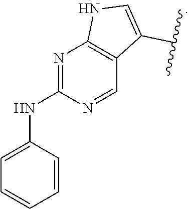 [SMILES: *c1c[nH]c2nc(Nc3ccccc3)ncc12]

Exemplary compounds of this embodiment include, but are not limited to,

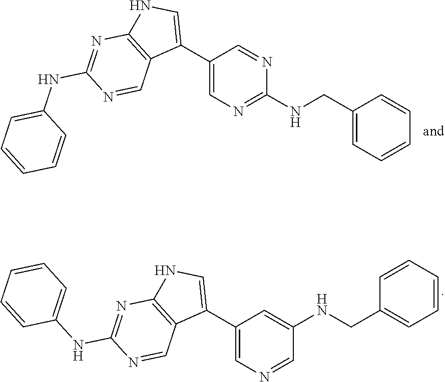 [SMILES: c1ccc(CNc2ncc(-c3c[nH]c4nc(Nc5ccccc5)ncc34)cn2)cc1 ; c1ccc(CNc2cncc(-c3c[nH]c4nc(Nc5ccccc5)ncc34)c2)cc1]

In a further embodiment of the compound of formula (I), X is

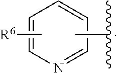

Exemplary compounds of this embodiment include, without limitation:

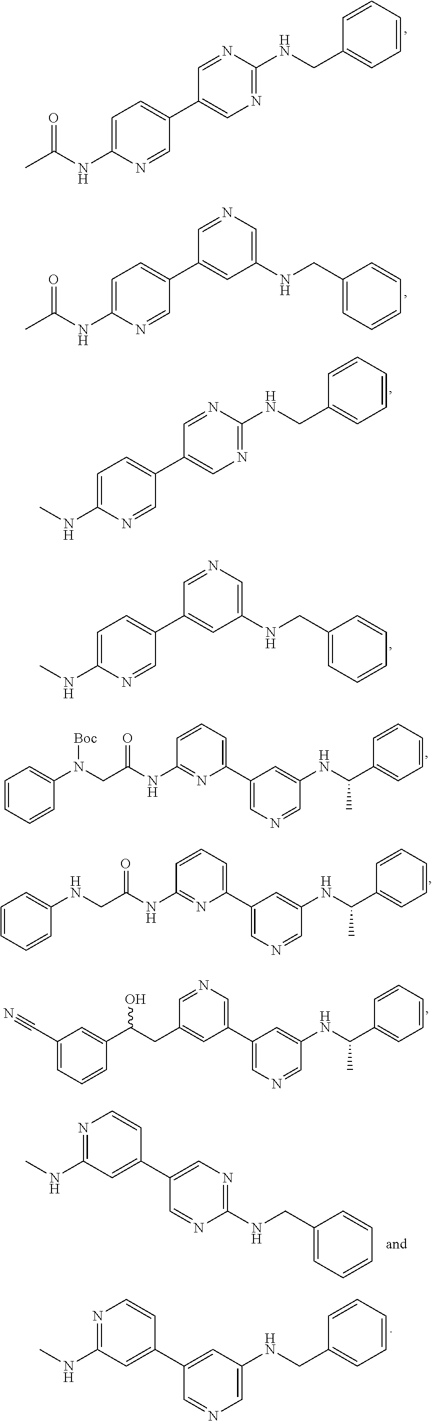

In yet another embodiment of the compound of formula (I), X is

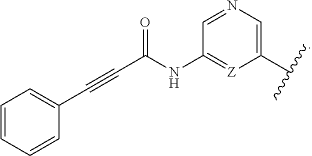

Exemplary compounds of this embodiment are

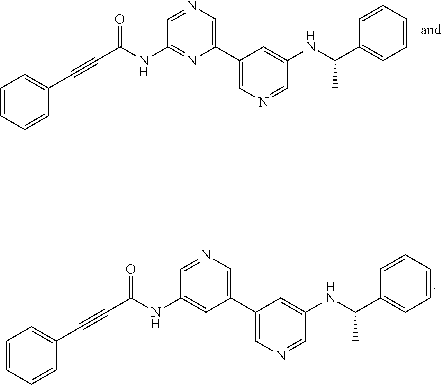 [SMILES: C[C@H](Nc1cncc(-c2cncc(NC(=O)C#Cc3ccccc3)n2)c1)c1ccccc1 ; C[C@H](Nc1cncc(-c2cncc(NC(=O)C#Cc3ccccc3)c2)c1)c1ccccc1]

In some embodiments of the compound of formula (I), X is

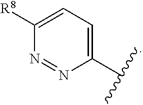

Exemplary compounds of this embodiment include, but are not limited to,

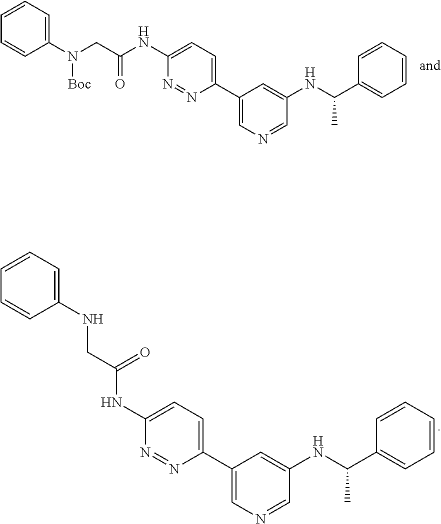 [SMILES: C[C@H](Nc1cncc(-c2ccc(NC(=O)CN(C(=O)OC(C)(C)C)c3ccccc3)nn2)c1)c1ccccc1 ; C[C@H](Nc1cncc(-c2ccc(NC(=O)CNc3ccccc3)nn2)c1)c1ccccc1]

In one embodiment of the compound of formula (I), X is

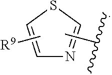

Compounds of this embodiment include, without limitation,

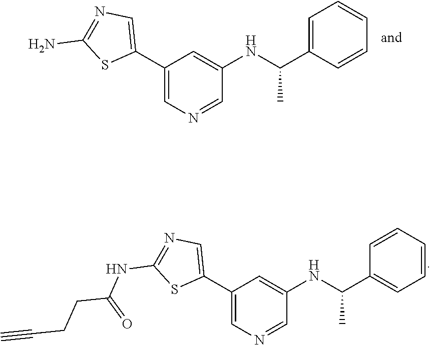 [SMILES: C[C@H](Nc1cncc(-c2cnc(N)s2)c1)c1ccccc1 ; C#CCCC(=O)Nc1ncc(-c2cncc(N[C@@H](C)c3ccccc3)c2)s1]

In a further embodiment of the compound of formula (I), X is

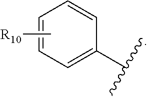

Compounds of this embodiment include, without limitation,

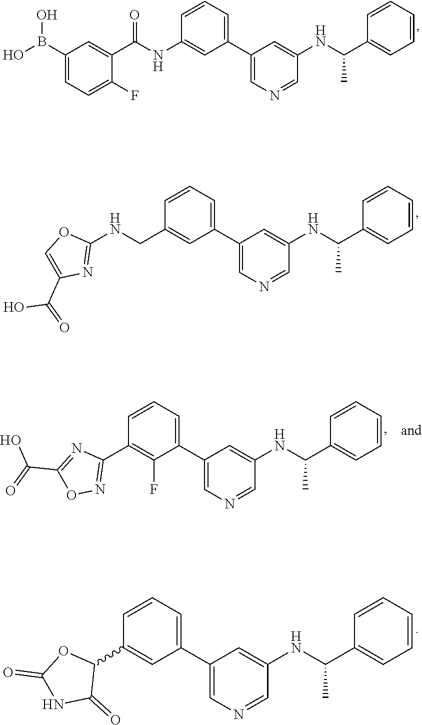 [SMILES: C[C@H](Nc1cncc(-c2cccc(NC(=O)c3cc(B(O)O)ccc3F)c2)c1)c1ccccc1 ; C[C@H](Nc1cncc(-c2cccc(CNc3nc(C(=O)O)co3)c2)c1)c1ccccc1 ; C[C@H](Nc1cncc(-c2cccc(-c3noc(C(=O)O)n3)c2F)c1)c1ccccc1 ; C[C@H](Nc1cncc(-c2cccc(C3OC(=O)NC3=O)c2)c1)c1ccccc1]

Compounds of formula (I) described herein have the ability to inhibit activity of a kinase in a cell. In particular, compounds of formula (I) have the ability to inhibit activity of a dual-specificity tyrosine phosphorylation-regulated kinase (DYRK), including the dual-specificity tyrosine phosphorylation-regulated kinase 1A (DYRK1A) and/or the dual-specificity tyrosine phosphorylation-regulated kinase 1B (DYRK1B).

Thus, another aspect of the present invention relates to a method of inhibiting activity of a kinase in a cell. This method involves contacting the cell with a compound of formula (I) under conditions effective to inhibit activity of the kinase in the cell.

In one embodiment, the kinase is a dual-specificity tyrosine phosphorylation-regulated kinase (DYRK). The kinase may be a dual-specificity tyrosine phosphorylation-regulated kinase 1A (DYRK1A) and/or a dual-specificity tyrosine phosphorylation-regulated kinase 1B (DYRK1B).

The cell may be a mammalian cell. Mammalian cells include cells from, for example, mice, hamsters, rats, cows, sheep, pigs, goats, horses, monkeys, dogs (e.g., Canis familiaris), cats, rabbits, guinea pigs, and primates, including humans. For example, the cell may be a human cell.

In one embodiment, the cell is a pancreatic beta cell. If needed, methods for determining whether a cell has a pancreatic beta cell phenotype are known in the art and include, without limitation, incubating the cell with glucose and testing whether insulin expression in the cell is increased or induced. Other methods include testing whether beta cell specific transcription factors are expressed, the detection of beta cell specific gene products with the help of RNA quantitative PCR, the transplantation of a candidate cell in diabetic mice, and subsequent testing of the physiologic response following said transplantation as well analyzing the cells with electron microscopy.

In another embodiment, the cell is a cancer cell.

In yet another embodiment, the cell is a neural cell.

Methods of the present invention may be carried out ex vivo or in vivo. When carried out ex vivo, a population of cells may be, according to one embodiment, provided by obtaining cells from a pancreas and culturing the cells in a liquid medium suitable for the in vitro or ex vivo culture of mammalian cells, in particular human cells. For example, and without limitation, a suitable and non-limiting culture medium may be based on a commercially available medium such as RPMI1640 from Invitrogen.

A further aspect of the present invention relates to a method of increasing cell proliferation in a population of pancreatic beta cells. This method involves contacting a population of pancreatic beta cells with a compound of formula (I) under conditions effective to increase cell proliferation in the population of pancreatic beta cells.

In one embodiment, cell proliferation in a population of pancreatic beta cells occurs by inhibiting both DYRK1A and DYRK1B together. In another embodiment, cell proliferation in a population of pancreatic beta cells occurs by inhibiting DYRK1A alone. See Ackeifi et al., “Pharmacologic and Genetic Approaches Define Human Pancreatic β Cell Mitogenic Targets of DYRK1A Inhibitors,” JCI Insight 5:e132594 (2020), which is hereby incorporated by reference in its entirety.

In one embodiment, contacting is carried out with a composition (i.e., a single composition) comprising the compound.

The method may further involve contacting the population of pancreatic beta cells with a transforming growth factor beta (TGFβ) superfamily signaling pathway inhibitor. In accordance with this embodiment, the method may be carried out with a composition comprising the compound and the TGFβ superfamily signaling pathway inhibitor. In another embodiment, the compound of formula (I) and the TGFβ superfamily signaling pathway inhibitor separately contact a population of pancreatic beta cells simultaneously or in sequence.

TGFβ superfamily signaling pathway inhibitors include small molecules and other (e.g., neutralizing monoclonal antibodies, synthetic/recombinant peptide inhibitors, and siRNA) inhibitors of the BMP family of receptors, activin and inhibin receptors, GDF11 receptors, and related receptors.

TGFβ superfamily signaling pathway inhibitors are also known in the art and include, without limitation, SB431542, SB505124, A-83-01, Decorin, soluble TGF-β receptor, Ierdelimumab, metelimumab, AP-12009, Follistatin, FLRG, GAST-1, GDF8 propeptide, MYO-029, Noggin, chordin, Cer/Dan, ectodin, and Sclerostin (see Tsuchida et al., “Inhibitors of the TGF-beta Superfamily and their Clinical Applications,” Mini Rev. Med Chem. 6(11):1255-61 (2006), which is hereby incorporated by reference in its entirety).

Other inhibitors of TGF-β signaling include, without limitation, 2-(3-(6-Methylpyridin-2-yl)-1H-pyrazol-4-yl)-1,5 napththyridine; [3-(Pyridin-2-yl)-4-(4-quinoyl)]-1H-pyrazole; 3-(6-Methylpyridin-2-yl)-4-(4-quinolyl)-1-phenylthiocarbamoyl-1H-pyrazole; SB-431542; SM16; SB-505124; and 2-(3-(6-Methylpyridin-2-yl)-1H-pyrazol-4-yl)-1,5 napththyridine (ALK5 Inhibitor II) (see U.S. Pat. No. 8,298,825, which is hereby incorporated by reference in its entirety).

Inhibitors of TGF-β signaling are described in Callahan et al., “Identification of Novel Inhibitors of the Transforming Growth Factor beta1 (TFG-beta1) Type 1 Receptor (ALK5),” J. Med Chem. 45:999-1001 (2002); Sawyer et al., “Synthesis and Activity of New Aryl- and Heteroaryl-Substituted Pyrazole Inhibitors of the Transforming Growth Factor-β Type I Receptor Kinase Domain,” J. Med Chem. 46:3953-3956 (2003); Gellibert et al., “Identification of 1,5-naphthyridine Derivatives as a Novel Series of Potent and Selective TGF-beta Type I Receptor Inhibitors,” J. Med Chem. 47:4494-4506 (2004); Tojo et al., “The ALK-5 Inhibitor A-83-01 Inhibits Smad Signaling and Epithelial-To-Mesenchymal Transition by Transforming Growth Factor-Beta,” Cancer Sci. 96:791-800 (2005); Valdimarsdottir et al., “Functions of the TGFβ Superfamily in Human Embryonic Stem Cells,” APMIS 113:773-389 (2005); Petersen et al., “Oral Administration of GW788388, an Inhibitor of TGF-β Type I and II Receptor Kinases, Decreases Renal Fibrosis,” Kidney International 73:705-715 (2008); Yingling et al., “Development of TGF-β Signalling Inhibitors for Cancer Therapy,” Nature Rev. Drug Disc. 3:1011-1022 (2004); Byfield et al., “SB-505124 is a Selective Inhibitor of Transforming Growth Factor-β Type I Receptors ALK4, ALK5, and ALK7,” Mol. Pharmacol. 65:744-752 (2004); Dumont et al., “Targeting the TGF Signaling Network in Human Neoplasia,” Cancer Cell 3:531-536 (2003); PCT Publication No. WO 2002/094833; PCT Publication No. WO 2004/026865; PCT Publication No. WO 2004/067530; PCT Publication No. WO 2009/032667; PCT Publication No. WO 2004/013135; PCT Publication No. WO 2003/097639; PCT Publication No. WO 2007/048857; PCT Publication No. WO 2007/018818; PCT Publication No. WO 2006/018967; PCT Publication No. WO 2005/039570; PCT Publication No. WO 2000/031135; PCT Publication No. WO 1999/058128; U.S. Pat. Nos. 6,509,318; 6,090,383; 6,419,928; 9,927,738; 7,223,766; 6,476,031; 6,419,928; 7,030,125; 6,943,191; U.S. Patent Application Publication No. 2005/0245520; U.S. Patent Application Publication No. 2004/0147574; U.S. Patent Application Publication No. 2007/0066632; U.S. Patent Application Publication No. 2003/0028905; U.S. Patent Application Publication No. 2005/0032835; U.S. Patent Application Publication No. 2008/0108656; U.S. Patent Application Publication No. 2004/015781; U.S. Patent Application Publication No. 2004/0204431; U.S. Patent Application Publication No. 2006/0003929; U.S. Patent Application Publication No. 2007/0155722; U.S. Patent Application Publication No. 2004/0138188 and U.S. Patent Application Publication No. 2009/0036382, which are hereby incorporated by reference in their entirety.

Exemplary inhibitors of TGF-β signaling include, but are not limited to, AP-12009 (TGF-β Receptor type II antisense oligonucleotide), Lerdelimumab (CAT 152, antibody against TGF-β Receptor type II) GC-1008 (antibody to all isoforms of human TGF-β), ID11 (antibody to all isoforms of murine TGF-β), soluble TGF-β, soluble TGF-β Receptor type II, dihydropyrroloimidazole analogs (e.g., SKF-104365), triarylimidazole analogs (e.g., SB-202620 (4-(4-(4-fluorophenyl)-5-(pyridin-4-yl)-1H-imidazol-2-yl)benzoic acid) and SB-203580 (4-(4-Fluorophenyl)-2-(4-methylsulfinyl phenyl)-5-(4-pyridyl)-1H-imidazole)), RL-0061425, 1,5-naphthyridine aminothiazole and pyrazole derivatives (e.g., 4-(6-methyl-pyridin-2-yl)-5-(1,5-naphthyridin-2-yl)-1,3-thiazole-2-amine and 2-[3-(6-methyl-pyridin-2-yl)-1H-pyrazole-4-yl]-1,5-naphthyridine), SB-431542 (4-(5-Benzol[1,3]dioxol-5-yl-4-pyridin-2-yl-1H-imidazol-2-yl)-benzamide), GW788388 (4-(4-(3-(pyridin-2-yl)-1H-pyrazol-4-yl)pyridin-2-yl)-N-(tetrahydro-2H-pyran-4-yl)benzamide), A-83-01 (3-(6-Methyl-2-pyridinyl)-N-phenyl-4-(4-quinolinyl)-1H-pyrazole-1-carbothioamide), Decorin, Lefty 1, Lefty 2, Follistatin, Noggin, Chordin, Cerberus, Gremlin, Inhibin, BIO (6-bromo-indirubin-3′-oxime), Smad proteins (e.g., Smad6, Smad7), and Cystatin C.

Inhibitors of TGF-β signaling also include molecules which inhibit TGF-β Receptor type I. Inhibitors of TGF-β Receptor type I include, but are not limited to, soluble TGF-β Receptor type I; AP-11014 (TGF-0 Receptor type I antisense oligonucleotide); Metelimumab (CAT 152, TGF-β Receptor type I antibody); LY550410; LY580276 (3-(4-fluorophenyl)-5,6-dihydro-2-(6-methylpyridin-2-yl)-4H-pyrrolo[1,2-b]pyrazole); LY364947 (4-[3-(2-Pyridinyl)-1H-pyrazol-4-yl]-quinoline); LY2109761; LY573636 (N-((5-bromo-2-thienyl)sulfonyl)-2,4-dichlorobenzamide); SB-505124 (2-(5-Benzo[1,3]dioxol-5-yl-2-tert-butyl-3H-imidazol-4-yl)-6-methylpyridine); SD-208 (2-(5-Chloro-2-fluorophenyl)-4-[(4-pyridyl)amino]pteridine); SD-093; KI2689; SM16; FKBP12 protein; and 3-(4-(2-(6-methylpyridin-2-yl)H-imidazo[1,2-a]pyridin-3-yl)quinolin-7-yloxy)-N,N-dimethylpropan-1-amine.

Inhibitors of TGF-β Receptor type I are described in Byfield and Roberts, “Lateral Signaling Enhances TGF-beta Response Complexity,” Trends Cell Biol. 14:107-111 (2004); Sawyer et al., “Synthesis and Activity of New Aryl- And Heteroaryl-Substituted 5,6-dihydro-4H-pyrrolo[1,2-b]pyrazole Inhibitors of the Transforming Growth Factor-Beta Type I Receptor Kinase Domain,” Bioorg. Med. Chem. Lett. 14:3581-3584 (2004); Sawyer et al., “Synthesis and Activity of New Aryl- and Heteroaryl-Substituted Pyrazole Inhibitors of the Transforming Growth Factor-β Type I Receptor Kinase Domain,” J. Med. Chem. 46:3953-3956 (2003); Byfield et al., “SB-505124 is a Selective Inhibitor of Transforming Growth Factor-β Type I Receptors ALK4, ALK5, and ALK7,” Mol. Pharmacol. 65:744-752 (2004); Gellibert et al., “Identification of 1,5-naphthyridine Derivatives as a Novel Series of Potent and Selective TGF-beta Type I Receptor Inhibitors,” J. Med. Chem. 47:4494-4506 (2004); Yingling et al., “Development of TGF-β Signalling Inhibitors for Cancer Therapy,” Nature Rev. Drug Disc. 3:1011-1022 (2004); Dumont et al., “Targeting the TGF Signaling Network in Human Neoplasia,” Cancer Cell 3:531-536 (2003); Tojo et al., “The ALK-5 Inhibitor A-83-01 Inhibits Smad Signaling and Epithelial-To-Mesenchymal Transition by Transforming Growth Factor-Beta,” Cancer Sci. 96:791-800 (2005); PCT Publication No. WO 2004/026871; PCT Publication No. WO 2004/021989; PCT Publication No. WO 2004/026307; PCT Publication No. WO 2000/012497; U.S. Pat. Nos. 5,731,424; 5,731,144; 7,151,169; U.S. Patent Application Publication No. 2004/00038856 and U.S. Patent Application Publication No. 2005/0245508, all of which are herein incorporated in their entirety.

In one embodiment, the TGFβ superfamily signaling pathway inhibitor includes compounds that interfere with TGFβ superfamily ligands, receptors, and/or downstream signaling molecules (e.g., SMADs) or nuclear targets (e.g., chromatin modifying complexes and transcription factors).

In one embodiment, the TGFβ superfamily signaling pathway inhibitor may be antisera that neutralize, e.g., TGFβ ligand.

In another embodiment, the TGFβ superfamily signaling pathway inhibitor is selected from the group consisting of an inhibitor of TGFβ/TGFβ receptor binding, activin or inhibin/activin receptor binding, and bone morphogenetic protein (BMP)/BMP receptor binding.

The TGFβ superfamily signaling pathway inhibitor may be an inhibitor of TGFβ/TGFβ receptor binding selected from the group consisting of LY364947 and GW788388.

The TGFβ superfamily signaling pathway inhibitor may be an inhibitor of activin or inhibin/activin receptor binding selected from the group consisting of SB431542 and Alk5 inhibitor II. Additional exemplary inhibitors of activin or inhibin/activin receptor binding may be selected from the group consisting of SB-505124, BYM388, follistatin, follistatin-related protein (FSRP), follistatin domains (i.e., Fs2, Fs12, Fs123), A-83-01, Cripto, GW788388, BAMBI, and Sotatercept (see Byfield et al., “SB-505124 is a Selective Inhibitor of Transforming Growth Factor-Beta Type I Receptors ALK4, ALK5, and ALK7,” Mol. Pharmacol. 65(3):744-52 (2004); Lach-Trifilieffa et al., “An Antibody Blocking Activin Type II Receptors Induces Strong Skeletal Muscle Hypertrophy and Protects from Atrophy,” Mol. Cell. Biol. 34(4):606-18 (2014); Zhang et al., “Inhibition of Activin Signaling Induces Pancreatic Epithelial Cell Expansion and Diminishes Terminal Differentiation of Pancreatic β-Cells,” Diabetes 53(8):2024-33 (2004); Harrington et al., “Structural Basis for the Inhibition of Activin Signalling by Follistatin,” EMBO J. 25(5):1035-45 (2006); Tojo et al., “The ALK-5 Inhibitor A-83-01 Inhibits Smad Signaling and Epithelial-to-Mesenchymal Transition by Transforming Growth Factor-Beta,” Cancer Sci. 96(11):790-800 (2005); Yan et al., “Human BAMBI Cooperates with Smad7 to Inhibit Transforming Growth Factor-Beta Signaling,” J. Biol. Chem. 284(44):30097-104 (2009); Tan et al., “Targeted Inhibition of Activin Receptor-Like Kinase 5 Signaling Attenuates Cardiac Dysfunction Following Myocardial Infarction,” Am. J. Physiol. Heart Circ. Physiol. 298 (5):H1415-25 (2010); and Gokoffski et al., “Activin and GDF11 Collaborate in Feedback Control of Neuroepithelial Stem Cell Proliferation and Fate,” Develop. 138(19):4131-42 (2011), which are hereby incorporated by reference in their entirety).

The TGFβ superfamily signaling pathway inhibitor may be an inhibitor of BMP/BMP receptor binding. An exemplary inhibitor of BMP/BMP receptor binding is LDN193189. Additional exemplary BMP inhibitors may be selected from the group consisting of noggin, sclerostin, chordin, CTGF, follistatin, gremlin, inhibin, DMH1, DMH2, Dorsomorphin, K02288, LDN212854, DM 3189, BMP-3, and BAMBI (see PCT Publication No. WO 2014018691 A1; Mohedas et al., “Development of an ALK2-Biased BMP Type I Receptor Kinase Inhibitor,” ACS Chem. Biol. 8(6):1291-302 (2013); and Yan et al., “Human BAMBI Cooperates with Smad7 to Inhibit Transforming Growth Factor-Beta Signaling,” J. Biol. Chem. 284(44):30097-104 (2009), which are hereby incorporated by reference in their entirety).

The TGFβ superfamily signaling pathway inhibitor may be a SMAD signaling pathway inhibitor. Exemplary SMAD signaling pathway inhibitors may be selected from the group including, without limitation, SMAD3 siRNA, SMAD 2/3 siRNA, PD169316, SB203580, SB202474, specific inhibitor of Smad3 (SIS3), HSc025, and SB525334 (see Qureshi et al., “Smad Signaling Pathway is a Pivotal Component of Tissue Inhibitor of Metalloproteinases-3 Regulation by Transforming Growth Factor Beta in Human Chondrocytes,” BBA Mol. Cell Res. 1783(9):1605-12 (2008); Hasegawa et al., “A Novel Inhibitor of Smad-Dependent Transcriptional Activation Suppresses Tissue Fibrosis in Mouse Models of Systemic Sclerosis,” Arthritis Rheum. 60(11):3465-75 (2009); and Ramdas et al., “Canonical Transforming Growth Factor-β Signaling Regulates Disintegrin Metalloprotease Expression in Experimental Renal Fibrosis via miR-29,” Am. J. Pathol. 183(6):1885-96 (2013), which are hereby incorporated by reference in their entirety).

Additional exemplary SMAD signaling pathway inhibitors include, without limitation, miR-100, LDN 193189, SMAD-binding peptide aptamers (e.g., Trx-FoxH1, Trx-Le1, Trx-CBP, Trx-SARA), pirfenidone, and LDN193189 (see Fu et al., “MicroRNA-100 Inhibits Bone Morphogenetic Protein-Induced Osteoblast Differentiation by Targeting Smad,” Eur. Rev. Med. Pharmacol. Sci. 20(18):3911-19 (2016); Boergermann et al., “Dorsomorphin and LDN-193189 Inhibit BMP-Mediated Smad, p38 and Akt signalling in C2C12 Cells,” Int. J. Biochem. Cell Biol. 42(11):1802-7 (2010); Cui et al., “Selective Inhibition of TGF-Responsive Genes by Smad-Interacting Peptide Aptamers from FoxH1, Lef1 and CBP,” Oncogene 24:3864-74 (2005); Zhao et al., “Inhibition of Transforming Growth Factor-Beta1-Induced Signaling and Epithelial-to-Mesenchymal Transition by the Smad-Binding Peptide Aptamer Trx-SARA,” Mol. Biol. Cell 17:3819-31 (2006); Li et al., “Oral Pirfenidone Protects Against Fibrosis by Inhibiting Fibroblast Proliferation and TGF-β Signaling in a Murine Colitis Model,” Biochem. Pharmacol. 117:57-67 (2016); and Cook et al., “BMP Signaling Balances Murine Myeloid Potential Through SMAD-Independent p38MAPK and NOTCH Pathways,” Blood 124(3):393-402 (2014), which are hereby incorporated by reference in their entirety).

The TGFβ superfamily signaling pathway inhibitor may be an inhibitor of the trithorax complex. Exemplary trithorax complex inhibitors include, without limitation, WDR5-0103, MI-1, MI-2, MI-2-2, MLS001171971-01, ML227, MCP-1, RBB5 siRNA, and MLL1 siRNA (see Senisterra et al., “Small-Molecule Inhibition of MLL Activity by Disruption of its Interaction with WDR5,” Biochem. J. 449(1):151-9 (2013); Cierpicki et al., “Challenges and Opportunities in Targeting the Menin-MLL Interaction,” Future Med. Chem. 6(4):447-62 (2014); Lee et al., “Roles of DPY30 in the Proliferation and Motility of Gastric Cancer Cells,” PLOS One 10(7):e0131863 (2015); and Zhou et al., “Combined Modulation of Polycomb and Trithorax Genes Rejuvenates R Cell Replication,” J. Clin. Invest. 123(11):4849-4858 (2013), which are hereby incorporated by reference in their entirety).

The TGFβ superfamily signaling pathway inhibitor may be an inhibitor of the polycomb repressive complex 2 (“PRC2”). Exemplary PRC2 inhibitors include GSK926, EPZ005687, GSK126, GSK343, E11, UNC1999, EPZ6438, Constellation Compound 3, EZH2 siRNA, and 3-deazaneplanocin A (see Verma et al., “Identification of Potent, Selective, Cell-Active Inhibitors of the Histone Lysine Methyltransferase EZH2,” ACS Med. Chem. Lett. 3:1091-6 (2012); Xu et al., “Targeting EZH2 and PRC2 Dependence as Novel Anticancer Therapy,” Exp. Hematol. 43:698-712 (2015); Knutson et al., “A Selective Inhibitor of EZH2 Blocks H3K27 Methylation and Kills Mutant Lymphoma Cells,” Nat. Chem. Biol. 8:890-6 (2012); Qi et al., “Selective Inhibition of Ezh2 by a Small Molecule Inhibitor Blocks Tumor Cells Proliferation,” Proc. Natl Acad. Sci. USA 109:21360-65 (2012); McCabe et al., “EZH2 Inhibition as a Therapeutic Strategy for Lymphoma with EZH2-Activating Mutations,” Nature 492:108-12 (2012); Nasveschuk et al., “Discovery and Optimization of Tetramethylpiperidinyl Benzamides as Inhibitors of EZH2,” ACS Med. Chem. Lett. 5:378-83 (2014); Brooun et al., “Polycomb Repressive Complex 2 Structure with Inhibitor Reveals a Mechanism of Activation and Drug Resistance,” Nature Comm. 7:11384 (2016); Fiskus et al., “Histone Deacetylase Inhibitors Deplete Enhancer of Zeste 2 and Associated Polycomb Repressive Complex 2 Proteins in Human Acute Leukemia Cells,” Mol. Cancer Ther. 5(12):3096-104 (2006); and Fiskus et al., “Combined Epigenetic Therapy with the Histone Methyltransferase EZH2 Inhibitor 3-Deazaneplanocin A and the Histone Deacetylase Inhibitor Panobinostat Against Human AML Cells,” Blood 114(13):2733-43 (2009), which are hereby incorporated by reference in their entirety.)

The method may further involve contacting the population of pancreatic beta cells with a glucagon-like peptide-1 receptor (“GLP1R”) agonist and/or a Dipeptidyl Peptidase IV (“DDP4”) inhibitor. In accordance with this embodiment, the method may be carried out with a composition comprising a compound according to formula (I) and the glucagon-like peptide-1 receptor (GLP1R) agonist and/or the DDP4 inhibitor, and, optionally, the TGFβ superfamily signaling pathway inhibitor. In another embodiment, the compound of formula (I), the GLP1R agonist and/or the DDP4 inhibitor, and, optionally, the TGFβ superfamily signaling pathway inhibitor each contact the population of pancreatic beta cells simultaneously or in sequence.

Glucagon-like peptide-1 receptor agonists mimic the effects of the incretin hormone GLP-1, which is released from the intestine in response to food intake. Their effects include increasing insulin secretion, decreasing glucagon release, increasing satiety, and slowing gastric emptying. An alternate approach to enhancing GLP1 concentrations in blood is prevention of its degradation by the enzyme DPP4. The GLP1 receptor agonists and the DDP4 inhibitors are among the most widely used drugs for the treatment of Type 2 diabetes (Campbell et al., “Pharmacology, Physiology and Mechanisms of Incretin Hormone Action,” Cell Metab. 17:819-37 (2013); Guo X-H., “The Value of Short- and Long-Acting Glucagon-Like Peptide Agonists in the Management of Type 2 Diabetes Mellitus: Experience with Exenatide,” Curr. Med. Res. Opinion 32(1):61-76 (2016); Deacon et al., “Dipeptidyl Peptidase-4 Inhibitors for the Treatment of Type 2 Diabetes: Comparison, Efficacy and Safety,” Expert Opinion on Pharmacotherapy 14:2047-58 (2013); Lovshin, “Glucagon-Like Peptide-1 Receptor Agonists: A Class Update for Treating Type 2 Diabetes,” Can. J. Diabetes 41:524-35 (2017); and Yang et al., “Lixisenatide Accelerates Restoration of Normoglycemia and Improves Human Beta Cell Function and Survival in Diabetic Immunodeficient NOD-scid IL2rg(null) RIP-DTR Mice Engrafted With Human Islets,” Diabetes Metab. Syndr. Obes. 8:387-98 (2015), which are hereby incorporated by reference in their entirety).

Suitable GLP1R agonists include, e.g. and without limitation, exenatide, liraglutide, exenatide LAR, taspoglutide, lixisenatide, albiglutide, dulaglutide, and semaglutide. Exenatide and Exenatide LAR are synthetic exendin-4 analogues obtained from the saliva of the Heloderma suspectum (lizard). Liraglutide is an acylated analogue of GLP-1 that self-associates into a heptameric structure that delays absorption from the subcutaneous injection site. Taspoglutide shares 3% homology with the native GLP-1 and is fully resistant to DPP-4 degradation. Lixisenatide is a human GLP1R agonist. Albiglutide is a long-acting GLP-1 mimetic, resistant to DPP-4 degradation. Dulaglutide is a long-acting GLP1 analogue. Semaglutide is a GLP1R agonist approved for the use of T2D. Clinically available GLP1R agonists include, e.g., exenatide, liraglutide, albiglutide, dulaglutide, lixisenatide, semaglutide.

In some embodiments of the methods and compositions of the present invention, the GLP1R agonist is selected from the group consisting of GLP1 (7-36), extendin-4, liraglutide, lixisenatide, semaglutide, and combinations thereof.

Additional suitable GLP1 agonists include, without limitation, disubstituted-7-aryl-5,5-bis(trifluoromethyl)-5,8-dihydropyrimido[4,5-d]pyrimidine-2,4 (1H,3H)-dione compounds and derivatives thereof, e.g., 7-(4-Chlorophenyl)-1,3-dimethyl-5,5-bis(trifluoromethyl)-5,8-dihydropyrimido[4,5-d]pyrimidine-2,4 (1H,3H)-dione (see, e.g., Nance et al., “Discovery of a Novel Series of Orally Bioavailable and CNS Penetrant Glucagon-like Peptide-1 Receptor (GLP-1R) Noncompetitive Antagonists Based on a 1,3-Disubstituted-7-aryl-5,5-bis(trifluoromethyl)-5,8-dihydropyrimido[4,5-d]pyrimidine-2,4 (1H,3H)-dione Core,” J. Med. Chem. 60:1611-1616 (2017), which is hereby incorporated by reference in its entirety).

Further suitable GLP1 agonists include positive allosteric modulators (“PAMS”) of GLP1R, e.g., (S)-2-cyclopentyl-N-((1-isopropylpyrrolidin-2-yl)methyl)-10-methyl-1-oxo-1,2-dihydropyrazino[1,2-a]indole-4-carboxamide; (R)-2-cyclopentyl-N-((1-isopropylpyrrolidin-2-yl)methyl)-10-methyl-1-oxo-1,2-dihydropyrazino[1,2-a]indole-4-carboxamide; 2-cyclopentyl-N—(((S)-1-isopropylpyrrolidin-2-yl)methyl)-10-methyl-1-oxo-1,2,3,4-tetrahydropyrazino[1,2-a]indole-4-carboxamide; N—(((S)-1-isopropylpyrrolidin-2-yl)methyl)-10-methyl-1-oxo-2-((S)-tetrahydrofuran-3-yl)-1,2-dihydropyrazino[1,2-a]indole-4-carboxamide; N—(((R)-1-isopropylpyrrolidin-2-yl)methyl)-10-methyl-1-oxo-2-((S)-tetrahydrofuran-3-yl)-1,2-dihydropyrazino[1,2-a]indole-4-carboxamide; (S)-2-cyclopentyl-8-fluoro-N-((1-isopropylpyrrolidin-2-yl)methyl)-10-methyl-1-oxo-1,2-dihydropyrazino[1,2-a]indole-4-carboxamide; (R)-2-cyclopentyl-8-fluoro-N-((1-isopropylpyrrolidin-2-yl)methyl)-10-methyl-1-oxo-1,2-dihydropyrazino[1,2-a]indole-4-carboxamide; (R)-2-cyclopentyl-N—(((S)-1-isopropylpyrrolidin-2-yl)methyl)-10-methyl-1-oxo-1,2,3,4-tetrahydropyrazino[1,2-a]indole-4-carboxamide; (S)-2-cyclopentyl-N—(((S)-1-isopropylpyrrolidin-2-yl)methyl)-10-methyl-1-oxo-1,2,3,4-tetrahydropyrazino[1,2-a]indole-4-carboxamide; (S)-10-chloro-2-cyclopentyl-N-((1-isopropylpyrrolidin-2-yl)methyl)-1-oxo-1,2-dihydropyrazino[1,2-a]indole-4-carboxamide; (R)-10-chloro-2-cyclopentyl-N-((1-isopropylpyrrolidin-2-yl)methyl)-1-oxo-1,2-dihydropyrazino[1,2-a]indole-4-carboxamide; (S)-10-bromo-2-cyclopentyl-N-((1-isopropylpyrrolidin-2-yl)methyl)-1-oxo-1,2-dihydropyrazino[1,2-a]indole-4-carboxamide; (R)-10-bromo-2-cyclopentyl-N-((1-isopropylpyrrolidin-2-yl)methyl)-1-oxo-1,2-dihydropyrazino[1,2-a]indole-4-carboxamide; (R)—N-((1-isopropylpyrrolidin-2-yl)methyl)-10-methyl-1-oxo-2-phenyl-1,2-dihydropyrazino[1,2-a]indole-4-carboxamide; (S)-10-cyano-2-cyclopentyl-N-((1-isopropylpyrrolidin-2-yl)methyl)-1-oxo-1,2-dihydropyrazino[1,2-a]indole-4-carboxamide; (S)-2-cyclopentyl-N-((1-isopropylpyrrolidin-2-yl)methyl)-1-oxo-10-vinyl-1,2-dihydropyrazino[1,2-a]indole-4-carboxamide; (S)—N-((1-isopropylpyrrolidin-2-yl)methyl)-10-methyl-2-(1-methyl-1H-pyrazol-4-yl)-1-oxo-1,2-dihydropyrazino[1,2-a]indole-4-carboxamide; (R)—N-((1-isopropylpyrrolidin-2-yl)methyl)-10-methyl-2-(1-methyl-1H-pyrazol-4-yl)-1-oxo-1,2-dihydropyrazino[1,2-a]indole-4-carboxamide; (S)—N-((1-isopropylpyrrolidin-2-yl)methyl)-10-methyl-1-oxo-2-(pyridin-3-yl)-1,2-dihydropyrazino[1,2-a]indole-4-carboxamide; (R)—N-((1-isopropylpyrrolidin-2-yl)methyl)-10-methyl-1-oxo-2-(pyridin-3-yl)-1,2-dihydropyrazino[1,2-a]indole-4-carboxamide; N-(azetidin-2-ylmethyl)-2-cyclopentyl-10-methyl-1-oxo-1,2-dihydropyrazino[1,2-a]indole-4-carboxamide; and 2-cyclopentyl-N-((1-isopropylazetidin-2-yl)methyl)-10-methyl-1-oxo-1,2-dihydropyrazino[1,2-a]indole-4-carboxamide; or pharmaceutically acceptable salts thereof (see PCT Publication No. WO 2017/117556, which is hereby incorporated by reference in its entirety).

Suitable DDP4 inhibitors include, without limitation, sitagliptin, vildagliptin, saxagliptin, alogliptin, teneligliptin, and anagliptin.

According to one embodiment, “pancreatic beta cells” are primary human pancreatic beta cells.

In one embodiment of carrying out this and other methods of the present invention, contacting does not induce beta cell death or DNA damage. Moreover, contacting may induce beta cell differentiation and increase glucose-stimulated insulin secretion.

In another embodiment, the method is carried out to enhance cell survival. For example, the method may be carried out to enhance cell survival of a treated population of cells relative to an untreated population of cells. Alternatively, the method may be carried out to decrease cell death or apoptosis of a treated population of cells relative to an untreated population of cells.

A further aspect of the present invention relates to a composition comprising a compound of formula (I) described herein and a carrier.

The composition may further comprise a transforming growth factor beta (TGFβ) superfamily signaling pathway inhibitor.

In another embodiment, the composition may further comprise a glucagon-like peptide-1 receptor (GLP1R) agonist or a Dipeptidyl Peptidase IV (DDP4) inhibitor.

The carrier may be a pharmaceutically-acceptable carrier.

While it may be possible for compounds of formula (I) to be administered as the raw chemical, they may also be administered as a pharmaceutical composition. In accordance with an aspect of the present invention, there is provided a pharmaceutical composition comprising a compound of formula (I) or a pharmaceutically acceptable salt or solvate thereof, together with one or more pharmaceutically carriers thereof and optionally one or more other therapeutic ingredients.

The carrier(s) must be “acceptable” in the sense of being compatible with the other ingredients of the formulation and not deleterious to the recipient thereof. Furthermore, notwithstanding the statements herein regarding the term “compound” including salts thereof as well, so that independent claims reciting “a compound” will be understood as referring to salts thereof as well, if in an independent claim reference is made to a compound or a pharmaceutically acceptable salt thereof, it will be understood that claims which depend from that independent claim which refer to such a compound also include pharmaceutically acceptable salts of the compound, even if explicit reference is not made to the salts in the dependent claim.

Formulations include those suitable for oral, parenteral (including subcutaneous, intradermal, intramuscular, intravenous, and intraarticular), rectal and topical (including dermal, buccal, sublingual, and intraocular) administration. The most suitable route may depend upon the condition and disorder of the recipient. The formulations may conveniently be presented in unit dosage form and may be prepared by any of the methods well known in the art of pharmacy. Such methods include the step of bringing into association a compound of formula (I) or a pharmaceutically acceptable salt or solvate thereof (“active ingredient”) with the carrier, which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations suitable for oral administration may be presented as discrete units such as capsules, cachets, or tablets each containing a predetermined amount of the active ingredient; as a powder or granules; as a solution or a suspension in an aqueous liquid or a nonaqueous liquid; or as an oil-in-water liquid emulsion or a water-in-oil liquid emulsion. The active ingredient may also be presented as a bolus, electuary, or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed, or controlled release of the active ingredient therein.

The pharmaceutical compositions may include a “pharmaceutically acceptable inert carrier,” and this expression is intended to include one or more inert excipients, which include, for example and without limitation, starches, polyols, granulating agents, microcrystalline cellulose, diluents, lubricants, binders, disintegrating agents, and the like. If desired, tablet dosages of the disclosed compositions may be coated by standard aqueous or nonaqueous techniques. “Pharmaceutically acceptable carrier” also encompasses controlled release means.

Pharmaceutical compositions may also optionally include other therapeutic ingredients, anti-caking agents, preservatives, sweetening agents, colorants, flavors, desiccants, plasticizers, dyes, and the like. Any such optional ingredient must be compatible with the compound of formula (I) to insure the stability of the formulation. The composition may contain other additives as needed including, for example, lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

Examples of excipients for use as the pharmaceutically acceptable carriers and the pharmaceutically acceptable inert carriers and the aforementioned additional ingredients include, but are not limited to, binders, fillers, disintegrants, lubricants, anti-microbial agents, and coating agents.

Dose ranges for adult humans vary, but may generally be from about 0.005 mg to 10 g/day orally. Tablets or other forms of presentation provided in discrete units may conveniently contain an amount of compound of formula (I) which is effective at such dosage or as a multiple of the same, for instance, units containing 5 mg to 500 mg, or around 10 mg to 200 mg. The precise amount of compound administered to a patient will be the responsibility of the attendant physician. However, the dose employed will depend on a number of factors, including the age and sex of the patient, the precise disorder being treated, and its severity.

A dosage unit (e.g., an oral dosage unit) can include from, for example, 1 to 30 mg, 1 to 40 mg, 1 to 100 mg, 1 to 300 mg, 1 to 500 mg, 2 to 500 mg, 3 to 100 mg, 5 to 20 mg, 5 to 100 mg (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg) of a compound described herein.

Additional information about pharmaceutical compositions and their formulation is described in Remington: The Science and Practice of Pharmacy, 20^{th} Edition, 2000, which is hereby incorporated by reference in its entirety.

The agents (i.e., compounds and pharmaceutically acceptable compositions described herein) can be administered, e.g., by intravenous injection, intramuscular injection, subcutaneous injection, intraperitoneal injection, topical, sublingual, intraarticular (in the joints), intradermal, buccal, ophthalmic (including intraocular), intranasaly (including using a cannula), or by other routes. The agents can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g., PCT Publication No. WO 97/11682, which is hereby incorporated by reference in its entirety) via a liposomal formulation (see, e.g., EP Patent No. 736299, PCT Publication No. WO 99/59550, and PCT Publication No. WO 97/13500, which are hereby incorporated by reference in their entirety), via formulations described in PCT Publication No. WO 03/094886 (which is hereby incorporated by reference in its entirety) or in some other form. The agents can also be administered transdermally (i.e., via reservoir-type or matrix-type patches, microneedles, thermal poration, hypodermic needles, iontophoresis, electroporation, ultrasound, or other forms of sonophoresis, jet injection, or a combination of any of the preceding methods (Prausnitz et al. Nature Reviews Drug Discovery 3:115 (2004), which is hereby incorporated by reference in its entirety). The agents can be administered locally.

The agents can be administered in the form a suppository or by other vaginal or rectal means. The agents can be administered in a transmembrane formulation as described in PCT Publication No. WO 90/07923, which is hereby incorporated by reference in its entirety. The agents can be administered non-invasively via the dehydrated particles described in U.S. Pat. No. 6,485,706, which is hereby incorporated by reference in its entirety. The agents can be administered in an enteric-coated drug formulation as described in PCT Publication No. WO 02/49621, which is hereby incorporated by reference in its entirety. The agents can be administered intranasaly using the formulation described in U.S. Pat. No. 5,179,079, which is hereby incorporated by reference in its entirety. Formulations suitable for parenteral injection are described in PCT Publication No. WO 00/62759, which is hereby incorporated by reference in its entirety. The agents can be administered using the casein formulation described in U.S. Patent Application Publication No. 2003/0206939 and PCT Publication No. WO 00/06108, which are hereby incorporated by reference in their entirety. The agents can be administered using the particulate formulations described in U.S. Patent Application Publication No. 20020034536, which is hereby incorporated by reference in its entirety.

The agents, alone or in combination with other suitable components, can be administered by pulmonary route utilizing several techniques including, but not limited to, intratracheal instillation (delivery of solution into the lungs by syringe), intratracheal delivery of liposomes, insufflation (administration of powder formulation by syringe or any other similar device into the lungs), and aerosol inhalation. Aerosols (e.g., jet or ultrasonic nebulizers, metered-dose inhalers (“MDIs”), and dry-Powder inhalers (“DPIs”)) can also be used in intranasal applications. Aerosol formulations are stable dispersions or suspensions of solid material and liquid droplets in a gaseous medium and can be placed into pressurized acceptable propellants, such as hydrofluoroalkanes (HFAs, i.e., HFA-134a and HFA-227, or a mixture thereof), dichlorodifluoromethane (or other chlorofluorocarbon propellants such as a mixture of Propellants 11, 12, and/or 114), propane, nitrogen, and the like. Pulmonary formulations may include permeation enhancers such as fatty acids, and saccharides, chelating agents, enzyme inhibitors (e.g., protease inhibitors), adjuvants (e.g., glycocholate, surfactin, span 85, and nafamostat), preservatives (e.g., benzalkonium chloride or chlorobutanol), and ethanol (normally up to 5% but possibly up to 20%, by weight). Ethanol is commonly included in aerosol compositions as it can improve the function of the metering valve and in some cases also improve the stability of the dispersion.

Pulmonary formulations may also include surfactants which include, but are not limited to, bile salts and those described in U.S. Pat. No. 6,524,557 and references therein, which are hereby incorporated by reference in their entirety. The surfactants described in U.S. Pat. No. 6,524,557, e.g., a C₈-C₁₆ fatty acid salt, a bile salt, a phospholipid, or alkyl saccharide are advantageous in that some of them also reportedly enhance absorption of the compound in the formulation.

Also suitable in the invention are dry powder formulations comprising a therapeutically effective amount of active compound blended with an appropriate carrier and adapted for use in connection with a dry-powder inhaler. Absorption enhancers that can be added to dry powder formulations include those described in U.S. Pat. No. 6,632,456, which is hereby incorporated by reference in its entirety. PCT Publication No. WO 02/080884, which is hereby incorporated by reference in its entirety, describes new methods for the surface modification of powders. Aerosol formulations may include those described in U.S. Pat. Nos. 5,230,884 and 5,292,499; PCT Publication Nos. WO 017/8694 and 01/78696; U.S. Patent Application Publication Nos. 2003/019437 and 2003/0165436; and PCT Publication No. WO 96/40089 (which includes vegetable oil), which are hereby incorporated by reference in their entirety. Sustained release formulations suitable for inhalation are described in U.S. Patent Application Publication Nos. 2001/0036481, 2003/0232019, and 2004/0018243 as well as in PCT Publication Nos. WO 01/13891, 02/067902, 03/072080, and 03/079885, which are hereby incorporated by reference in their entirety.

Pulmonary formulations containing microparticles are described in PCT Publication No. WO 03/015750, U.S. Patent Application Publication No. 2003/0008013, and PCT Publication No. WO 00/00176, which are hereby incorporated by reference in their entirety. Pulmonary formulations containing stable glassy state powder are described in U.S. Patent Application Publication No. 2002/0141945 and U.S. Pat. No. 6,309,671, which are hereby incorporated by reference in their entirety. Other aerosol formulations are described in EP Patent No. 1338272; PCT Publication Nos. WO 90/09781 and WO 91/04011; and U.S. Pat. Nos. 5,348,730, 6,436,367, 6,294,153, and 6,290,987, which are hereby incorporated by reference in their entirety, which describe a liposomal based formulation that can be administered via aerosol or other means.

Powder formulations for inhalation are described in U.S. Patent Application Publication No. 2003/0053960 and PCT Publication No. WO 01/60341, which are hereby incorporated by reference in their entirety. The agents can be administered intranasally as described in U.S. Patent Application Publication No. 2001/0038824, which is hereby incorporated by reference in its entirety.

Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy such as Sprowls' American Pharmacy and Remington's The Science and Practice of Pharmacy.

Other devices for generating aerosols employ compressed gases, usually hydrofluorocarbons and chlorofluorocarbons, which are mixed with the medicament and any necessary excipients in a pressurized container. These devices are likewise described in standard textbooks such as Sprowls and Remington.

The agent can be incorporated into a liposome to improve half-life. The agent can also be conjugated to polyethylene glycol (“PEG”) chains. Methods for pegylation and additional formulations containing PEG-conjugates (i.e., PEG-based hydrogels, PEG modified liposomes) can be found in Harris and Chess, Nature Reviews Drug Discovery 2:214-221, which is hereby incorporated by reference in its entirety, and the references therein. The agent can be administered via a nanocochleate or cochleate delivery vehicle (BioDelivery Sciences International). The agents can be delivered transmucosally (i.e., across a mucosal surface such as the vagina, eye, or nose) using formulations such as that described in U.S. Pat. No. 5,204,108, which is hereby incorporated by reference in its entirety. The agents can be formulated in microcapsules as described in PCT Publication No. WO 88/01165, which is hereby incorporated by reference in its entirety. The agent can be administered intra-orally using the formulations described in U.S. Patent Application Publication No. 2002/0055496; PCT Publication No. WO 00/47203; and U.S. Pat. No. 6,495,120, which are hereby incorporated by reference in their entirety. The agent can be delivered using nanoemulsion formulations described in PCT Publication No. WO 01/91728, which is hereby incorporated by reference in its entirety.

Another aspect of the present invention relates to a method of treating a subject for a condition associated with an insufficient level of insulin secretion. This method involves administering to a subject in need of treatment for a condition associated with an insufficient level of insulin secretion a compound or composition of the present invention.

In one embodiment, the treatment methods of the present invention are carried out under conditions effective to increase pancreatic beta cell mass in the subject to treat the subject for an insufficient level of insulin secretion.

In one embodiment, the compound or composition may be administered with or coincident with a TGFβ superfamily signaling pathway inhibitor. Suitable transforming growth factor beta (TGFβ) superfamily signaling pathway inhibitors are described in detail above.

In another embodiment, the compound or composition may be administered with or coincident with a glucagon-like peptide-1 receptor (GLP1R) agonist or a Dipeptidyl Peptidase IV (DDP4) inhibitor. Suitable glucagon-like peptide-1 receptor (GLP1R) agonists or Dipeptidyl Peptidase IV (DDP4) inhibitors are described in detail above. In accordance with this embodiment, the administering is carried out under conditions effective to cause a synergistic increase in pancreatic beta cell mass in the subject to treat the subject for an insufficient level of insulin secretion.

As used herein, a condition associated with an insufficient level of insulin secretion means a condition where a subject produces a lower plasma level of insulin than is required to maintain normal glucose levels in the blood such that the subject with the condition associated with insufficient insulin secretion becomes hyperglycemic. In such a condition, the pancreatic beta cells of the afflicted subject secrete an insufficient level of insulin to maintain the presence of a normal concentration of glucose in the blood (i.e., normoglycemica).

According to one embodiment, one of the conditions associated with an insufficient level of insulin secretion is insulin resistance. Insulin resistance is a condition in which a subject's cells become less sensitive to the glucose-lowering effects of insulin. Insulin resistance in muscle and fat cells reduces glucose uptake (and, therefore, local storage of glucose as glycogen and triglycerides), whereas insulin resistance in liver cells results in reduced glycogen synthesis and storage and a failure to suppress glucose production and release into the blood. Insulin resistance normally refers to reduced glucose-lowering effects of insulin. However, other functions of insulin can also be affected. For example, insulin resistance in fat cells reduces the normal effects of insulin on lipids and results in reduced uptake of circulating lipids and increased hydrolysis of stored triglycerides. Increased mobilization of stored lipids in these cells elevates free fatty acids in the blood plasma. Elevated blood fatty-acid concentrations, reduced muscle glucose uptake, and increased liver glucose production all contribute to elevated blood glucose levels. If insulin resistance exists, more insulin needs to be secreted by the pancreas. If this compensatory increase does not occur, blood glucose concentrations increase and type II diabetes occurs.

According to another embodiment, one of the conditions associated with an insufficient level of insulin secretion is diabetes. Diabetes can be divided into two broad types of diseases: type I (“T1D”) and type II (“T2D”). The term “diabetes” also refers herein to a group of metabolic diseases in which patients have high blood glucose levels, including type I diabetes (T1D), type II diabetes (T2D), gestational diabetes, congenital diabetes, maturity onset diabetes (MODY), cystic fibrosis-related diabetes, hemochromatosis-related diabetes, drug-induced diabetes (e.g., steroid diabetes), and several forms of monogenic diabetes.

Thus, in one embodiment, the subject has been diagnosed as having one or more of type I diabetes (T1D), type II diabetes (T2D), gestational diabetes, congenital diabetes, maturity onset diabetes (MODY), cystic fibrosis-related diabetes, hemochromatosis-related diabetes, drug-induced diabetes, or monogenic diabetes.

According to another embodiment, a condition associated with an insufficient level of insulin secretion is metabolic syndrome. Metabolic syndrome is generally used to define a constellation of abnormalities that is associated with increased risk for the development of type II diabetes and atherosclerotic vascular disease. Related conditions and symptoms include, but are not limited to, fasting hyperglycemia (diabetes mellitus type II or impaired fasting glucose, impaired glucose tolerance, or insulin resistance), high blood pressure; central obesity (also known as visceral, male-pattern or apple-shaped adiposity), meaning overweight with fat deposits mainly around the waist; decreased HDL cholesterol; and elevated triglycerides.

In one embodiment, the subject has been diagnosed as having metabolic syndrome or insulin resistance.

Other conditions that may be associated with an insufficient level of insulin secretion include, without limitation, hyperuricemia, fatty liver (especially in concurrent obesity) progressing to non-alcoholic fatty liver disease, polycystic ovarian syndrome (in women), and acanthosis nigricans.

Related disorders may also be treated pursuant to the treatment methods of the present invention including, without limitation, any disease associated with a blood or plasma glucose level outside the normal range, such as hyperglycemia. Consequently, the term “related disorders” includes impaired glucose tolerance (IGT), impaired fasting glucose (IFG), insulin resistance, metabolic syndrome, postprandial hyperglycemia, and overweight/obesity. Such related disorders can also be characterized by an abnormal blood and/or plasma insulin level.

The methods described herein may be carried out to treat a subject with conditions associated with beta cell failure or deficiency. Such conditions include, without limitation, type I diabetes (T1D), type II diabetes (T2D), gestational diabetes, congenital diabetes, maturity onset diabetes (MODY), cystic fibrosis-related diabetes, hemochromatosis-related diabetes, drug-induced diabetes, or monogenic diabetes. Drug induced diabetes relates to a condition that is caused through the use of drugs that are toxic to beta cells (e.g., steroids, antidepressants, second generation antipsychotics, and immunosuppressive). Exemplary immunosuppressive drugs include, but are not limited to, members of the cortisone family (e.g., prednisone and dexamethasome), rapamycin/sirolimus, everolimus, and calciuneurin inhibitors (e.g., FK-506/tacrolimus).

Additional conditions associated with beta cell deficiency include, without limitation, pancreatectomy, partial pancreatectomy, pancreas transplantation, pancreatic islet transplantation, and pancreatitis (inflammation of the digestive enzyme-producing cells of the pancreas), which can cause post-pancreatitis diabetes.

The methods described herein may be carried out to treat a subject at risk of developing Type II Diabetes. For example, a patient at risk of developing Type II Diabetes has pre-diabetes/metabolic syndrome. The patient at risk of developing Type II Diabetes may have been treated with a psychoactive drug, including but not limited to a selective serotonin reuptake inhibitors (“SSRI”) for depression, obsessive compulsive disorder (“OCD”), etc.

In carrying out treatment methods, a compound of formula (I) or composition containing such compound and a TGFβ superfamily signaling pathway inhibitor are administered under conditions effective to increase pancreatic beta cell mass in the subject to treat the subject for a condition associated with an insufficient level of insulin secretion.

A compound or composition described herein and/or TGFβ superfamily signaling pathway inhibitor may be administered to increase pancreatic beta cell mass in the subject, which will result in an increased level of insulin secretion in the subject.

The compound and/or composition and TGFβ superfamily signaling pathway inhibitor may be formulated as separate pharmaceutical compositions or a single pharmaceutical composition comprising both the compound of formula (I) and TGFβ superfamily signaling pathway inhibitor. Such pharmaceutical composition(s) may comprise a therapeutically effective amount of the compound of formula (I) and/or TGFβ superfamily signaling pathway inhibitor.

Thus, a combination or combinatorial therapy or treatment of a compound of formula (I) and TGFβ superfamily signaling pathway inhibitor may be administered. The terms “combination” or “combinatorial therapy” or “combinatory treatment” mean a treatment where at least two compounds are co-administered to a subject to cause a biological effect, in this case a synergistic effect. In a combinatorial therapy, the at least two drugs may be administered together or separately, at the same time or sequentially. Simultaneous administration is not required, as long as the drugs produce a synergistic effect in the subject to improve the subject's conditions. Also, the at least two drugs may be administered through different routes and protocols. As a result, although they may be formulated together, the drugs of a combination may also be formulated separately.

A further aspect relates to a method of treating a subject for a neurological disorder. This method involves administering to a subject in need of treatment for a neurological disorder a compound of formula (I) under conditions effective to treat the subject for the condition.

The subject may have diabetes and/or has been diagnosed as having one or more of Down's Syndrome and a neurodegenerative disease.

In carrying out the treatment methods, administering of compounds to a subject may involve administering pharmaceutical compositions containing the compound(s) (i.e., a DYRK1A inhibitor of formula (I) and TGFβ superfamily signaling pathway inhibitor) in therapeutically effective amounts, which means an amount of compound effective in treating the stated conditions and/or disorders in the subject. Such amounts generally vary according to a number of factors well within the purview of ordinarily skilled artisans. These include, without limitation: the particular subject, as well as its age, weight, height, general physical condition, and medical history, the particular compound used, as well as the carrier in which it is formulated and the route of administration selected for it; the length or duration of treatment; and the nature and severity of the condition being treated.

Administering typically involves administering pharmaceutically acceptable dosage forms, which means dosage forms of compounds described herein and includes, for example, tablets, dragees, powders, elixirs, syrups, liquid preparations, including suspensions, sprays, inhalants tablets, lozenges, emulsions, solutions, granules, capsules, and suppositories, as well as liquid preparations for injections, including liposome preparations. Techniques and formulations generally may be found in Remington's Pharmaceutical Sciences, Mack Publishing Co., Easton, Pa., latest edition, which is hereby incorporated by reference in its entirety.

In carrying out treatment methods, the drug (i.e., a compound of formula (I) and, optionally, a TGFβ superfamily signaling pathway inhibitor) may be contained, in any appropriate amount, in any suitable carrier substance. The drug may be present in an amount of up to 99% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols.

Pharmaceutical compositions may be formulated to release the active drug substantially immediately upon administration or at any predetermined time or time period after administration.

Controlled release formulations include (i) formulations that create a substantially constant concentration of the drug(s) within the body over an extended period of time; (ii) formulations that after a predetermined lag time create a substantially constant concentration of the drug(s) within the body over an extended period of time; (iii) formulations that sustain drug(s) action during a predetermined time period by maintaining a relatively, constant, effective drug level in the body with concomitant minimization of undesirable side effects associated with fluctuations in the plasma level of the active drug substance; (iv) formulations that localize drug(s) action by, e.g., spatial placement of a controlled release composition adjacent to or in the diseased tissue or organ; and (v) formulations that target drug(s) action by using carriers or chemical derivatives to deliver the drug to a particular target cell type.

Administration of drugs in the form of a controlled release formulation is especially preferred in cases in which the drug has (i) a narrow therapeutic index (i.e., the difference between the plasma concentration leading to harmful side effects or toxic reactions and the plasma concentration leading to a therapeutic effect is small; in general, the therapeutic index (“TI”) is defined as the ratio of median lethal dose (LD₅₀) to median effective dose (ED₅₀)); (ii) a narrow absorption window in the gastro-intestinal tract; or (iii) a very short biological half-life so that frequent dosing during a day is required in order to sustain the plasma level at a therapeutic level.

Any of a number of strategies can be pursued to obtain controlled release in which the rate of release outweighs the rate of metabolism of the drug in question. Controlled release may be obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner (single or multiple unit tablet or capsule compositions, oil solutions, suspensions, emulsions, microcapsules, microspheres, nanoparticles, patches, and liposomes).

Thus, administering may be carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes. Compounds may be administered alone or with suitable pharmaceutical carriers, and can be in solid or liquid form, such as tablets, capsules, powders, solutions, suspensions, or emulsions.

The subject may be a mammalian subject. In one embodiment, the subject is a human subject. Suitable human subjects include, without limitation, children, adults, and elderly subjects having a beta-cell and/or insulin deficiency.

The subject may be bovine, ovine, porcine, feline, equine, murine, canine, lapine, etc.

The administering step may increase the number of proliferating pancreatic beta cells in the subject by at least about 5%, 6%, 7%, or more.

Administering may increase glucose-stimulated insulin secretion in pancreatic beta cells of the subject.

The designation of a compound is meant to designate the compound per se, as well as any pharmaceutically acceptable salt, hydrate, isomer, racemate, ester, or ether thereof. The designation of a compound is meant to designate the compound as specifically designated per se, as well as any pharmaceutically acceptable salt thereof.

Within the context of the present disclosure, by “treating” it is meant preventive or curative treatment.

Treatment may designate, in particular, the correction, decrease in the rate of change, or reduction of an impaired glucose homeostasis. The level of glucose in blood fluctuates throughout the day. Glucose levels are usually lower in the morning, before the first meal of the day and rise after meals for some hours. Consequently, the term treatment includes the control of blood glucose level by increasing or decreasing blood glucose level depending on the condition of the subject and the daytime in order to reach normal glucose levels. The term treatment more particularly includes a temporary or persistent reduction of blood glucose level in a subject having diabetes or a related disorder. The term “treatment” or “treating” also designates an improvement in insulin release (e.g., by pancreatic beta cells).

As used herein, the phrase “control of blood glucose level” refers to the normalization or the regulation of the blood or plasma glucose level in a subject having abnormal levels (i.e., levels that are below or above a known reference, median, or average value for a corresponding subject with a normal glucose homeostasis).

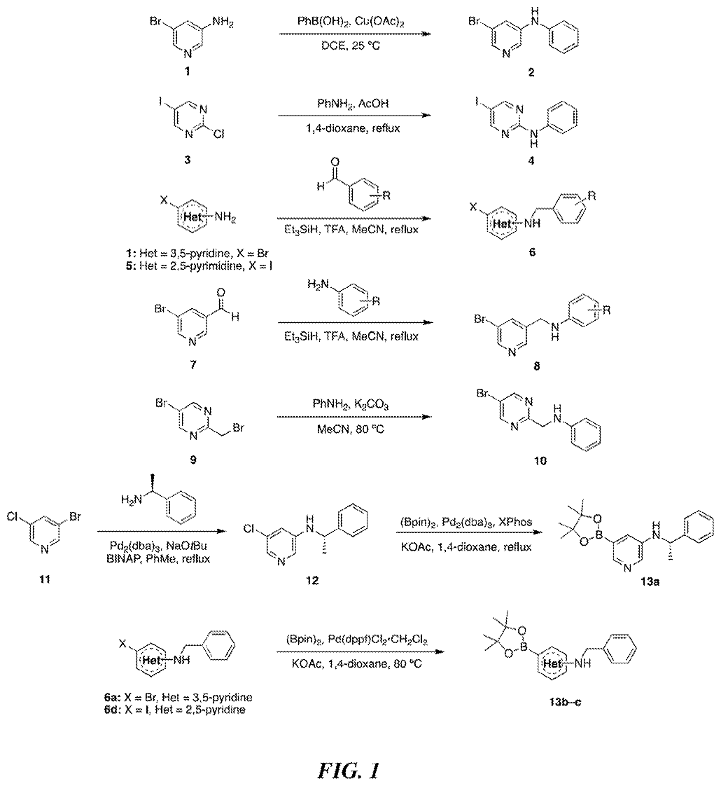

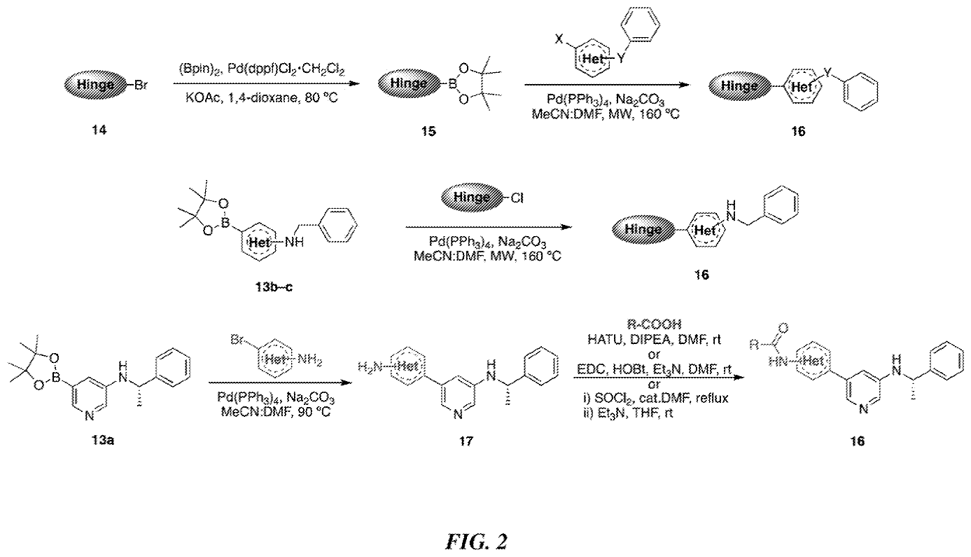

Compounds referred to herein in the examples are referenced by names and also numbers (e.g., 1). Structures corresponding to the numbers are identified in FIGS. 1-2, and in Table 1. For example, compound (1) is shown in FIG. 1.

### EXAMPLES

### Materials and Methods

All reactions involving air-sensitive reagents were carried out with magnetic stirring and in oven-dried glassware with rubber septa under argon unless otherwise stated. All commercially available chemicals and reagent grade solvents were used without further purification unless otherwise specified. Thin-layer chromatography (TLC) was performed on Baker-Flex® silica gel plates (IB2-F) using UV-light (254 and 365 nm) detection or visualizing agents (ninhydrin or phosphomolybdic acid stain) and flash chromatography was carried out on silica gel (230-400 mesh) using Teledyne Isco CombiFlash® Rf. NMR spectra were acquired at room temperature using a Bruker spectrometer at 600, 500, and 400 MHz for ¹H. Chemical shifts (δ) are given in parts per million (ppm) with reference to solvent signals [¹H-NMR: CDCl₃ (7.26 ppm), CD₃OD (3.30 ppm), DMSO-d₆ (2.49 ppm). Signal patterns are reported as s (singlet), d (doublet), t (triplet), q (quartet), quin (quintet), sex (sextet), sep (septet), m (multiplet), br (broad), dd (doublet of doublets), dt (doublet of triplets), td (triplet of doublets), and tt (triplet of triplets). Coupling constants (J) are given in Hz. LCMS analysis was conducted on an Agilent Technologies G1969A high-resolution API-TOF mass spectrometer attached to an Agilent Technologies 1200 HPLC system. Samples were ionized by electrospray ionization (ESI) in positive mode and reported as m z (relative intensity) for the molecular ion [M].

### Example 1—Synthesis of Pyridine and Pyrimidin Based DYRK1A Inhibitor Starting Materials

### 5-Bromo-N-phenylpyridin-3-amine (Compound 2)

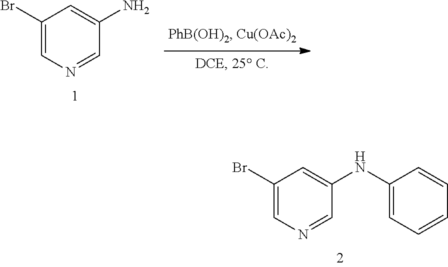 [SMILES: Nc1cncc(Br)c1 ; Brc1cncc(Nc2ccccc2)c1]

Compound 1 (100.0 mg, 0.58 mmol), phenylboronic acid (77.5 mg, 0.64 mmol), and copper (II) acetate (10.5 mg, 0.06 mmol) were dissolved in anhydrous 1,2-dichloroethane (4 mL). The reaction was stirred at room temperature for 1 h in opened-vial for 16 h then the second portion of phenylboronic acid (77.5 mg, 0.64 mmol) was added. The reaction mixture was stirred at room temperature for another 24 h at room temperature. The reaction mixture was filtered through a celite pad. Water was added and the aqueous layer was extracted with EtOAc (2×10 mL). The combined organic extracts were washed with brine, dried over anhydrous Na₂SO₄, filtered, and concentrated. The residue was purified by column chromatography on silica gel (EtOAc/hexane, 20:80 to 30:70) to afford compound 2 (64.4 mg, 45%) as a yellow solid; ¹H NMR (CDCl₃, 600 MHz) 8.27 (1H, s), 8.19 (1H, s), 7.55 (1H, s), 7.37 (2H, t, J=7.3 Hz), 7.14 (2H, d, J=7.3 Hz), 7.10 (1H, t, J=7.3 Hz), 5.84 (1H, br); HRMS (ESI-TOF) m/z: [M+H]⁺ calculated for C₁₁H₁₀⁷⁹BrN₂ and C₁₁H₁₀⁸¹BrN₂ 249.0022, 251.0002; found 249.0013, 250.9993.

### 5-Iodo-N-phenylpyrimidin-2-amine (Compound 4)

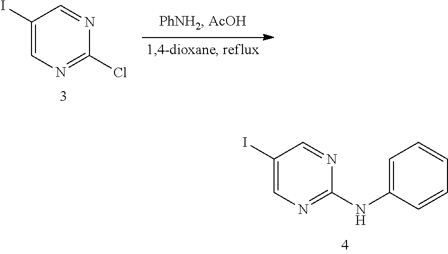 [SMILES: Clc1ncc(I)cn1 ; Ic1cnc(Nc2ccccc2)nc1]

To a solution of compound 3 (100.0 mg, 0.42 mmol) and aniline (0.06 mL, 0.64 mmol) in 1,4-dioxane (2 mL) was added acetic acid (2 mL). The resulting mixture was refluxed for 24 h. After being quenched by the addition of water, the aqueous layer was extracted with EtOAc (2×20 mL). The combined organic extracts were washed with brine, dried over anhydrous Na₂SO₄, filtered, and concentrated. The residue was purified by column chromatography on silica gel (EtOAc/hexane, 10:90) to afford compound 4 (110 mg, 89%) as a brown solid; ¹H NMR (CDCl₃, 500 MHz) 8.53 (2H, s), 7.56 (2H, d, J=7.6 Hz), 7.36 (2H, t, J=7.6 Hz), 7.25 (1H, br), 7.08 (1H, t, J=7.6 Hz); HRMS (ESI-TOF) m/z: [M+H]⁺ calculated for C₁₀H₉IN₃ 297.9836; found 297.9832.

### N-Benzyl-5-bromopyridin-3-amine (Compound 6a)

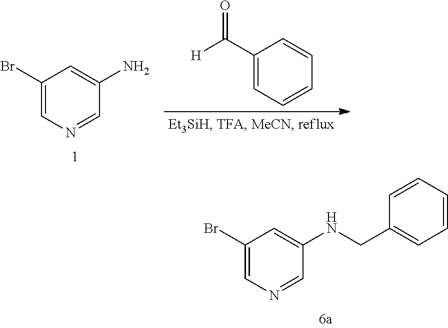 [SMILES: Nc1cncc(Br)c1 ; O=Cc1ccccc1 ; Brc1cncc(NCc2ccccc2)c1]

To a solution of 5-bromopyridin-3-amine (compound 1) (330.0 mg, 1.91 mmol) in anhydrous acetonitrile (10 mL) was added benzaldehyde (0.19 mL, 1.91 mmol), triethylsilane (1.62 mL, 10.11 mmol), and trifluoroacetic acid (0.82 mL, 10.68 mmol) under nitrogen. The resulting mixture was refluxed for 16 h. After being quenched with sat. NaHCO₃ (5 mL), water and EtOAc were added. The layers were separated. The combined organic phases were washed with brine, dried over anhydrous Na₂SO₄, filtered, and concentrated. The residue was purified by column chromatography on silica gel (EtOAc/hexane, 40:60) to afford compound 6a (377.5 mg, 75%) as a yellow solid; ¹H NMR (CDCl₃, 600 MHz) 7.96 (1H, d, J=2.4 Hz), 7.93 (1H, d, J=2.4 Hz), 7.37-7.29 (5H, m), 7.00 (1H, d, J=2.4 Hz), 4.49 (1H, br), 4.29 (2H, s); HRMS (ESI-TOF) m/z: [M+H]⁺ calculated for C₁₂H₁₂⁷⁹BrN₂ and C₁₂H₁₂⁸¹BrN₂ 263.0178, 265.0158; found 263.0156, 265.0138.

### 5-Bromo-N-(2-fluorobenzyl)pyridin-3-amine (Compound 6b)

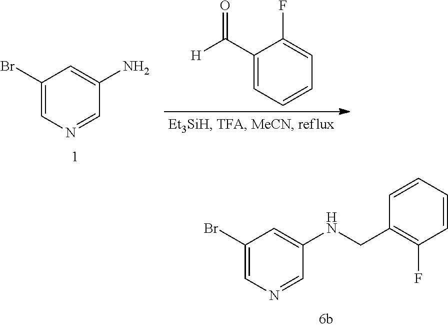 [SMILES: Nc1cncc(Br)c1 ; O=Cc1ccccc1F ; Fc1ccccc1CNc1cncc(Br)c1]

The title compound was prepared from compound 1 and 2-fluorobenzaldehyde following method for preparation of compound 6a and purified by column chromatography on silica gel (EtOAc/hexane, 20:80 to 30:70) to give compound 6b (98%) as a yellow oil; 1H NMR (CDCl₃, 600 MHz) 8.00 (1H, s), 7.97 (1H, d, J=2.4 Hz), 7.34-7.27 (2H, m), 7.13 (1H, t, J=7.3 Hz), 7.09 (1H, t, J=8.5 Hz), 7.04 (1H, s), 7.34 (1H, t, J=7.3 Hz), 4.39 (2H, d, J=4.9 Hz), 4.25 (1H, br); HRMS (ESI-TOF) m/z: [M+H]⁺ calculated for C₁₂H₁₁⁷⁹BrFN₂ and C₁₂H₁₁⁸¹BrFN₂ 281.0064, 283.0064; found 281.0428, 283.0411.

### 5-Bromo-N-(2-chlorobenzyl)pyridin-3-amine (Compound 6c)

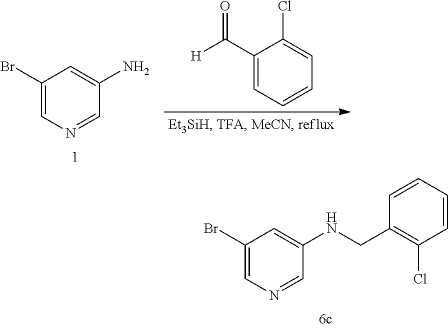 [SMILES: Nc1cncc(Br)c1 ; O=Cc1ccccc1Cl ; Clc1ccccc1CNc1cncc(Br)c1]

The title compound was prepared from compound 1 and 2-chlorobenzaldehyde following method for preparation of compound 6a and purified by column chromatography on silica gel (EtOAc/hexane, 20:80 to 30:70) to give compound 6c (96%) as a yellow solid; 1H NMR (CDCl₃, 600 MHz) 7.99 (1H, s), 7.95 (1H, s), 7.40 (1H, t, J=3.7 Hz), 7.34 (1H, t, J=3.7 Hz), 7.25-7.24 (2H, m), 7.00 (1H, s), 6.99 (2H, s), 4.42 (3H, s); HRMS (ESI-TOF) m/z: [M+H]⁺ calculated for C₁₂H₁₁⁷⁹BrClN₂ and C₁₂H₁₁⁸¹BrClN₂ 296.9789, 298.9760; found 297.0093, 299.0080.

### N-Benzyl-5-iodopyrimidin-2-amine (Compound 6d)

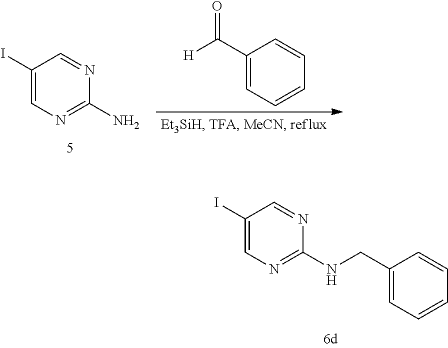 [SMILES: Nc1ncc(I)cn1 ; O=Cc1ccccc1 ; Ic1cnc(NCc2ccccc2)nc1]

The title compound was prepared from compound 5 and benzaldehyde following method for preparation of compound 6a and purified by column chromatography on silica gel (EtOAc/hexane, 10:90) to afford compound 6d (119.4 mg, 80%) as a white solid; 1H NMR (CDCl₃, 600 MHz) 8.25 (2H, s), 7.36-7.32 (4H, m), 7.31-7.28 (1H, m), 6.08 (1H, br), 4.58 (2H, d, J=6.1 Hz); HRMS (ESI-TOF) m/z: [M+H]⁺ calculated for C₁₁H₁₁IN₃ 311.9992; found 311.9981.

### N-(2-Fluorobenzyl)-5-iodopyrimidin-2-amine (Compound 6e)

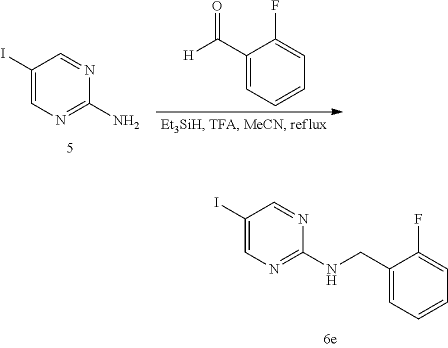 [SMILES: Nc1ncc(I)cn1 ; O=Cc1ccccc1F ; Fc1ccccc1CNc1ncc(I)cn1]

The title compound was prepared from compound 5 and 2-fluorobenzaldehyde following method for preparation of compound 6a and purified by column chromatography on silica gel (EtOAc/hexane, 10:90) to give compound 6e (90%) as a white solid; ¹H NMR (CDCl₃, 600 MHz) 8.36 (2H, s), 7.36 (1H, t, J=7.3 Hz), 7.28-7.24 (1H, m), 7.10-7.04 (2H, m), 5.74 (1H, br), 4.65 (2H, d, J=4.9 Hz); HRMS (ESI-TOF) m/z: [M+H]⁺ calculated for C₁₁H₁₀FIN₃ 329.9898; found 329.9903.

### N-(3-Fluorobenzyl)-5-iodopyrimidin-2-amine (Compound 6f)

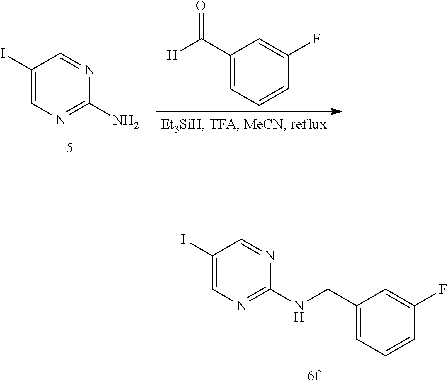 [SMILES: Nc1ncc(I)cn1 ; O=Cc1cccc(F)c1 ; Fc1cccc(CNc2ncc(I)cn2)c1]

The title compound was prepared from compound 5 and 3-fluorobenzaldehyde following method for preparation of compound 6a and purified by column chromatography on silica gel (EtOAc/hexane, 10:90 to 20:80) to give compound 6f (73%) as a white solid; 1H NMR (CDCl₃, 600 MHz) 8.38 (2H, s), 7.29 (1H, q, J=6.1 Hz), 7.10 (1H, d, J=7.3 Hz), 7.03 (1H, d, J=9.8 Hz), 6.96 (1H, td, J=8.5, 2.4 Hz), 5.62 (1H, br), 4.60 (2H, d, J=6.1 Hz); HRMS (ESI-TOF) m/z: [M+H]⁺ calculated for C₁₁H₁₀FIN₃ 329.9898; found 329.9891.

### N-(4-Fluorobenzyl)-5-iodopyrimidin-2-amine (Compound 6g)

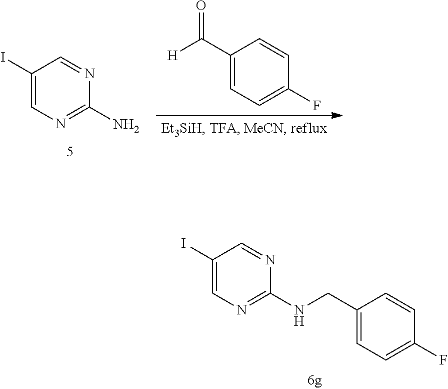 [SMILES: Nc1ncc(I)cn1 ; O=Cc1ccc(F)cc1 ; Fc1ccc(CNc2ncc(I)cn2)cc1]

The title compound was prepared from compound 5 and 4-fluorobenzaldehyde following method for preparation of compound 6a and purified by column chromatography on silica gel (EtOAc/hexane, 10:90 to 15:85) to give compound 6g (91%) as a pale yellow solid; ¹H NMR (CDCl₃, 600 MHz) 8.36 (2H, s), 7.30 (2H, dd, J=8.5, 6.1 Hz), 7.02 (2H, t, J=8.5 Hz), 5.67 (1H, br), 4.56 (2H, d, J=6.1 Hz); HRMS (ESI-TOF) m/z: [M+H]⁺ calculated for C₁₁H₁₀FIN₃ 329.9898; found 329.9903.

### N-(2-Chlorobenzyl)-5-iodopyrimidin-2-amine (Compound 6h)

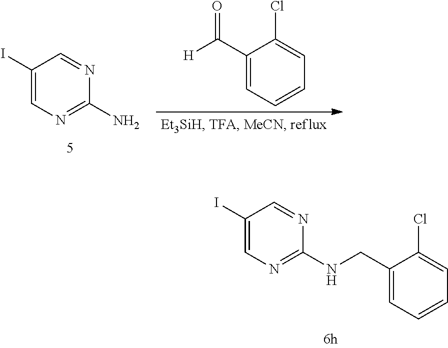 [SMILES: Nc1ncc(I)cn1 ; O=Cc1ccccc1Cl ; Clc1ccccc1CNc1ncc(I)cn1]

The title compound was prepared from compound 5 and 2-chlorobenzaldehyde following method for preparation of compound 6a and purified by column chromatography on silica gel (EtOAc/hexane, 10:90) to give compound 6h (88%) as a white solid; ¹H NMR (CDCl₃, 600 MHz) 8.34 (2H, s), 7.40-7.37 (2H, m), 7.23-7.22 (2H, m), 7.03 (1H, d, J=9.8 Hz), 5.94 (1H, br), 4.68 (2H, d, J=4.9 Hz); HRMS (ESI-TOF) m/z: [M+H]⁺ calculated for C₁₁H₁₀ClIN₃ 345.9602; found 345.9594.

### N-(3-Chlorobenzyl)-5-iodopyrimidin-2-amine (Compound 6i)

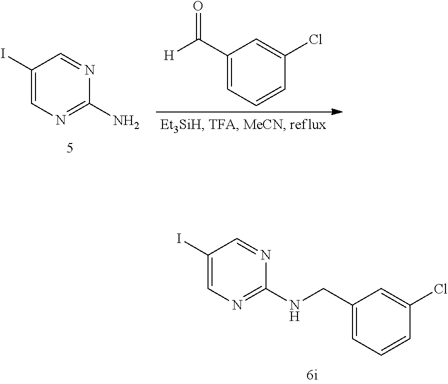 [SMILES: Nc1ncc(I)cn1 ; O=Cc1cccc(Cl)c1 ; Clc1cccc(CNc2ncc(I)cn2)c1]

The title compound was prepared from compound 5 and 3-chlorobenzaldehyde following method for preparation of compound 6a and purified by column chromatography on silica gel (EtOAc/hexane, 10:90 to 20:80) to give compound 6i (89%) as a white solid; ¹H NMR (CDCl₃, 600 MHz) 8.38 (2H, s), 7.38-7.25 (3H, m), 7.20 (1H, d, J=6.1 Hz), 5.70 (1H, br), 4.58 (2H, d, J=6.1 Hz); HRMS (ESI-TOF) m/z: [M+H]⁺ calculated for C₁₁H₁₀ClIN₃ 345.9602; found 345.9619.

### N-(4-Chlorobenzyl)-5-iodopyrimidin-2-amine (Compound 6j)

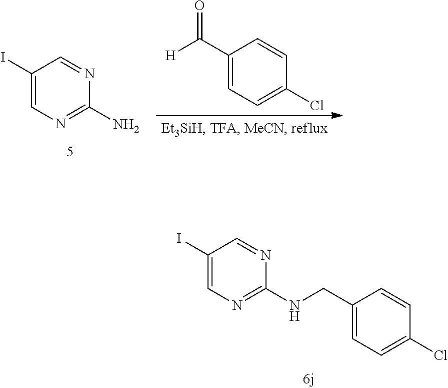 [SMILES: Nc1ncc(I)cn1 ; O=Cc1ccc(Cl)cc1 ; Clc1ccc(CNc2ncc(I)cn2)cc1]

The title compound was prepared from compound 5 and 4-chlorobenzaldehyde following method for preparation of compound 6a and purified by column chromatography on silica gel (EtOAc/hexane, 10:90) to give compound 6j (86%) as a white solid; ¹H NMR (CDCl₃, 600 MHz) 8.36 (2H, s), 7.30 (2H, d, J=8.5 Hz), 7.26 (2H, d, J=8.5 Hz), 5.71 (1H, br), 4.56 (2H, d, J=6.1 Hz); HRMS (ESI-TOF) m/z: [M+H]⁺ calculated for C₁₁H₁₀ClIN₃ 345.9602; found 345.9657.

### 5-Iodo-N-(3-methoxybenzyl)pyrimidin-2-amine (Compound 6k)

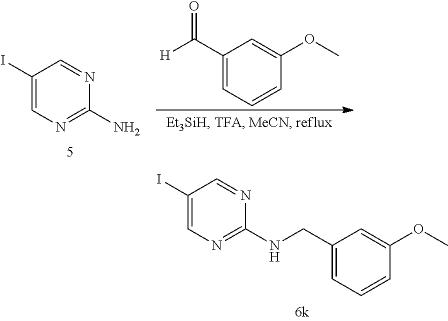

The title compound was prepared from compound 5 and 3-methoxybenzaldehyde following method for preparation of compound 6a and purified by column chromatography on silica gel (EtOAc/hexane, 30:70 to 40:60) to give compound 6k (70%) as a white solid; 1H NMR (CDCl₃, 600 MHz) 8.39 (2H, s), 7.25 (1H, d, J=7.3 Hz), 6.91 (1H, d, J=7.3 Hz), 6.87 (1H, s), 6.82 (1H, d, J=6.1 Hz), 5.47 (1H, br), 4.57 (2H, d, J=6.1 Hz), 3.80 (3H, s); HRMS (ESI-TOF) m/z: [M+H]⁺ calculated for C₁₂H₁₃IN₃O 342.0098; found 342.0322.

### 5-Iodo-N-(4-methoxybenzyl)pyrimidin-2-amine (Compound 61)

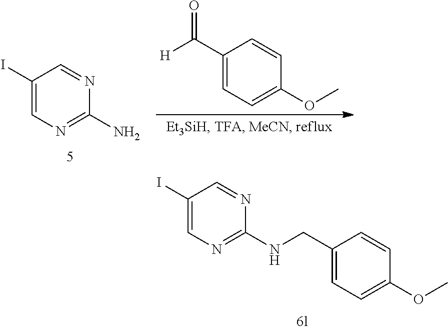

The title compound was prepared from compound 5 and 4-methoxybenzaldehyde following method for preparation of compound 6a and purified by column chromatography on silica gel (EtOAc/hexane, 30:70) to give compound 61 (83%) as a white solid; 1H NMR (CDCl₃, 600 MHz) 8.33 (2H, br), 7.26 (2H, d, J=7.3 Hz), 6.88 (2H, d, J=8.5 Hz), 5.83 (1H, br), 4.50 (2H, d, J=6.1 Hz), 3.81 (3H, s); HRMS (ESI-TOF) m/z: [M+H]⁺ calculated for C₁₂H₁₃IN₃O 342.0098; found 342.0271.

### 5-Iodo-N-(4-(trifluoromethoxy)benzyl)pyrimidin-2-amine (Compound 6m)

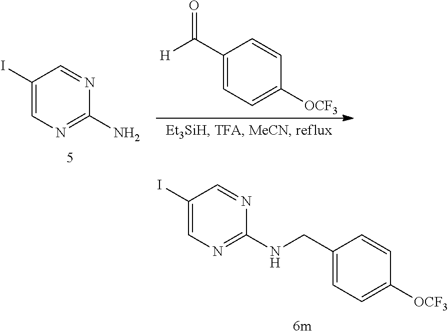 [SMILES: Nc1ncc(I)cn1 ; O=Cc1ccc(OC(F)(F)F)cc1 ; FC(F)(F)Oc1ccc(CNc2ncc(I)cn2)cc1]

The title compound was prepared from compound 5 and 4-trifluoromethoxybenzaldehyde following method for preparation of compound 6a and purified by column chromatography on silica gel (EtOAc/hexane, 20:80) to give compound 6m (80%) as a white solid; 1H NMR (CDCl₃, 500 MHz) 8.40 (2H, s), 7.35 (2H, d, J=8.6 Hz), 7.18 (2H, d, J=8.1 Hz), 5.47 (1H, br), 4.61 (2H, d, J=6.1 Hz); HRMS (ESI-TOF) m/z: [M+H]⁺ calculated for C₁₂H₁₀F₃IN₃O 395.9815; found 395.9840.

### 5-Iodo-N-(2-(trifluoromethyl)benzyl)pyrimidin-2-amine (Compound 6n)

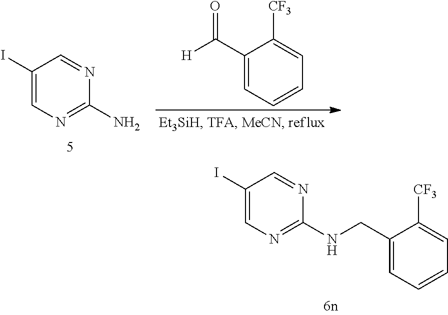 [SMILES: Nc1ncc(I)cn1 ; O=Cc1ccccc1C(F)(F)F ; FC(F)(F)c1ccccc1CNc1ncc(I)cn1]

The title compound was prepared from compound 5 and 2-trifluoromethylbenzaldehyde following method for preparation of compound 6a and purified by column chromatography on silica gel (EtOAc/hexane, 20:80) to give compound 6n (82%) as a white solid; 1H NMR (CDCl₃, 500 MHz) 8.40 (2H, s), 7.67 (1H, d, J=7.6 Hz), 7.58 (1H, d, J=7.6 Hz), 7.50 (1H, t, J=7.6 Hz), 7.38 (1H, t, J=7.6 Hz), 5.49 (1H, br), 4.82 (2H, d, J=6.6 Hz); HRMS (ESI-TOF) m/z: [M+H]⁺ calculated for C₁₂H₁₀F₃IN₃ 379.9866; found 379.9858.

### 5-Iodo-N-(3-(trifluoromethyl)benzyl)pyrimidin-2-amine (Compound 6o)

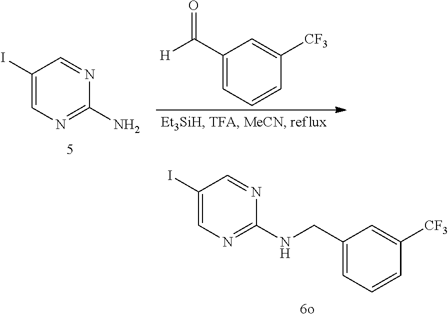 [SMILES: Nc1ncc(I)cn1 ; O=Cc1cccc(C(F)(F)F)c1 ; FC(F)(F)c1cccc(CNc2ncc(I)cn2)c1]

The title compound was prepared from compound 5 and 3-trifluoromethylbenzaldehyde following method for preparation of compound 6a and purified by column chromatography on silica gel (EtOAc/hexane, 20:80) to give compound 6o (94%) as a white solid; ¹H NMR (CDCl₃, 500 MHz) 8.34 (2H, s), 7.59 (1H, s), 7.55-7.51 (2H, m), 7.45 (1H, t, J=7.6 Hz), 5.85 (1H, br), 4.66 (2H, d, J=6.1 Hz); HRMS (ESI-TOF) m/z: [M+H]⁺ calculated for C₁₂H₁₀F₃IN₃ 379.9866; found 379.9871.

### 5-Iodo-N-(4-(trifluoromethyl)benzyl)pyrimidin-2-amine (Compound 6p)

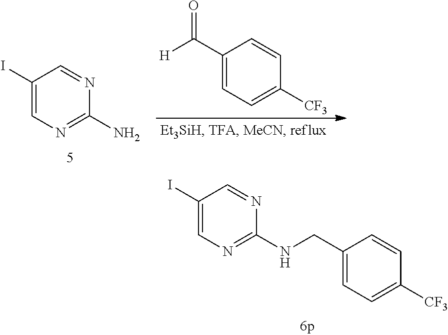 [SMILES: Nc1ncc(I)cn1 ; O=Cc1ccc(C(F)(F)F)cc1 ; FC(F)(F)c1ccc(CNc2ncc(I)cn2)cc1]

The title compound was prepared from compound 5 and 4-trifluoromethylbenzaldehyde following method for preparation of compound 6a and purified by column chromatography on silica gel (EtOAc/hexane, 20:80) to give compound 6p (80%) as a white solid; ¹H NMR (CDCl₃, 500 MHz) 8.40 (2H, s), 7.58 (2H, d, J=8.1 Hz), 7.44 (2H, d, J=8.1 Hz), 5.54 (1H, br), 4.67 (2H, d, J=6.1 Hz); HRMS (ESI-TOF) m/z: [M+H]⁺ calculated for C₁₂H₁₀F₃IN₃ 379.9866; found 379.9882.

### N-((5-Bromopyridin-3-yl)methyl)aniline (Compound 8a)

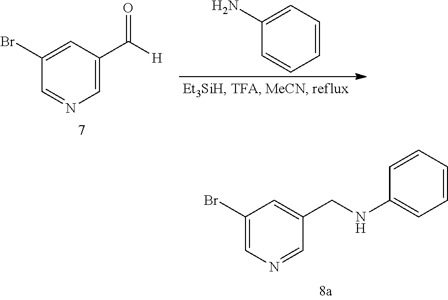 [SMILES: O=Cc1cncc(Br)c1 ; Nc1ccccc1 ; Brc1cncc(CNc2ccccc2)c1]

The title compound was prepared from compound 7 and aniline following method for preparation of compound 6a and purified by column chromatography on silica gel (EtOAc/hexane, 20:80) to give compound 8a (86%) as a yellow oil; ¹H NMR (CDCl₃, 600 MHz) 8.56 (1H, d, J=2.4 Hz), 8.54 (1H, s), 7.86 (1H, s), 7.19 (2H, t, J=7.3 Hz), 6.76 (1H, t, J=7.3 Hz), 6.01 (2H, d, J=7.3 Hz), 4.37 (2H, d, J=4.9 Hz), 4.12 (1H, Br); HRMS (ESI-TOF) m/z: [M+H]⁺ calculated for C₁₂H₁₂⁷⁹Br₂ and C₁₂H₁₂⁸¹Br₂ 263.0179, 265.0158; found 263.0362, 265.0345.

### N-((5-Bromopyridin-3-yl)methyl)-4-chloroaniline (Compound 8b)

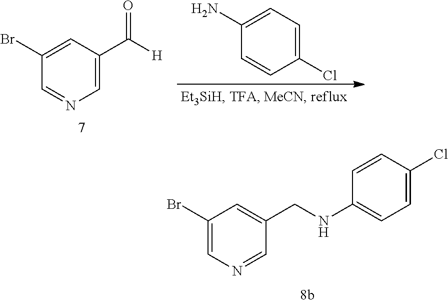 [SMILES: O=Cc1cncc(Br)c1 ; Nc1ccc(Cl)cc1 ; Clc1ccc(NCc2cncc(Br)c2)cc1]

The title compound was prepared from compound 7 and 4-chloroaniline following method for preparation of compound 6a and purified by column chromatography on silica gel (EtOAC/hexane, 20:80 to 30:70) to give compound 8b (82%) as a yellow solid; ¹H NMR (CDCl₃, 500 MHz) 8.60 (1H, d, J=2.0 Hz), 8.53 (1H, s), 7.83 (1H, s), 7.13 (2H, d, J=8.6 Hz), 6.53 (2H, d, J=8.6 Hz), 4.34 (2H, d, J=5.6 Hz), 4.12 (1H, br); HRMS (ESI-TOF) m/z: [M+H]⁺ calculated for C₁₂H₁₁⁷⁹BrClN₂ and C₁₂H₁₁⁸¹BrClN₂ 296.9789, 298.9769; found 293.0273, 295.0256.

### N-((5-Bromopyrimidin-2-yl)methyl)aniline (Compound 10)

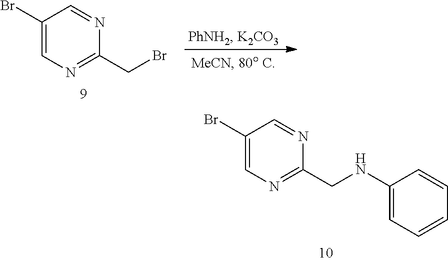 [SMILES: BrCc1ncc(Br)cn1 ; Brc1cnc(CNc2ccccc2)nc1]

To a mixture of compound 9 (130.0 mg, 0.52 mmol) and K₂CO₃ (143.0 mg, 1.03 mmol) in anhydrous acetonitrile (1 mL) was added aniline (0.05 mL, 0.52 mmol) under nitrogen. The reaction mixture was stirred at 80° C. for 4 h. After being quenched by the addition of water, the aqueous layer was extracted with EtOAc (2×15 mL). The combined organic extracts were washed with brine, dried over anhydrous Na₂SO₄, filtered, and concentrated. The residue was purified by column chromatography on silica gel (EtOAc/hexane, 20:80 to 30:70) to give compound 10 (81.3 mg, 60%) as a yellow solid; ¹H NMR (CDCl₃, 600 MHz) 8.78 (2H, s), 7.20 (2H, t, J=7.3 Hz), 6.76-6.71 (3H, m), 4.94 (1H, br), 4.54 (2H, s); HRMS (ESI-TOF) m/z: [M+H]⁺ calculated for C₁₁H₁₁⁷⁹BrN₃ and C₁₁H₁₁⁸¹BrN₃ 264.0131, 266.0111; found 264.0117, 266.0096.

### (S)-5-Chloro-N-(1-phenylethyl)pyridin-3-amine (Compound 12)

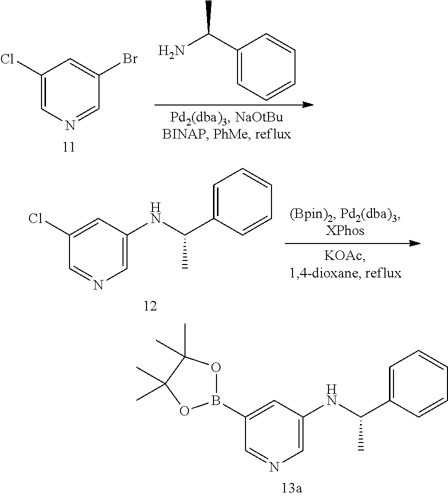

To a solution of compound 11 (1000 mg, 5.20 mmol), Pd₂(dba)₃ (95.2 mg, 0.10 mmol), BINAP (129.4 mg, 0.21 mmol), and Na^{t}OBu (749.1 mg, 7.80 mmol) in anhydrous toluene (40 mL) was added (S)-(−)-1-phenylethylamine (0.84 mL, 6.50 mmol). The resulting mixture was purged with nitrogen and stirred at 110° C. for 16 h. After being quenched with water (20 mL), the aqueous layer was extracted with EtOAc (3×40 mL). The combined organic extracts were washed with brine, dried over anhydrous Na₂SO₄, filtered, and concentrated. The residue was and purified by column chromatography on silica gel (EtOAc/hexane, 20:80 to 30:70) to give compound 12 (919.5 mg, 76%) as a light brown solid; ¹H NMR (CDCl₃, 500 MHz) 7.84 (1H, d, J=2.5 Hz), 7.83 (1H, d, J=2.0 Hz), 7.35-7.31 (4H, m), 7.27-7.24 (1H, m), 6.70 (1H, t, J=2.0 Hz), 4.45 (1H, quin, J=6.1 Hz), 4.26 (1H, br), 1.54 (3H, d, J=7.1 Hz); HRMS (ESI-TOF) m/z: [M+H]⁺ calculated for C₁₃H₁₄ClN₂ 233.0841; found 233.1067.

### (S)—N-(1-Phenylethyl)-5-(4,4,5,5-tetramethyl-1,3,2-dioxaborolan-2-yl)pyridin-3-amine (Compound 13a)

To a solution of compound 12 (353 mg, 1.52 mmol), bis(pinacolato)diboron (770 mg, 3.03 mmol), Pd₂(dba)₃ (83.3 mg, 0.09 mmol) and XPhos (86.8 mg, 0.18 mmol) in 1,4-dioxane (15 mL) was added KOAc (447 mg, 4.55 mmol) under nitrogen. The resulting mixture was stirred at 100° C. for 20 h. The reaction was allowed to cool to room temperature and filtered through a celite pad. The solvent was removed in vacuo. The product (compound 13a) was used in next step without further purification; HRMS (ESI-TOF) m/z: [M−C₆H₉]⁺ calculated for C₁₃H₁₆BN₂O₂ 243.1299; found 243.1271.

### N-Benzyl-5-(4,4,5,5-tetramethyl-1,3,2-dioxaborolan-2-yl)pyridin-3-amine (Compound 13b)

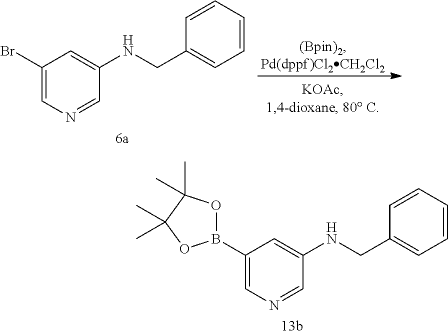 [SMILES: Brc1cncc(NCc2ccccc2)c1 ; CC1(C)OB(c2cncc(NCc3ccccc3)c2)OC1(C)C]

To a solution of compound 6a (35 mg, 0.13 mmol), bis(pinacolato)diboron (51 mg, 0.20 mmol), and Pd(dppf)Cl₂·CH₂Cl₂ (12 mg, 0.02 mmol) in 1,4-dioxane (2 mL) was added KOAc (39 mg, 0.4 mmol) under nitrogen. The resulting mixture was stirred at 80° C. for 16 h. The reaction was allowed to cool to room temperature and filtered through a celite pad. The solvent was removed in vacuo. The product (compound 13b) was used in next step without further purification; HRMS (ESI-TOF) m/z: [M−C₆H₉]⁺ calculated for C₁₂H₁₄BN₂O₂ 229.1143; found 229.1621.

### N-Benzyl-5-(4,4,5,5-tetramethyl-1,3,2-dioxaborolan-2-yl)pyrimidin-2-amine (Compound 13c)

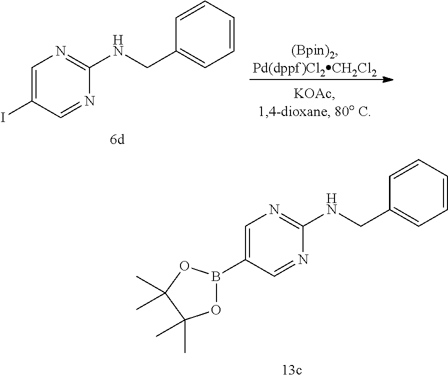 [SMILES: Ic1cnc(NCc2ccccc2)nc1 ; CC1(C)OB(c2cnc(NCc3ccccc3)nc2)OC1(C)C]

The title compound was prepared from compound 6d following method for preparation of compound 13b. The product (compound 13c) was used in next step without further purification; HRMS (ESI-TOF) m/z: [M−C₆H₉]⁺ calculated for C₁₁H₁₃BN₃O₂ 230.1095; found 230.1200.

### 6-(4,4,5,5-Tetramethyl-1,3,2-dioxaborolan-2-yl)imidazo[1,2-a]pyridine (Compound 15a)

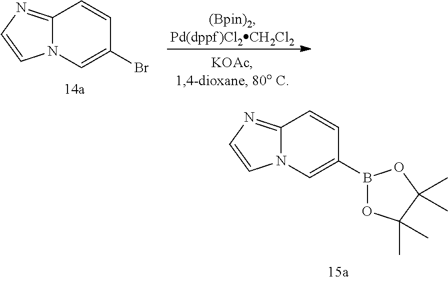 [SMILES: Brc1ccc2nccn2c1 ; CC1(C)OB(c2ccc3nccn3c2)OC1(C)C]

The title compound was prepared from compound 14a following method for preparation of compound 13b. The product (compound 15a) was used in next step without further purification; HRMS (ESI-TOF) m/z: [M−C₆H₉]⁺ calculated for C₇H₈BN₂O₂ 163.0673; found 163.0679.

### 6-(4,4,5,5-Tetramethyl-1,3,2-dioxaborolan-2-yl)-1H-benzo[d]imidazol-2-amine (Compound 15b)

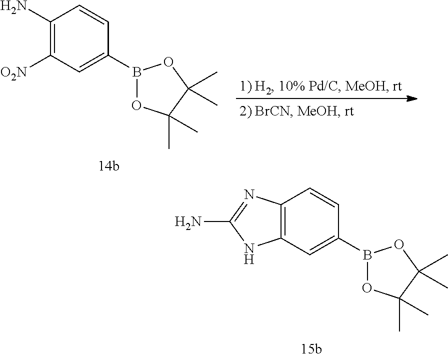 [SMILES: CC1(C)OB(c2ccc(N)c([N+](=O)[O-])c2)OC1(C)C ; CC1(C)OB(c2ccc3nc(N)[nH]c3c2)OC1(C)C]

To a solution of compound 14b (100 mg, 0.38 mmol) in MeOH (10 mL) was added 10% Pd/C (10 mg). The resulting mixture was purged with hydrogen gas and stirred under hydrogen atmosphere at room temperature for 16 h. The reaction was filtered through a celite pad and solvent was removed in vacuo. The crude product from the first step was dissolved in MeOH (5 mL) then cyanogen bromide (40.1 mg, 0.38 mmol) was added. The reaction mixture was stirred at room temperature for 3 h. After being quenched with water (10 mL), the aqueous layer was extracted with EtOAc (2×10 mL). The combined organic extracts were washed with brine, dried over anhydrous Na₂SO₄, filtered, and concentrated. The residue was and purified by column chromatography on silica gel (MeOH/CH₂Cl₂, 10:90 to 15:85) to give compound 15b (36.4 mg, 37%) as a brown solid; HRMS (ESI-TOF) m/z: [M+H]⁺ calculated for C₁₃H₁₉BN₃O₂ 260.1565; found 260.1575.

### 5-(4,4,5,5-Tetramethyl-1,3,2-dioxaborolan-2-yl)-1H-indazol-3-amine (Compound 15c)

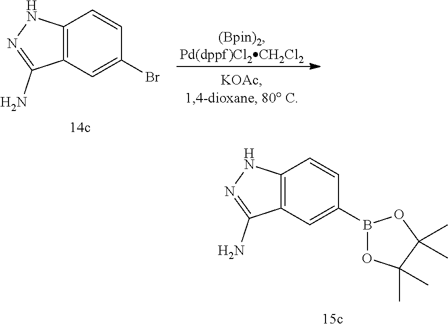 [SMILES: Nc1n[nH]c2ccc(Br)cc12 ; CC1(C)OB(c2ccc3[nH]nc(N)c3c2)OC1(C)C]

The title compound was prepared from compound 14c following method for preparation of compound 13b and purified by column chromatography on silica gel (EtOAc/hexane, 50:50 to 80:20) to give 15c (48%) as a brown solid; ¹H NMR (DMSO-d₆, 600 MHz) 11.47 (1H, s), 8.15 (1H, s), 7.47 (1H, d, J=8.5 Hz), 7.16 (1H, d, J=7.3 Hz), 5.46 (2H, br), 1.29 (12H, s); HRMS (ESI-TOF) m/z: [M+H]⁺ calculated for C₁₃H₁₉BN₃O₂ 260.1565; found 260.1592.

### 6-(4,4,5,5-Tetramethyl-1,3,2-dioxaborolan-2-yl)-1H-indazol-3-amine (Compound 15d)

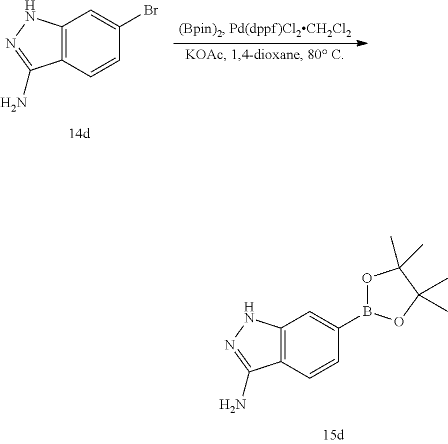 [SMILES: Nc1n[nH]c2cc(Br)ccc12 ; CC1(C)OB(c2ccc3c(N)n[nH]c3c2)OC1(C)C]

The title compound was prepared from compound 14d following method for preparation of compound 13b and purified by column chromatography on silica gel (EtOAc/hexane, 60:40 to 80:20) to give compound 15d (73%) as a brown solid; ¹H NMR (CDCl₃, 500 MHz) 7.81 (1H, s), 7.56 (1H, d, J=8.1 Hz), 7.48 (1H, d, J=8.1 Hz), 1.37 (12H, s); HRMS (ESI-TOF) m/z: [M+H]⁺ calculated for C₁₃H₁₉BN₃O₂ 260.1565; found 260.1678.

### 5-(4,4,5,5-Tetramethyl-1,3,2-dioxaborolan-2-yl)pyridin-2 (1H)-one (Compound 15e)

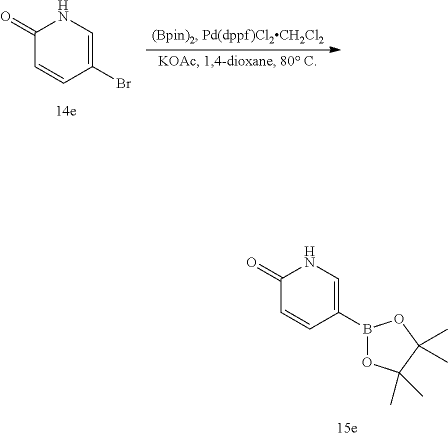 [SMILES: O=c1ccc(Br)c[nH]1 ; CC1(C)OB(c2ccc(=O)[nH]c2)OC1(C)C]

The title compound was prepared from compound 14e following method for preparation of compound 13b. The product (compound 15e) was used in next step without further purification; HRMS (ESI-TOF) m/z: [M+H]⁺ calculated for C₁₁H₁₇BNO₃ 222.1296; found 222.1301.

### 6-(4,4,5,5-Tetramethyl-1,3,2-dioxaborolan-2-yl)quinolin-2 (1H)-one (Compound 15f)

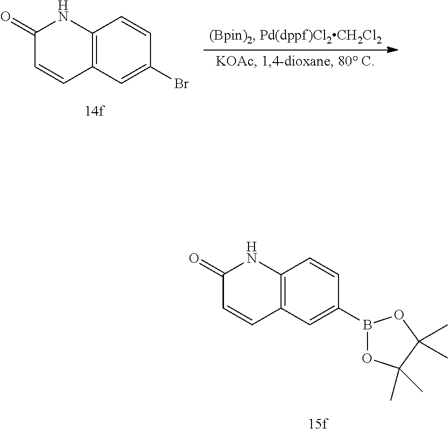 [SMILES: O=c1ccc2cc(Br)ccc2[nH]1 ; CC1(C)OB(c2ccc3[nH]c(=O)ccc3c2)OC1(C)C]

The title compound was prepared from compound 14f following method for preparation of compound 13b and purified by column chromatography on silica gel (EtOAc/hexane, 80:20) to give compound 15f (89%) as a brown solid; 1H NMR (CDCl₃, 500 MHz) 12.59 (1H, br), 8.04 (1H, s), 7.91 (1H, d, J=8.1 Hz), 7.83 (1H, d, J=9.1 Hz), 7.42 (1H, d, J=8.1), 6.71 (1H, d, J=9.7 Hz), 1.36 (12H, s); HRMS (ESI-TOF) m/z: [M+H]⁺ calculated for C₁₅H₁₉BNO₃ 272.1453; found 272.1484.

### 5-(4,4,5,5-Tetramethyl-1,3,2-dioxaborolan-2-yl)quinolin-2 (1H)-one (Compound 15g)

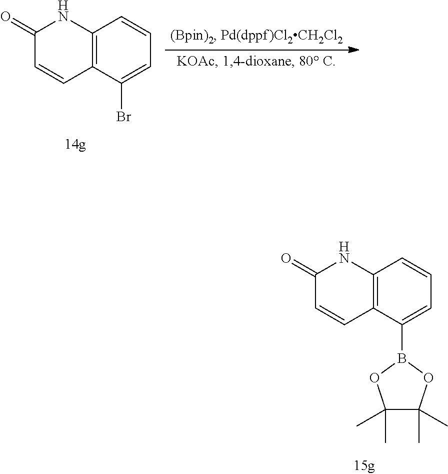 [SMILES: O=c1ccc2c(Br)cccc2[nH]1 ; CC1(C)OB(c2cccc3[nH]c(=O)ccc23)OC1(C)C]

The title compound was prepared from compound 14g following method for preparation of compound 13b and purified by column chromatography on silica gel (EtOAc/hexane, 30:70 to 60:40) to give compound 15g (99%) as a brown solid; 1H NMR (CDCl₃, 500 MHz) 11.97 (1H, s), 8.78 (1H, d, J=9.7 Hz), 7.77 (1H, t, J=4.1 Hz), 7.50 (2H, d, J=4.1 Hz), 6.76 (1H, d, J=9.7 Hz), 1.40 (12H, s); HRMS (ESI-TOF) m/z: [M+H]⁺ calculated for C₁₅H₁₉BNO₃ 272.1453; found 272.1492

### 5-Bromo-N-phenyl-7H-pyrrolo[2,3-d]pyrimidin-2-amine (Compound 14h-1)

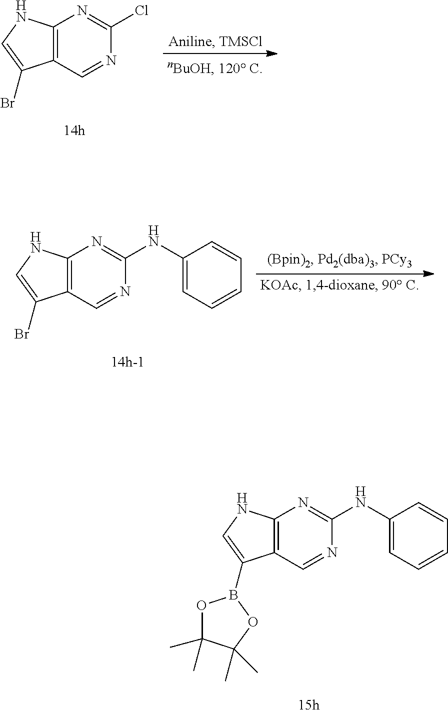 [SMILES: Clc1ncc2c(Br)c[nH]c2n1 ; Brc1c[nH]c2nc(Nc3ccccc3)ncc12 ; CC1(C)OB(c2c[nH]c3nc(Nc4ccccc4)ncc23)OC1(C)C]

To a solution of compound 14h (500 mg, 2.15 mmol) in ⁿBuOH (15 mL) was added aniline (0.4 mL, 4.30 mmol) and TMSCl (0.52 mL, 4.09 mmol). The resulting mixture was purged with nitrogen and stirred at 120° C. for 24 h. After being quenched with water (15 mL), the aqueous layer was extracted with EtOAc (2×20 mL). The combined organic extracts were washed with brine, dried over anhydrous Na₂SO₄, filtered, and concentrated. The residue was and purified by column chromatography on silica gel (EtOAc/hexane, 30:70) to give compound 14h-1 (199.8 mg, 32%) as a yellow solid; ¹H NMR (CD₃OD, 500 MHz) 8.50 (1H, s), 7.72 (2H, d, J=7.6 Hz), 7.26 (2H, t, J=7.6 Hz), 7.13 (1H, s), 6.94 (1H, t, J=7.1 Hz); HRMS (ESI-TOF) m/z: [M+H]⁺ calculated for C₁₂H₁₀⁷⁹BrN₄ and C₁₂H₁₀⁸¹BrN₄ 289.0083, 291.0063; found 289.0096, 291.0076.

### N-Phenyl-5-(4,4,5,5-tetramethyl-1,3,2-dioxaborolan-2-yl)-7H-pyrrolo[2,3-d]pyrimidin-2-amine (Compound 15h)

To a solution of compound 14h-1 (147.7 mg, 0.51 mmol), bis(pinacolato)diboron (155.7 mg, 0.61 mmol), Pd₂(dba)₃ (28.1 mg, 0.03 mmol) and tricyclohexylphosphine (28.6 mg, 0.01 mmol) in 1,4-dioxane (5 mL) was added KOAc (82.7 mg, 0.84 mmol) under nitrogen. The resulting mixture was stirred at 90° C. for 43 h. After being quenched with water (5 mL), the aqueous layer was extracted with EtOAc (2×20 mL). The combined organic extracts were washed with brine, dried over anhydrous Na₂SO₄, filtered, and concentrated. The residue was and purified by column chromatography on silica gel (EtOAc/hexane, 25:75) to give compound 15h (68.2 mg, 40%) as a yellow solid; ¹H NMR (CDCl₃, 500 MHz) 8.90 (1H, s), 7.69 (2H, d, J=7.6 Hz), 7.41 (1H, s), 7.33 (2H, t, J=7.6 Hz), 7.01 (1H, t, J=7.6 Hz), 1.36 (12H, s); HRMS (ESI-TOF) m/z: [M+H]⁺ calculated for C₁₃H₂₂BN₄O₂ 337.1830; found 337.1826.

### N-Methyl-4-(4,4,5,5-tetramethyl-1,3,2-dioxaborolan-2-yl)pyridin-2-amine (Compound 15i)

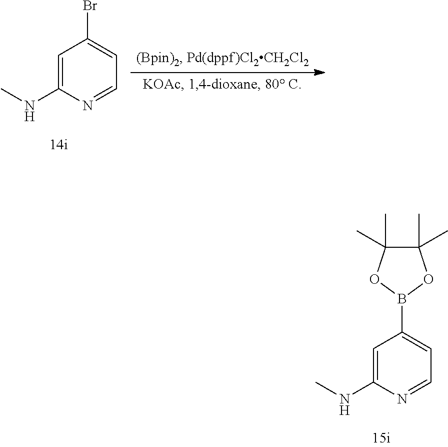

The title compound was prepared from compound 14i following method for preparation of compound 13b. The product (compound 15i) was used in next step without further purification; HRMS (ESI-TOF) m/z: [M−C₆H₉]⁺ calculated for C₆H₁₀BN₂O₂ 153.0830; found 153.0856.

### Di-tert-butyl 6-bromo-1H-indazole-1,3-dicarboxylate (Compound 14j-1)

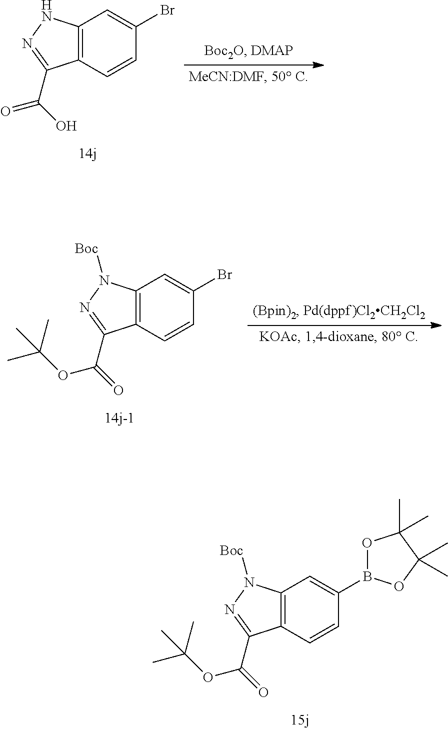 [SMILES: O=C(O)c1n[nH]c2cc(Br)ccc12 ; CC(C)(C)OC(=O)c1nn(C(=O)OC(C)(C)C)c2cc(Br)ccc12 ; CC(C)(C)OC(=O)c1nn(C(=O)OC(C)(C)C)c2cc(B3OC(C)(C)C(C)(C)O3)ccc12]

To a solution of compound 14j (200 mg, 0.83 mmol) in anhydrous MeCN (7 mL) anhydrous DMF (5 mL) was added di-tert-butyl dicarbonate (724 mg, 0.3.32 mmol) and DMAP (20.3 mg, 0.17 mmol) under nitrogen. The resulting mixture was stirred at 50° C. for 16 h. After being quenched with 1 N HCl (2 mL), the aqueous layer was extracted with EtOAc (2×10 mL). The combined organic extracts were washed with brine, dried over anhydrous Na₂SO₄, filtered, and concentrated. The residue was and purified by column chromatography on silica gel (EtOAc/hexane, 15:85) to give a mixture of compound 14j-1 (209.2 mg, 63.5%) as a white solid; ¹H NMR (CDCl₃, 600 MHz) 8.43 (1H, s), 8.04 (1H, d, J=8.5 Hz), 7.50 (1H, d, J=8.5 Hz), 1.72 (9H, s), 1,67 (9H, s); HRMS (ESI-TOF) m/z: [M−C₈H₁₇]⁺ calculated for C₉H₆⁷⁹BrN₂O₄, C₉H₆⁸¹BrN₂O₄ 284.9505, 286.9485; found 284.9512, 286.9489.

### Di-tert-butyl 6-(4,4,5,5-tetramethyl-1,3,2-dioxaborolan-2-yl)-1H-indazole-1,3-dicarboxylate (Compound 15j)

The title compound was prepared from compound 14j-1 following method for preparation of compound 13b and purified by column chromatography on silica gel (EtOAc/hexane, 10:90) to give compound 15j (66%) as a white solid; ¹H NMR (CDCl₃, 600 MHz) 8.70 (1H, s), 8.18 (1H, d, J=7.3 Hz), 7.80 (1H, d, J=7.3 Hz), 1.73 (9H, s), 1.69 (9H, s), 1.36 (12H, s); HRMS (ESI-TOF) m/z: [M+Na]⁺ calculated for C₂₃H₃₃BN₂NaO₆ 467.23.29; found 467.2307.

### (S)-5-(2-Nitrothiazol-5-yl)-N-(1-phenylethyl)pyridin-3-amine (Compound 19)

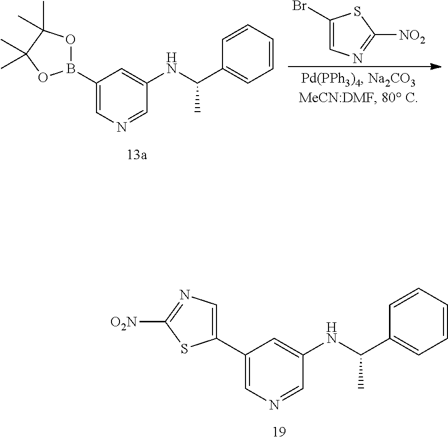 [SMILES: C[C@H](Nc1cncc(B2OC(C)(C)C(C)(C)O2)c1)c1ccccc1 ; O=[N+]([O-])c1ncc(Br)s1 ; C[C@H](Nc1cncc(-c2cnc([N+](=O)[O-])s2)c1)c1ccccc1]

To a solution of compound 13a (70 mg, 0.22 mmol), 5-bromo-2-nitrothiazole (38 mg, 0.18 mmol) and Pd(PPh₃)₄ (21 mg, 0.02 mmol) in anhydrous MeCN (1.5 mL) and anhydrous DMF (1.5 mL) was added 1 M Na₂CO₃ (0.36 mL, 0.36 mmol) under nitrogen. The resulting mixture was stirred at 80° C. for 3 h. After being quenched with water (5 mL), the aqueous layer was extracted with EtOAc (2×10 mL). The combined organic extracts were washed with brine, dried over anhydrous Na₂SO₄, filtered, and concentrated. The residue was and purified by column chromatography on silica gel (EtOAc/hexane, 50:50 to 70:30) to give compound 19 (33.3 mg, 57%) as a brown oil; ¹H NMR (CDCl₃, 400 MHz) 8.13 (1H, br), 8.17 (1H, s), 7.83 (1H, s), 7.39-7.36 (4H, m), 7.30-7.26 (2H, m), 6.81 (1H, s), 4.53 (1H, q, J=6.0 Hz), 4.42 (1H, br), 1.60 (3H, d, J=6.7 Hz); HRMS (ESI-TOF) m/z: [M+H]⁺ calculated for C₁₆H₁₆N₄O₂S 327.0910; found 327.0927.

### 2-((tert-Butoxycarbonyl)(phenyl)amino)acetic acid (Compound 21)

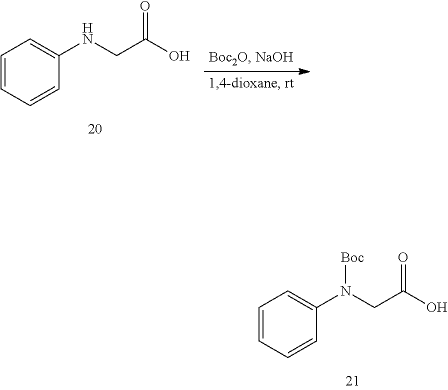 [SMILES: O=C(O)CNc1ccccc1 ; CC(C)(C)OC(=O)N(CC(=O)O)c1ccccc1]

To a suspension of compound 20 (300 mg, 1.98 mmol) in 1,4-dioxane (6 mL) was added 1 N NaOH (2.2 mL, 2.2 mmol) and di-tert-butyl dicarbonate (520 mg, 2.38 mmol) at 0° C. under nitrogen. The reaction was stirred at room temperature for 24 h. After completion of reaction, the reaction was adjusted to pH 5 by addition of 1 N HCl. The aqueous layer was extracted with EtOAc (2×10 mL). The combined organic extracts were washed with brine, dried over anhydrous Na₂SO₄, filtered, and concentrated. The residue was and purified by column chromatography on silica gel (MeOH/CH₂Cl₂, 5:95) to afford 21 (186.2 mg, 37%) as a brown oil; ¹H NMR (CDCl₃, 600 MHz) 7.34-7.26 (4H, m), 7.21 (1H, t, J=7.3 Hz), 4.34 (2H, s), 1.44 (9H, s); HRMS (ESI-TOF) m/z: [M−C₄H₇]⁺ calculated for C₉H₁₀NO₄ 196.0604; found 196.0608.

### Example 2—Synthesis of Pyridine and Pyrimidine Based DYRK1A Inhibitors

### N-Benzyl-5-(imidazo[1,2-a]pyridin-6-yl)pyridin-3-amine (Compound 16a)

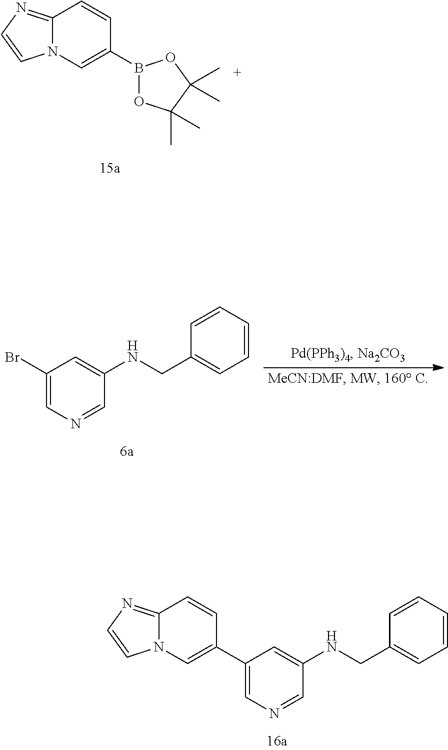 [SMILES: CC1(C)OB(c2ccc3nccn3c2)OC1(C)C ; Brc1cncc(NCc2ccccc2)c1 ; c1ccc(CNc2cncc(-c3ccc4nccn4c3)c2)cc1]

To a solution of compound 15a (35 mg, 0.13 mmol), compound 6a (43 mg, 0.27 mmol) and Pd(PPh₃)₄ (15 mg, 0.01 mmol) in anhydrous MeCN (1 mL) and anhydrous DMF (1 mL) was added 1 M Na₂CO₃ (0.27 mL, 0.27 mmol) under nitrogen. The resulting mixture was irradiated in microwave at 160° C. for 15 min. After being quenched with water (5 mL), the aqueous layer was extracted with EtOAc (2×10 mL). The combined organic extracts were washed with brine, dried over anhydrous Na₂SO₄, filtered, and concentrated. The residue was and purified by column chromatography on silica gel (MeOH/CH₂Cl₂, 5:95 to 10:90) to give compound 16a (10.7 mg, 27%) as a light brown solid; ¹H NMR (CDCl₃, 500 MHz) 8.26 (1H, s), 8.17 (1H, s), 8.10 (1H, s), 7.67-7.64 (3H, m), 7.39-7.31 (6H, m), 7.00 (1H, s), 4.42 (2H, d, J=3.0 Hz), 4.30 (1H, br); HRMS (ESI-TOF) m/z: [M+H]⁺ calculated for C₁₉H₁₇N₄ 301.1448; found 301.1454.

### N-Benzyl-5-(imidazo[1,2-a]pyridin-6-yl)pyrimidin-2-amine (Compound 16b)

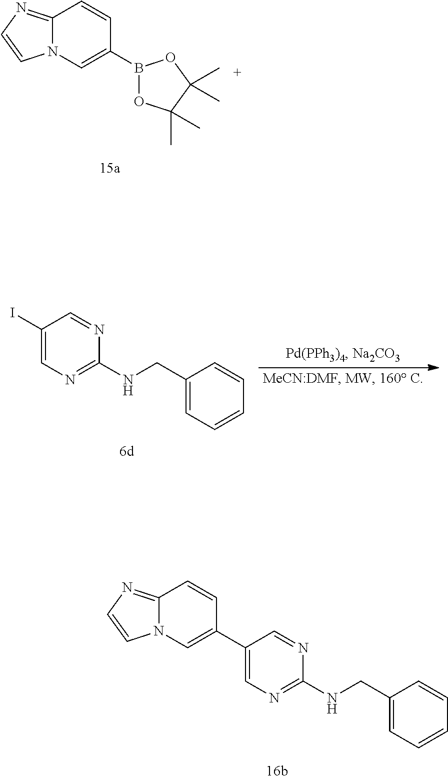 [SMILES: CC1(C)OB(c2ccc3nccn3c2)OC1(C)C ; Ic1cnc(NCc2ccccc2)nc1 ; c1ccc(CNc2ncc(-c3ccc4nccn4c3)cn2)cc1]

The title compound was prepared from compound 15a and compound 6d following method for preparation of compound 16a and purified by column chromatography on silica gel (MeOH/CH₂Cl₂, 5:95) to give compound 16b (66%) as a light brown solid; ¹H NMR (DMSO-d₆, 400 MHz) 8.84 (1H, s), 8.63 (2H, s), 7.95 (1H, t, J=6.7 Hz), 7.89 (1H, s), 7.62-7.58 (2H, m), 7.51 (1H, dd, J=9.7, 2.0 Hz), 7.33-7.28 (4H, m), 7.23-7.19 (1H, m), 4.55 (2H, d, J=6.7 Hz); HRMS (ESI-TOF) m/z: [M+Na]⁺ calculated for C₁₉H₁₆N₅ 302.1400; found 302.1430.

### 6-(5-(Benzylamino)pyridin-3-yl)-1H-benzo[d]imidazol-2-amine (Compound 16c)

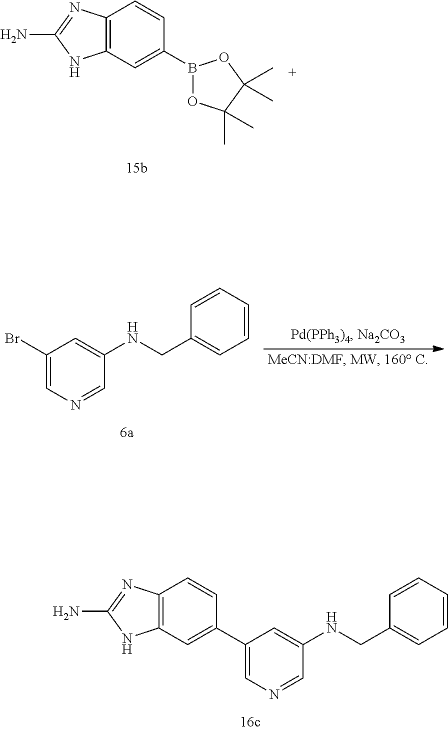 [SMILES: CC1(C)OB(c2ccc3nc(N)[nH]c3c2)OC1(C)C ; Brc1cncc(NCc2ccccc2)c1 ; Nc1nc2ccc(-c3cncc(NCc4ccccc4)c3)cc2[nH]1]

To a solution of compound 15b (39 mg, 0.15 mmol), compound 6a (39.7 mg, 0.15 mmol) and Pd(PPh₃)₄ (17.4 mg, 0.02 mmol) in anhydrous MeCN (1 mL) and anhydrous DMF (1 mL) was added 1 M Na₂CO₃ (0.32 mL, 0.32 mmol) under nitrogen. The resulting mixture was irradiated in microwave at 160° C. for 15 min. After being quenched with water (5 mL), the aqueous layer was extracted with EtOAc (2×10 mL). The combined organic extracts were washed with brine, dried over anhydrous Na₂SO₄, filtered, and concentrated. The residue was and purified by column chromatography on silica gel (MeOH/CH₂Cl₂, 5:95 to 10:90) to give compound 16c (21.1 mg, 44.3%) as a brown solid; 1H NMR (DMSO-d₆, 500 MHz) 10.75 (1H, br), 7.98 (1H, d, J=1.5 Hz), 7.88 (1H, d, J=2.5 Hz), 7.39 (2H, d, J=7.6 Hz), 7.33 (2H, t, J=7.6 Hz), 7.25-7.21 (2H, m), 7.13-7.12 (1H, d, J=8.1 Hz), 7.06-7.05 (2H, m), 6.50 (1H, t, J=5.6 Hz), 6.20 (2H, br), 4.36 (2H, d, J=5.6 Hz); HRMS (ESI-TOF) m/z: [M+H]⁺ calculated for C₁₉H₁₈N₅ 316.1557; found 316.1560.

### 6-(2-(Benzylamino)pyrimidin-5-yl)-1H-benzo[d]imidazol-2-amine (Compound 16d)

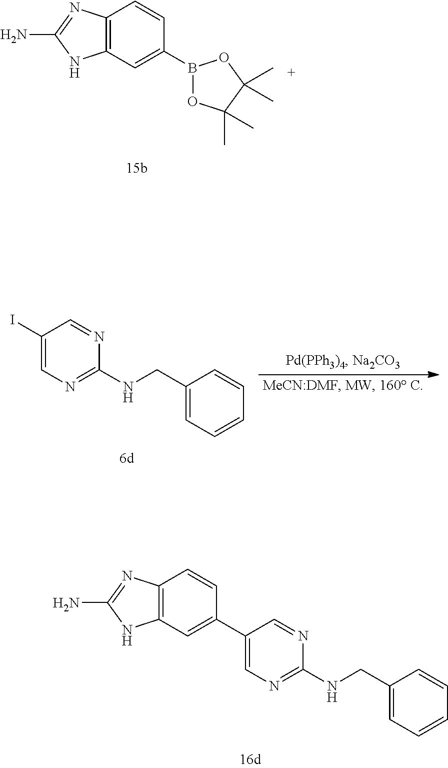 [SMILES: CC1(C)OB(c2ccc3nc(N)[nH]c3c2)OC1(C)C ; Ic1cnc(NCc2ccccc2)nc1 ; Nc1nc2ccc(-c3cnc(NCc4ccccc4)nc3)cc2[nH]1]

The title compound was prepared from compound 15b and compound 6d following method for preparation of compound 16c and purified by column chromatography on silica gel (MeOH/CH₂Cl₂, 10:90 to 15:85) to give compound 16d (12%) as a brown solid; 1H NMR (CDCl₃, 500 MHz) 10.74 (1H, br), 8.52 (2H, s), 7.67 (1H, t, J=6.1 Hz), 7.34-7.27 (5H, m), 7.20 (1H, t, J=7.1 Hz), 7.12 (1H, d, J=7.6 Hz), 7.06 (1H, d, J=6.6 Hz), 6.16 (2H, br), 4.54 (2H, d, J=6.6 Hz); HRMS (ESI-TOF) m/z: [M+H]⁺ calculated for C₁₈H₁₇N₆ 317.1509; found 317.1513.

### 5-(5-(Benzylamino)pyridin-3-yl)-1H-indazol-3-amine (Compound 16e)

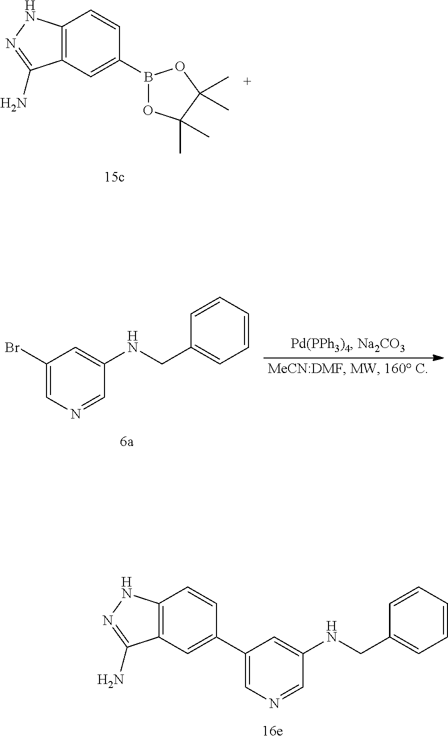 [SMILES: CC1(C)OB(c2ccc3[nH]nc(N)c3c2)OC1(C)C ; Brc1cncc(NCc2ccccc2)c1 ; Nc1n[nH]c2ccc(-c3cncc(NCc4ccccc4)c3)cc12]

The title compound was prepared from compound 15c and compound 6a following method for preparation of compound 16c and purified by column chromatography on silica gel (MeOH/CH₂Cl₂, 5:95 to 10:90) to give compound 16e (69%) as a pale yellow solid; 1H NMR (DMSO-d₆, 500 MHz) 11.43 (1H, s), 8.04 (1H, s), 7.96 (1H, s), 7.91 (1H, d, J=2.5 Hz), 7.44-7.39 (3H, m), 7.33 (2H, t, J=7.6 Hz), 7.28 (1H, d, J=8.6 Hz), 7.23 (1H, t, J=7.6 Hz), 7.12 (1H, s), 6.54 (1H, t, J=5.6 Hz), 5.38 (2H, s), 4.38 (2H, d, J=6.1 Hz); HRMS (ESI-TOF) m/z: [M+H]⁺ calculated for C₁₉H₁₈N₅ 316.1557; found 316.1562.

### 5-(2-(Benzylamino)pyrimidin-5-yl)-1H-indazol-3-amine (Compound 16f)

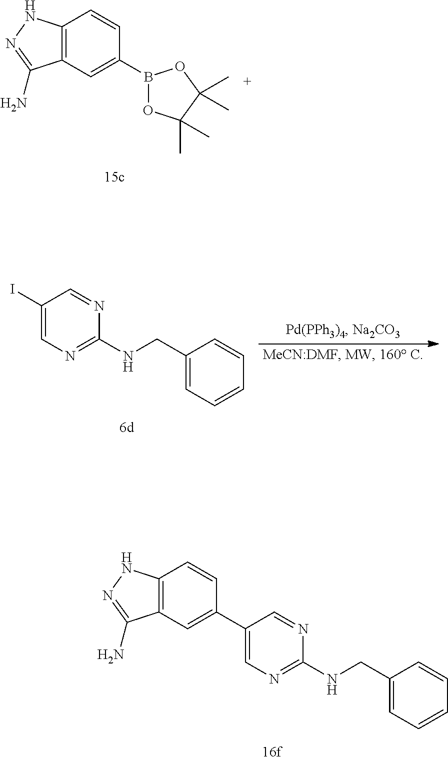 [SMILES: CC1(C)OB(c2ccc3[nH]nc(N)c3c2)OC1(C)C ; Ic1cnc(NCc2ccccc2)nc1 ; Nc1n[nH]c2ccc(-c3cnc(NCc4ccccc4)nc3)cc12]

The title compound was prepared from compound 15c and compound 6d following method for preparation of compound 16c and purified by column chromatography on silica gel (MeOH/CH₂Cl₂, 5:95 to 10:90) to give compound 16f (22%) as a brown solid; 1H NMR (DMSO-d₆, 500 MHz) 11.39 (1H, s), 8.56 (2H, s), 7.89 (1H, s), 7.75 (1H, t, J=6.6 Hz), 7.46 (1H, dd, J=8.6, 1.5 Hz), 7.34-7.26 (5H, m), 7.20 (1H, t, J=7.1 Hz), 5.33 (2H, br), 4.55 (2H, d, J=6.1 Hz); HRMS (ESI-TOF) m/z: [M+H]⁺ calculated for C₁₈H₁₇N₆ 317.1509; found 317.1478.

### 6-(5-(Benzylamino)pyridin-3-yl)-1H-indazol-3-amine (Compound 16g)

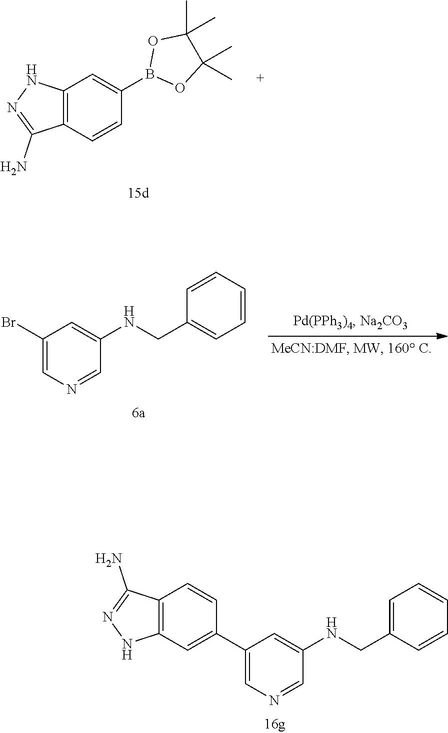 [SMILES: CC1(C)OB(c2ccc3c(N)n[nH]c3c2)OC1(C)C ; Brc1cncc(NCc2ccccc2)c1 ; Nc1n[nH]c2cc(-c3cncc(NCc4ccccc4)c3)ccc12]

The title compound was prepared from compound 15d and compound 6a following method for preparation of compound 16c and purified by column chromatography on silica gel (MeOH/CH₂Cl₂, 5:95 to 10:90) to give compound 16g (58%) as a light brown solid; 1H NMR (DMSO-d₆, 500 MHz) 11.43 (1H, s), 8.04 (1H, d, J=2.0 Hz), 7.96 (1H, d, J=2.5 Hz), 7.72 (1H, d, J=8.1 Hz), 7.40 (2H, d, J=7.1 Hz), 7.35-7.32 (3H, m), 7.23 (1H, t, J=7.1 Hz), 7.13 (1H, t, J=2.0 Hz), 7.08 (1H, d, J=7.6 Hz), 6.58 (1H, t, J=6.1 Hz), 5.34 (2H, br), 4.38 (2H, d, J=6.1 Hz); HRMS (ESI-TOF) m/z: [M+H]⁺ calculated for C₁₉H₁₈N₅ 316.1557; found 316.1590.

### 6-(2-(Benzylamino)pyrimidin-5-yl)-1H-indazol-3-amine (Compound 16h)

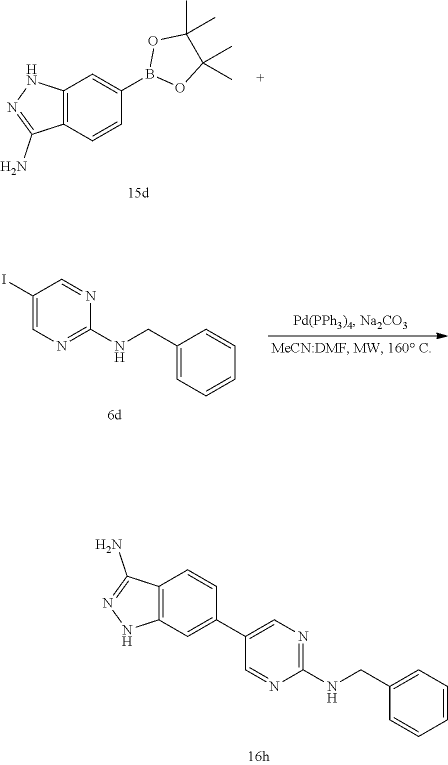 [SMILES: CC1(C)OB(c2ccc3c(N)n[nH]c3c2)OC1(C)C ; Ic1cnc(NCc2ccccc2)nc1 ; Nc1n[nH]c2cc(-c3cnc(NCc4ccccc4)nc3)ccc12]

The title compound was prepared from compound 15d and compound 6d following method for preparation of compound 16c and purified by column chromatography on silica gel (MeOH/CH₂Cl₂, 5:95 to 10:90) to give compound 16h (35%) as a yellow solid; 1H NMR (DMSO-d₆, 600 MHz) 11.41 (1H, s), 8.63 (2H, s), 7.87 (1H, t, J=6.1 Hz), 7.71 (1H, d, J=8.5 Hz), 7.36 (1H, s), 7.33-7.29 (4H, m), 7.21 (1H, t, J=7.3 Hz), 7.13 (1H, d, J=8.5 Hz), 5.34 (2H, br), 4.55 (2H, d, J=6.1 Hz); HRMS (ESI-TOF) m/z: [M+H]⁺ calculated for C₁₈H₁₇N₆ 317.1509; found 317.1603.

### N-Benzyl-5-(1H-indazol-5-yl)pyridin-3-amine (Compound 16i)

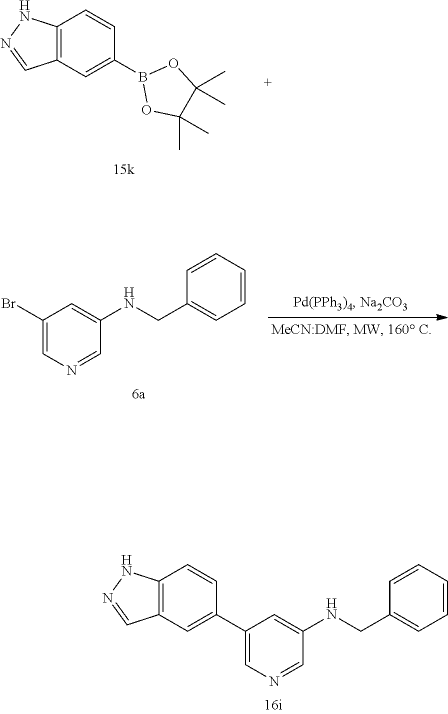 [SMILES: CC1(C)OB(c2ccc3[nH]ncc3c2)OC1(C)C ; Brc1cncc(NCc2ccccc2)c1 ; c1ccc(CNc2cncc(-c3ccc4[nH]ncc4c3)c2)cc1]

The title compound was prepared from compound 15k and compound 6a following method for preparation of compound 16c and purified by column chromatography on silica gel (EtOAc/hexane, 60:40 to 90:10) to give compound 16i (42%) as a pale yellow solid; 1H NMR (DMSO-d₆, 500 MHz) 8.10 (1H, s), 8.06 (1H, s), 7.95-7.94 (2H, m), 7.60 (1H, d, J=8.1 Hz), 7.55 (1H, d, J=8.6 Hz), 7.40 (2H, d, J=7.1 Hz), 7.33 (2H, t, J=7.1 Hz), 7.23 (1H, t, J=7.1 Hz), 7.14 (1H, s), 6.56 (1H, t, J=6.1 Hz), 4.39 (2H, d, J=6.1 Hz); HRMS (ESI-TOF) m/z: [M+H]⁺ calculated for C₁₉H₁₇N₄ 301.1448; found 301.1798.

### N-Benzyl-5-(1H-indazol-5-yl)pyrimidin-2-amine (Compound 16j)

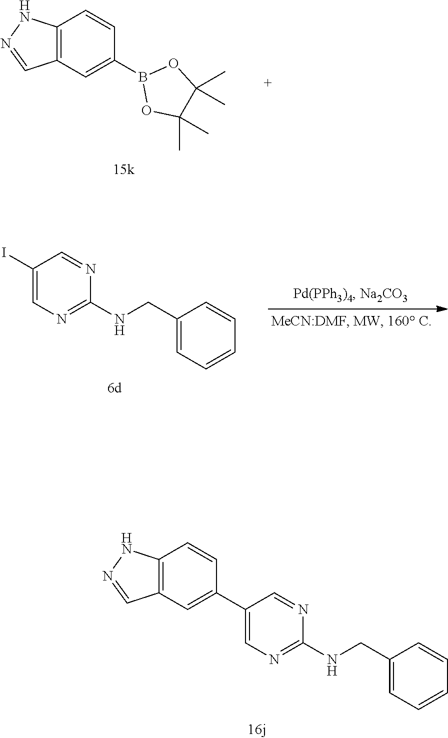 [SMILES: CC1(C)OB(c2ccc3[nH]ncc3c2)OC1(C)C ; Ic1cnc(NCc2ccccc2)nc1 ; c1ccc(CNc2ncc(-c3ccc4[nH]ncc4c3)cn2)cc1]

The title compound was prepared from compound 15k and compound 6d following method for preparation of compound 16c and purified by column chromatography on silica gel (EtOAc/hexane, 40:60 to 60:40) to give compound 16j (43%) as a white solid; 1H NMR (DMSO-d₆, 500 MHz) 8.62 (2H, s), 8.07 (1H, s), 7.95 (1H, s), 7.79 (1H, t, J=6.6 Hz), 7.58 (2H, s), 7.34-7.28 (4H, m), 7.21 (1H, t, J=7.1 Hz), 4.56 (2H, d, J=6.6 Hz); HRMS (ESI-TOF) m/z: [M+H]⁺ calculated for C₁₈H₁₆N₅ 302.1400; found 302.1399.

### 5′-(Benzylamino)-[3,3′-bipyridin]-6 (1H)-one (Compound 16k)

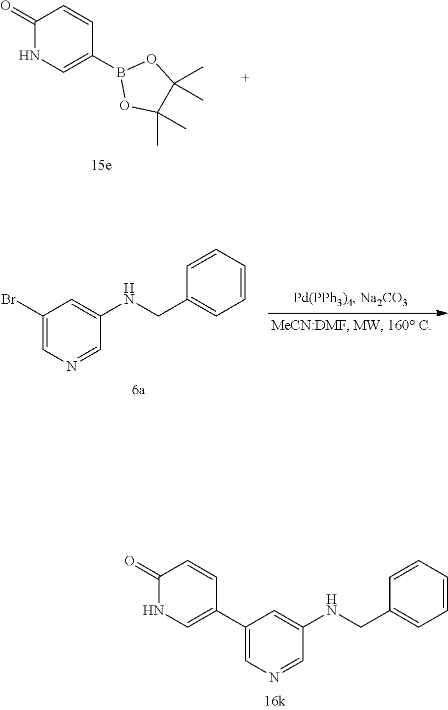 [SMILES: CC1(C)OB(c2ccc(=O)[nH]c2)OC1(C)C ; Brc1cncc(NCc2ccccc2)c1 ; O=c1ccc(-c2cncc(NCc3ccccc3)c2)c[nH]1]

The title compound was prepared from compound 15e and compound 6a following method for preparation of compound 16a and purified by column chromatography on silica gel (MeOH/CH₂Cl₂, 5:95 to 10:90) to give compound 16k (38%) as a light brown solid; 1H NMR (DMSO-d₆, 500 MHz) 11.81 (1H, br), 7.92 (1H, d, J=2.0 Hz), 7.89 (1H, d, J=2.5 Hz), 7.71 (1H, dd, J=9.7, 2.5 Hz), 7.63 (1H, br), 7.37 (2H, d, J=7.1 Hz), 7.32 (2H, t, J=7.6 Hz), 7.22 (1H, t, J=7.1 Hz), 7.00 (1H, t, J=2.0 Hz), 6.50 (1H, t, J=6.1 Hz), 6.40 (1H, d, J=9.2 Hz), 4.35 (2H, d, J=6.1 Hz); HRMS (ESI-TOF) m/z: [M+H]⁺ calculated for C₁₇H₁₆N₃O 278.1288; found 278.1297.

### 5-(2-(Benzylamino)pyrimidin-5-yl)pyridin-2 (1H)-one (Compound 161)

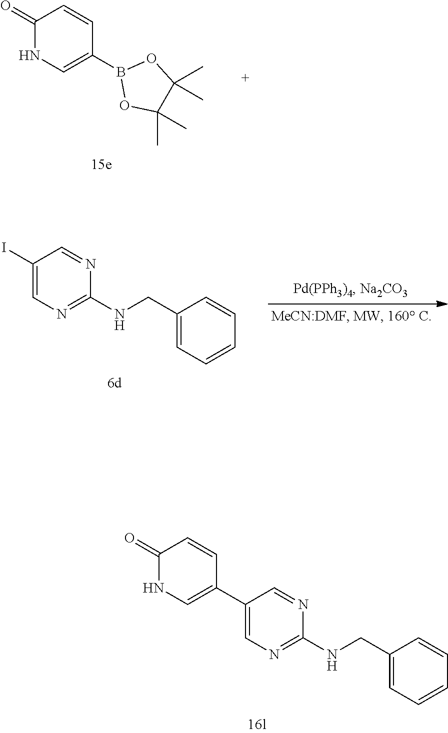 [SMILES: CC1(C)OB(c2ccc(=O)[nH]c2)OC1(C)C ; Ic1cnc(NCc2ccccc2)nc1 ; O=c1ccc(-c2cnc(NCc3ccccc3)nc2)c[nH]1]

The title compound was prepared from compound 15e and compound 6d following method for preparation of compound 16a and purified by column chromatography on silica gel (MeOH/CH₂Cl₂, 5:95 to 10:90) to give compound 161 (15%) as a brown solid; 1H NMR (DMSO-d₆, 500 MHz) 11.77 (1H, br), 8.47 (2H, s), 7.77 (1H, t, J=6.1 Hz), 7.72 (1H, dd, J=9.7, 2.5 Hz), 7.62 (1H, br), 7.30-7.27 (4H, m), 7.20-7.18 (1H, m), 6.39 (1H, d, J=9.7 Hz), 4.51 (2H, d, J=6.6 Hz); HRMS (ESI-TOF) m/z: [M+H]⁺ calculated for C₁₆H₁₅N₄O 279.1240; found 279.1597.

### 6-(5-(Benzylamino)pyridin-3-yl)quinolin-2 (1H)-one (Compound 16m)

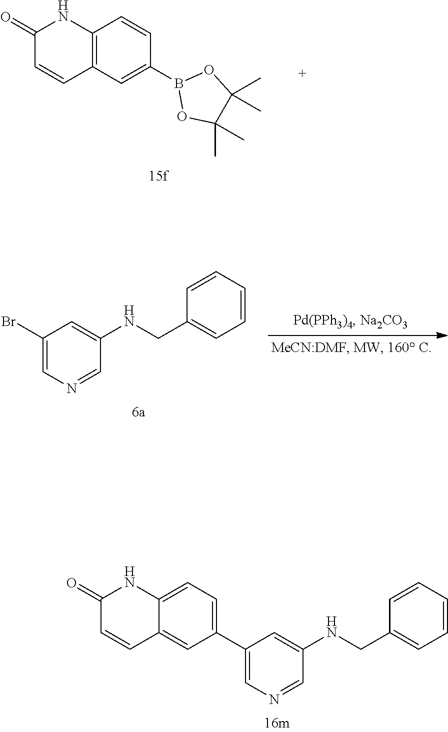 [SMILES: CC1(C)OB(c2ccc3[nH]c(=O)ccc3c2)OC1(C)C ; Brc1cncc(NCc2ccccc2)c1 ; O=c1ccc2cc(-c3cncc(NCc4ccccc4)c3)ccc2[nH]1]

The title compound was prepared from compound 15f and compound 6a following method for preparation of compound 16c and purified by column chromatography on silica gel (MeOH/CH₂Cl₂, 15:85 to 20:80) to give compound 16m (59%) as a light brown solid; 1H NMR (DMSO-d₆, 500 MHz) 11.78 (1H, s), 8.07 (1H, d, J=1.5 Hz), 7.96 (1H, d, J=2.5 Hz), 7.93 (1H, d, J=9.7 Hz), 7.90 (1H, d, J=1.5 Hz), 7.72 (1H, dd, J=8.4, 1.5 Hz), 7.40 (2H, d, J=7.6 Hz), 7.37-7.32 (3H, m), 7.23 (1H, t, J=7.6 Hz), 7.15 (1H, t, J=2.0 Hz), 6.58 (1H, t, J=5.6 Hz), 6.52 (1H, dd, J=9.7, 1.5 Hz), 4.38 (2H, d, J=5.6 Hz); HRMS (ESI-TOF) m/z: [M+H]⁺ calculated for C₂₁H₁₈N₃O 328.1444; found 328.1460.

### 6-(2-(Benzylamino)pyrimidin-5-yl)quinolin-2 (1H)-one (Compound 16n)

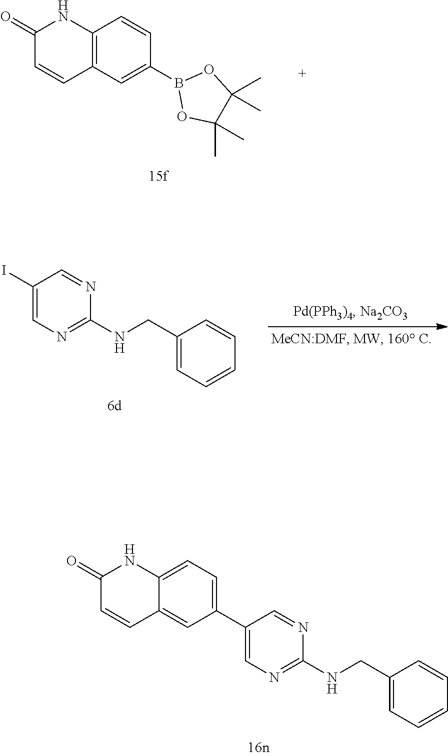 [SMILES: CC1(C)OB(c2ccc3[nH]c(=O)ccc3c2)OC1(C)C ; Ic1cnc(NCc2ccccc2)nc1 ; O=c1ccc2cc(-c3cnc(NCc4ccccc4)nc3)ccc2[nH]1]

The title compound was prepared from compound 15f and compound 6d following method for preparation of compound 16c and purified by column chromatography on silica gel (MeOH/CH₂Cl₂, 2.5:97.5 to 5:95) to give compound 16n (18%) as a white solid; ¹H NMR (DMSO-d₆, 500 MHz) 11.75 (1H, s), 8.63 (2H, s), 7.91-7.86 (3H, m), 7.76 (1H, dd, J=8.6, 2.0 Hz), 7.35-7.28 (5H, m), 7.20 (1H, t, J=7.6 Hz), 6.51 (1H, dd, J=9.4, 2.0 Hz), 4.55 (2H, d, J=6.6 Hz); HRMS (ESI-TOF) m/z: [M+H]⁺ calculated for C₂₀H₁₇N₄O 329.1397; found 329.1409.

### 5-(5-(Benzylamino)pyridin-3-yl)quinolin-2 (1H)-one (Compound 160)

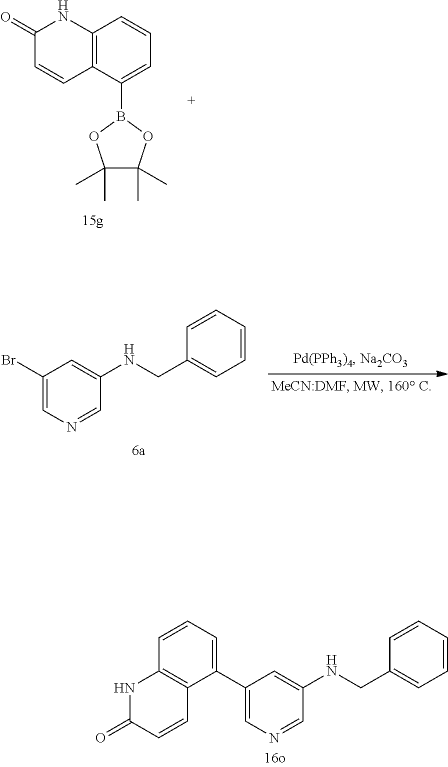 [SMILES: CC1(C)OB(c2cccc3[nH]c(=O)ccc23)OC1(C)C ; Brc1cncc(NCc2ccccc2)c1 ; O=c1ccc2c(-c3cncc(NCc4ccccc4)c3)cccc2[nH]1]

The title compound was prepared from compound 15f and compound 6a following method for preparation of compound 16c and purified by column chromatography on silica gel (MeOH/CH₂Cl₂, 5:95) to give compound 16o (58%) as a yellow solid; 1H NMR (DMSO-d₆, 500 MHz) 11.82 (1H, s), 8.07 (1H, d, J=2.5 Hz), 7.74 (1H, d, J=1.5 Hz), 7.54-7.48 (2H, m), 7.38-7.32 (5H, m), 7.25 (1H, t, J=7.1 Hz), 7.05 (1H, d, J=7.1 Hz), 6.83 (1H, t, J=2.0 Hz), 6.74 (1H, t, J=6.1 Hz), 6.35 (1H, dd, J=9.7, 1.5 Hz), 4.35 (2H, d, J=5.6 Hz); HRMS (ESI-TOF) m/z: [M+H]⁺ calculated for C₂₁H₁₈N₃O 328.1444; found 328.1580.

### 5-(2-(Benzylamino)pyrimidin-5-yl)quinolin-2 (1H)-one (Compound 16p)

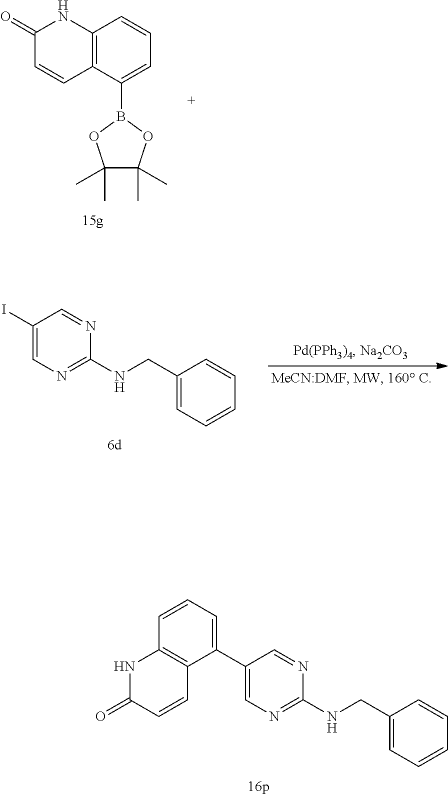 [SMILES: CC1(C)OB(c2cccc3[nH]c(=O)ccc23)OC1(C)C ; Ic1cnc(NCc2ccccc2)nc1 ; O=c1ccc2c(-c3cnc(NCc4ccccc4)nc3)cccc2[nH]1]

The title compound was prepared from compound 15f and compound 6d following method for preparation of compound 16c and purified by column chromatography on silica gel (MeOH/CH₂Cl₂, 5:95) to give compound 16p (36%) as a white solid; 1H NMR (DMSO-d₆, 500 MHz) 11.82 (1H, s), 8.35 (2H, s), 7.98 (1H, t, J=6.1 Hz), 7.75 (1H, d, J=9.7 Hz), 7.52 (1H, t, J=7.6 Hz), 7.35-7.28 (5H, m), 7.22 (1H, t, J=7.1 Hz), 7.10 (1H, d, J=6.6 Hz), 6.47 (1H, dd, J=9.7, 1.5 Hz), 4.57 (2H, d, J=6.1 Hz); HRMS (ESI-TOF) m/z: [M+H]⁺ calculated for C₂₀H₁₇N₄O 329.1397; found 329.1469.

### N-benzyl-5-(1H-indazol-5-yl)pyridin-3-amine (Compound 16q)

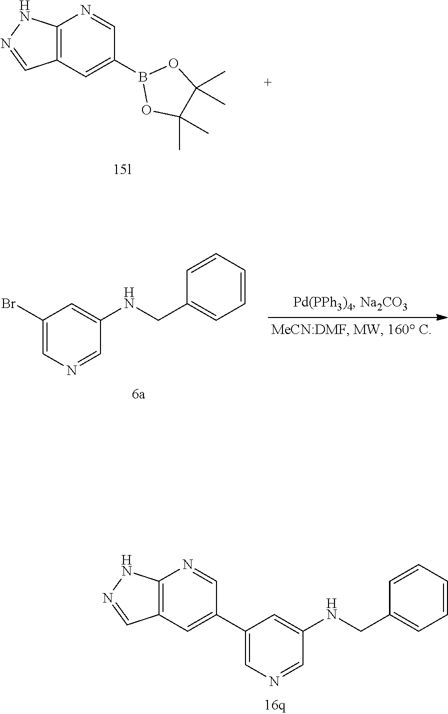 [SMILES: CC1(C)OB(c2cnc3[nH]ncc3c2)OC1(C)C ; Brc1cncc(NCc2ccccc2)c1 ; c1ccc(CNc2cncc(-c3cnc4[nH]ncc4c3)c2)cc1]

The title compound was prepared from compound 151 and compound 6a following method for preparation of compound 16c and purified by column chromatography on silica gel (MeOH/CH₂Cl₂, 2.5:97.5 to 5:95) to give compound 16q (70%) as a pale yellow solid; 1H NMR (DMSO-d₆, 500 MHz) 8.73 (1H, d, J=2.0 Hz), 8.41 (1H, d, J=1.5 Hz), 8.18 (1H, s), 8.10 (1H, d, J=1.5 Hz), 8.01 (1H, d, J=2.5 Hz), 7.40 (2H, d, J=7.1 Hz), 7.33 (2H, t, J=7.6 Hz), 7.24-7.20 (2H, m), 6.63 (1H, t, J=5.6 Hz), 4.40 (2H, d, J=5.6 Hz); HRMS (ESI-TOF) m/z: [M+H]⁺ calculated for C₁₈H₁₆N₅ 302.1400; found 302.1562.

### N-Benzyl-5-(1H-pyrazolo[3,4-b]pyridin-5-yl)pyrimidin-2-amine (Compound 16r)

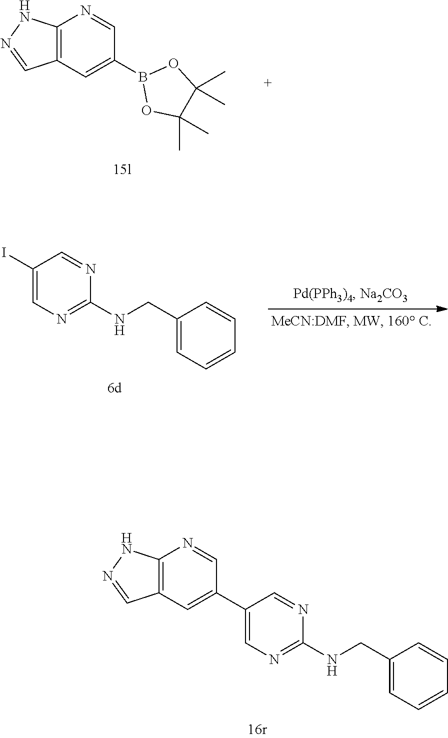 [SMILES: CC1(C)OB(c2cnc3[nH]ncc3c2)OC1(C)C ; Ic1cnc(NCc2ccccc2)nc1 ; c1ccc(CNc2ncc(-c3cnc4[nH]ncc4c3)cn2)cc1]

The title compound was prepared from compound 151 and compound 6d following method for preparation of compound 16c and purified by column chromatography on silica gel (MeOH/CH₂Cl₂, 2.5:97.5 to 5:95) to give compound 16r (37%) as a pale yellow solid; 1H NMR (DMSO-d₆, 500 MHz) 8.77 (1H, s), 8.68 (2H, s), 8.41 (1H, s), 7.90 (1H, t, J=6.1 Hz), 7.34-7.28 (5H, m), 7.21 (1H, t, J=7.1 Hz), 4.56 (2H, d, J=6.6 Hz); HRMS (ESI-TOF) m/z: [M+H]⁺ calculated for C₁₇H₁₅N₆ 303.1353; found 303.1376.

### 2-Chloro-N-(5-methyl-1H-pyrazol-3-yl)pyrimidin-4-amine (Compound 15m)

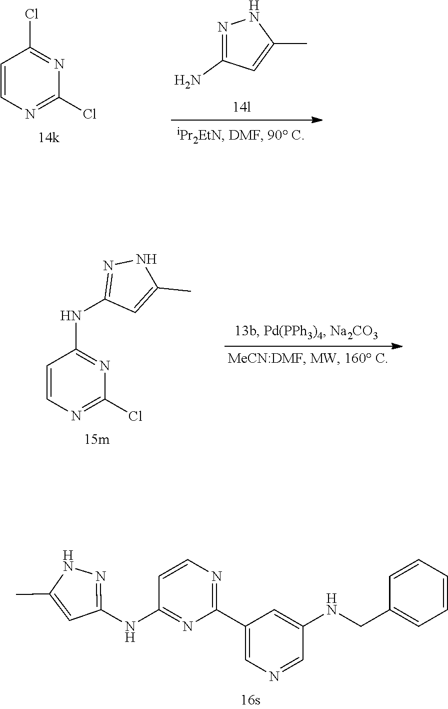

To a solution of compound 14k (400.0 mg, 2.69 mmol) and compound 141 (287.0 mg, 2.95 mmol) in anhydrous DMF (5 mL) was added diisopropylethylamine (1.9 mL, 10.7 mmol). The resulting mixture was heated at 90° C. for 16 h. After being quenched by the addition of water, the aqueous layer was extracted with EtOAc (2×20 mL). The combined organic extracts were washed with brine, dried over anhydrous Na₂SO₄, filtered, and concentrated. The residue was purified by column chromatography on silica gel (MeOH/CH₂Cl₂, 5:95 to 10:90) to afford compound 15m (271.1 mg, 48%) as a light brown solid; ¹H NMR (DMSO-d₆, 500 MHz) 12.10 (1H, s), 10.25 (1H, s), 8.14 (1H, s); HRMS (ESI-TOF) m/z: [M+H]⁺ calculated for C₈H₉ClN₅ 210.0541; found 210.0645.

### 2-(5-(Benzylamino)pyridin-3-yl)-N-(5-methyl-1H-pyrazol-3-yl)pyrimidin-4-amine (Compound 16s)

The title compound was prepared from compound 15m and compound 13b following method for preparation of compound 16a and purified by column chromatography on silica gel (MeOH/CH₂Cl₂, 5:95 to 10:90) to give compound 16s (31%) as a brown solid; 1H NMR (DMSO-d₆, 500 MHz) 12.00 (1H, s), 9.84 (1H, s), 8.67 (1H, s), 8.31 (1H, d, J=5.6 Hz), 8.22 (1H, d, J=5.6 Hz), 8.06 (1H, d, J=2.5 Hz), 7.79 (1H, s), 7.38 (2H, d, J=7.1 Hz), 7.32 (2H, t, J=8.1 Hz), 7.23 (1H, t, J=6.6 Hz), 6.67 (1H, t, J=5.6 Hz), 4.38 (2H, d, J=5.6 Hz), 2.23 (3H, s); HRMS (ESI-TOF) m/z: [M+H]⁺ calculated for C₂₀H₂₀N₇ 358.1775; found 358.1739.

### N^{2′}-Benzyl-N⁴-(5-methyl-1H-pyrazol-3-yl)-[2,5′-bipyrimidine]-2′,4-diamine (Compound 16t)

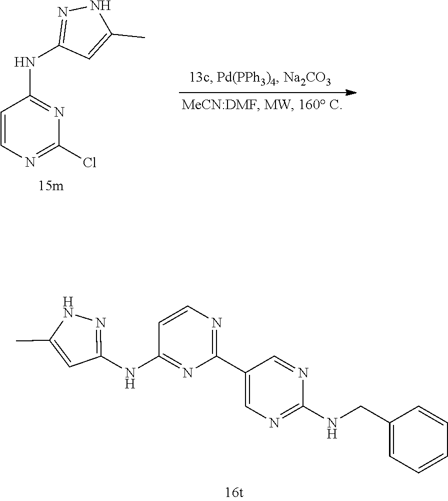 [SMILES: Cc1cc(Nc2ccnc(Cl)n2)n[nH]1 ; Cc1cc(Nc2ccnc(-c3cnc(NCc4ccccc4)nc3)n2)n[nH]1]

The title compound was prepared from compound 15m and compound 13c following method for preparation of compound 16a and purified by column chromatography on silica gel (MeOH/CH₂Cl₂, 5:95) to give compound 16t (13%) as a brown solid; 1H NMR (DMSO-d₆, 500 MHz) 11.98 (1H, s), 9.79 (1H, s), 9.07 (2H, s), 8.25 (1H, d, J=5.6 Hz), 8.15 (1H, t, J=6.1 Hz), 7.32-7.28 (5H, m), 7.21 (1H, t, J=6.6 Hz), 4.59 (2H, d, J=6.1 Hz), 2.23 (3H, s); HRMS (ESI-TOF) m/z: [M+H]⁺ calculated for C₁₉H₁₉N₈ 359.1727; found 359.1705.

### 5-(5-(Benzylamino)pyridin-3-yl)-N-phenyl-7H-pyrrolo[2,3-d]pyrimidin-2-amine (Compound 16u)

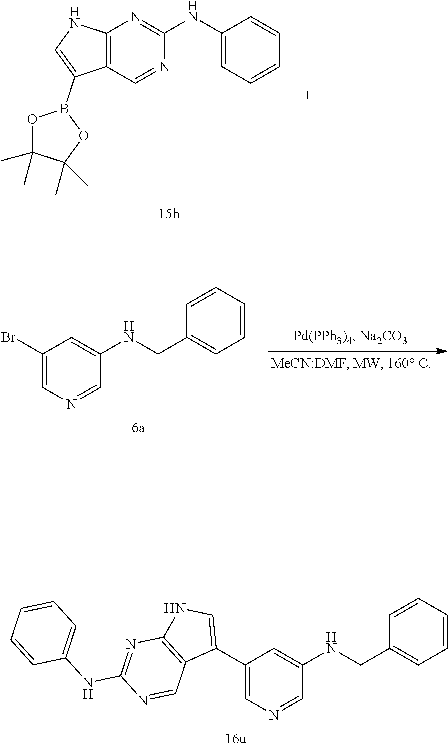 [SMILES: CC1(C)OB(c2c[nH]c3nc(Nc4ccccc4)ncc23)OC1(C)C ; Brc1cncc(NCc2ccccc2)c1 ; c1ccc(CNc2cncc(-c3c[nH]c4nc(Nc5ccccc5)ncc34)c2)cc1]

The title compound was prepared from compound 15h and compound 6a following method for preparation of compound 16a and purified by column chromatography on silica gel (EtOAc/hexane, 40:60 to 70:30) to give compound 16u (51%) as a light brown solid; 1H NMR (DMSO-d₆, 500 MHz) 11.78 (1H, s), 9.39 (1H, s), 8.78 (1H, s), 8.13 (1H, d, J=1.5 Hz), 7.88 (1H, d, J=2.5 Hz), 7.85 (1H, s), 7.84 (1H, s), 7.57 (1H, d, J=2.0 Hz), 7.41 (2H, d, J=7.6 Hz), 7.34 (2H, t, J=7.1 Hz), 7.27-7.23 (3H, m), 7.17 (1H, t, J=2.0 Hz), 6.89 (1H, t, J=7.6 Hz), 6.53 (1H, t, J=6.1 Hz), 4.38 (2H, d, J=6.1 Hz); HRMS (ESI-TOF) m/z: [M+H]⁺ calculated for C₂₄H₂₁N₆ 393.1822; found 393.1820.

### 5-(2-(Benzylamino)pyrimidin-5-yl)-N-phenyl-7H-pyrrolo[2,3-d]pyrimidin-2-amine (Compound 16v)

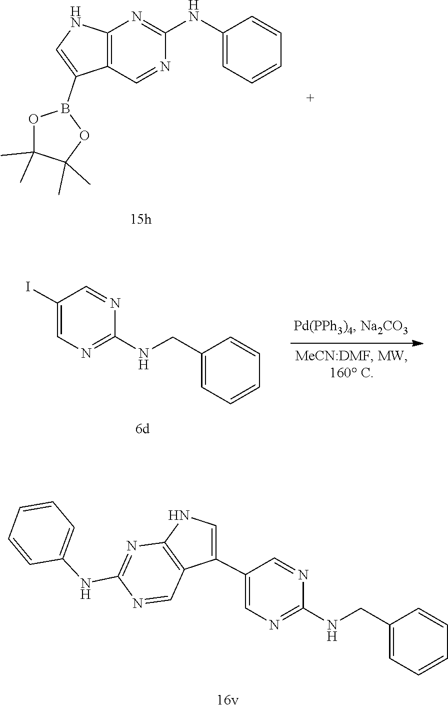 [SMILES: CC1(C)OB(c2c[nH]c3nc(Nc4ccccc4)ncc23)OC1(C)C ; Ic1cnc(NCc2ccccc2)nc1 ; c1ccc(CNc2ncc(-c3c[nH]c4nc(Nc5ccccc5)ncc34)cn2)cc1]

The title compound was prepared from compound 15h and compound 6d following method for preparation of compound 16a and purified by column chromatography on silica gel (EtOAc/hexane, 40:60 to 60:40) to give compound 16v (37%) as a pale yellow solid; 1H NMR (DMSO-d₆, 500 MHz) 11.70 (1H, s), 9.37 (1H, s), 8.97 (1H, s), 8.67 (2H, s), 7.84 (2H, d, J=7.6 Hz), 7.73 (1H, t, J=6.6 Hz), 7.51 (1H, d, J=2.5 Hz), 7.34-7.20 (7H, m), 6.88 (1H, t, J=7.6 Hz), 4.55 (2H, d, J=6.6 Hz); HRMS (ESI-TOF) m/z: [M+H]⁺ calculated for C₂₃H₂₀N₇ 394.1775; found 394.1799.

### N-(5′-(Benzylamino)-[3,3′-bipyridin]-6-yl)acetamide (Compound 16w)

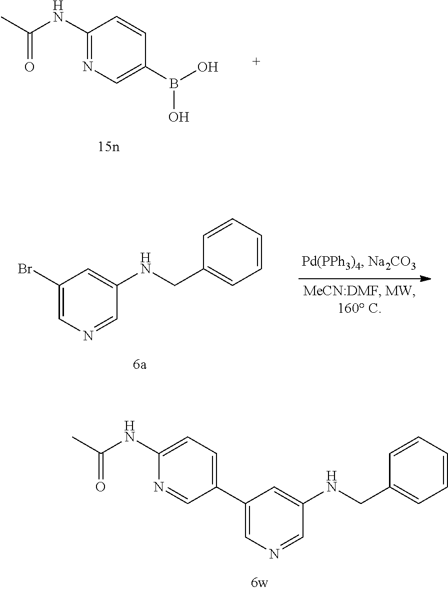 [SMILES: CC(=O)Nc1ccc(B(O)O)cn1 ; Brc1cncc(NCc2ccccc2)c1 ; CC(=O)Nc1ccc(-c2cncc(NCc3ccccc3)c2)cn1]

The title compound was prepared from compound 15n and compound 6a following method for preparation of compound 16c and purified by column chromatography on silica gel (EtOAc/hexane, 80:20) to give compound 16w (34%) as a pale yellow solid; 1H NMR (DMSO-d₆, 500 MHz) 10.55 (1H, s), 8.52 (1H, d, J=2.5 Hz), 8.12 (1H, d, J=8.6 Hz), 8.05 (1H, d, J=1.5 Hz), 8.00-7.98 (2H, m), 7.93 (2H, d, J=7.6 Hz), 7.33 (2H, t, J=7.6 Hz), 7.23 (1H, t, J=6.6 Hz), 7.14 (1H, s), 6.60 (1H, t, J=6.1 Hz), 4.38 (2H, d, J=6.1 Hz), 2.10 (3H, s); HRMS (ESI-TOF) m/z: [M+H]⁺ calculated for C₁₉H₁₉N₄O 319.1553; found 319.1599.

### N-(5-(2-(Benzylamino)pyrimidin-5-yl)pyridin-2-yl)acetamide (Compound 16x)

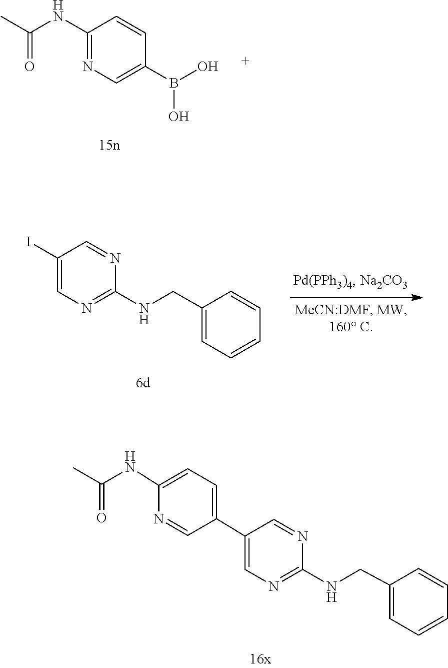 [SMILES: CC(=O)Nc1ccc(B(O)O)cn1 ; Ic1cnc(NCc2ccccc2)nc1 ; CC(=O)Nc1ccc(-c2cnc(NCc3ccccc3)nc2)cn1]

The title compound was prepared from compound 15n and compound 6d following method for preparation of compound 16c and purified by column chromatography on silica gel (MeOH/CH₂Cl₂, 5:95 to 10:90) to give compound 16x (43%) as a white solid; 1H NMR (DMSO-d₆, 500 MHz) 10.50 (1H, s), 8.63 (2H, s), 8.56 (1H, d, J=2.0 Hz), 8.09 (1H, d, J=8.6 Hz), 8.01 (1H, dd, J=8.4, 2.5 Hz), 7.91 (1H, t, J=6.1 Hz), 7.32-7.28 (4H, m), 7.20 (1H, t, J=6.6 Hz), 4.54 (2H, d, J=6.6 Hz), 2.09 (3H, s); HRMS (ESI-TOF) m/z: [M+H]⁺ calculated for C₁₈H₁₈N₅O 320.1506; found 320.1590.

### N⁵-Benzyl-N^{6′}-methyl-[3,3′-bipyridine]-5,6′-diamine (Compound 16y)

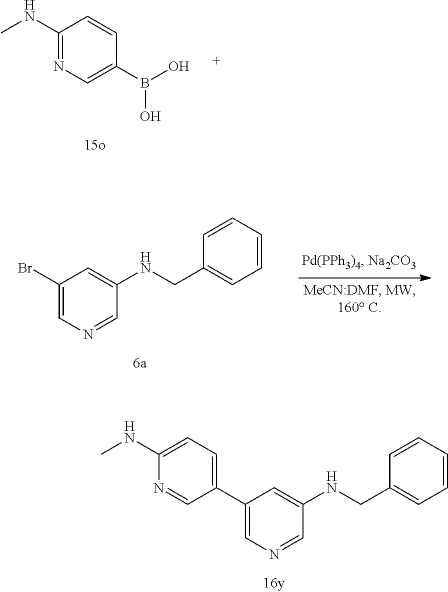 [SMILES: CNc1ccc(B(O)O)cn1 ; Brc1cncc(NCc2ccccc2)c1 ; CNc1ccc(-c2cncc(NCc3ccccc3)c2)cn1]

The title compound was prepared from compound 15n and compound 6a following method for preparation of compound 16a and purified by column chromatography on silica gel (MeOH/CH₂Cl₂, 2:98 to 4:96) to give compound 16y (13%) as a brown solid; ¹H NMR (CD₃OD, 500 MHz) 8.10 (1H, d, J=1.0 Hz), 7.90 (1H, s), 7.84 (1H, s), 7.62 (1H, dd, J=8.6, 1.5 Hz), 7.39 (2H, d, J=7.1 Hz), 7.32 (2H, t, J=7.1 Hz), 7.23 (1H, t, J=7.6 Hz), 7.08 (1H, s), 6.57 (1H, d, J=8.6 Hz), 4.40 (2H, s), 2.88 (3H, s); HRMS (ESI-TOF) m/z: [M+H]⁺ calculated for C₁₈H₁₉N₄ 291.1604; found 291.1615.

### N-Benzyl-5-(6-(methylamino)pyridin-3-yl)pyrimidin-2-amine (Compound 16z)

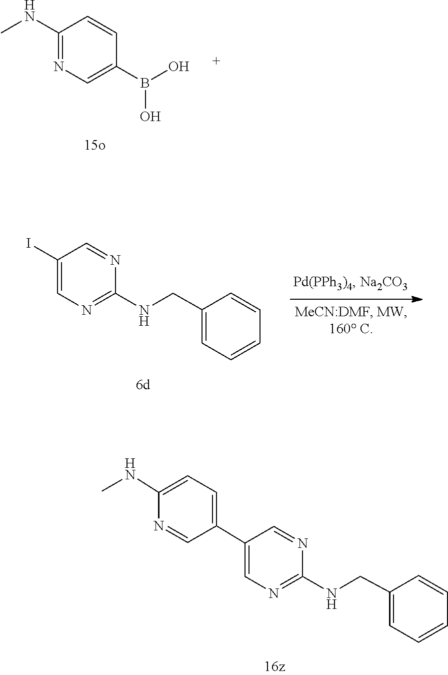 [SMILES: CNc1ccc(B(O)O)cn1 ; Ic1cnc(NCc2ccccc2)nc1 ; CNc1ccc(-c2cnc(NCc3ccccc3)nc2)cn1]

The title compound was prepared from compound 15n and compound 6a following method for preparation of compound 16a and purified by column chromatography on silica gel (EtOAc/hexane, 50:50 to 80:20) to give compound 16z (7%) as a white solid; 1H NMR (DMSO-d₆, 500 MHz) 8.49 (2H, s), 8.21 (1H, d, J=2.0 Hz), 7.71 (1H, t, J=6.6 Hz), 7.61 (1 H, dd, J=8.6, 2.5 Hz), 7.32-7.27 (4H, m), 7.19 (1H, t, J=6.6 Hz), 6.54-6.47 (2H, m), 4.52 (2H, d, J=6.1 Hz), 2.77 (3H, d, J=5.1 Hz); HRMS (ESI-TOF) m/z: [M+H]⁺ calculated for C₁₇H₁₈N₅ 292.1557; found 292.1588.

### 5-(((1H-1,2,4-Triazol-5-yl)amino)methyl)-N-benzylpyrimidin-2-amine (Compound 16aa)

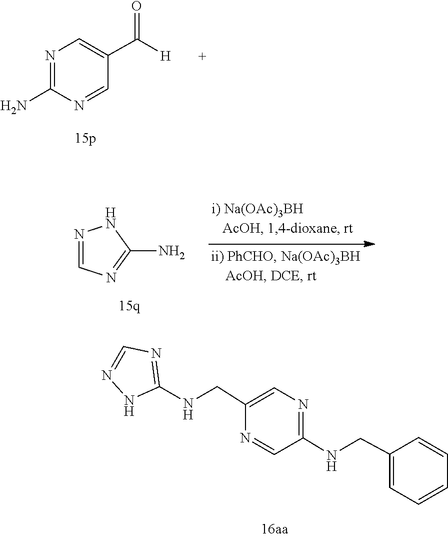 [SMILES: Nc1ncc(C=O)cn1 ; Nc1ncn[nH]1 ; c1ccc(CNc2cnc(CNc3ncn[nH]3)cn2)cc1]

To a solution of compound 15p (100.0 mg, 0.81 mmol) and sodium triacetoxyborohydride (516.0 mg, 2.44 mmol) in 1,4-dioxane (4 mL) was added acetic acid (2 mL) and compound 15q (75.1 mg, 0.89 mmol) under nitrogen. The resulting mixture was stirred at room temperature for 18 h. Sodium triacetoxyborohydride (172 mg, 0.81 mmol) was added and the reaction was stirred for another 2 h. After being quenched by the addition of MeOH (5 mL), the solvent was removed in vacuo. The crude product was dissolved in DCE (5 mL) then sodium triacetoxyborohydride (516 mg, 2.44 mmol), benzaldezyde (0.16 mL, 1.62 mmol) and AcOH (2 mL) were added, respectively. The reaction mixture was stirred at room temperature for 16 h. After being quenched by the addition of water, aqueous layer was extracted with EtOAc (2×20 mL). The combined organic extracts were washed with brine, dried over anhydrous Na₂SO₄, filtered, and concentrated. The residue was purified by column chromatography on silica gel (MeOH/CH₂Cl₂, 5:95 to 10:90) to afford compound 16aa (25.8 mg, 11%) as a white solid; 1H NMR (DMSO-d₆, 400 MHz) 12.17 (1H, br), 8.23 (2H, s), 7.35 (1H, br), 7.27-7.26 (4H, m), 7.20-7.16 (1H, m), 6.84 (1H, br), 4.56 (2H, d, J=6.7 Hz); HRMS (ESI-TOF) m/z: [M+H]⁺ calculated for C₁₄H₁₆N₇ 282.1462; found 282.1453.

### N⁵-Benzyl-N^{2′}-methyl-[3,4′-bipyridine]-2′,5-diamine (Compound 16ab)

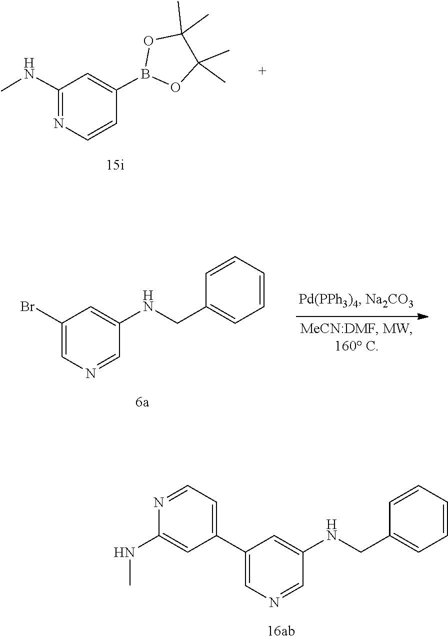

The title compound was prepared from compound 15i and compound 6a following method for preparation of compound 16a and purified by column chromatography on silica gel (MeOH/CH₂Cl₂, 2.5:97.5 to 5:95) to give compound 16ab (60%) as a white solid; 1H NMR (DMSO-d₆, 600 MHz) 8.00 (2H, s), 7.91 (1H, s), 7.39-7.31 (4H, m), 7.24-7.22 (1H, m), 6.99 (1H, s), 6.69-6.65 (2H, m), 6.55 (1H, s), 6.52-6.51 (1H, m), 4.36 (2H, d, J=6.1 Hz), 2.78 (3H, d, J=4.9 Hz); HRMS (ESI-TOF) m/z: [M+H]⁺ calculated for C₁₈H₁₉N₄ 291.1604; found 291.1590.

### N-Benzyl-5-(2-(methylamino)pyridin-4-yl)pyrimidin-2-amine (Compound 16ac)

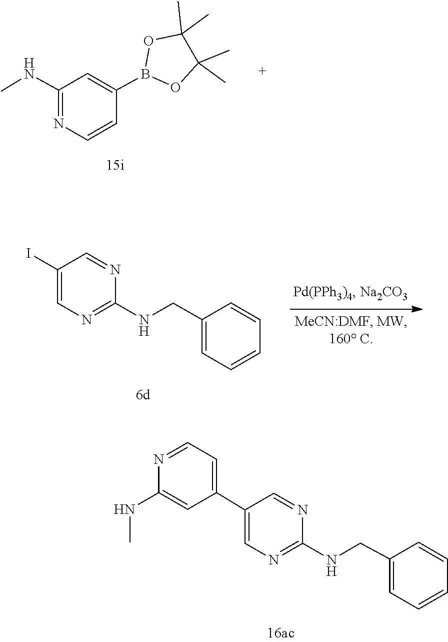

The title compound was prepared from compound 15i and compound 6d following method for preparation of compound 16a and purified by column chromatography on silica gel (MeOH/CH₂Cl₂, 2.5:97.5 to 5:95) to give compound 16ac (41%) as a light brown solid; 1H NMR (DMSO-d₆, 600 MHz) 8.62 (2H, s), 8.04 (1H, t, J=6.1 Hz), 7.97 (1H, d, J=4.9 Hz), 7.30-7.28 (4H, m), 7.20 (1H, t, J=6.1 Hz), 6.74 (1H, d, J=4.9 Hz), 6.59 (1H, s), 6.41 (1H, q, J=4.9 Hz), 4.54 (2H, d, J=6.1 Hz), 2.78 (3H, d, J=4.9 Hz); HRMS (ESI-TOF) m/z: [M+H]⁺ calculated for C₁₇H₁₈N₅ 292.1557; found 292.1936.

### tert-Butyl 6-(2-(benzylamino)pyrimidin-5-yl)-1H-indazole-3-carboxylate (Compound 16ad)

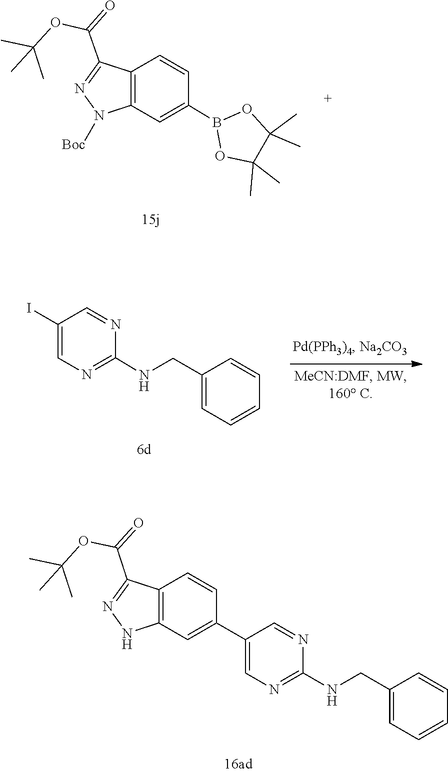

The title compound was prepared from compound 15j and compound 6d following method for preparation of compound 16c and purified by column chromatography on silica gel (EtOAc/hexane, 35:65 to 50:50) to give compound 16ad (53%) as a yellow solid; 1H NMR (CDCl₃, 600 MHz) 8.62 (2H, s), 8.23 (1H, d, J=8.5 Hz), 7.79 (1H, s), 7.44 (1H, d, J=8.5 Hz), 7.40 (2, d, J=7.3 Hz), 7.35 (2H, t, J=7.3 Hz), 7.29 (1H, t, J=7.3 Hz), 5.72 (1H, t, J=4.9 Hz), 4.72 (2H, d, J=6.1 Hz), 1.73 (9H, s); HRMS (ESI-TOF) m/z: [M+H]⁺ calculated for C₂₃H₂₄N₅O₂ 402.1925; found 402.1918.

### 6-(2-(Benzylamino)pyrimidin-5-yl)-1H-indazole-3-carboxylic acid hydrochloride (Compound 16ae)

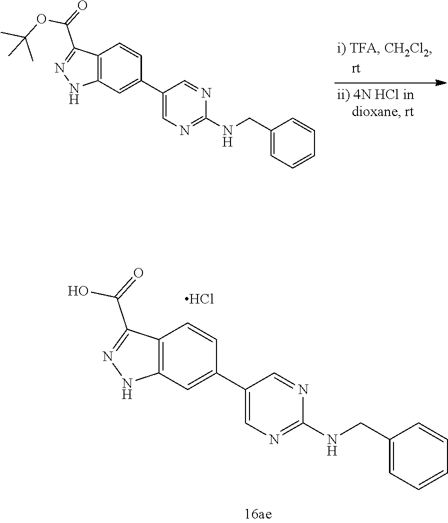 [SMILES: CC(C)(C)OC(=O)c1n[nH]c2cc(-c3cnc(NCc4ccccc4)nc3)ccc12 ; O=C(O)c1n[nH]c2cc(-c3cnc(NCc4ccccc4)nc3)ccc12 ; Cl]

To a solution of compound 16ad (19.9 mg, 0.05 mmol) in anhydrous CH₂Cl₂ (3 mL) was added trifluoroacetic acid (0.3 mL). The resulting mixture was stirred at room temperature for 16 h. The solvent was removed in vacuo then 4 N HCl in 1,4-dioxane (1 mL) was added. The reaction was stirred for 5 min then solvent was removed. Toluene was added then removed under vacuum. The crude product was washed with CH₂Cl₂ and hexane to give compound 16ae (12.1 mg, 64%) as a brown solid; ¹H NMR (DMSO-d₆, 600 MHz) 8.72 (2H, s), 8.09 (1H, d, J=8.5 Hz), 8.03 (1H, br), 7.79 (1H, s), 7.54 (1H, d, J=8.5 Hz), 7.34-7.29 (5H, m), 7.21 (1H, t, J=6.1 Hz), 4.57 (2H, s); HRMS (ESI-TOF) m/z: [M+H]⁺ calculated for C₁₉H₁₆N₅O₂ 346.1299; found 402.1918.

### N-((5-(1H-Indazol-5-yl)pyridin-3-yl)methyl)aniline (Compound 16af)

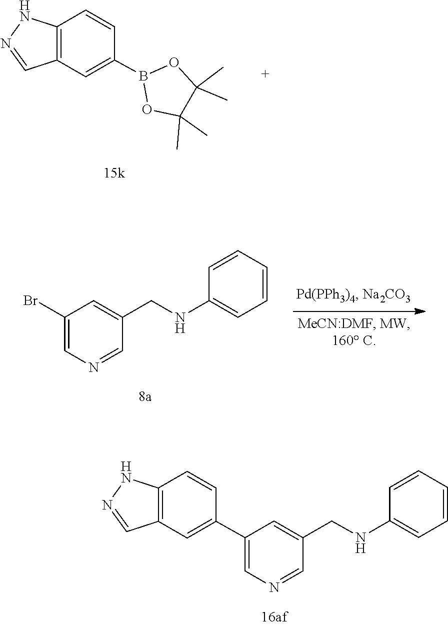 [SMILES: CC1(C)OB(c2ccc3[nH]ncc3c2)OC1(C)C ; Brc1cncc(CNc2ccccc2)c1 ; c1ccc(NCc2cncc(-c3ccc4[nH]ncc4c3)c2)cc1]

The title compound was prepare from compound 15k and compound 8a following method for preparation of compound 16c and purified by column chromatography on silica gel (EtOAc/hexane, 80:20) to give compound 16af (77%) as a white solid; 1H NMR (CDCl₃, 600 MHz) 8.81 (1H, d, J=2.0 Hz), 8.62 (1H, d, J=1.5 Hz), 8.15 (1H, s), 7.94 (2H, d, J=5.1 Hz), 7.61 (1H, d, J=4.1 Hz), 7.22-7.18 (3H, m), 6.76 (1H, t, J=7.6 Hz), 6.68 (2H, d, J=7.6 Hz), 4.46 (2H, d, J=4.6 Hz), 4.11 (1H, br); HRMS (ESI-TOF) m/z: [M+H]⁺ calculated for C₁₉H₁₇N₄ 301.1448; found 301.1497.

### 5-(1H-Indazol-5-yl)-N-phenylpyridin-3-amine (Compound 16ag)

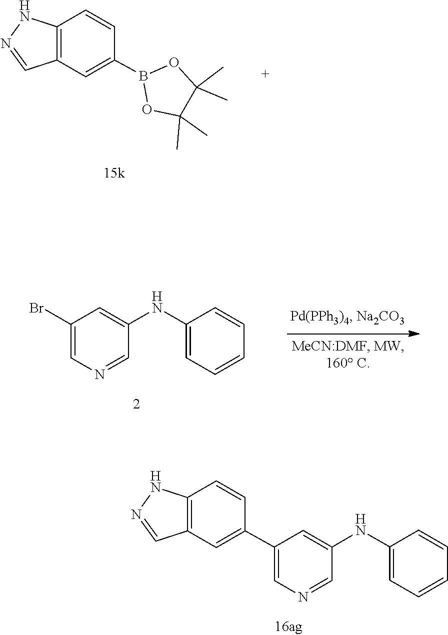 [SMILES: CC1(C)OB(c2ccc3[nH]ncc3c2)OC1(C)C ; Brc1cncc(Nc2ccccc2)c1 ; c1ccc(Nc2cncc(-c3ccc4[nH]ncc4c3)c2)cc1]

The title compound was prepared from compound 15k and compound 2 following method for preparation of compound 16c and purified by column chromatography on silica gel (MeOH/CH₂Cl₂, 2.5:97.5 to 5:95) to give compound 16ag (11%) as a white solid; ¹H NMR (DMSO-d₆, 500 MHz) 8.43 (1H, s), 8.34 (1H, d, J=1.5 Hz), 8.31 (1H, d, J=2.0 Hz), 8.13 (1H, s), 8.03 (1H, s), 7.66 (1H, t, J=2.0 Hz), 7.63 (2H, s), 7.29 (2H, t, J=7.6 Hz), 7.17 (2H, d, J=7.6 Hz), 6.90 (1H, t, J=7.1 Hz); HRMS (ESI-TOF) m/z: [M+H]⁺ calculated for C₁₉H₁₅N₄ 287.1291; found 287.1320.

### N-((5-(1H-Indazol-5-yl)pyridin-3-yl)methyl)-4-chloroaniline (Compound 16ah)

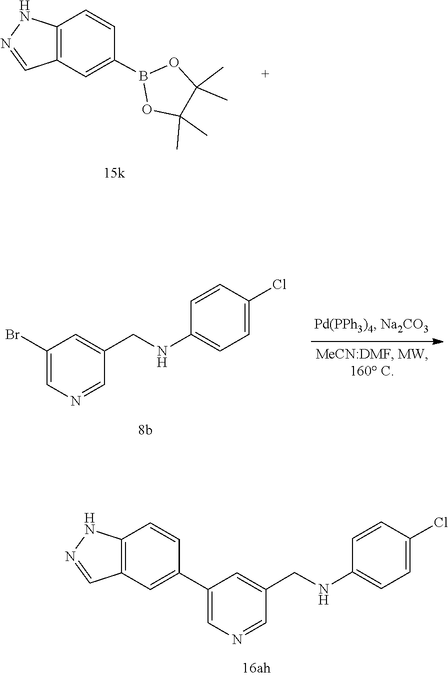 [SMILES: CC1(C)OB(c2ccc3[nH]ncc3c2)OC1(C)C ; Clc1ccc(NCc2cncc(Br)c2)cc1 ; Clc1ccc(NCc2cncc(-c3ccc4[nH]ncc4c3)c2)cc1]

The title compound was prepared from compound 15k and compound 8b following method for preparation of compound 16c and purified by column chromatography on silica gel (EtOAc/hexane, 80:20) to give compound 16ah (65%) as a pale yellow solid; ¹H NMR (CDCl₃, 500 MHz) 8.82 (1H, s), 8.60 (1H, s), 8.15 (1H, s), 7.95 (1H, s), 7.91 (1H, s), 7.61 (2H, s), 7.14 (2H, d, J=8.6 Hz), 6.59 (2H, d, J=8.6 Hz), 4.43 (2H, s), 4.13 (1H, br); HRMS (ESI-TOF) m/z: [M+H]⁺ calculated for C₁₉H₁₆ClN₄ 335.1058; found 335.1083.

### 5-(5-((Phenylamino)methyl)pyridin-3-yl)-1H-indazol-3-amine (Compound 16ai)

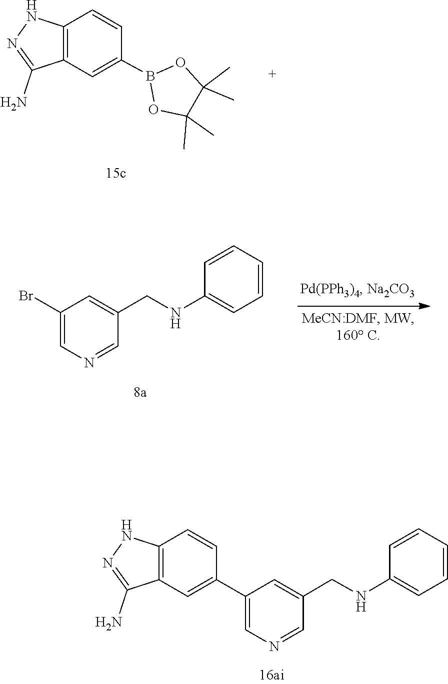 [SMILES: CC1(C)OB(c2ccc3[nH]nc(N)c3c2)OC1(C)C ; Brc1cncc(CNc2ccccc2)c1 ; Nc1n[nH]c2ccc(-c3cncc(CNc4ccccc4)c3)cc12]

The title compound was prepared from compound 15c and compound 8a following method for preparation of compound 16c and purified by column chromatography on silica gel (MeOH/CH₂Cl₂, 2.5:97.5) to give compound 16ai (56%) as a yellow solid; 1H NMR (DMSO-d₆, 600 MHz) 11.50 (1H, s), 8.73 (1H, s), 8.49 (1H, s), 8.06 (1H, s), 8.00 (1H, s), 7.55 (1H, d, J=8.5 Hz), 7.32 (1H, d, J=8.5 Hz), 7.05 (2H, t, J=8.5 Hz), 6.62 (2H, d, J=8.5 Hz), 6.52 (1H, t, J=7.3 Hz), 6.28 (1H, t, J=6.1 Hz), 5.44 (2H, s), 4.35 (2H, d, J=6.1 Hz); HRMS (ESI-TOF) m/z: [M+H]⁺ calculated for C₁₉H₁₈N₅ 316.1557; found 316.1568.

### 6-(2-((Phenylamino)methyl)pyrimidin-5-yl)-1H-indazol-3-amine (Compound 16aj)

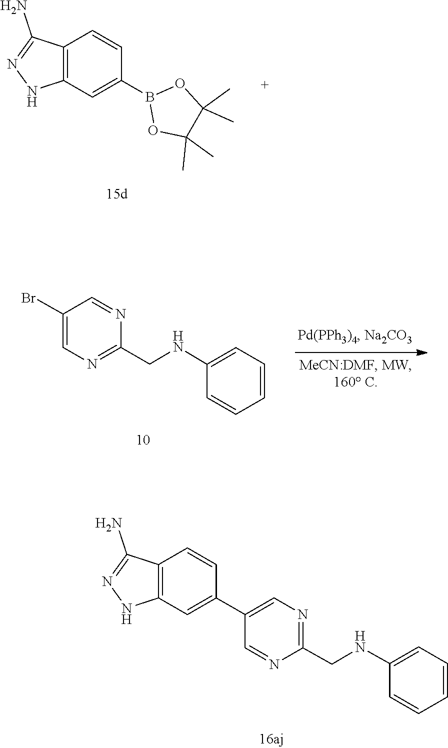 [SMILES: CC1(C)OB(c2ccc3c(N)n[nH]c3c2)OC1(C)C ; Brc1cnc(CNc2ccccc2)nc1 ; Nc1n[nH]c2cc(-c3cnc(CNc4ccccc4)nc3)ccc12]

The title compound was prepared from compound 15c and compound 10 following method for preparation of compound 16c and purified by column chromatography on silica gel (MeOH/CH₂Cl₂, 2.5:97.5 to 5:95) to give compound 16aj (58%) as a light brown solid; 1H NMR (DMSO-d₆, 500 MHz) 11.57 (1H, s), 9.11 (2H, s), 7.81 (1H, d, J=8.1 Hz), 7.57 (1H, s), 7.28 (1H, d, J=8.1 Hz), 7.05 (2H, t, J=8.1 Hz), 6.66 (2H, d, J=8.1 Hz), 6.52 (1H, t, J=7.1 Hz), 6.22 (1H, t, J=6.1 Hz), 5.40 (2H, s), 4.49 (2H, d, J=6.1 Hz); RMS (ESI-TOF) m/z: [M+H]⁺ calculated for C₁₈H₁₇N₆ 316.1509; found 316.0662.

### 6-(2-(Phenylamino)pyrimidin-5-yl)-1H-indazol-3-amine (Compound 16ak)

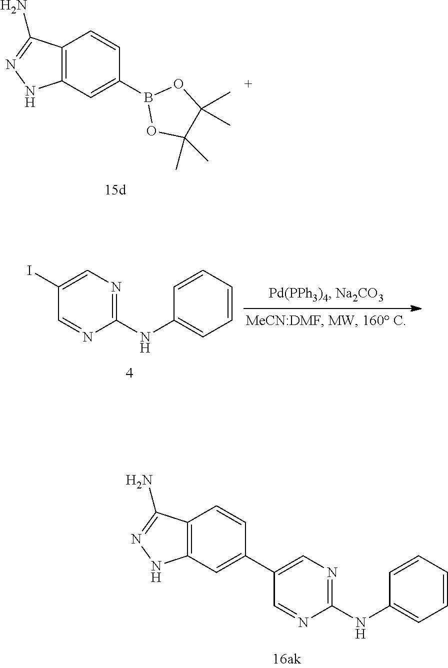 [SMILES: CC1(C)OB(c2ccc3c(N)n[nH]c3c2)OC1(C)C ; Ic1cnc(Nc2ccccc2)nc1 ; Nc1n[nH]c2cc(-c3cnc(Nc4ccccc4)nc3)ccc12]

The title compound was prepared from compound 15d and compound 4 following method for preparation of compound 16c and purified by column chromatography on silica gel (MeOH/CH₂Cl₂, 2.5:97.5 to 5:95) to give compound 16ak (49%) as a brown solid; 1H NMR (DMSO-d₆, 600 MHz) 11.48 (1H, s), 9.77 (1H, s), 8.84 (2H, s), 7.80 (2H, d, J=8.5 Hz), 7.76 (1H, d, J=7.3 Hz), 7.47 (1H, s), 7.29 (2H, t, J=7.3 Hz), 7.22 (1H, d, J=8.5 Hz), 6.95 (1H, t, J=7.3 Hz), 5.38 (2H, s); HRMS (ESI-TOF) m/z: [M+H]⁺ calculated for C₁₇H₁₅N₆ 303.1353; found 303.1409.

### 5-(5-(Phenylamino)pyridin-3-yl)-1H-indazol-3-amine (Compound 16al)

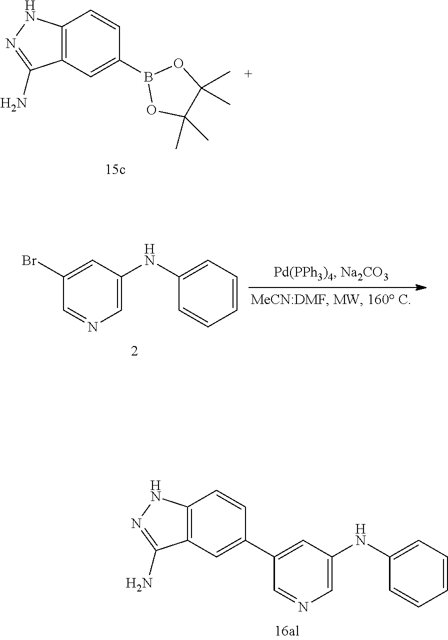 [SMILES: CC1(C)OB(c2ccc3[nH]nc(N)c3c2)OC1(C)C ; Brc1cncc(Nc2ccccc2)c1 ; Nc1n[nH]c2ccc(-c3cncc(Nc4ccccc4)c3)cc12]

The title compound was prepared from compound 15c and compound 2 following method for preparation of compound 16c and purified by column chromatography on silica gel (MeOH/CH₂Cl₂, 5:95) to give compound 16al (54%) as an off-white solid; 1H NMR (DMSO-d₆, 600 MHz) 11.48 (1H, s), 8.44 (1H, s), 8.31 (1H, d, J=2.4 Hz), 8.26 (1H, d, J=2.4 Hz), 8.01 (1H, s), 7.64 (1H, t, J=4.9 Hz), 7.52 (1H, d, J=7.3 Hz), 7.32-7.27 (3H, m), 7.15 (2H, d, J=8.5 Hz), 6.89 (1H, t, J=7.3 Hz), 5.44 (2H, s); HRMS (ESI-TOF) m/z: [M+H]⁺ calculated for C₁₈H₁₆N₅ 302.1400; found 302.1401.

### 5-(5-((2-Fluorobenzyl)amino)pyridin-3-yl)-1H-indazol-3-amine (Compound 16am)

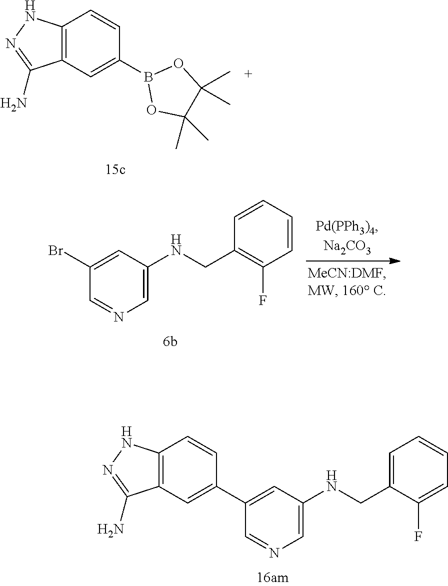 [SMILES: CC1(C)OB(c2ccc3[nH]nc(N)c3c2)OC1(C)C ; Fc1ccccc1CNc1cncc(Br)c1 ; Nc1n[nH]c2ccc(-c3cncc(NCc4ccccc4F)c3)cc12]

The title compound was prepared from compound 15c and compound 6b following method for preparation of compound 16c and purified by column chromatography on silica gel (MeOH/CH₂Cl₂, 5:95) to give compound 16am (24%) as a yellow solid; ¹H NMR (DMSO-d₆, 600 MHz) 11.45 (1H, s), 8.06 (1H, s), 7.96 (1H, s), 7.92 (1H, d, J=2.4 Hz), 7.45 (2H, d, J=7.3 Hz), 7.31-7.28 (2H, m), 7.21-7.14 (3H, m), 6.50 (1H, t, J=6.1 Hz), 5.41 (2H, s), 4.42 (2H, d, J=6.1 Hz); HRMS (ESI-TOF) m/z: calculated for C₁₉H₁₇FN₅ 334.1463; found 334.1449.

### 5-(5-((2-Chlorobenzyl)amino)pyridin-3-yl)-1H-indazol-3-amine (Compound 16an)

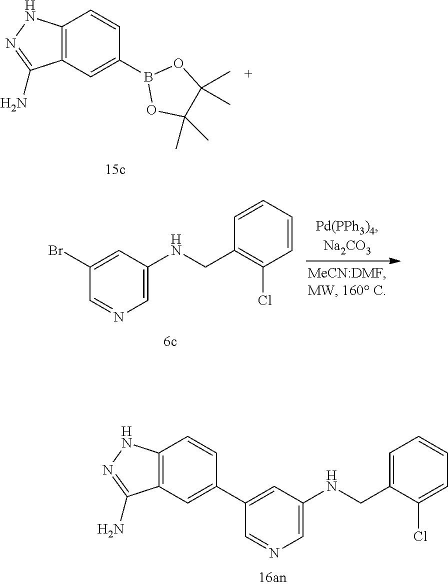 [SMILES: CC1(C)OB(c2ccc3[nH]nc(N)c3c2)OC1(C)C ; Clc1ccccc1CNc1cncc(Br)c1 ; Nc1n[nH]c2ccc(-c3cncc(NCc4ccccc4Cl)c3)cc12]

The title compound was prepared from compound 15c and compound 6c following method for preparation of compound 16c and purified by column chromatography on silica gel (MeOH/CH₂Cl₂, 5:95) to give compound 16an (39%) as a yellow solid; ¹H NMR (DMSO-d₆, 600 MHz) 11.44 (1H, s), 8.06 (1H, s), 7.96 (1H, s), 7.88 (1H, d, J=2.4 Hz), 7.47-7.39 (3H, m), 7.34-7.26 (3H, m), 7.09 (1H, s), 6.58 (1H, t, J=6.1 Hz), 5.40 (2H, s), 4.44 (2H, d, J=6.1 Hz); HRMS (ESI-TOF) m/z: calculated for C₁₉H₁₇ClN₅ 350.1167; found 350.1165.

### (S)-5-(5-((1-Phenylethyl)amino)pyridin-3-yl)-1H-indazol-3-amine (Compound 16ao)

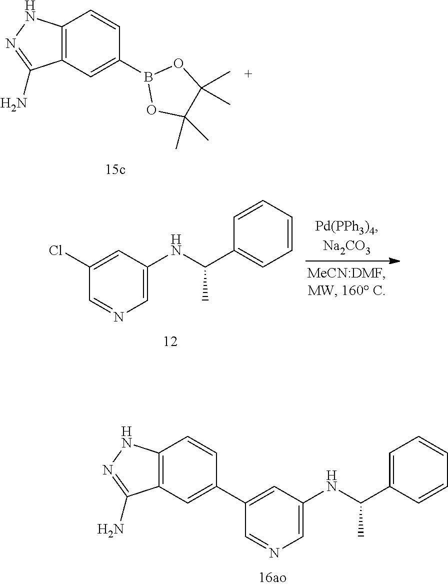 [SMILES: CC1(C)OB(c2ccc3[nH]nc(N)c3c2)OC1(C)C ; C[C@H](Nc1cncc(Cl)c1)c1ccccc1 ; C[C@H](Nc1cncc(-c2ccc3[nH]nc(N)c3c2)c1)c1ccccc1]

The title compound was prepared from compound 15c and compound 12 following method for preparation of compound 16c and purified by column chromatography on silica gel (MeOH/CH₂Cl₂, 5:95) to give compound 16ao (26%) as a yellow solid; 1H NMR (DMSO-d₆, 600 MHz) 11.43 (1H, s), 7.97 (1H, s), 7.90 (1H, s), 7.81 (1H, d, J=2.4 Hz), 7.41 (2H, d, J=8.5 Hz), 7.34 (1H, d, J=8.5 Hz), 7.30 (2H, t, J=7.3 Hz), 7.26 (1H, d, J=9.8 Hz), 7.18 (1H, t, J=7.3 Hz), 7.02 (1H, s), 6.50 (1H, d, J=7.3 Hz), 5.39 (2H, s), 4.61 (1H, quint, J=4.9 Hz), 1.46 (3H, d, J=6.1 Hz); HRMS (ESI-TOF) m/z: calculated for C₂₀H₂₀N₅ 330.1713; found 330.1710.

### N-(2-Fluorobenzyl)-5-(1H-indazol-5-yl)pyridin-3-amine (Compound 16ap)

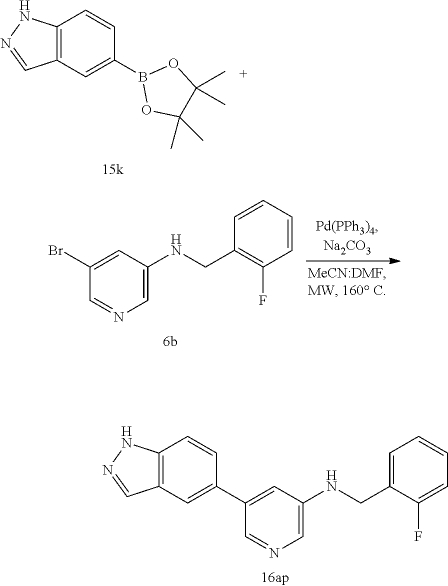 [SMILES: CC1(C)OB(c2ccc3[nH]ncc3c2)OC1(C)C ; Fc1ccccc1CNc1cncc(Br)c1 ; Fc1ccccc1CNc1cncc(-c2ccc3[nH]ncc3c2)c1]

The title compound was prepared from compound 15k and compound 6b following method for preparation of compound 16c and purified by column chromatography on silica gel (MeOH/CH₂Cl₂, 5:95) to give compound 16ap (50%) as a brown solid; 1H NMR (DMSO-d₆, 600 MHz) 13.12 (1H, s), 8.11 (1H, s), 8.09 (1H, s), 7.96 (2H, s), 7.61-7.56 (2H, m), 7.44 (1H, t, J=8.5 Hz), 7.31-7.29 (1H, m), 7.22-7.15 (3H, m), 6.53 (1H, t, J=6.1 Hz), 4.43 (2H, d, J=6.1 Hz); HRMS (ESI-TOF) m/z: calculated for C₁₉H₁₆FN₄ 319.1354; found 319.1424.

### N-(2-Chlorobenzyl)-5-(1H-indazol-5-yl)pyridin-3-amine (Compound 16aq)

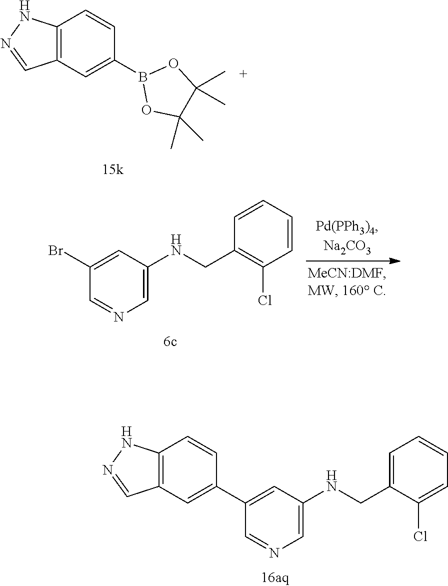 [SMILES: CC1(C)OB(c2ccc3[nH]ncc3c2)OC1(C)C ; Clc1ccccc1CNc1cncc(Br)c1 ; Clc1ccccc1CNc1cncc(-c2ccc3[nH]ncc3c2)c1]

The title compound was prepared from compound 15k and compound 6c following method for preparation of compound 16c and purified by column chromatography on silica gel (MeOH/CH₂Cl₂, 5:95) to give compound 16aq (44%) as a white solid; 1H NMR (DMSO-d₆, 600 MHz) 13.12 (1H, s), 8.09 (1H, s), 8.06 (1H, s), 7.96 (2H, s), 7.60-7.52 (4H, m), 7.46 (1H, t, J=7.3 Hz), 7.30-7.28 (1H, m), 7.13 (1H, s), 6.60-6.58 (1H, m), 4.46 (2H, d, J=6.1 Hz); HRMS (ESI-TOF) m/z: calculated for C₁₉H₁₆ClN₄ 335.1058; found 335.1076.

### (S)-5-(1H-Indazol-5-yl)-N-(1-phenylethyl)pyridin-3-amine (Compound 16ar)

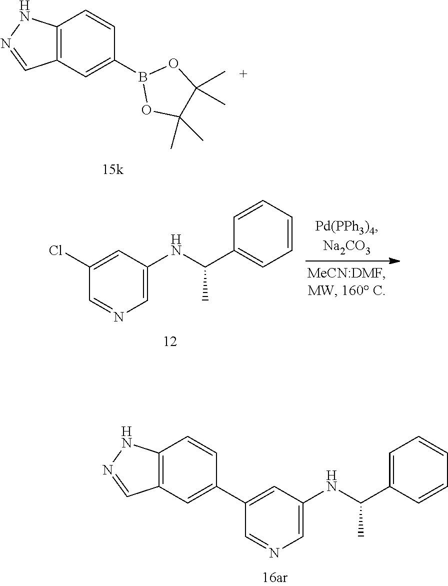 [SMILES: CC1(C)OB(c2ccc3[nH]ncc3c2)OC1(C)C ; C[C@H](Nc1cncc(Cl)c1)c1ccccc1 ; C[C@H](Nc1cncc(-c2ccc3[nH]ncc3c2)c1)c1ccccc1]

The title compound was prepared from compound 15k and compound 12 following method for preparation of compound 16c and purified by column chromatography on silica gel (MeOH/CH₂Cl₂, 5:95) to give compound 16ar (28%) as a yellow solid; ¹H NMR (DMSO-d₆, 600 MHz) 13.12 (1H, s), 8.09 (1H, s), 8.00 (1H, s), 7.87-7.86 (2H, m), 7.58 (1H, d, J=8.5 Hz), 7.47 (1H, d, J=8.5 Hz), 7.42 (2H, d, J=7.3 Hz), 7.31 (2H, t, J=8.5 Hz), 7.18 (1H, t, J=7.3 Hz), 7.03 (1H, s), 6.54 (1H, d, J=7.3 Hz), 4.64 (1H, quint, J=6.1 Hz), 1.46 (3H, d, J=7.3 Hz); HRMS (ESI-TOF) m/z: calculated for C₂₀H₁₉N₄ 315.1604; found 315.1781.

### 5-(5-(((4-Chlorophenyl)amino)methyl)pyridin-3-yl)-1H-indazol-3-amine (Compound 16as)

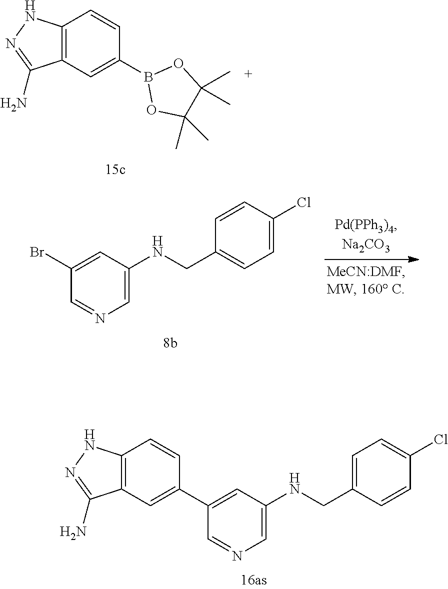 [SMILES: CC1(C)OB(c2ccc3[nH]nc(N)c3c2)OC1(C)C ; Clc1ccc(CNc2cncc(Br)c2)cc1 ; Nc1n[nH]c2ccc(-c3cncc(NCc4ccc(Cl)cc4)c3)cc12]

The title compound was prepared from compound 15c and compound 8b following method for preparation of compound 16c and purified by column chromatography on silica gel (MeOH/CH₂Cl₂, 5:95) to give compound 16as (48%) as an off-white solid; 1H NMR (DMSO-d₆, 600 MHz) 11.50 (1H, s), 8.74 (1H, d, J=2.4 Hz), 8.48 (1H, s), 8.07 (1H, s), 8.00 (1H, s), 7.55 (1H, d, J=7.3 Hz), 7.32 (1H, d, J=8.5 Hz), 7.08 (2H, d, J=8.5 Hz), 6.63 (2H, d, J=8.5 Hz), 6.50 (1H, t, J=7.3 Hz), 5.44 (2H, s), 4.34 (2H, d, J=4.9 Hz); HRMS (ESI-TOF) m/z: calculated for C₁₉H₁₇ClN₅ 350.1167; found 350.1163.

### 6-(2-((3-Methoxybenzyl)amino)pyrimidin-5-yl)-1H-indazol-3-amine (Compound 16at)

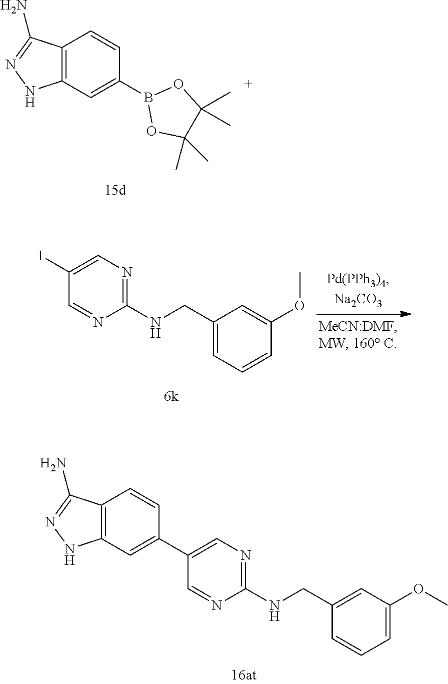 [SMILES: CC1(C)OB(c2ccc3c(N)n[nH]c3c2)OC1(C)C ; COc1cccc(CNc2ncc(I)cn2)c1 ; COc1cccc(CNc2ncc(-c3ccc4c(N)n[nH]c4c3)cn2)c1]

The title compound was prepared from compound 15d and compound 6k following method for preparation of compound 16c and purified by column chromatography on silica gel (MeOH/CH₂Cl₂, 5:95) to give compound 16at (30%) as an off-white solid; 1H NMR (DMSO-d₆, 600 MHz) 11.41 (1H, s), 8.83 (2H, s), 7.85 (1H, t, J=6.1 Hz), 7.71 (1H, d, J=8.5 Hz), 7.36 (1H, s), 7.21 (1H, t, J=8.5 Hz), 7.13 (1H, d, J=7.3 Hz), 6.90-6.89 (2H, m), 6.78 (1H, d, J=8.5 Hz), 5.34 (2H, s), 4.52 (2H, d, J=6.1 Hz), 3.71 (3H, s); HRMS (ESI-TOF) m/z: calculated for C₁₉H₁₉N₆O 347.1615; found 347.1310.

### 6-(2-((4-Methoxybenzyl)amino)pyrimidin-5-yl)-1H-indazol-3-amine (Compound 16au)

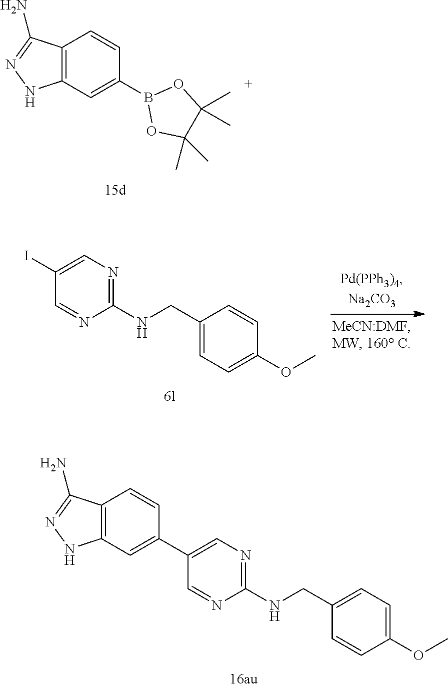 [SMILES: CC1(C)OB(c2ccc3c(N)n[nH]c3c2)OC1(C)C ; COc1ccc(CNc2ncc(I)cn2)cc1 ; COc1ccc(CNc2ncc(-c3ccc4c(N)n[nH]c4c3)cn2)cc1]

The title compound was prepared from compound 15d and compound 61 following method for preparation of compound 16c and purified by column chromatography on silica gel (MeOH/CH₂Cl₂, 5:95) to give compound 16au (24%) as a light-brown solid; 1H NMR (DMSO-d₆, 600 MHz) 11.40 (1H, s), 8.62 (2H, s), 7.79 (1H, t, J=8.5 Hz), 7.71 (1H, d, J=7.3 Hz), 7.35 (1H, s), 7.25 (2H, d, J=8.5 Hz), 7.13 (1H, d, J=8.5 Hz), 6.86 (2H, d, J=8.5 Hz), 5.34 (2H, s), 4.47 (2H, d, J=6.1 Hz), 3.71 (3H, s); HRMS (ESI-TOF) m/z: calculated for C₁₉H₁₉N₆O 347.1615; found 347.1477.

### 6-(2-((2-Chlorobenzyl)amino)pyrimidin-5-yl)-1H-indazol-3-amine (Compound 16av)

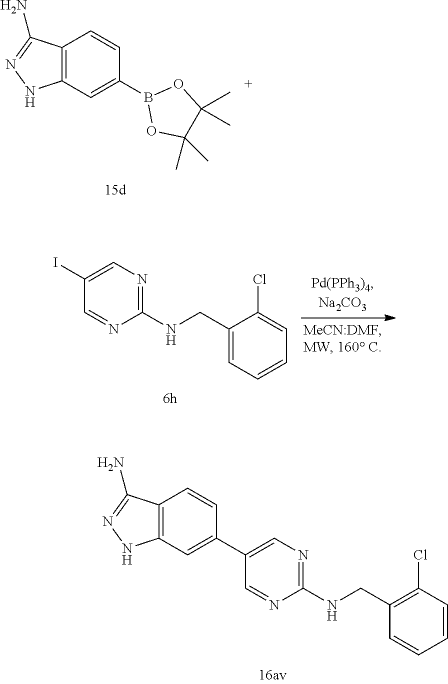 [SMILES: CC1(C)OB(c2ccc3c(N)n[nH]c3c2)OC1(C)C ; Clc1ccccc1CNc1ncc(I)cn1 ; Nc1n[nH]c2cc(-c3cnc(NCc4ccccc4Cl)nc3)ccc12]

The title compound was prepared from compound 15d and compound 6h following method for preparation of compound 16c and purified by column chromatography on silica gel (MeOH/CH₂Cl₂, 5:95) to give compound 16av (37%) as a brown solid; 1H NMR (DMSO-d₆, 600 MHz) 11.41 (1H, s), 8.65 (2H, s), 7.88 (1H, t, J=6.1 Hz), 7.71 (1H, d, J=8.5 Hz), 7.44 (1H, d, J=8.5 Hz), 7.36-7.26 (4H, m), 7.13 (1H, d, J=6.1 Hz), 5.34 (2H, s), 4.61 (2H, d, J=6.1 Hz); HRMS (ESI-TOF) m/z: calculated for C₁₈H₁₆ClN₆ 351.1119; found 351.1116.

### 6-(2-((3-Chlorobenzyl)amino)pyrimidin-5-yl)-1H-indazol-3-amine (Compound 16aw)

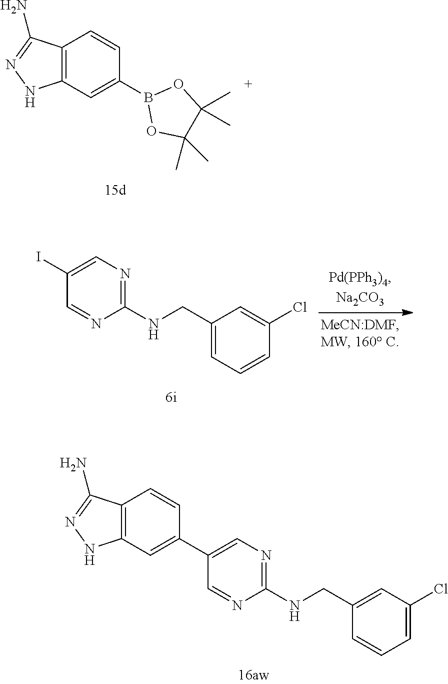 [SMILES: CC1(C)OB(c2ccc3c(N)n[nH]c3c2)OC1(C)C ; Clc1cccc(CNc2ncc(I)cn2)c1 ; Nc1n[nH]c2cc(-c3cnc(NCc4cccc(Cl)c4)nc3)ccc12]

The title compound was prepared from compound 15d and compound 6i following method for preparation of compound 16c and purified by column chromatography on silica gel (MeOH/CH₂Cl₂, 5:95) to give compound 16aw (19%) as a brown solid; 1H NMR (DMSO-d₆, 600 MHz) 11.41 (1H, s), 8.64 (2H, s), 7.92 (1H, t, J=6.1 Hz), 7.71 (1H, d, J=8.5 Hz), 7.36-7.26 (5H, m), 7.13 (1H, d, J=8.5 Hz), 5.34 (2H, s), 4.55 (2H, d, J=6.1 Hz); HRMS (ESI-TOF) m/z: calculated for C₁₈H₁₆ClN₆ 351.1119; found 351.1147.

### 6-(2-((4-Chlorobenzyl)amino)pyrimidin-5-yl)-1H-indazol-3-amine (Compound 16ax)

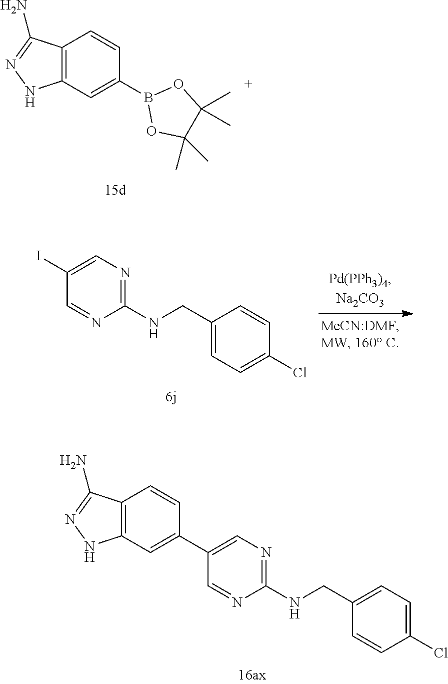 [SMILES: CC1(C)OB(c2ccc3c(N)n[nH]c3c2)OC1(C)C ; Clc1ccc(CNc2ncc(I)cn2)cc1 ; Nc1n[nH]c2cc(-c3cnc(NCc4ccc(Cl)cc4)nc3)ccc12]

The title compound was prepared from compound 15d and compound 6j following method for preparation of compound 16c and purified by column chromatography on silica gel (MeOH/CH₂Cl₂, 5:95 to 10:90) to give compound 16ax (41%) as a yellow solid; 1H NMR (DMSO-d₆, 600 MHz) 11.41 (1H, s), 8.63 (2H, s), 7.90 (1H, t, J=7.3 Hz), 7.71 (1H, d, J=8.5 Hz), 7.37-7.33 (5H, m), 7.12 (1H, d, J=9.8 Hz), 5.34 (2H, s), 4.53 (2H, d, J=6.1 Hz); HRMS (ESI-TOF) m/z: calculated for C₁₈H₁₆ClN₆ 351.1119; found 351.1113.

### 6-(2-((2-Fluorobenzyl)amino)pyrimidin-5-yl)-1H-indazol-3-amine (Compound 16ay)

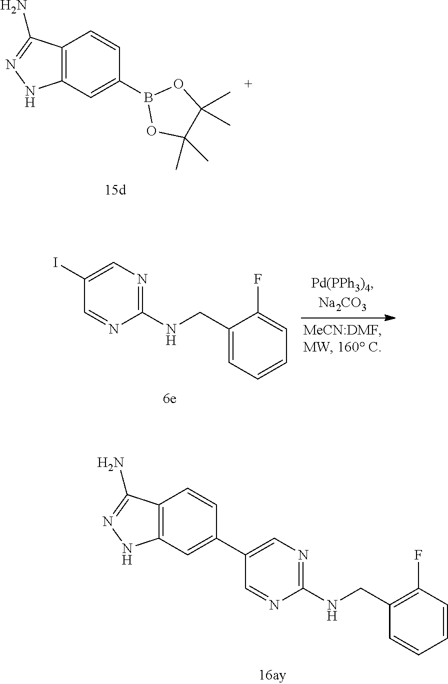 [SMILES: CC1(C)OB(c2ccc3c(N)n[nH]c3c2)OC1(C)C ; Fc1ccccc1CNc1ncc(I)cn1 ; Nc1n[nH]c2cc(-c3cnc(NCc4ccccc4F)nc3)ccc12]

The title compound was prepared from compound 15d and compound 6e following method for preparation of compound 16a and purified by column chromatography on silica gel (MeOH/CH₂Cl₂, 5:95) to give compound 16ay (37%) as a light-brown solid; 1H NMR (DMSO-d₆, 600 MHz) 11.41 (1H, s), 8.64 (2H, s), 7.84 (1H, t, J=7.3 Hz), 7.71 (1H, d, J=8.5 Hz), 7.36-7.34 (2H, m), 7.28-7.27 (1H, m), 7.18-7.12 (3H, m), 5.34 (2H, s), 4.60 (2H, d, J=6.1 Hz); HRMS (ESI-TOF) m/z: calculated for C₁₈H₁₆FN₆ 355.1415; found 335.1389.

### 6-(2-((3-Fluorobenzyl)amino)pyrimidin-5-yl)-1H-indazol-3-amine (Compound 16az)

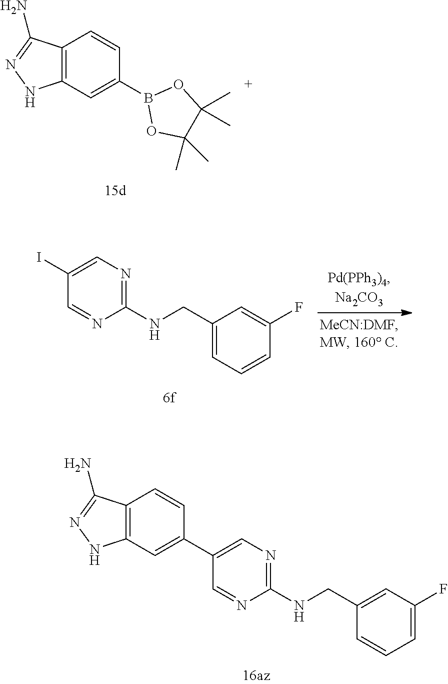 [SMILES: CC1(C)OB(c2ccc3c(N)n[nH]c3c2)OC1(C)C ; Fc1cccc(CNc2ncc(I)cn2)c1 ; Nc1n[nH]c2cc(-c3cnc(NCc4cccc(F)c4)nc3)ccc12]

The title compound was prepared from compound 15d and compound 6f following method for preparation of compound 16a and purified by column chromatography on silica gel (MeOH/CH₂Cl₂, 5:95 to 10:90) to give compound 16az (28%) as a light-brown solid; 1H NMR (DMSO-d₆, 600 MHz) 11.41 (1H, s), 8.64 (2H, s), 7.91 (1H, t, J=6.1 Hz), 7.71 (1H, d, J=8.5 Hz), 7.36-7.33 (2H, m), 7.17 (1H, d, J=7.3 Hz), 7.12 (2H, t, J=8.5 Hz), 7.03 (1H, td, J=9.8, 2.4 Hz), 5.34 (2H, s), 4.56 (2H, d, J=6.1 Hz); HRMS (ESI-TOF) m/z: calculated for C₁₈H₁₆FN₆ 355.1415; found 335.1409.

### 6-(2-((4-Fluorobenzyl)amino)pyrimidin-5-yl)-1H-indazol-3-amine (Compound 16ba)

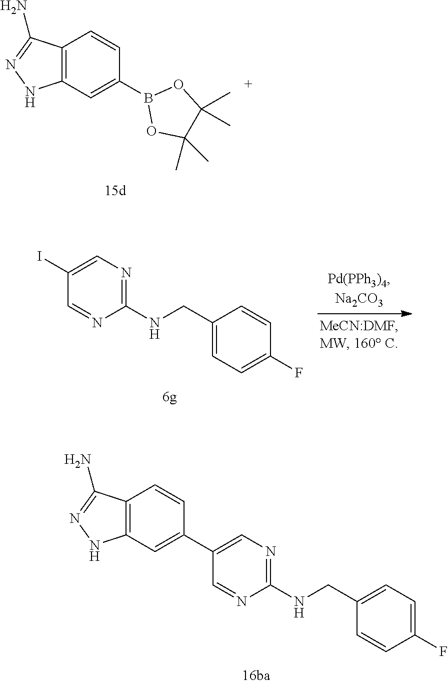 [SMILES: CC1(C)OB(c2ccc3c(N)n[nH]c3c2)OC1(C)C ; Fc1ccc(CNc2ncc(I)cn2)cc1 ; Nc1n[nH]c2cc(-c3cnc(NCc4ccc(F)cc4)nc3)ccc12]

The title compound was prepared from compound 15d and compound 6g following method for preparation of compound 16a and purified by column chromatography on silica gel (MeOH/CH₂Cl₂, 5:95 to 10:90) to give compound 16ba (19%) as a light-brown solid; 1H NMR (DMSO-d₆, 600 MHz) 11.41 (1H, s), 8.63 (2H, s), 7.88 (1H, t, J=6.1 Hz), 7.71 (1H, d, J=7.3 Hz), 7.37-7.35 (3H, m), 7.14-7.11 (3H, m), 5.34 (2H, s), 4.53 (2H, d, J=6.1 Hz); HRMS (ESI-TOF) m/z: calculated for C₁₈H₁₆FN₆ 355.1415; found 335.1417.

### 6-(2-((4-(Trifluoromethoxy)benzyl)amino)pyrimidin-5-yl)-1H-indazol-3-amine (Compound 16bb)

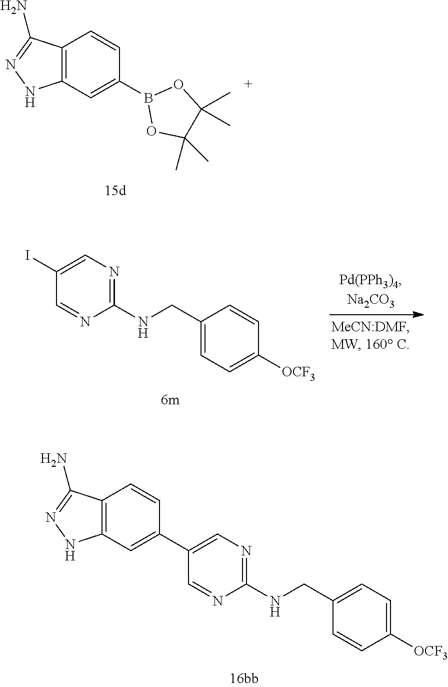 [SMILES: CC1(C)OB(c2ccc3c(N)n[nH]c3c2)OC1(C)C ; FC(F)(F)Oc1ccc(CNc2ncc(I)cn2)cc1 ; Nc1n[nH]c2cc(-c3cnc(NCc4ccc(OC(F)(F)F)cc4)nc3)ccc12]

The title compound was prepared from compound 15d and compound 6m following method for preparation of compound 16a and purified by column chromatography on silica gel (MeOH/CH₂Cl₂, 5:95 to 10:90) to give compound 16bb (18%) as a light-brown solid; 1H NMR (DMSO-d₆, 500 MHz) 11.39 (1H, s), 8.63 (2H, s), 7.90 (1H, t, J=6.6 Hz), 7.71 (1H, d, J=8.1 Hz), 7.44 (2H, d, J=8.1 Hz), 7.36 (1H, s), 7.30 (2H, d, J=8.1 Hz), 7.13 (1H, d, J=8.1 Hz), 5.32 (2H, s), 4.57 (2H, d, J=6.6 Hz); HRMS (ESI-TOF) m/z: calculated for C₁₉H₁₆F₃N₆O 401.1332; found 401.1294.

### 6-(2-((2-(Trifluoromethyl)benzyl)amino)pyrimidin-5-yl)-1H-indazol-3-amine (Compound 16bc)

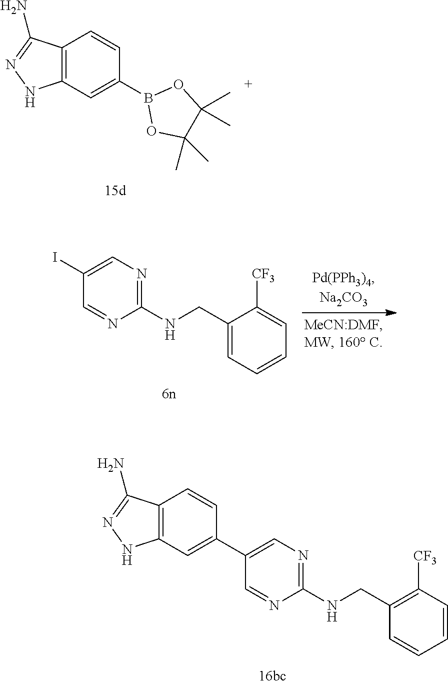 [SMILES: CC1(C)OB(c2ccc3c(N)n[nH]c3c2)OC1(C)C ; FC(F)(F)c1ccccc1CNc1ncc(I)cn1 ; Nc1n[nH]c2cc(-c3cnc(NCc4ccccc4C(F)(F)F)nc3)ccc12]

The title compound was prepared from compound 15d and compound 6n following method for preparation of compound 16a and purified by column chromatography on silica gel (MeOH/CH₂Cl₂, 5:95 to 10:90) to give compound 16bc (36%) as a light-brown solid; 1H NMR (DMSO-d₆, 500 MHz) 11.40 (1H, s), 8.66 (2H, s), 7.89 (1H, t, J=6.1 Hz), 7.71 (2H, dd, J=6.7, 2.5 Hz), 7.62 (1H, t, J=7.6 Hz), 7.53 (1H, d, J=8.1 Hz), 7.44 (1H, t, J=7.6 Hz), 7.37 (1H, s), 7.14 (1H, d, J=8.6 Hz), 5.32 (2H, s), 4.76 (2H, d, J=6.1 Hz); HRMS (ESI-TOF) m/z: calculated for C₁₉H₁₆F₃N₆ 385.1383; found 385.1404.

### 6-(2-((3-(Trifluoromethyl)benzyl)amino)pyrimidin-5-yl)-1H-indazol-3-amine (Compound 16bd)

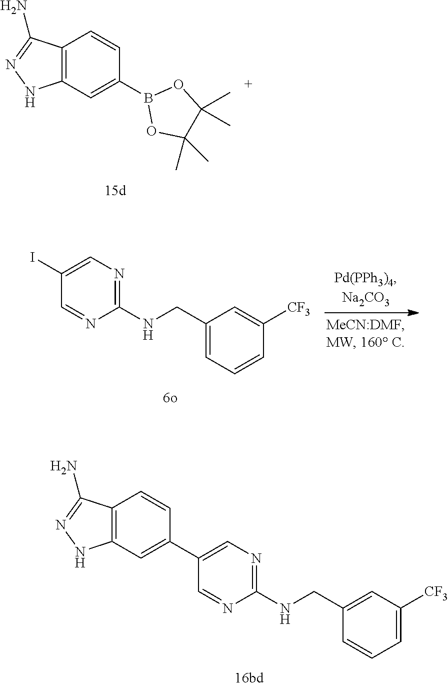 [SMILES: CC1(C)OB(c2ccc3c(N)n[nH]c3c2)OC1(C)C ; FC(F)(F)c1cccc(CNc2ncc(I)cn2)c1 ; Nc1n[nH]c2cc(-c3cnc(NCc4cccc(C(F)(F)F)c4)nc3)ccc12]

The title compound was prepared from compound 15d and compound 6o following method for preparation of compound 16a and purified by column chromatography on silica gel (MeOH/CH₂Cl₂, 5:95) to give compound 16bd (39%) as a light-brown solid; 1H NMR (DMSO-d₆, 500 MHz) 11.39 (1H, s), 8.64 (2H, s), 7.95 (1H, t, J=6.6 Hz), 7.71 (1H, d, J=8.1 Hz), 7.68 (1H, s), 7.64 (1H, d, J=7.6 Hz), 7.59-7.53 (2H, m), 7.36 (1H, s), 7.13 (1H, d, J=8.1 Hz), 5.32 (2H, s), 4.64 (2H, d, J=6.1 Hz); HRMS (ESI-TOF) m/z: calculated for C₁₉H₁₆F₃N₆ 385.1383; found 385.1424.

### 6-(2-((4-(Trifluoromethyl)benzyl)amino)pyrimidin-5-yl)-1H-indazol-3-amine (Compound 16be)

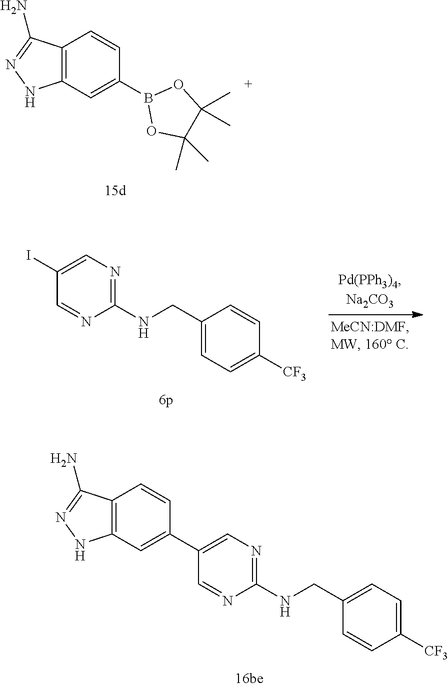 [SMILES: CC1(C)OB(c2ccc3c(N)n[nH]c3c2)OC1(C)C ; FC(F)(F)c1ccc(CNc2ncc(I)cn2)cc1 ; Nc1n[nH]c2cc(-c3cnc(NCc4ccc(C(F)(F)F)cc4)nc3)ccc12]

The title compound was prepared from compound 15d and compound 6p following method for preparation of compound 16a and purified by column chromatography on silica gel (MeOH/CH₂Cl₂, 5:95) to give compound 16be (23%) as a brown solid; ¹H NMR (DMSO-d₆, 500 MHz) 11.40 (1H, s), 8.63 (2H, s), 7.96 (1H, t, J=6.1 Hz), 7.71 (1H, d, J=8.6 Hz), 7.67 (2H, d, J=8.1 Hz), 7.54 (2H, d, J=7.6 Hz), 7.36 (1H, s), 7.13 (1H, d, J=8.6 Hz), 5.32 (2H, s), 4.63 (2H, d, J=6.6 Hz); HRMS (ESI-TOF) m/z: calculated for C₁₉H₁₆F₃N₆ 385.1383; found 385.1390.

### (S)-5-(3-Aminophenyl)-N-(1-phenylethyl)pyridin-3-amine (Compound 17a)

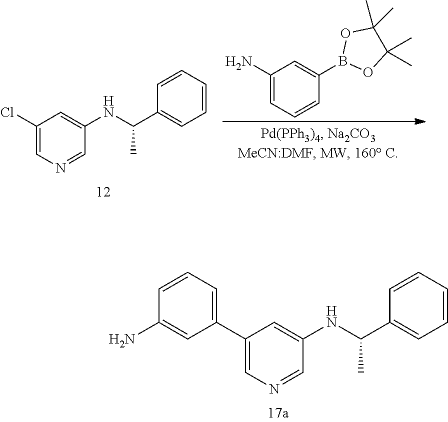 [SMILES: C[C@H](Nc1cncc(Cl)c1)c1ccccc1 ; CC1(C)OB(c2cccc(N)c2)OC1(C)C ; C[C@H](Nc1cncc(-c2cccc(N)c2)c1)c1ccccc1]

The title compound was prepared from compound 12 and 3-aminophenylboronic acid pinacol ester following method for preparation of compound 16a and purified by column chromatography on silica gel (MeOH/CH₂Cl₂, 5:95) to give compound 17a (42%) as a yellow oil; 1H NMR (CDCl₃, 500 MHz) 8.10 (1H, s), 7.94 (1H, s), 7.38-7.32 (4H, m), 7.25-7.14 (3H, m), 6.89 (1H, s), 6.79 (1H, t, J=7.1 Hz), 6.71 (1H, t, J=1.5 Hz), 6.66 (1H, dd, J=8.1, 1.5 Hz), 4.55 (1H, q, J=6.6 Hz), 3.70 (2H, br), 1.57 (3H, d, J=6.6 Hz); HRMS (ESI-TOF) m/z: calculated for C₁₉H₂₀N₃ 290.1652; found 290.1686.

### (S)-(4-Fluoro-3-((3-(5-((I-phenylethyl)amino)pyridin-3-yl)phenyl)carbamoyl)phenyl)boronic acid (Compound 16bf)

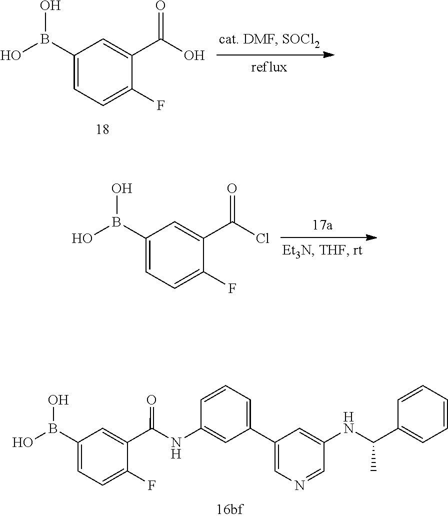 [SMILES: O=C(O)c1cc(B(O)O)ccc1F ; O=C(Cl)c1cc(B(O)O)ccc1F ; C[C@H](Nc1cncc(-c2cccc(NC(=O)c3cc(B(O)O)ccc3F)c2)c1)c1ccccc1]

To a solution of compound 18 (19.0 mg, 0.10 mmol) in thionyl chloride (2 mL) was catalytic amount of DMF under nitrogen. The resulting mixture was refluxed for 24 h. The excess thionyl chloride was removed in vacuo. The crude product was dissolved in THF (3 mL) then compound 17a (30 mg, 0.10 mmol) was added. The reaction was purged with nitrogen then triethylamine (0.1 mL, 0.72 mmol) was added dropwise at 0° C. The reaction was stirred at room temperature for 3 h. After being quenched by the addition of 1 N HCl, the aqueous layer was extracted with EtOAc (2×10 mL). The combined organic extracts were washed with brine, dried over anhydrous Na₂SO₄, filtered, and concentrated. The crude product was washed with CH₂Cl₂ and hexane afford compound 16bf (110 mg, 93%) as a brown solid; 1H NMR (DMSO-d₆, 400 MHz) 8.23 (2H, br), 8.13 (1H, d, J=1.3 Hz), 8.10 (1H, dd, J=7.7, 1.3 Hz), 8.00-7.96 (1H, m), 7.90 (1H, d, J=2.0 Hz), 7.69 (1H, s), 7.50 (1H, t, J=8.0 Hz), 7.44 (2H, d, J=7.3 Hz), 7.38-7.30 (4H, m), 7.23 (1H, t, J=7.3 Hz), 4.78 (1H, quint, J=6.7 Hz), 1.50 (3H, d, J=6.7 Hz); HRMS (ESI-TOF) m/z: calculated for C₂₆H₂₄BFN₃O₃ 456.1889; found 456.2099.

### (S)-5-(5-((1-Phenylethyl)amino)pyridin-3-yl)thiazol-2-amine (Compound 17b)

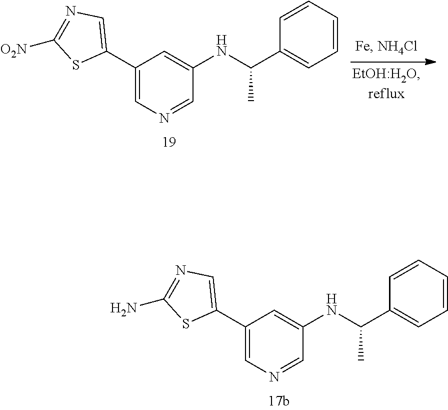 [SMILES: C[C@H](Nc1cncc(-c2cnc([N+](=O)[O-])s2)c1)c1ccccc1 ; C[C@H](Nc1cncc(-c2cnc(N)s2)c1)c1ccccc1]

To a solution of compound 19 (24 mg, 0.07 mmol) in EtOH (3 mL) and water (0.6 mL) was added ammonium chloride (21 mg, 0.39 mmol) and iron (21 mg, 0.37 mmol). The resulting mixture was refluxed at 85° C. for 1.5 h. The reaction was filtered through a Celite pad and the solvent was removed under vacuum. The reaction was diluted with EtOAc (5 mL) and water (10 mL). The aqueous layer was extracted with EtOAc (2×10 mL). The combined organic extracts were washed with brine, dried over anhydrous Na₂SO₄, filtered, and concentrated. The residue was and purified by column chromatography on silica gel (MeOH/CH₂Cl₂, 5:95) to give compound 17b (33.3 mg, 57%) as a gray solid; ¹H NMR (CDCl₃, 400 MHz) 7.97 (1H, d, J=2.0 Hz), 7.84 (1H, d, J=2.7 Hz), 7.35-7.34 (4H, m), 7.26-7.22 (1H, m), 7.16 (1H, s), 6.72 (1H, t, J=2.0 Hz), 4.94 (2H, br), 4.54-4.48 (1H, m), 4.17 (1H, br), 1.56 (3H, d, J=6.7 Hz); HRMS (ESI-TOF) m/z: [M+H]⁺ calculated for C₁₆H₁₇N₄S 297.1168; found 297.1160.

### (S)—N-(5-(5-((1-Phenylethyl)amino)pyridin-3-yl)thiazol-2-yl)pent-4-ynamide (Compound 16bg)

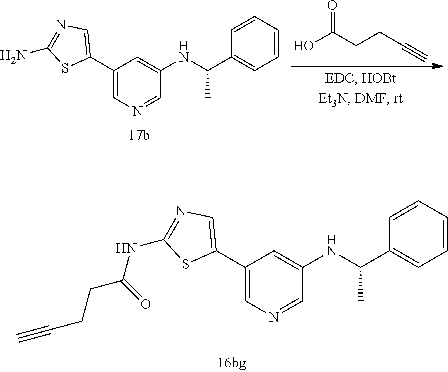

To a solution of compound 17b (11.1 mg, 0.04 mmol), 4-pentynoic acid (4.0 mg, 0.04 mmol) and HOBt (6.3 mg, 0.04 mmol) in anhydrous DMF (2 mL) was added triethylamine (5.7 μL, 0.04 mmol) and EDC (7.9 mg, 0.04 mmol) under nitrogen. The resulting mixture was stirred at room temperature for 16 h. After being quenched with water (5 mL), the aqueous layer was extracted with EtOAc (2×10 mL). The combined organic extracts were washed with brine, dried over anhydrous Na₂SO₄, filtered, and concentrated. The residue was and purified by column chromatography on silica gel (MeOH/CH₂Cl₂, 5:95) to give compound 16bg (9.5 mg, 67%) as a gray solid; ¹H NMR* (DMSO-d₆, 400 MHz) 12.24 (1H, s), 7.95 (1H, d, J=2.0 Hz), 7.82 (1H, d, J=2.7 Hz), 7.74 (1H, s), 7.40 (2H, d, J=6.7 Hz), 7.30 (2H, t, J=8.0 Hz), 7.18 (1H, t, J=7.3 Hz), 6.93 (1H, t, J=2.0 Hz), 6.60 (1H, d, J=7.3 Hz), 4.59 (1H, quint, J=6.7 Hz), 2.81 (1H, t, J=2.7 Hz), 2.65 (2H, t, J=7.3 Hz), 1.44 (3H, d, J=6.7 Hz); HRMS (ESI-TOF) m/z: [M+H]⁺ calculated for C₂₁H₂₁N₄O₂S 377.1431; found 377.1411. *Two protons are hidden in DMSO-d₆ peak.

### (S)-6-(5-((1-Phenylethyl)amino)pyridin-3-yl)pyridazin-3-amine (Compound 17c)

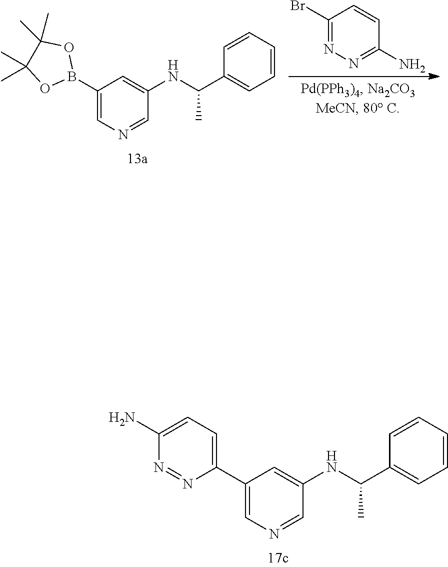 [SMILES: C[C@H](Nc1cncc(B2OC(C)(C)C(C)(C)O2)c1)c1ccccc1 ; Nc1ccc(Br)nn1 ; C[C@H](Nc1cncc(-c2ccc(N)nn2)c1)c1ccccc1]

The title compound was prepared from compound 13a and 6-bromopyridazin-3-amine following method for preparation of compound 19 and heated at 80° C. for 16 h. The reaction was purified by column chromatography on silica gel (MeOH/CH₂Cl₂, 5:95 to 10:90) to give compound 17c (31%) as a yellow oil; ¹H NMR (CDCl₃, 600 MHz) 8.32 (1H, s), 7.99 (1H, d, J=2.4 Hz), 7.53-7.51 (2H, m), 7.37 (2H, d, J=7.3 Hz), 7.32 (2H, t, J=7.3 Hz), 7.23 (1H, t, J=7.3 Hz), 6.79 (1H, d, J=8.5 Hz), 4.83 (2H, br), 4.62 (1H, q, J=6.1 Hz), 4.23 (1H, br), 1.57 (3H, d, J=6.1 Hz); HRMS (ESI-TOF) m/z: calculated for C₁₇H₁₈N₅ 292.1557; found 292.1562.

### (S)—N^{5′}-(1-Phenylethyl)-[2,3′-bipyridine]-5′,6-diamine (Compound 17d)

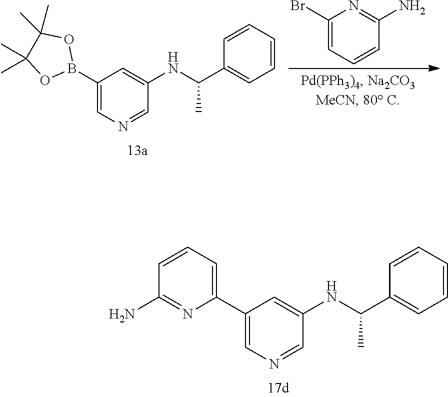 [SMILES: C[C@H](Nc1cncc(B2OC(C)(C)C(C)(C)O2)c1)c1ccccc1 ; Nc1cccc(Br)n1 ; C[C@H](Nc1cncc(-c2cccc(N)n2)c1)c1ccccc1]

The title compound was prepared from compound 13a and 6-bromopyridin-2-amine following method for preparation of compound 19 and heated at 80° C. for 16 h. The reaction was purified by column chromatography on silica gel (MeOH/CH₂Cl₂, 5:95) to give compound 17d (52%) as a yellow oil; ¹H NMR (CDCl₃, 600 MHz) 8.38 (1H, s), 7.91 (1H, s), 7.46 (1H, t, J=7.3 Hz), 7.39-7.31 (3H, m), 7.23 (1H, t, J=7.3 Hz), 6.95 (1H, d, J=7.3 Hz), 6.45 (1H, d, J=8.5 Hz), 4.59 (2H, q, J=7.3 Hz), 4.50 (2H, br), 1.56 (3H, d, J=6.1 Hz); HRMS (ESI-TOF) m/z: calculated for C₁₈H₁₉N₄ 291.1604; found 291.1613.

### (S)-tert-Butyl (2-oxo-2-((6-(5-((1-phenylethyl)amino)pyridin-3-yl)pyridazin-3-yl)amino)ethyl)(phenyl)carbamate (compound 16bh)

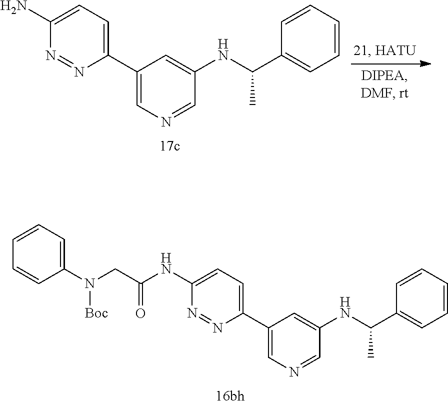 [SMILES: C[C@H](Nc1cncc(-c2ccc(N)nn2)c1)c1ccccc1 ; C[C@H](Nc1cncc(-c2ccc(NC(=O)CN(C(=O)OC(C)(C)C)c3ccccc3)nn2)c1)c1ccccc1]

To a solution of compound 17c (12.5 mg, 0.04 mmol), compound 21 (8.3 mg, 0.03 mmol) and HATU (18.8 mg, 0.05 mmol) in anhydrous DMF (2 mL) was added diisopropylethylamine (12 μL, 0.07 mmol). The resulting mixture was stirred at room temperature for 20 h. After being quenched with water (5 mL), the aqueous layer was extracted with EtOAc (2×10 mL). The combined organic extracts were washed with brine, dried over anhydrous Na₂SO₄, filtered, and concentrated. The residue was and purified by column chromatography on silica gel (MeOH/CH₂Cl₂, 5:95) to give compound 16bh (10.3 mg, 60%) as a yellow solid; 1H NMR (CDCl₃, 600 MHz) 10.11 (1H, br), 8.59 (1H, d, J=9.8 Hz), 8.43 (1H, s), 8.04 (1H, d, J=2.4 Hz), 7.76 (1H, d, J=8.5 Hz), 7.48 (1H, s), 7.37-7.31 (8H, m), 7.25-7.21 (2H, m), 4.65-4.61 (3H, m), 4.30 (1H, br), 1.57 (3H, d, J=6.1 Hz), 1.44 (9H, s); HRMS (ESI-TOF) m/z: [M+H]⁺ calculated for C₃₀H₃₂N₆O₃ 525.2609; found 525.2867.

### (S)-tert-Butyl (2-oxo-2-((5′-((1-phenylethyl)amino)-[2,3′-bipyridin]-6-yl)amino)ethyl)(phenyl)carbamate (Compound 16bi)

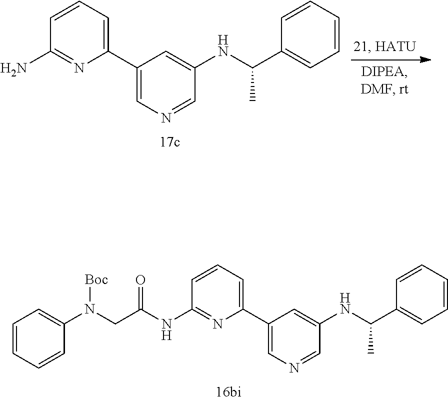 [SMILES: C[C@H](Nc1cncc(-c2cccc(N)n2)c1)c1ccccc1 ; C[C@H](Nc1cncc(-c2cccc(NC(=O)CN(C(=O)OC(C)(C)C)c3ccccc3)n2)c1)c1ccccc1]

The title compound was prepared from compound 17c and compound 21 following method for preparation of compound 16bh and purified by column chromatography on silica gel (MeOH/CH₂Cl₂, 5:95) to give compound 16bi (40%) as a yellow oil; 1H NMR (CDCl₃, 600 MHz) 8.53 (1H, s), 8.44 (1H, s), 8.17 (1H, d, J=8.5 Hz), 7.99 (1H, s), 7.74 (1H, t, J=8.5 Hz), 7.38-7.33 (11H, m), 4.60 (1H, q, J=6.1 Hz), 4.42 (2H, s), 4.33 (1H, s), 1.59 (3H, d, J=4.9 Hz), 1.48 (9H, s); RMS (ESI-TOF) m/z: calculated for C₃₁H₃₄N₅O₃ 524.2656; found 524.2729.

### (S)-2-(Phenylamino)-N-(6-(5-((I-phenylethyl)amino)pyridin-3-yl)pyridazin-3-yl)acetamide (Compound 16bj)

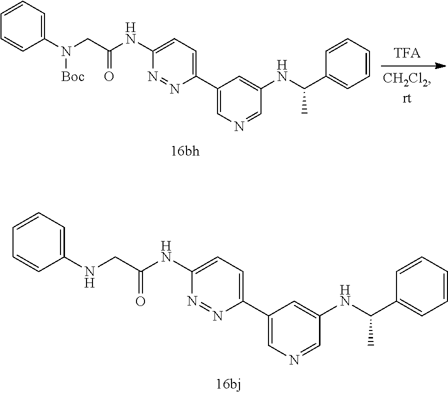 [SMILES: C[C@H](Nc1cncc(-c2ccc(NC(=O)CN(C(=O)OC(C)(C)C)c3ccccc3)nn2)c1)c1ccccc1 ; C[C@H](Nc1cncc(-c2ccc(NC(=O)CNc3ccccc3)nn2)c1)c1ccccc1]

To a solution of compound 16bh (7.7 mg, 0.02 mmol) in anhydrous CH₂Cl₂ (3 mL) was slowly added trifluoroacetic acid (0.3 mL). The resulting mixture was stirred at room temperature for 16 h. After being quenched with sat. NaHCO₃, the aqueous layer was extracted with EtOAc (2×10 mL). The combined organic extracts were washed with brine, dried over anhydrous Na₂SO₄, filtered, and concentrated. The residue was and purified by column chromatography on silica gel (MeOH/CH₂Cl₂, 5:95 to 10:90) to give compound 16bj (4.9 mg, 79%) as a white solid; 1H NMR (CDCl₃, 600 MHz) 10.42 (1H, s), 8.63 (1H, d, J=8.5 Hz), 8.40 (1H, s), 8.04 (1H, d, J=2.4 Hz), 7.79 (1H, d, J=9.8 Hz), 7.48 (1H, s), 7.33-7.29 (4H, m), 7.23-7.17 (3H, m), 6.80 (1H, t, J=7.3 Hz), 6.66 (2H, d, J=8.5 Hz), 4.83 (1H, br), 4.55 (1H, quint, J=4.9 Hz), 4.25 (1H, s), 4.12 (2H, s), 1.52 (3H, d, J=6.1 Hz); HRMS (ESI-TOF) m/z: [M+H]⁺ calculated for C₂₅H₂₅N₆O 425.2084; found 425.2254.

### (S)-2-(Phenylamino)-N-(5′-((1-phenylethyl)amino)-[2,3′-bipyridin]-6-yl)acetamide (Compound 16bk)

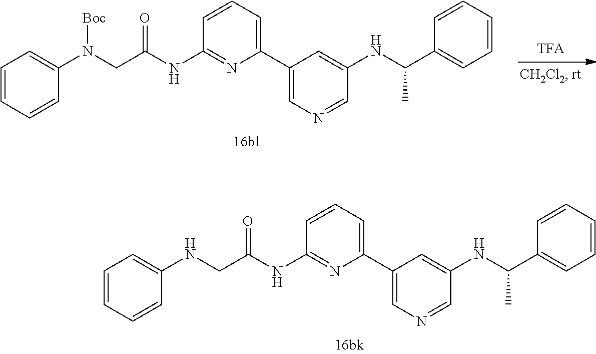 [SMILES: C[C@H](Nc1cncc(-c2cccc(NC(=O)CN(C(=O)OC(C)(C)C)c3ccccc3)n2)c1)c1ccccc1 ; C[C@H](Nc1cncc(-c2cccc(NC(=O)CNc3ccccc3)n2)c1)c1ccccc1]

The title compound was prepared from compound 16bi following method for preparation of compound 16bj and purified by column chromatography on silica gel (MeOH/CH₂Cl₂, 5:95) to give compound 16bk (82%) as a yellow solid; 1H NMR (CDCl₃, 600 MHz) 9.12 (1H, s), 8.38 (1H, s), 8.24 (1H, d, J=7.3 Hz), 7.94 (1H, s), 7.75 (1H, t, J=8.5 Hz), 7.34-7.31 (4H, m), 7.29-7.26 (5H, m), 7.20 (1H, t, J=7.3 Hz), 6.88 (1H, t, J=7.3 Hz), 6.73 (2H, d, J=8.0 Hz), 4.55 (1H, q, J=7.3 Hz), 4.39 (1H, br), 4.20 (1H, br), 3.98 (3H, s), 1.55 (3H, d, J=7.3 Hz); RMS (ESI-TOF) m/z: calculated for C₂₆H₂₆N₅O 424.2132; found 424.2512.

### N-Benzyl-5-(1H-indazol-6-yl)pyrimidin-2-amine (Compound 16bl)

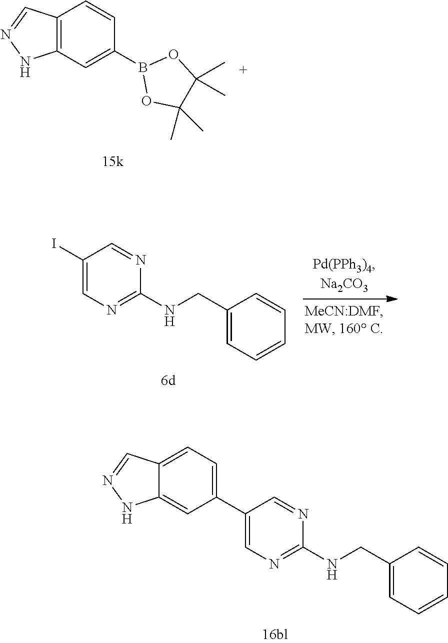 [SMILES: CC1(C)OB(c2ccc3cn[nH]c3c2)OC1(C)C ; Ic1cnc(NCc2ccccc2)nc1 ; c1ccc(CNc2ncc(-c3ccc4cn[nH]c4c3)cn2)cc1]

The title compound was prepared from compound 15k and compound 6d following method for preparation of compound 16c and purified by column chromatography on silica gel (EtOAc/Hexane, 70:30) to give compound 16bl (53%) as a white solid; 1H NMR (DMSO-d₆, 600 MHz) 13.10 (1H, s), 8.67 (2H, s), 8.05 (1H, s), 7.92 (1H, t, J=6.1 Hz), 7.80 (1H, d, J=8.5 Hz), 7.67 (1H, s), 7.36-7.29 (5H, m), 7.21 (1H, t, J=7.3 Hz), 4.56 (2H, d, J=6.1 Hz); RMS (ESI-TOF) m/z: calculated for C₁₈H₁₆N₅ 302.1400; found 302.1405.

### N-Benzyl-5-(1H-indazol-6-yl)pyridin-3-amine (Compound 16bm)

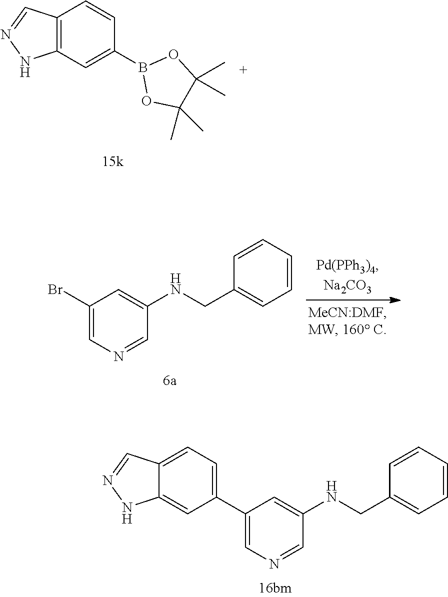 [SMILES: CC1(C)OB(c2ccc3cn[nH]c3c2)OC1(C)C ; Brc1cncc(NCc2ccccc2)c1 ; c1ccc(CNc2cncc(-c3ccc4cn[nH]c4c3)c2)cc1]

The title compound was prepared from compound 15k and compound 6a following method for preparation of compound 16c and purified by column chromatography on silica gel (EtOAc/Hexane, 70:30) to give compound 16bm (65%) as a pale yellow solid; 1H NMR (DMSO-d₆, 500 MHz) 8.08 (2H, s), 7.99 (1H, d, J=2.6 Hz), 7.81 (1H, t, J=8.3 Hz), 7.64 (1H, s), 7.41-7.39 (2H, m), 7.35-7.30 (3H, m), 7.24 (1H, d, J=7.1 Hz), 7.16 (1H, s), 6.61 (1H, t, J=6.1 Hz), 4.39 (2H, d, J=5.6 Hz); HRMS (ESI-TOF) m/z: calculated for C₁₉H₁₇N₄ 301.1448; found 301.1538.

### N-(5-Bromopyridin-3-yl)-3-phenylpropiolamide (Compound 23)

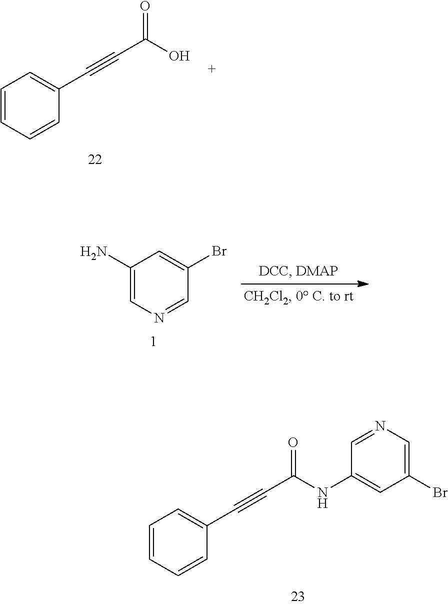 [SMILES: O=C(O)C#Cc1ccccc1 ; Nc1cncc(Br)c1 ; O=C(C#Cc1ccccc1)Nc1cncc(Br)c1]

To a solution of compound 22 (38.4 mg, 0.26 mmol) in anhydrous CH₂Cl₂ (3 mL) was added compound 1 (50.0 mg, 0.29 mmol), DCC (54.2 mg, 0.26 mmol) and DMAP (3.2 mg, 0.03 mmol) at 0° C. under nitrogen. The resulting mixture was stirred at 0° C. for 1 h then the temperature was raised to room temperature and stirred for 16 h. The reaction was filtered through a Celite pad and solvent was removed in vacuum. The residue was purified by column chromatography on silica gel (EtOAc/hexane, 15:85 to 30:70) to give compound 23 (28.4 mg, 36%) as a yellow solid; 1H NMR (CDCl₃, 600 MHz) δ 8.67-8.49 (3H, m), 8.44 (1H, s), 7.54 (2H, d, J=7.6 Hz), 7.45 (1H, t, J=7.6 Hz), 7.37 (2H, t, J=7.6 Hz); HRMS (ESI-TOF) m/z: [M+H]⁺ calculated for C₁₄H₁₀⁷⁹BrN₂O, C₁₄H₁₀⁸¹BrN₂O 300.9971, 302.9951; found 301.0059, 303.0041.

### (S)-3-Phenyl-N-(5′-((1-phenylethyl)amino)-[3,3′-bipyridin]-5-yl)propiolamide (Compound 16bn)

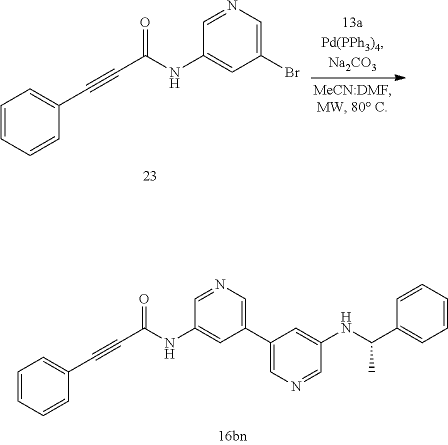 [SMILES: O=C(C#Cc1ccccc1)Nc1cncc(Br)c1 ; C[C@H](Nc1cncc(-c2cncc(NC(=O)C#Cc3ccccc3)c2)c1)c1ccccc1]

The title compound was prepared from compound 13a and compound 23 following method for preparation of compound 19 and heated at 80° C. for 16 h. The reaction was purified by column chromatography on silica gel (MeOH/CH₂Cl₂, 5:95) to give 16bn (29%) as a brown solid; ¹H NMR (DMSO-d₆, 600 MHz) δ 11.19 (1H, s), 8.76 (1H, d, J=2.3 Hz), 8.47 (1H, d, J=2.0 Hz), 8.23 (1H, t, J=2.2 Hz), 7.96 (2H, dd, J=10.5, 2.2 Hz), 7.68 (2H, dt, J=7.0, 1.4 Hz), 7.60-7.54 (1H, m), 7.52 (2H, dd, J=8.2, 6.8 Hz), 7.49-7.40 (2H, m), 7.32 (2H, t, J=7.6 Hz), 7.20 (1H, t, J=7.3 Hz), 7.06 (1H, t, J=2.3 Hz), 6.70 (1H, d, J=7.2 Hz), 4.64 (1H, quint, J=6.8 Hz), 1.47 (3H, d, J=6.7 Hz); LC-MS (ESI-TOF) m/z: [M+H]⁺ calculated for C₂H₂₃N₄O 419.1866; found 419.1.

### N-(3-Bromophenyl)-2-(1,3-dioxoisoindolin-2-yl)acetamide (Compound 25)

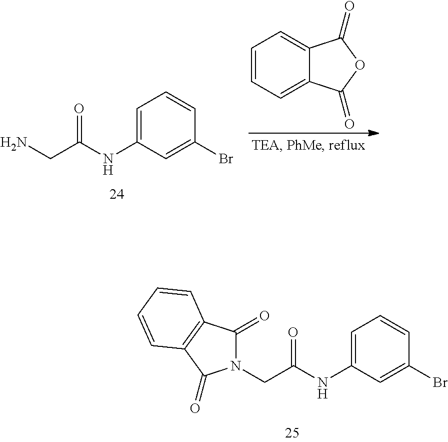 [SMILES: NCC(=O)Nc1cccc(Br)c1 ; O=C1OC(=O)c2ccccc21 ; O=C(CN1C(=O)c2ccccc2C1=O)Nc1cccc(Br)c1]

To a solution of compound 24 (100.0 mg, 0.44 mmol) in anhydrous toluene (5 mL) was added phthalic anhydride (71.1 mg, 0.48 mmol) and triethylamine (0.25 mmol). The resulting mixture was stirred at 80° C. for 2 h. After being quenched with H₂O, the aqueous layer was extracted with EtOAc (2×10 mL). The combined organic extracts were washed with brine, dried over anhydrous Na₂SO₄, filtered, and concentrated. The residue was purified by column chromatography on silica gel (EtOAc/hexane, 30:70 to 50:50) to give compound 25 (108.1 mg, 69%) as a white solid; ¹H NMR (CDCl₃, 600 MHz) δ 7.94 (2H, dd, J=5.4, 3.1 Hz), 7.81-7.78 (3H, m), 7.64 (1H, br), 7.45-7.40 (1H, m), 7.27 (1H, d, J=8.1 Hz), 7.20 (1H, t, J=8.1 Hz), 4.53 (2H, s); LC-MS (ESI-TOF) m/z: [M+H]⁺ calculated for C₁₆H₁₂⁷⁹BrN₂O₃, C₁₆H₁₂⁸¹BrN₂O₃ 359.0026, 361.0005; found 359.1, 361.1.

### (S)-2-Amino-N-(3-(5-((1-phenylethyl)amino)pyridin-3-yl)phenyl)acetamide (Compound 16bo)

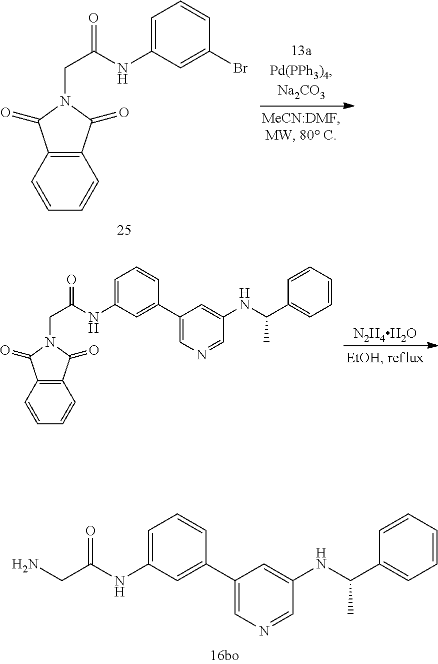

To a solution of compound 25 (39.6 mg, 0.11 mmol), compound 13a (39.3 mg, 0.12 mmol), and Pd(PPh₃)₄ (12.7 mg, 0.01 mmol) in anhydrous MeCN (1 mL) and anhydrous DMF (1 mL) was added 1 M Na₂CO₃ (0.22 mL, 0.22 mmol) under nitrogen. The resulting mixture was stirred at 80° C. for 16 h. After being quenched with water (5 mL), the aqueous layer was extracted with EtOAc (2×5 mL). The combined organic extracts were washed with brine, dried over anhydrous Na₂SO₄, filtered, and concentrated. The residue was and purified by column chromatography on silica gel (MeOH/CH₂Cl₂, 5:95) to give crude (S)-2-(1,3-dioxoisoindolin-2-yl)-N-(3-(5-((1-phenylethyl)amino)pyridin-3-yl)phenyl)acetamide (108.1 mg, 69%) as a yellow solid; LC-MS (ESI-TOF) m/z: [M+H]⁺ calculated for C₂₉H₂₅N₄O₃ 477.1921; found 477.3. The crude product was dissolved in EtOH (2 mL) then hydrazine monohydrate (0.1 mL) was added. The resulting mixture was refluxed for 1 h. The solvent was removed in vacuum. The residue was purified by column chromatography on silica gel (MeOH/CH₂Cl₂, 5:95 to 10:90) to give compound 16bo (3.3 mg, 9% over 2 steps) as a yellow solid, 1H NMR (MeOD, 600 MHz) δ δ 7.90 (1H, s), 7.84-7.80 (1H, m), 7.77 (1H, t, J=2.0 Hz), 7.58-7.52 (1H, m), 7.41-7.39 (2H, m), 7.36 (1H, t, J=7.9 Hz), 7.31 (2H, t, J=7.7 Hz), 7.23-7.08 (2H, m), 7.07 (1H, t, J=2.3 Hz), 4.56 (1H, q, J=6.7 Hz), 3.55 (2H, s), 1.54 (3H, d, J=6.7 Hz); LC-MS (ESI-TOF) m/z: [M+H]⁺ calculated for C₂₁H₂₃N₄O 347.1866; found 347.1.

### tert-Butyl (2-oxo-2-(phenylamino)ethyl)(phenyl)carbamate (Compound 26)

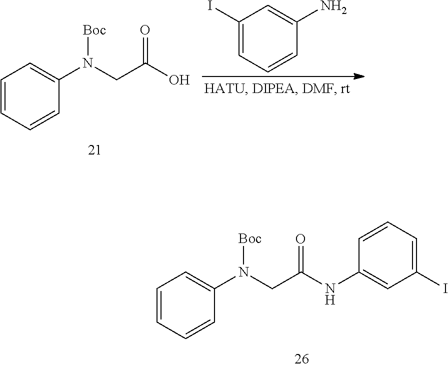 [SMILES: CC(C)(C)OC(=O)N(CC(=O)O)c1ccccc1 ; Nc1cccc(I)c1 ; CC(C)(C)OC(=O)N(CC(=O)Nc1cccc(I)c1)c1ccccc1]

The title compound was prepared from compound 13a and 3-iodoaniline following the method for preparation of compound 16bh and stirred at room temperature for 5 h. The reaction was purified by column chromatography on silica gel (EtOAc/hexane, 30:70) to give compound 26 (80%) as a white solid; ¹H NMR (CDCl₃, 600 MHz) δ 8.48 (s, 1H), 7.96 (t, J=1.9 Hz, 1H), 7.41 (ddd, J=7.6, 5.0, 1.8 Hz, 2H), 7.38-7.32 (m, 2H), 7.32-7.28 (m, 2H), 7.23 (td, J=7.2, 1.4 Hz, 1H), 7.00 (t, J=8.0 Hz, 1H), 4.33 (s, 2H), 1.45 (s, 9H); LC-MS (ESI-TOF) m/z: [M−C₅H₈O₂]⁺ calculated for C₁₄H₁₄1N₂O 353.0; found 353.1.

### N-(3-Iodophenyl)-2-(phenylamino)acetamide (Compound 27)

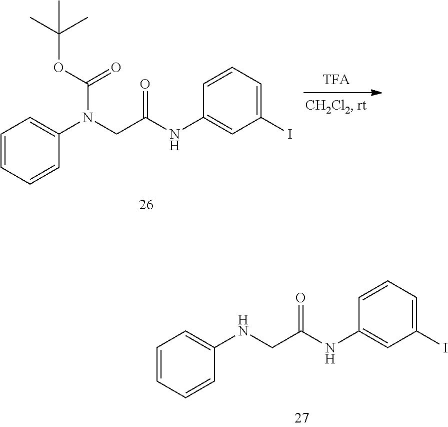 [SMILES: CC(C)(C)OC(=O)N(CC(=O)Nc1cccc(I)c1)c1ccccc1 ; O=C(CNc1ccccc1)Nc1cccc(I)c1]

The title compound was prepared from compound 26 according to the method for preparation of compound 16bj. The reaction was purified by column chromatography on silica gel (EtOAc/hexane, 20:80) to give compound 27 (86%) as a yellow solid; 1H NMR (CDCl₃, 600 MHz) δ 8.51 (s, 1H), 7.84 (t, J=1.9 Hz, 1H), 7.45 (ddd, J=8.2, 2.1, 1.0 Hz, 1H), 7.36 (dt, J=7.9, 1.3 Hz, 1H), 7.20-7.14 (m, 2H), 6.95 (t, J=8.0 Hz, 1H), 6.80 (tt, J=7.4, 1.1 Hz, 1H), 6.61 (dt, J=8.5, 1.3 Hz, 2H), 3.82 (s, 2H); LC-MS (ESI-TOF) m/z: [M+H]⁺ calculated for C₁₄H₁₄IN₂O 353.0; found 353.2.

### N¹-(3-Iodophenyl)-N²-phenylethane-1,2-diamine (Compound 28)

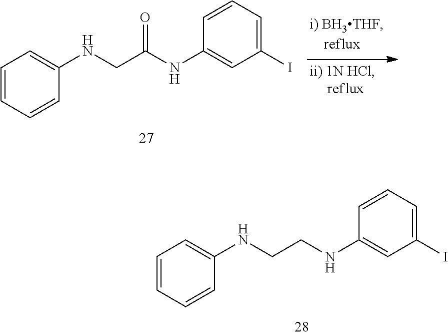 [SMILES: O=C(CNc1ccccc1)Nc1cccc(I)c1 ; Ic1cccc(NCCNc2ccccc2)c1]

Compound 27 (54.3 mg, 0.15 mmol) was dissolved in 1 M BH₃·THF (4 mL) then the resulting mixture was purged with nitrogen and refluxed for 4 h. Solvent was removed in vacuum. 1 N HCl (3 mL) was added to a crude product and refluxed for 30 min. Sat. NaHCO₃ was added to quenched the reaction. The aqueous layer was extracted with EtOAc (2×10 mL). The combined organic extracts were washed with brine, dried over anhydrous Na₂SO₄, filtered, and concentrated. The residue was purified by column chromatography on silica gel (EtOAc/hexane, 20:80) to give compound 28 (45.0 mg, 86%) as a yellow oil; ¹H NMR (CDCl₃, 600 MHz) δ 7.18-7.09 (m, 2H), 6.96 (dt, J=7.8, 1.2 Hz, 1H), 6.90 (t, J=2.0 Hz, 1H), 6.80 (t, J=7.9 Hz, 1H), 6.72-6.66 (m, 1H), 6.62-6.57 (m, 2H), 6.50 (ddd, J=8.2, 2.4, 0.9 Hz, 1H), 3.37-3.30 (m, 2H), 3.29-3.18 (m, 2H); LC-MS (ESI-TOF) m/z: [M+H]⁺ calculated for C₁₄H₁₆IN₂ 339.0; found 339.0.

### (S)—N¹-phenyl-N²-(3-(5-((1-phenylethyl)amino)pyridin-3-yl)phenyl)ethane-1,2-diamine (Compound 16 bp)

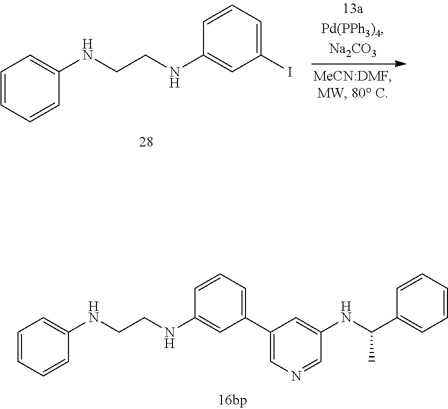 [SMILES: Ic1cccc(NCCNc2ccccc2)c1 ; C[C@H](Nc1cncc(-c2cccc(NCCNc3ccccc3)c2)c1)c1ccccc1]

The title compound was prepared from compound 28 and compound 13a according to the method for preparation of compound 19. The reaction was purified by column chromatography on silica gel (EtOAc/hexane, 50:50 to 80:20) to give compound 16 bp (58%) as a yellow solid; 1H NMR (CDCl₃, 600 MHz) δ 8.25 (s, 1H), 8.05 (s, 1H), 7.93-7.86 (m, 2H), 7.69 (ddt, J=12.1, 6.9, 1.3 Hz, 2H), 7.64-7.62 (m, 1H), 7.59-7.52 (m, 2H), 7.50-7.37 (m, 7H), 7.11-7.06 (m, 1H), 4.57 (d, J=6.8 Hz, 1H), 4.33 (s, 1H), 4.04 (dd, J=26.1, 7.5 Hz, 4H), 3.83-3.74 (s, 2H), 1.71 (d, J=6.7 Hz, 3H); LC-MS (ESI-TOF) m/z: [M+H]⁺ calculated for C₂₇H₂₉N₄ 409.2; found 409.5.

### (S)-2-(Phenylamino)-N-(3-(5-((1-phenylethyl)amino)pyridin-3-yl)phenyl)acetamide (compound 16bq)

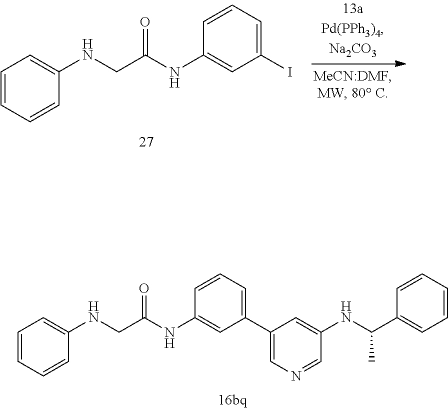 [SMILES: O=C(CNc1ccccc1)Nc1cccc(I)c1 ; C[C@H](Nc1cncc(-c2cccc(NC(=O)CNc3ccccc3)c2)c1)c1ccccc1]

The title compound was prepared from compound 27 and compound 13a according to the method for preparation of compound 19. The reaction was purified by column chromatography on silica gel (MeOH/CH₂Cl₂, 5:95) to give compound 16bq (58%) as a yellow solid, 1H NMR (CDCl₃, 600 MHz) δ 88.79 (s, 1H), 8.03 (s, 1H), 8.00-7.97 (m, 1H), 7.73-7.65 (m, 1H), 7.62 (dd, J=8.1, 2.1 Hz, 1H), 7.60-7.54 (m, 1H), 7.48 (tdd, J=9.4, 6.7, 4.1 Hz, 1H), 7.44-7.30 (m, 4H), 7.28-7.20 (m, 3H), 7.13 (d, J=7.7 Hz, 1H), 7.04 (s, 1H), 6.89 (t, J=7.4 Hz, 1H), 6.77-6.73 (m, 2H), 4.54 (q, J=6.7 Hz, 1H), 3.97 (s, 2H), 1.61 (d, J=6.7 Hz, 3H); LC-MS (ESI-TOF) m/z: [M+H]⁺ calculated for C₂₇H₂₇N₄O 423.2; found 423.3.

### (S)-3-Phenyl-N-(3-(5-((1-phenylethyl)amino)pyridin-3-yl)phenyl)propenamide (Compound 16br)

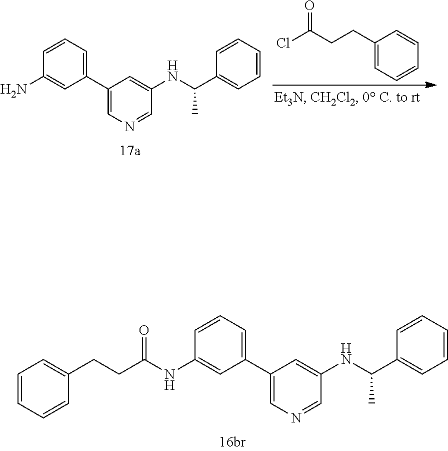

To a solution of compound 17a (13.5 mg, 0.05 mmol) and triethylamine (0.02 mL, 0.14 mmol) in anhydrous CH₂Cl₂ (3 mL) was added 3-phenylpropanoyl chloride (0.01 mL, 0.07 mmol) at 0° C. under nitrogen. The resulting mixture was stirred at 0° C. for 15 min then the temperature was raised to room temperature and stirred for 3 h. After being quenched with water (5 mL), the aqueous layer was extracted with EtOAc (2×5 mL). The combined organic extracts were washed with brine, dried over anhydrous Na₂SO₄, filtered, and concentrated. The residue was purified by column chromatography on silica gel (MeOH/CH₂Cl₂, 10:9) to give compound 16br (8.5 mg, 43%) as a pale yellow solid; 1H NMR (DMSO-d₆, 600 MHz) δ 10.02 (s, 1H), 7.98 (s, 1H), 7.87 (d, J=2.4 Hz, 2H), 7.52 (d, J=8.2 Hz, 1H), 7.44-7.40 (m, 2H), 7.37 (t, J=7.9 Hz, 1H), 7.35-7.24 (m, 6H), 7.23-7.16 (m, 4H), 4.71-4.48 (m, 1H), 2.93 (t, J=7.7 Hz, 2H), 2.65 (t, J=7.7 Hz, 2H), 1.47 (d, J=6.7 Hz, 3H); HRMS (ESI-TOF) m/z: [M+H]⁺ calculated for C₂₈H₂₈N₃O 422.2227; found 422.2317.

### Example 3—DYRK1A Binding Assays

Compounds were tested for DYRK1A binding activity at DiscoverX. DiscoverX uses proprietary KINOMEscan® Assay (Fabian et al., “A Small Molecule-kinase Interaction Map for Clinical Kinase Inhibitors,” Nat. Biotechnol. 23(3):329-336 (2005), which is hereby incorporated by reference in its entirety). Compounds were screened for DYRK1A activity at a single concentration of 3 μM in duplicates. Similarly, the dissociation constant K_{d} of the hit compounds from the initial screening was determined at DiscoverX using their proprietary KINOMEscan® Assay. K_{d} values are determined using eleven serial three fold dilutions with the highest concentration of 60 μM. The results of the binding assay are displayed in Table 1.

TABLE 1
DYRK1A Inhibition Activity

| Compound | STRUCTURE | DYRK1A IC₅₀ (nM) |
|---|---|---|
| 16a | 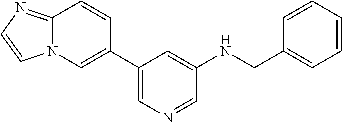 [SMILES: c1ccc(CNc2cncc(-c3ccc4nccn4c3)c2)cc1] | 32.8 |
| 16b | 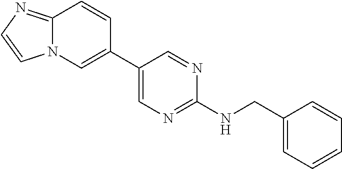 [SMILES: c1ccc(CNc2ncc(-c3ccc4nccn4c3)cn2)cc1] | 2250 |
| 16d | 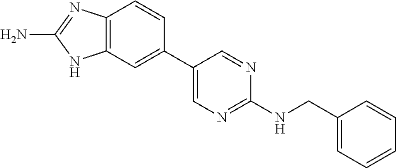 [SMILES: Nc1nc2ccc(-c3cnc(NCc4ccccc4)nc3)cc2[nH]1] | NI |
| 16c | 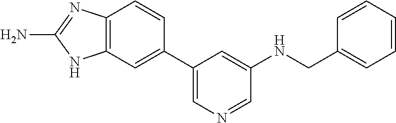 [SMILES: Nc1nc2ccc(-c3cncc(NCc4ccccc4)c3)cc2[nH]1] | 97.4 |
| 16e | 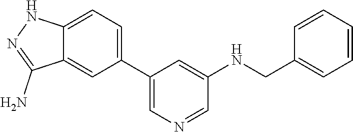 [SMILES: Nc1n[nH]c2ccc(-c3cncc(NCc4ccccc4)c3)cc12] | 0.31 |
| 16f | 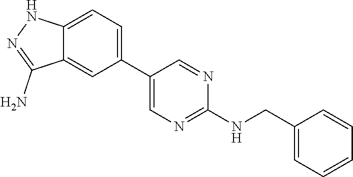 [SMILES: Nc1n[nH]c2ccc(-c3cnc(NCc4ccccc4)nc3)cc12] | 23.9 |
| 16h | 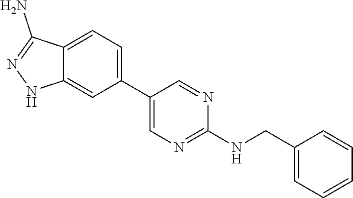 [SMILES: Nc1n[nH]c2cc(-c3cnc(NCc4ccccc4)nc3)ccc12] | 6.06 |
| 16g | 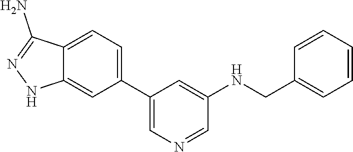 [SMILES: Nc1n[nH]c2cc(-c3cncc(NCc4ccccc4)c3)ccc12] | 144 |
| 16j | 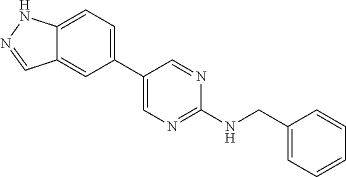 [SMILES: c1ccc(CNc2ncc(-c3ccc4[nH]ncc4c3)cn2)cc1] | 156 |
| 16i | 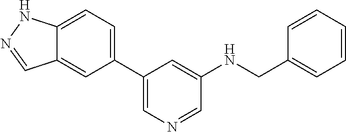 [SMILES: c1ccc(CNc2cncc(-c3ccc4[nH]ncc4c3)c2)cc1] | 0.23 |
| 16l | 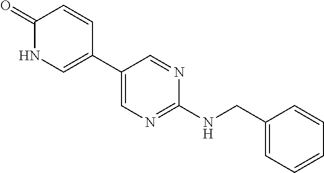 [SMILES: O=c1ccc(-c2cnc(NCc3ccccc3)nc2)c[nH]1] | 1930 |
| 16k | 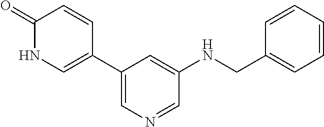 [SMILES: O=c1ccc(-c2cncc(NCc3ccccc3)c2)c[nH]1] | 8300 |
| 16n | 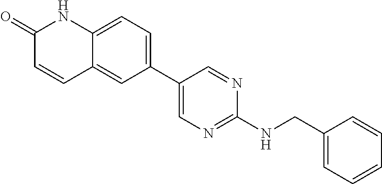 [SMILES: O=c1ccc2cc(-c3cnc(NCc4ccccc4)nc3)ccc2[nH]1] | NI |
| 16m | 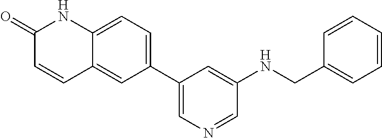 [SMILES: O=c1ccc2cc(-c3cncc(NCc4ccccc4)c3)ccc2[nH]1] | 311 |
| 16aa | 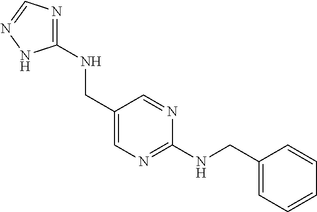 [SMILES: c1ccc(CNc2ncc(CNc3ncn[nH]3)cn2)cc1] | NI |
| 16r | 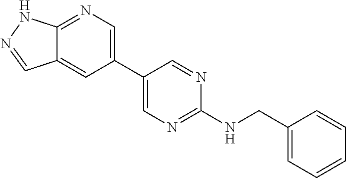 [SMILES: c1ccc(CNc2ncc(-c3cnc4[nH]ncc4c3)cn2)cc1] | NI |
| 16q | 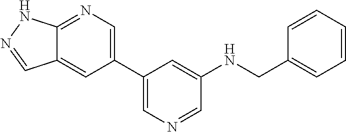 [SMILES: c1ccc(CNc2cncc(-c3cnc4[nH]ncc4c3)c2)cc1] | 0.46 |
| 16p | 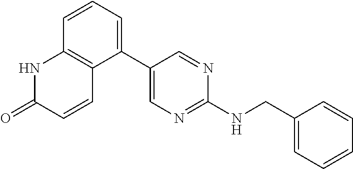 [SMILES: O=c1ccc2c(-c3cnc(NCc4ccccc4)nc3)cccc2[nH]1] | 16100 |
| 16o | 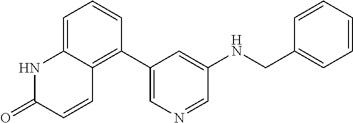 [SMILES: O=c1ccc2c(-c3cncc(NCc4ccccc4)c3)cccc2[nH]1] | 537 |
| 16t | 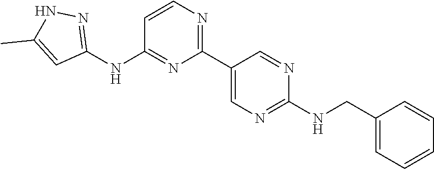 [SMILES: Cc1cc(Nc2ccnc(-c3cnc(NCc4ccccc4)nc3)n2)n[nH]1] | 7160 |
| 16s | 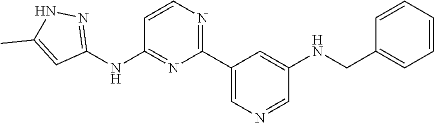 [SMILES: Cc1cc(Nc2ccnc(-c3cncc(NCc4ccccc4)c3)n2)n[nH]1] | 707 |
| 16v | 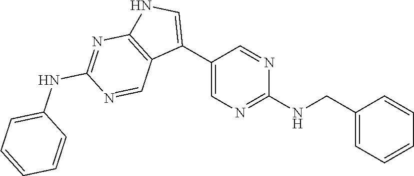 [SMILES: c1ccc(CNc2ncc(-c3c[nH]c4nc(Nc5ccccc5)ncc34)cn2)cc1] | NI |
| 16u | 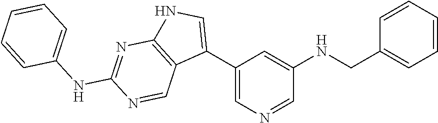 [SMILES: c1ccc(CNc2cncc(-c3c[nH]c4nc(Nc5ccccc5)ncc34)c2)cc1] | 3.85 |
| 16x | 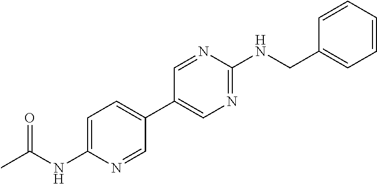 [SMILES: CC(=O)Nc1ccc(-c2cnc(NCc3ccccc3)nc2)cn1] | NI |
| 16w | 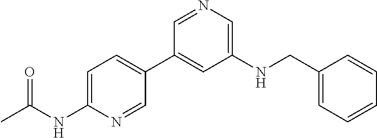 [SMILES: CC(=O)Nc1ccc(-c2cncc(NCc3ccccc3)c2)cn1] | NI |
| 16z | 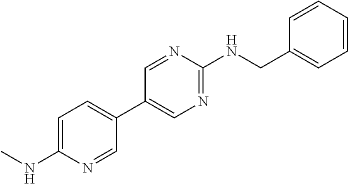 [SMILES: CNc1ccc(-c2cnc(NCc3ccccc3)nc2)cn1] | NI |
| 16y | 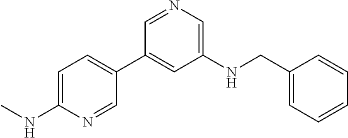 [SMILES: CNc1ccc(-c2cncc(NCc3ccccc3)c2)cn1] | NI |
| 16af | 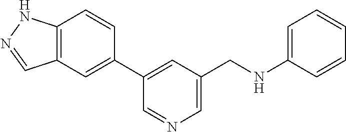 [SMILES: c1ccc(NCc2cncc(-c3ccc4[nH]ncc4c3)c2)cc1] | 3.18 |
| 16ah | 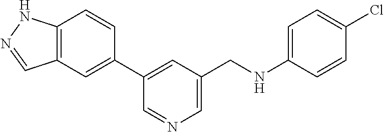 [SMILES: Clc1ccc(NCc2cncc(-c3ccc4[nH]ncc4c3)c2)cc1] | 3.04 |
| 16ag | 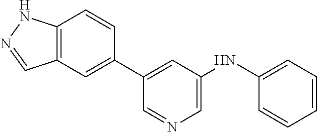 [SMILES: c1ccc(Nc2cncc(-c3ccc4[nH]ncc4c3)c2)cc1] | 2.88 |
| 16aj | 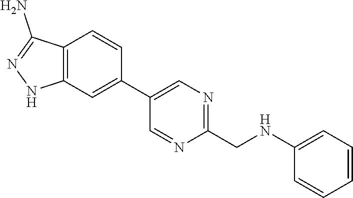 [SMILES: Nc1n[nH]c2cc(-c3cnc(CNc4ccccc4)nc3)ccc12] | 185 |
| 16ak | 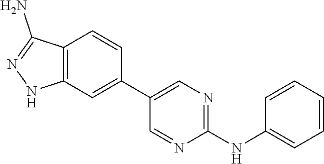 [SMILES: Nc1n[nH]c2cc(-c3cnc(Nc4ccccc4)nc3)ccc12] | 4.32 |
| 16ai | 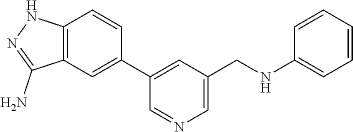 [SMILES: Nc1n[nH]c2ccc(-c3cncc(CNc4ccccc4)c3)cc12] | 3.8 |
| 16ac | 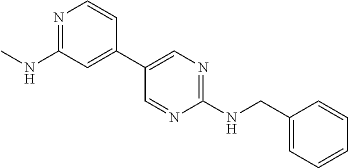 [SMILES: CNc1cc(-c2cnc(NCc3ccccc3)nc2)ccn1] | 4100 |
| 16ab | 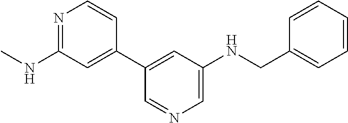 [SMILES: CNc1cc(-c2cncc(NCc3ccccc3)c2)ccn1] | 329 |
| 16al | 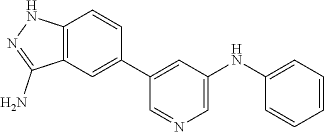 [SMILES: Nc1n[nH]c2ccc(-c3cncc(Nc4ccccc4)c3)cc12] | 4.06 |
| 16am | 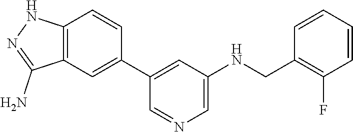 [SMILES: Nc1n[nH]c2ccc(-c3cncc(NCc4ccccc4F)c3)cc12] | 1.17 |
| 16an | 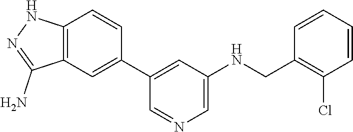 [SMILES: Nc1n[nH]c2ccc(-c3cncc(NCc4ccccc4Cl)c3)cc12] | 1.26 |
| 16ao | 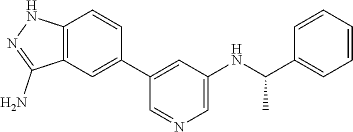 [SMILES: C[C@H](Nc1cncc(-c2ccc3[nH]nc(N)c3c2)c1)c1ccccc1] | 0.464 |
| 16ap | 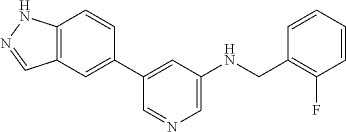 [SMILES: Fc1ccccc1CNc1cncc(-c2ccc3[nH]ncc3c2)c1] | 0.53 |
| 16aq | 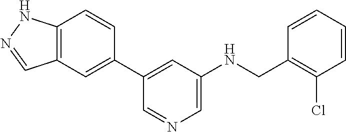 [SMILES: Clc1ccccc1CNc1cncc(-c2ccc3[nH]ncc3c2)c1] | 0.824 |
| 16ar | 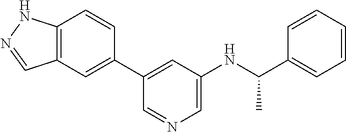 [SMILES: C[C@H](Nc1cncc(-c2ccc3[nH]ncc3c2)c1)c1ccccc1] | 0.295 |
| 16as | 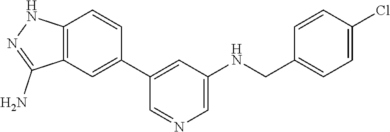 [SMILES: Nc1n[nH]c2ccc(-c3cncc(NCc4ccc(Cl)cc4)c3)cc12] | 6.83 |
| 16at | 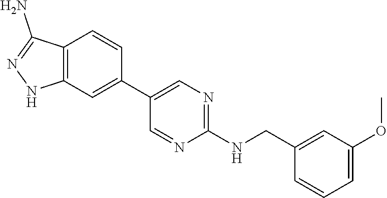 [SMILES: COc1cccc(CNc2ncc(-c3ccc4c(N)n[nH]c4c3)cn2)c1] | 8.43 |
| 16au | 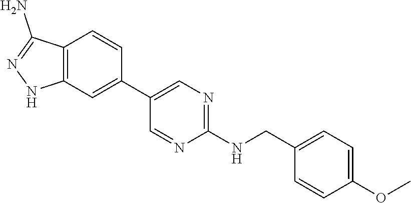 [SMILES: COc1ccc(CNc2ncc(-c3ccc4c(N)n[nH]c4c3)cn2)cc1] | 2.23 |
| 16av | 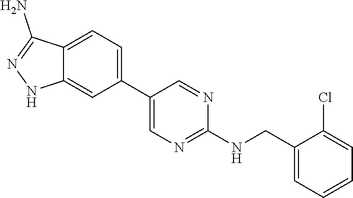 [SMILES: Nc1n[nH]c2cc(-c3cnc(NCc4ccccc4Cl)nc3)ccc12] | 19.1 |
| 16aw | 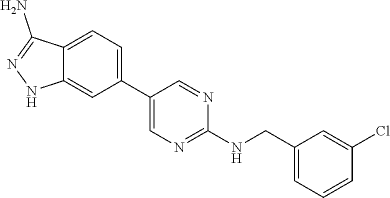 [SMILES: Nc1n[nH]c2cc(-c3cnc(NCc4cccc(Cl)c4)nc3)ccc12] | 14.5 |
| 16ax | 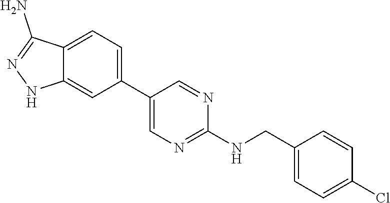 [SMILES: Nc1n[nH]c2cc(-c3cnc(NCc4ccc(Cl)cc4)nc3)ccc12] | 13.2 |
| 16ay | 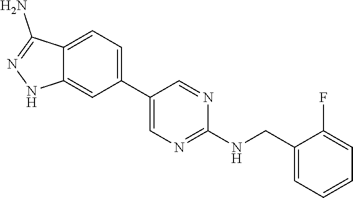 [SMILES: Nc1n[nH]c2cc(-c3cnc(NCc4ccccc4F)nc3)ccc12] | 6.11 |
| 16az | 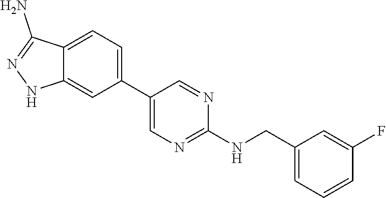 [SMILES: Nc1n[nH]c2cc(-c3cnc(NCc4cccc(F)c4)nc3)ccc12] | 7.53 |
| 16ba | 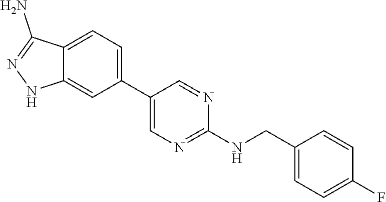 [SMILES: Nc1n[nH]c2cc(-c3cnc(NCc4ccc(F)cc4)nc3)ccc12] | 5.07 |
| 16ad | 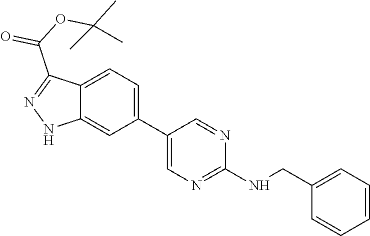 [SMILES: CC(C)(C)OC(=O)c1n[nH]c2cc(-c3cnc(NCc4ccccc4)nc3)ccc12] | 811 |
| 16ae | 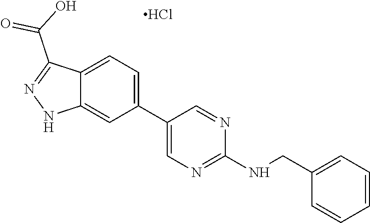 [SMILES: O=C(O)c1n[nH]c2cc(-c3cnc(NCc4ccccc4)nc3)ccc12 ; Cl] | 2190 |
| 16bb | 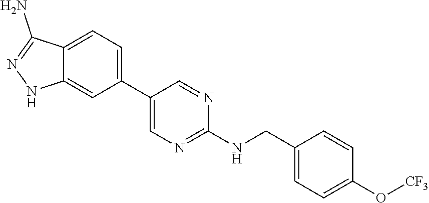 [SMILES: Nc1n[nH]c2cc(-c3cnc(NCc4ccc(OC(F)(F)F)cc4)nc3)ccc12] | 61.3 |
| 16bc | 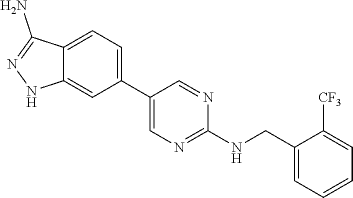 [SMILES: Nc1n[nH]c2cc(-c3cnc(NCc4ccccc4C(F)(F)F)nc3)ccc12] | 24.9 |
| 16bd | 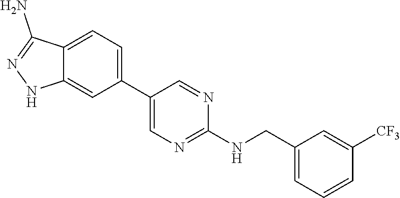 [SMILES: Nc1n[nH]c2cc(-c3cnc(NCc4cccc(C(F)(F)F)c4)nc3)ccc12] | 33.6 |
| 16be | 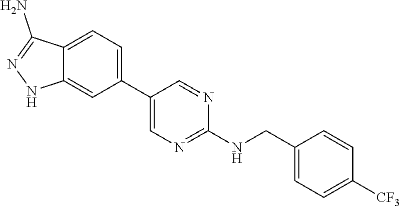 [SMILES: Nc1n[nH]c2cc(-c3cnc(NCc4ccc(C(F)(F)F)cc4)nc3)ccc12] | 24.1 |
| | 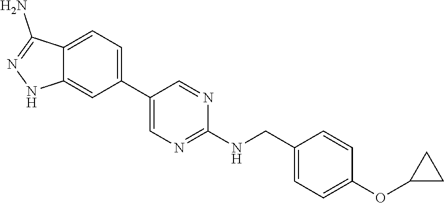 [SMILES: Nc1n[nH]c2cc(-c3cnc(NCc4ccc(OC5CC5)cc4)nc3)ccc12] | |
| | 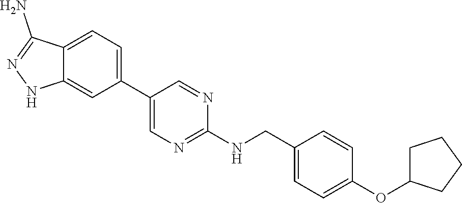 [SMILES: Nc1n[nH]c2cc(-c3cnc(NCc4ccc(OC5CCCC5)cc4)nc3)ccc12] | |
| | 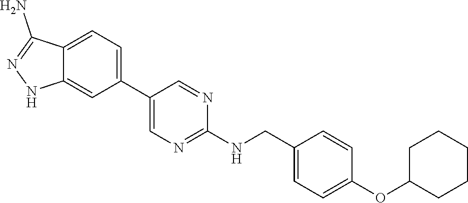 [SMILES: Nc1n[nH]c2cc(-c3cnc(NCc4ccc(OC5CCCCC5)cc4)nc3)ccc12] | |
| | 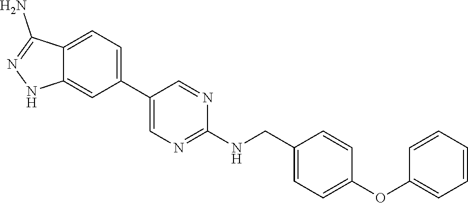 [SMILES: Nc1n[nH]c2cc(-c3cnc(NCc4ccc(Oc5ccccc5)cc4)nc3)ccc12] | |
| | 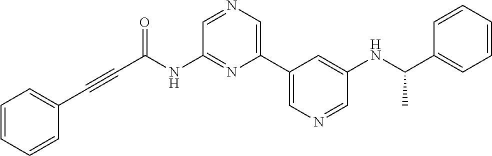 [SMILES: C[C@H](Nc1cncc(-c2cncc(NC(=O)C#Cc3ccccc3)n2)c1)c1ccccc1] | |
| 16bh | 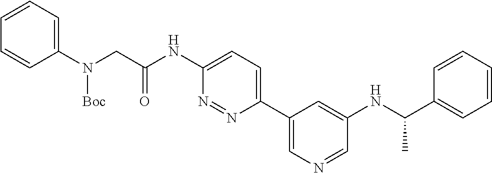 [SMILES: C[C@H](Nc1cncc(-c2ccc(NC(=O)CN(C(=O)OC(C)(C)C)c3ccccc3)nn2)c1)c1ccccc1] | NI |
| 16bj | 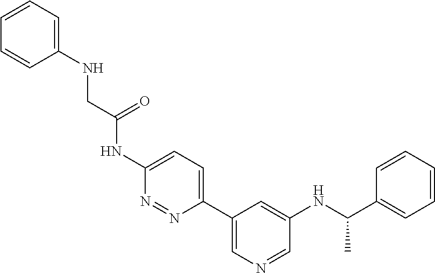 [SMILES: C[C@H](Nc1cncc(-c2ccc(NC(=O)CNc3ccccc3)nn2)c1)c1ccccc1] | NI |
| 17b | 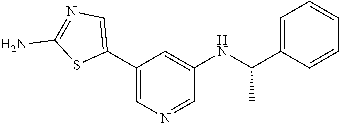 [SMILES: C[C@H](Nc1cncc(-c2cnc(N)s2)c1)c1ccccc1] | 94.2 |
| 16bg | 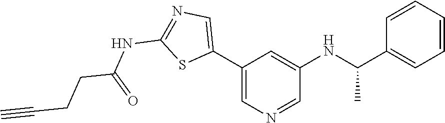 [SMILES: C#CCCC(=O)Nc1ncc(-c2cncc(N[C@@H](C)c3ccccc3)c2)s1] | 1830 |
| 16bi | 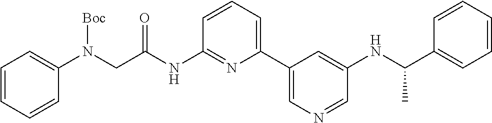 [SMILES: C[C@H](Nc1cncc(-c2cccc(NC(=O)CN(C(=O)OC(C)(C)C)c3ccccc3)n2)c1)c1ccccc1] | 927 |
| 16bk | 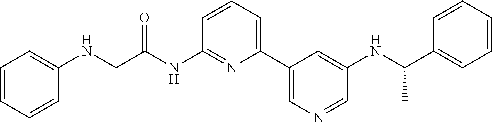 [SMILES: C[C@H](Nc1cncc(-c2cccc(NC(=O)CNc3ccccc3)n2)c1)c1ccccc1] | 13.8 |
| 16bf | 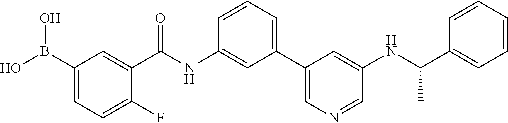 [SMILES: C[C@H](Nc1cncc(-c2cccc(NC(=O)c3cc(B(O)O)ccc3F)c2)c1)c1ccccc1] | 33.9 |
| | 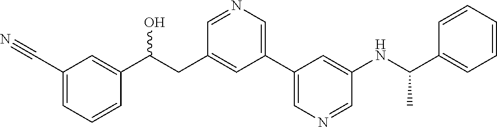 [SMILES: C[C@H](Nc1cncc(-c2cncc(CC(O)c3cccc(C#N)c3)c2)c1)c1ccccc1] | |
| | 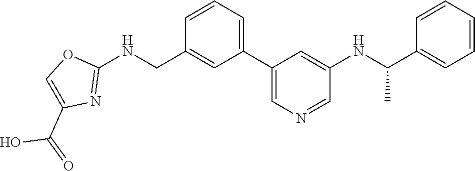 [SMILES: C[C@H](Nc1cncc(-c2cccc(CNc3nc(C(=O)O)co3)c2)c1)c1ccccc1] | |
| | 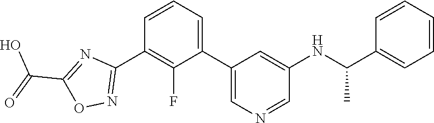 [SMILES: C[C@H](Nc1cncc(-c2cccc(-c3noc(C(=O)O)n3)c2F)c1)c1ccccc1] | |
| | 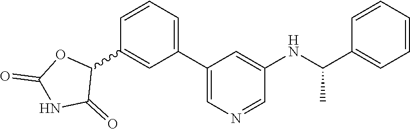 [SMILES: C[C@H](Nc1cncc(-c2cccc(C3OC(=O)NC3=O)c2)c1)c1ccccc1] | |
| 16bl | 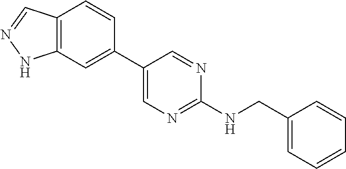 [SMILES: c1ccc(CNc2ncc(-c3ccc4cn[nH]c4c3)cn2)cc1] | 21.4 |
| 16bm | 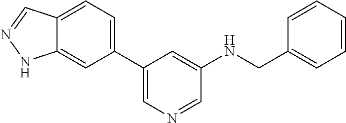 [SMILES: c1ccc(CNc2cncc(-c3ccc4cn[nH]c4c3)c2)cc1] | 39.8 |
| 16bn | 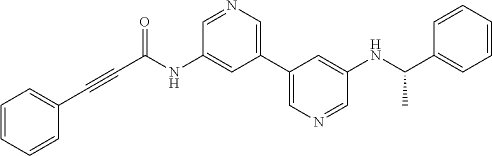 [SMILES: C[C@H](Nc1cncc(-c2cncc(NC(=O)C#Cc3ccccc3)c2)c1)c1ccccc1] | 198 |
| 16bo | 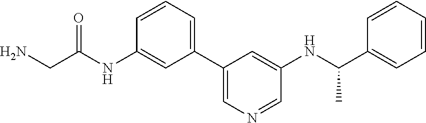 [SMILES: C[C@H](Nc1cncc(-c2cccc(NC(=O)CN)c2)c1)c1ccccc1] | nd |
| 16bp | 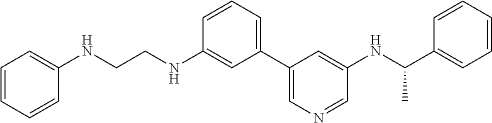 [SMILES: C[C@H](Nc1cncc(-c2cccc(NCCNc3ccccc3)c2)c1)c1ccccc1] | nd |
| 16bq | 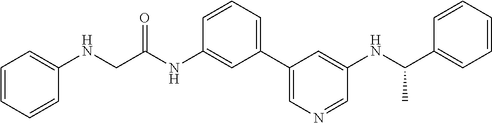 [SMILES: C[C@H](Nc1cncc(-c2cccc(NC(=O)CNc3ccccc3)c2)c1)c1ccccc1] | 87.4 |
| 16br | 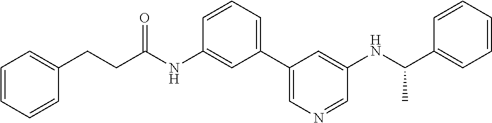 [SMILES: C[C@H](Nc1cncc(-c2cccc(NC(=O)CCc3ccccc3)c2)c1)c1ccccc1] | 443 |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A compound of formula (I) having the following structure: 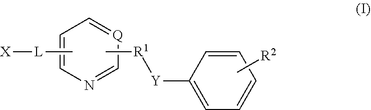
or a stereoisomer, pharmaceutically acceptable salt, oxide, or solvate thereof, wherein
X is selected from the group consisting of 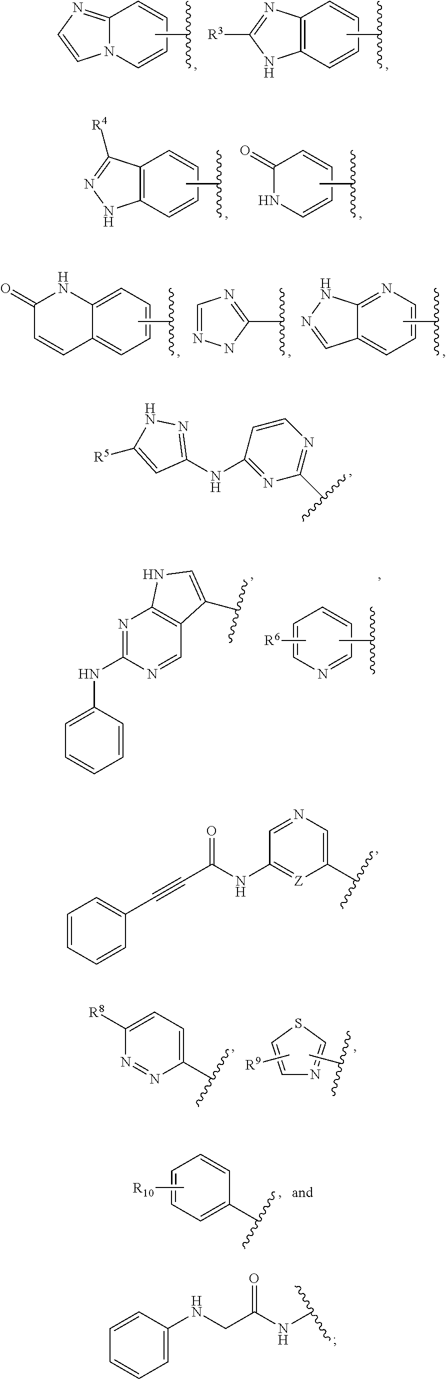
L is selected from a group consisting of a bond, 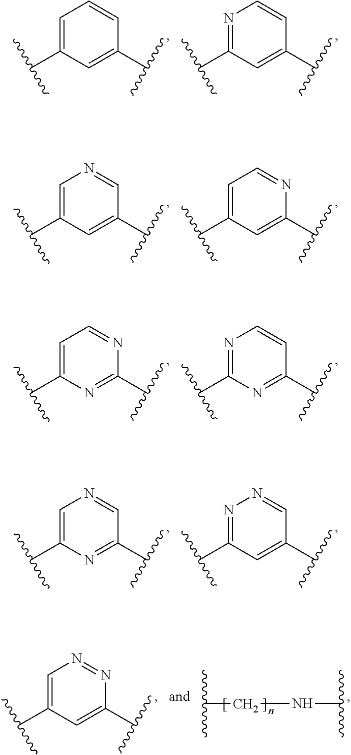
wherein n is an integer between 0-6;
Q is selected form the group consisting of CH or N;
R¹ is optionally present, and when present is selected from the group consisting of NH or branched or unbranched C₁-C₆ alkyl;
Y is selected from the group consisting of branched or unbranched C₁-C₆ alkyl and NH;
R² is absent or present, and when present is selected from the group consisting of one or more of halogen, alkyl, alkoxy, CF₃, OPh, OCF₃, CN, CONH₂, and COOCH₃;
R³ is selected from the group consisting of C₁-C₆ alkoxy and NH₂;
R⁴ is selected from the group consisting of H, NH₂, NHPh, COOC(CH₃)₃, COOH, CONH₂, CONHCH₃, NHCONH₂, and CF₃;
R⁵ is branched or unbranched C₁-C₆ alkyl;
R⁶ is selected from the group consisting of 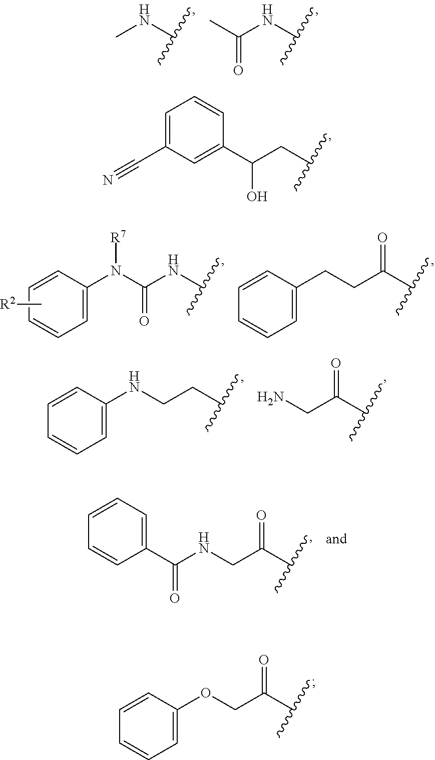
R⁷ is selected from the group consisting of H and Boc;
R⁸ is 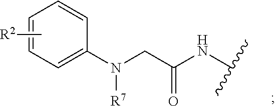
R⁹ is selected from the group consisting of NH₂ and 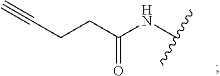 [SMILES: *NC(=O)CCC#C]
R¹⁰ is one or more of halogen, 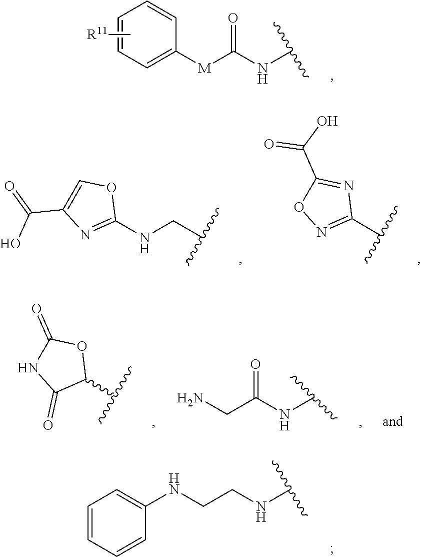
R¹¹ is one or more of halogen and 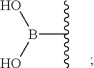 [SMILES: *B(O)O]
Z is CH or N; and
M is optionally present and when present is —NHCH₂— or —CH₂CH₂—.

2. The compound according to claim 1, wherein X is

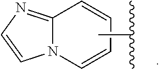

3. The compound according to claim 2, having a chemical structure

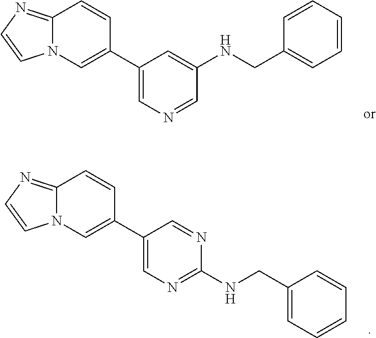 [SMILES: c1ccc(CNc2cncc(-c3ccc4nccn4c3)c2)cc1 ; c1ccc(CNc2ncc(-c3ccc4nccn4c3)cn2)cc1]

4. The compound according to claim 1, wherein X is

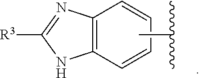

5. The compound according to claim 4, having a chemical structure of

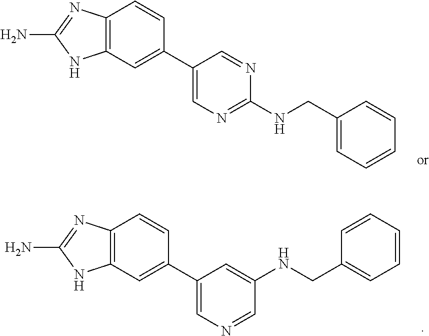

6. The compound according to claim 1, wherein X is

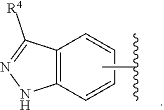

7. The compound according to claim 6, having a chemical structure of

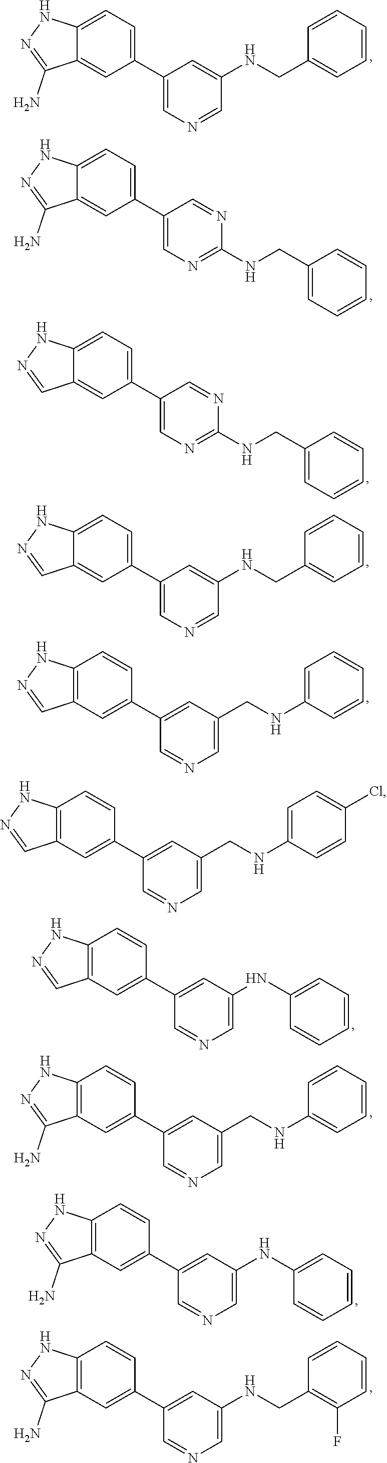

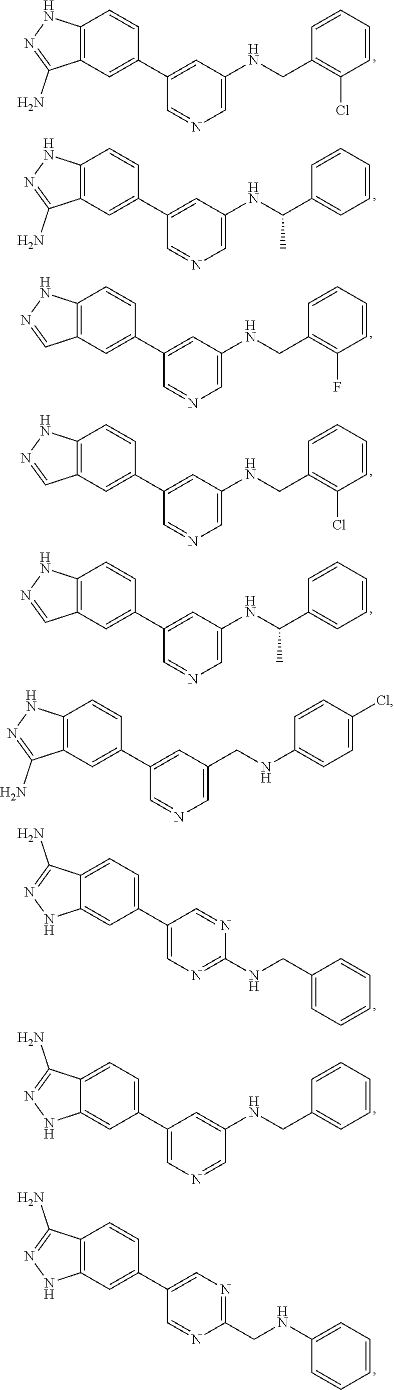

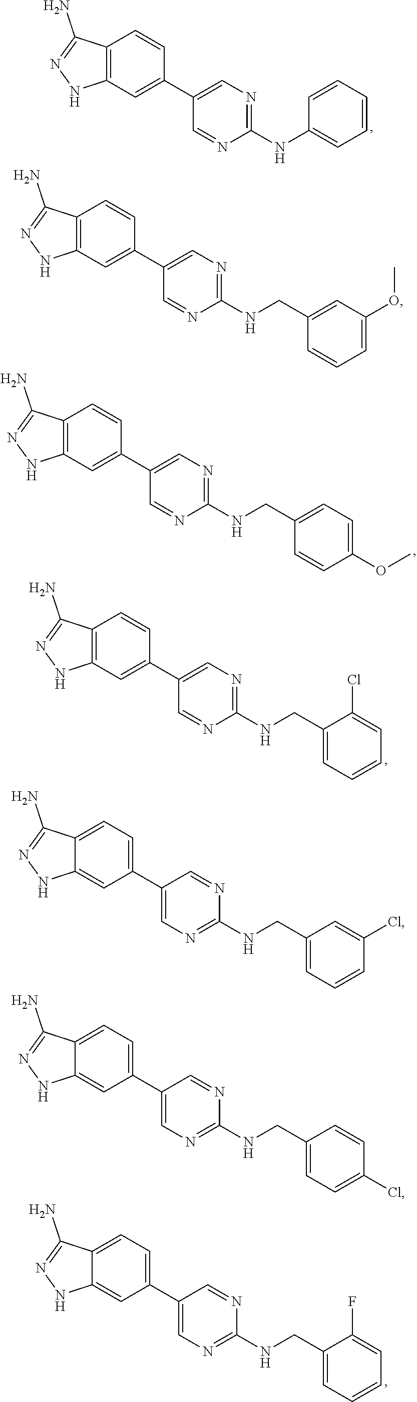

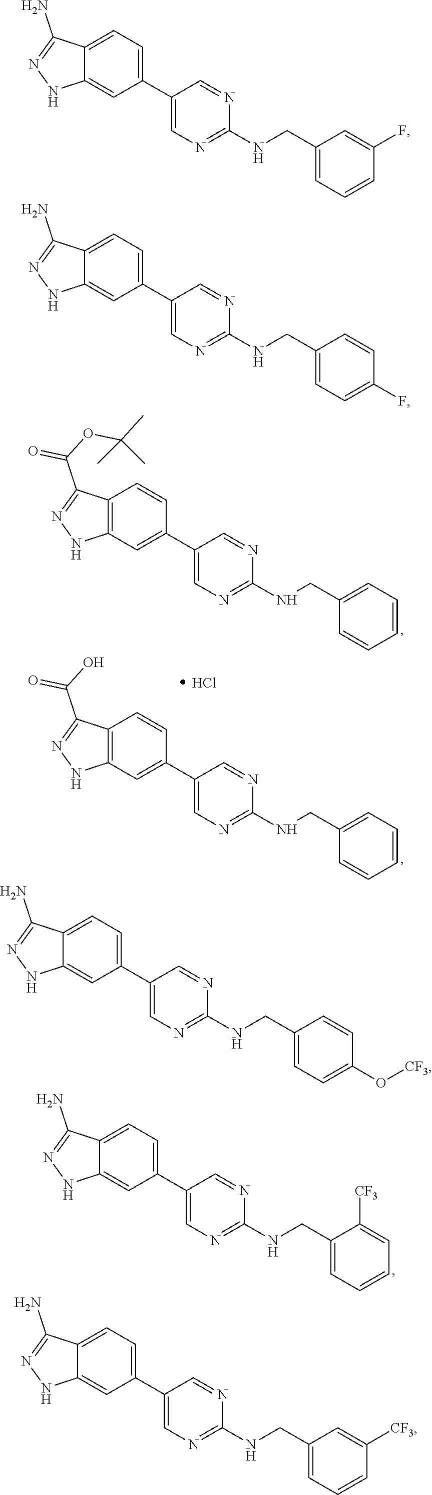

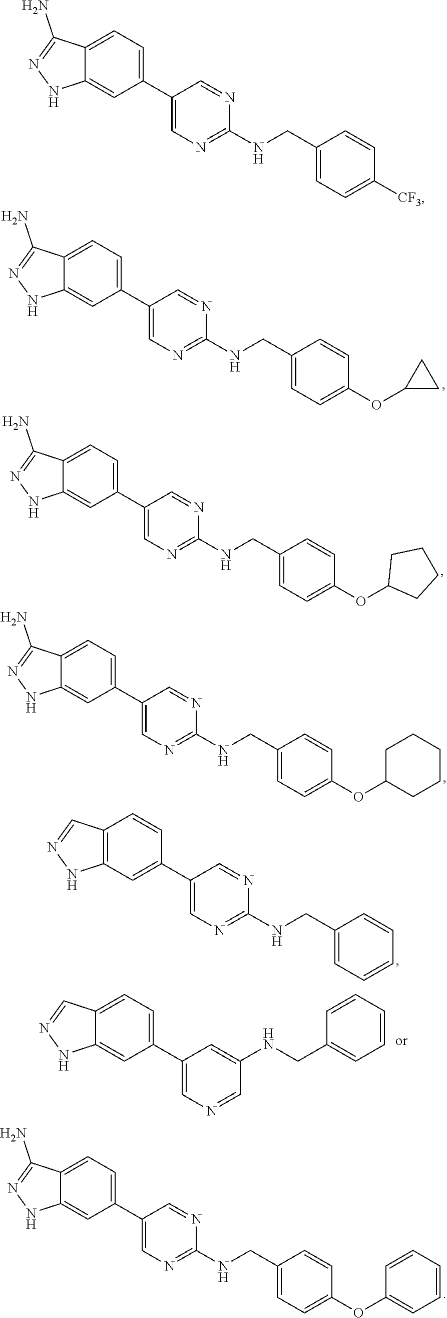

8. The compound according to claim 1, wherein X is

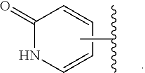

9. The compound according to claim 8, having a chemical structure of

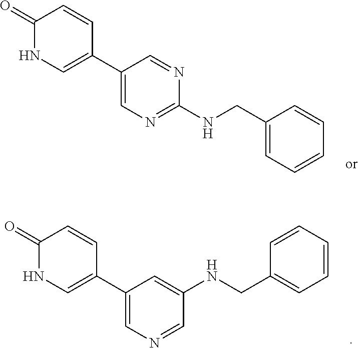 [SMILES: O=c1ccc(-c2cnc(NCc3ccccc3)nc2)c[nH]1 ; O=c1ccc(-c2cncc(NCc3ccccc3)c2)c[nH]1]

10. The compound according to claim 1, wherein X is

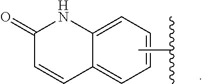

11. The compound according to claim 10, having a chemical structure of

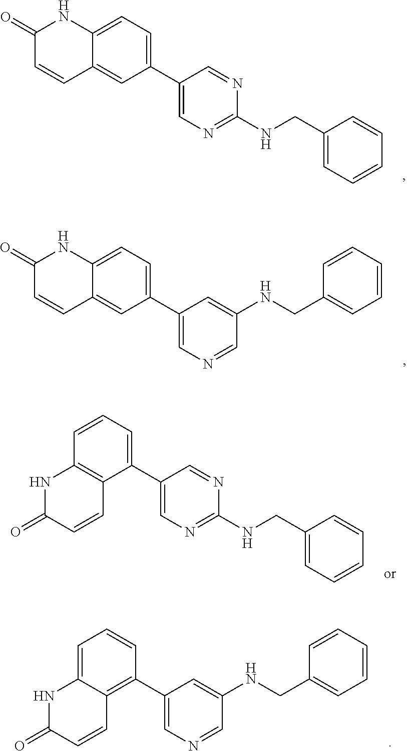 [SMILES: O=c1ccc2cc(-c3cnc(NCc4ccccc4)nc3)ccc2[nH]1 ; O=c1ccc2cc(-c3cncc(NCc4ccccc4)c3)ccc2[nH]1 ; O=c1ccc2c(-c3cnc(NCc4ccccc4)nc3)cccc2[nH]1 ; O=c1ccc2c(-c3cncc(NCc4ccccc4)c3)cccc2[nH]1]

12. The compound according to claim 1, wherein X is

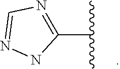 [SMILES: *c1ncn[nH]1]

13. The compound according to claim 12, having a chemical structure of

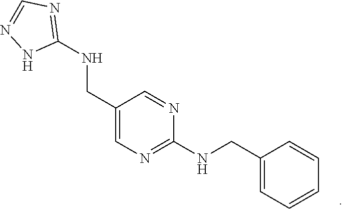 [SMILES: c1ccc(CNc2ncc(CNc3ncn[nH]3)cn2)cc1]

14. The compound according to claim 1, wherein X is

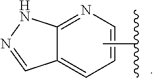

15. The compound according to claim 14, having a chemical structure of

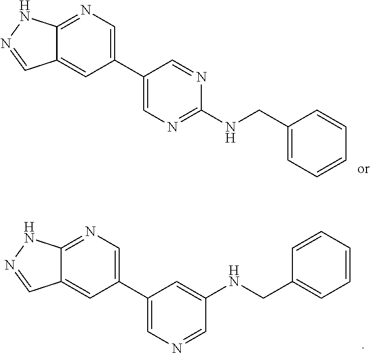 [SMILES: c1ccc(CNc2ncc(-c3cnc4[nH]ncc4c3)cn2)cc1 ; c1ccc(CNc2cncc(-c3cnc4[nH]ncc4c3)c2)cc1]

16. The compound according to claim 1, wherein X is

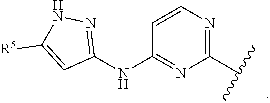

17. The compound according to claim 16, having a chemical structure of

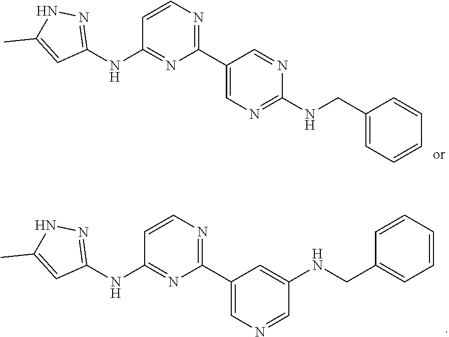 [SMILES: Cc1cc(Nc2ccnc(-c3cnc(NCc4ccccc4)nc3)n2)n[nH]1 ; Cc1cc(Nc2ccnc(-c3cncc(NCc4ccccc4)c3)n2)n[nH]1]

18. The compound according to claim 1, wherein X is

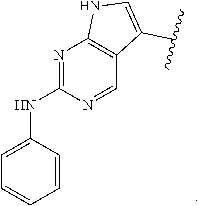 [SMILES: *c1c[nH]c2nc(Nc3ccccc3)ncc12]

19. The compound according to claim 18, having a chemical structure of

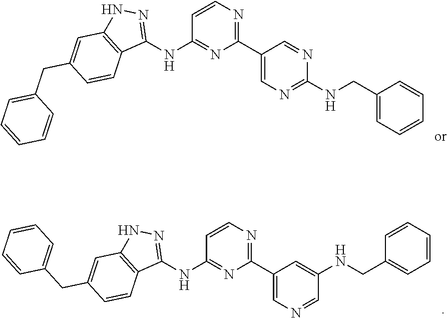 [SMILES: c1ccc(CNc2ncc(-c3nccc(Nc4n[nH]c5cc(Cc6ccccc6)ccc45)n3)cn2)cc1 ; c1ccc(CNc2cncc(-c3nccc(Nc4n[nH]c5cc(Cc6ccccc6)ccc45)n3)c2)cc1]

20. The compound according to claim 1, wherein X is

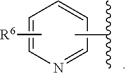

21. The compound according to claim 20, having a chemical structure of

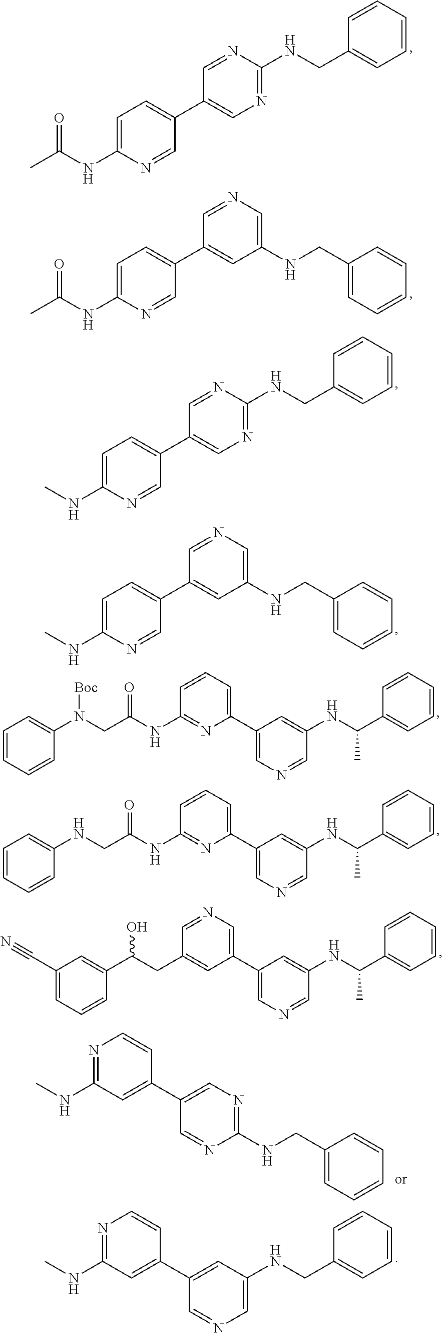

22. The compound according to claim 1, wherein X is

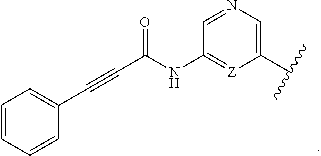

23. The compound according to claim 22, having a chemical structure of

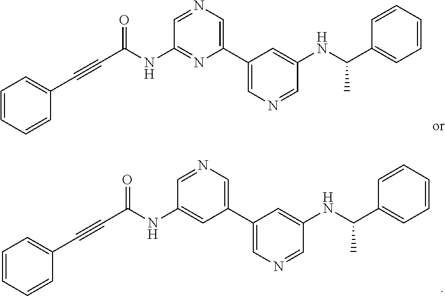 [SMILES: C[C@H](Nc1cncc(-c2cncc(NC(=O)C#Cc3ccccc3)n2)c1)c1ccccc1 ; C[C@H](Nc1cncc(-c2cncc(NC(=O)C#Cc3ccccc3)c2)c1)c1ccccc1]

24. The compound according to claim 1, wherein X is

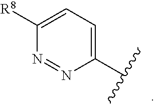

25. The compound according to claim 24, having a chemical structure of

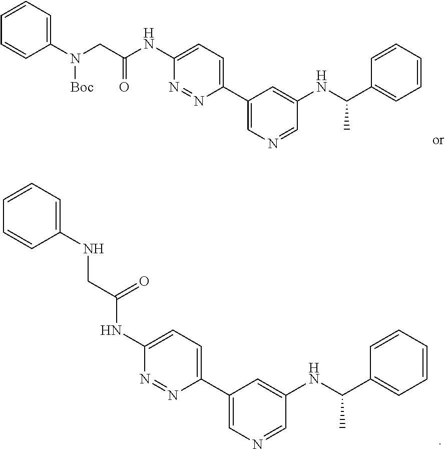 [SMILES: C[C@H](Nc1cncc(-c2ccc(NC(=O)CN(C(=O)OC(C)(C)C)c3ccccc3)nn2)c1)c1ccccc1 ; C[C@H](Nc1cncc(-c2ccc(NC(=O)CNc3ccccc3)nn2)c1)c1ccccc1]

26. The compound according to claim 1, wherein X is

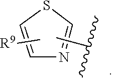

27. The compound according to claim 26, having a chemical structure of

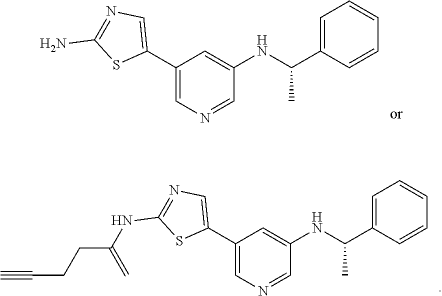 [SMILES: C[C@H](Nc1cncc(-c2cnc(N)s2)c1)c1ccccc1 ; C#CCCC(=C)Nc1ncc(-c2cncc(N[C@@H](C)c3ccccc3)c2)s1]

28. The compound according to claim 1, wherein X is

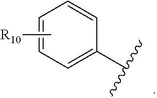

29. The compound according to claim 28, having a chemical structure of

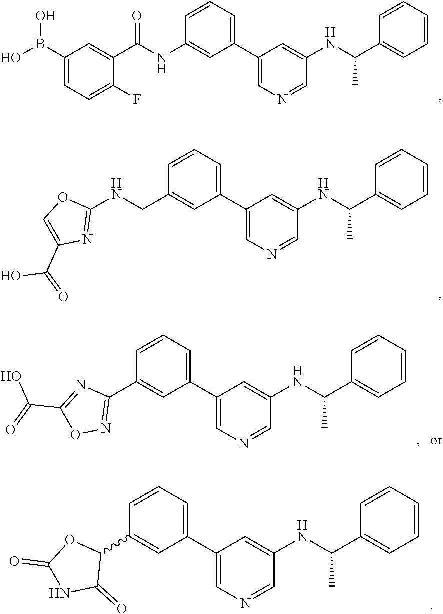 [SMILES: C[C@H](Nc1cncc(-c2cccc(NC(=O)c3cc(B(O)O)ccc3F)c2)c1)c1ccccc1 ; C[C@H](Nc1cncc(-c2cccc(CNc3nc(C(=O)O)co3)c2)c1)c1ccccc1 ; C[C@H](Nc1cncc(-c2cccc(-c3noc(C(=O)O)n3)c2)c1)c1ccccc1 ; C[C@H](Nc1cncc(-c2cccc(C3OC(=O)NC3=O)c2)c1)c1ccccc1]

30. A method of inhibiting activity of a kinase in a cell, said method comprising:
contacting the cell with a compound according to any one of claims 1-29 under conditions effective to inhibit activity of the kinase in the cell.

31. The method according to claim 30, wherein the kinase is a dual-specificity tyrosine phosphorylation-regulated kinase (DYRK).

32. The method according to claim 31, wherein the kinase is dual-specificity tyrosine phosphorylation-regulated kinase 1A (DYRK1A).

33. The method according to claim 30, wherein said method is carried out ex vivo.

34. The method according to claim 30, wherein said method is carried out in vivo.

35. A method of increasing cell proliferation in a population of pancreatic beta cells, said method comprising:
contacting a population of pancreatic beta cells with a compound according to any one of claims 1-29 under conditions effective to increase cell proliferation in the population of pancreatic beta cells.

36. The method according to claim 35 further comprising:
contacting the population of pancreatic beta cells with a transforming growth factor beta (TGFβ) superfamily signaling pathway inhibitor.

37. The method according claim 35 or claim 36 further comprising:
contacting the population of pancreatic beta cells with a glucagon-like peptide-1 receptor (GLP1R) agonist, a Dipeptidyl Peptidase IV (DDP4) inhibitor, or a combination thereof.

38. The method according to any one of claims 35-37, wherein said method is carried out ex vivo.

39. The method according to any one of claims 35-37, wherein said method is carried out in vivo.

40. The method according to any one of claims 35-37, wherein said method is carried out with a composition comprising both the compound and the TGFβ superfamily signaling pathway inhibitor.

41. The method according to any one of claims 35-37, wherein the TGFβ superfamily signaling pathway inhibitor is selected from the group consisting of an inhibitor of TGFβ/TGFβ receptor binding, activin or inhibin/activin receptor binding, and bone morphogenetic protein (BMP)/BMP receptor binding.

42. The method according to any one of claims 35-37, wherein the TGFβ superfamily signaling pathway inhibitor is an inhibitor of activin or inhibin/activin receptor binding selected from the group consisting of SB431542 and Alk5 inhibitor II.

43. The method according to any one of claims 35-37, wherein the TGFβ superfamily signaling pathway inhibitor is a SMAD signaling pathway inhibitor.

44. The method according to claim 35, wherein said method is carried out with a composition comprising the compound and the glucagon-like peptide-1 receptor (GLP1R) agonist, Dipeptidyl Peptidase IV (DDP4) inhibitor, or a combination of the GLPR1 agonist and DPP4 inhibitor.

45. The method according to claim 35 or claim 44, wherein the GLP1R agonist is selected from the group consisting of GLP1 analogs, extendin-4, liraglutide, lixisenatide, semaglutide, and combinations thereof.

46. The method according to claim 35 or claim 44, wherein the DDP4 is selected from the group consisting of sitagliptin, vildagliptin, saxagliptin, alogliptin, teneligliptin, and anagliptin.

47. The method according to any one of claims 35-46, wherein said pancreatic beta cells are primary human pancreatic beta cells.

48. The method according to any one of claims 35-47, wherein said contacting does not induce beta cell death or DNA damage.

49. The method according to any one of claims 35-48, wherein said contacting induces beta cell differentiation.

50. The method according to any one of claims 35-49, wherein said contacting increases glucose-stimulated insulin secretion.

51. A composition comprising:
a compound according to any one of claims 1-29 and
a carrier.

52. The composition according to claim 5 further comprising:
a transforming growth factor beta (TGFβ) superfamily signaling pathway inhibitor.

53. The composition according to claim 51 or claim 52 further comprising:
a glucagon-like peptide-1 receptor (GLP1R) agonist, a Dipeptidyl Peptidase IV (DDP4) inhibitor, or a combination thereof.

54. The composition according to any one of claims 51-53, wherein the carrier is a pharmaceutically-acceptable carrier.

55. A method of treating a subject for a condition associated with insufficient insulin secretion, said method comprising:
administering to a subject in need of treatment for a condition associated with an insufficient level of insulin secretion a compound of any one of claims 1-29 under conditions effective to treat the subject for the condition.

56. The method according to claim 55 further comprising:
administering a transforming growth factor beta (TGFβ) superfamily signaling pathway inhibitor.

57. The method according to claim 55 or claim 56 further comprising:
administering a glucagon-like peptide-1 receptor (GLP1R) agonist, a Dipeptidyl Peptidase IV (DDP4) inhibitor, or a combination thereof.

58. The method according to any one of claims 55-57, wherein said administering is carried out under conditions effective to increase pancreatic beta cell mass in the subject.

59. The method according to any one of claims 55-57, wherein the subject has been diagnosed as having one or more of type I diabetes (T1D), type II diabetes (T2D), gestational diabetes, congenital diabetes, maturity onset diabetes (MODY), cystic fibrosis-related diabetes, hemochromatosis-related diabetes, drug-induced diabetes, or monogenic diabetes.

60. The method according to any one of claims 55-57, wherein the subject has been diagnosed as having metabolic syndrome or insulin resistance.

61. The method according to any one of claims 55-57, wherein the subject has had pancreatitis, a pancreatectomy, pancreas transplantation, or pancreatic islet transplantation.

62. The method according to any one of claims 55-57, wherein said administering is carried out orally, transdermally, parenterally, subcutaneously, intravenously, intramuscularly, or intraperitoneally.

63. The method according to any one of claims 55-57, wherein the subject is a mammalian subject.

64. The method according to any one of claims 55-57, wherein the subject is a human subject.

65. A method of treating a subject for a neurological disorder, said method comprising:
administering to a subject in need of treatment for a neurological disorder a compound of any one of claims 1-29 under conditions effective to treat the subject for the condition.

66. The method according to claim 65 further comprising:
administering a transforming growth factor beta (TGFβ) superfamily signaling pathway inhibitor.

67. The method according to claim 65 or claim 66 further comprising:
administering a glucagon-like peptide-1 receptor (GLP1R) agonist, a Dipeptidyl Peptidase IV (DDP4) inhibitor, or a combination thereof.

68. The method according to any one of claims 65-67, wherein the subject has been diagnosed as having one or more of diabetes, Down's Syndrome, or a neurodegenerative disease.

69. The method according to any one of claims 65-67, wherein said administering is carried out orally, transdermally, parenterally, subcutaneously, intravenously, intramuscularly, or intraperitoneally.

70. The method according to any one of claims 65-67, wherein the subject is a mammalian subject.

71. The method according to any one of claims 65-67, wherein the subject is a human subject.
